(19) United States
(12) Patent Application Publication
(10) Pub. No.: US 2026/0132196 A1
(43) Pub. Date: May 14, 2026

(54) POLYPEPTIDE CONSTRUCTS SELECTIVELY BINDING TO CLDN6 AND CD3

(71) Applicants: AMGEN RESEARCH (MUNICH) GMBH, Munich (DE); AMGEN INC., Thousand Oaks, CA (US)

(72) Inventors: Christoph Dahlhoff, Munich (DE); Tobias Raum, Munich (DE); Jonas Anlahr, Munich (DE); Claudia Blümel, Munich (DE); Lars Gaedtke, Munich (DE); Silke Quaglia, Munich (DE); Jonas Honer, Munich (DE); Julie Bailis, Thousand Oaks, CA (US); Elizabeth Dang Pham, Thousand Oaks, CA (US); Christopher M. Murawsky, Thousand Oaks, CA (US); Benjamin M. Alba, South San Francisco, CA (US)

(21) Appl. No.: 18/251,210

(22) PCT Filed: Nov. 8, 2021
(86) PCT No.: PCT/EP2021/080863

§ 371 Date: Oct. 20, 2023

Related U.S. Application Data
(60) Provisional application No. 63/110,817, filed on Nov. 6, 2020.
(60) Provisional application No. 63/139,419, filed on Jan. 20, 2021.

(51) Int. Cl. C07K 16/28; A61K 39/00; A61P 35/00; A61P 37/04

(52) U.S. Cl. CPC C07K 16/28; A61P 35/00; A61P 37/04; C07K 16/2809; A61K 2039/505; C07K 2317/31; C07K 2317/33; C07K 2317/565; C07K 2317/567; C07K 2317/622; C07K 2317/734; C07K 2317/92; C07K 2317/94

(57) ABSTRACT

The present invention relates to a polypeptide or a polypeptide construct comprising a domain which binds to Claudin 6 (CLDN6) and another domain which binds to CD3. Moreover, the invention provides a polynucleotide encoding the construct, a vector comprising said polynucleotide and a host cell transformed or transfected with said polynucleotide or vector. Furthermore, the invention provides a process for producing the construct of the invention, a medical use of said construct and a kit comprising said construct.

This application includes a sequence listing submitted electronically by the file name 55920 Sub SeqListing txt; Size: 1,410,902 bytes; Created Aug. 19, 2025, and is incorporated herein by reference.

The present invention relates to polypeptides/polypeptide constructs comprising a domain comprising a paratope which binds to Claudin 6 (CLDN6) and another domain which comprises a paratope that binds to CD3. Moreover, the invention provides polynucleotides encoding the polypeptides/polypeptide constructs, vectors comprising said polynucleotides and host cells transformed or transfected with said polynucleotides or vectors. Furthermore, the invention provides processes for producing the polypeptides/polypeptide constructs of the invention, medical uses of said polypeptides/polypeptide constructs and kits comprising said constructs.

### BACKGROUND OF THE INVENTION

Claudins are key structural and functional components of epithelial tight junctions located between two adjacent cells, which regulate cell-cell permeability, maintain ion homeostasis, and support cell adhesion and polarity. Claudins are tetraspan transmembrane proteins of 22-27 kDa that multimerize within or across cell membranes to form a protective barrier. The 24 claudin proteins that have been reported differ in terms of their tissue localization and expression and by their interactions with other proteins.

Claudin 6 (CLDN6) was initially identified in a similarity search for further genes and proteins that belong to the Claudin family of genes and proteins (Morita et al., Proc. Natl. Acad. Sci. USA, Vol. 96, pp. 511-516, 1999). Claudin 6 mRNA expression was not detected in adult tissues, but only in embryonic tissues. Subsequently, mRNA and protein expression were detected in various tumors and tumor cell lines. Consistent with this finding, Claudin-6 is regarded as a carcino-embryonic transmembrane protein, which is absent from normal adult human tissue. CLDN6 expression is abberantly activated in various cancer types, such as ovarian, lung, gastric, breast, germ cell, and pediatric cancers (Stadler et al., Onocoimmunology 2016, Vol. 5, No. 3, e1091555 and therein cited reference, e.g., Micke et al, Int. J. Cancer 2014:2206-14; Rendón-Huerta et al., J. Gastrointest. Cancer 2010; 41: 52-59; Ushiku et al., Histopathology 2012, 61:1043-56); Ben-David et al., Nat. Commun. 2013; 4:1992; Birks et al., BRAIN PATHOL. 2010; 20:140-50), Sullivan et al., Am. J. Surg. Pathol. 2012; 36:73-80).

CLDN6 is a 220 amino acid protein with two extracellular loops (ECL), which has substantial sequence identity to CLDN9 with only three amino acid residues that are different in the two ECLs.

The expression of CLDN6 in multiple tumor types, with normal tissue expression restricted to fetal development, has led to the consideration of CLDN6 as a therapeutic target in various types of cancer, for example, in ovarian and non-small cell lung cancer (NSCLC) and other indications.

Ovarian and NSCLC cancers remain indications with high unmet medical need.

Ovarian cancer is the seventh most-common cancer worldwide. In 2018 there were 295,414 new cases and 184,799 deaths worldwide, with a higher mortality in countries of the Northern hemisphere than in Asia or Africa. (Bray et al., CA Cancer J Clin 2018). Typical first-line treatment involves surgery and combination chemotherapy that includes platinum and paclitaxel or docetaxel. More recently, the anti-VEGF antibody bevacizumab and PARP inhibitors have been approved as maintenance therapy following first-line chemotherapy. However, despite initial response, up to 70% of patients experience disease recurrence due to development of chemoresistance and/or tumor immune evasion. Ovarian tumors are characterized by a highly immunosuppressive tumor microenvironment; while there is evidence that ovarian tumors can be immunogenic, immune checkpoint therapies that have changed the standard of care in other solid tumor types have shown limited durability in ovarian cancer (Rodriguez et al., Cancers 2018). Despite the advancement of multiple novel therapies and combinations to clinical testing in ovarian cancer, the 5-year survival rate remains low and there remains an urgent need for therapies that can enable durable response.

Lung cancer is one of the most common cancers worldwide, with over 2 million new cases and 1.7 million deaths reported in 2018 (Bray et al., CA: A Cancer Journal for Clinicians 2018). Non-small cell lung cancer (NSCLC) comprises the majority (85%) of lung cancer cases and is often associated with smoking and environmental exposures such as asbestos (Zappa and Mousa, Transl Lung Cancer Res 2016). The recommended first-line treatment for NSCLC is immune checkpoint blockade with platinum doublet chemotherapy for patients whose tumors express PD-L1, although targeted therapy may be preferred for initial treatment of tumors with driver mutations (Ettinger et al., JNCCN, 2019). While these advances are promising and in the case of immune checkpoint blockade have enabled long-term durable response for some patients (Santini and Hellman, Cancer J 2018), further evaluation of immunotherapy combinations and the advancement of additional, new therapies are needed for treatment of most patients.

New therapies with the potential to provide durable response to a larger patient population are therefore still needed for the treatment of ovarian cancer and/or NSCLC, and particularly to any type of cancer that expresses CLDN6, more particularly for the treatment of cancer patients in second line treatment or higher, such as patients that have previously received chemotherapy or immunotherapy and who have relapsing disease.

Bispecific (and multispecific) constructs comprising one antigen-binding (more precisely, an epitope-binding) domain that binds to CD3 on a T cell and one antigen-binding (more precisely, an epitope-binding) domain that binds to a protein expressed on a target cell directly connect T cells to target cells to induce T cell redirected lysis. This mechanism of action is distinct from chemotherapy, targeted therapy and other immunotherapy in that it can work with any CD3-positive T cell, independent of a costimulatory activating signal (Klinger et al., Immunol Reviews 2016).

The expression of CLDN6 on the cell surface of germ cell tumors, ovarian cancer, and non-small cell lung cancer provides a basis for targeting these tumor types with CLDN6×CD3 polypeptides/polypeptide constructs. Furthermore, CLDN6×CD3 polypeptides/polypeptide constructs has the potential to target additional tumor types that express CLDN6, and particularly to any type of cancer that expresses CLDN6, more particularly for the treatment of cancer patients in second line treatment or higher, such as patients that have previously received chemotherapy or immunotherapy and who have relapsing disease.

### DETAILED DESCRIPTION OF THE INVENTION

The present invention provides new polypeptides/polypeptide construct as compounds that selectively and, preferably, specifically bind to CLDN6 (SEQ ID NO: 1) or any isoforms thereof, compositions comprising such compounds, methods of treatment and prevention of neoplastic diseases using the herein disclosed products, kits comprising the herein disclosed products, products for the use as a medicament, particularly for the use in the treatment and prevention of neoplastic diseases. The amino acid sequence of human CLDN6 and related information may be found in the UniProt database under accession number P56747.

### Compounds of the Invention

In one aspect, the present invention provides a polypeptide/polypeptide construct comprising, or consisting of, a domain, which binds to CLDN6 (SEQ ID NO: 1 or a fragment thereof or a variant of the amino acid sequence) on the surface of a target cell and a domain, which binds to CD3 on the surface of a T cell, the binding to both, CLDN6 and CD3, allow the T cell's activation. The binding of the polypeptide construct according to the invention engages T cells, i.e., binds to CD3, and brings a T cell and the target cell into close contact to allow activated T cells to induce cytotoxic/cytolytic mechanisms resulting in the destruction of the target cell (T cell-dependent cytotoxicity).

Further, the present invention provides a polypeptide/polypeptide construct comprising, or consisting of, a domain, which comprises a paratope (i.e., an antigen-binding domain, more particularly an epitope-binding structure), which binds to CLDN6, wherein optionally the domain comprising a paratope (i.e., an antigen-binding (epitope-binding) structure) of the polypeptides/polypeptide constructs of the invention is capable of binding CLDN6 on the surface of a cell that expresses CLDN6 binds to the E1A and/or the E2B regions of CLDN6 (SEQ ID NO: 1). Thus, the present invention provides a polypeptide/polypeptide construct comprising, or consisting of, a domain which binds to CLDN6, wherein optionally the domain is capable of binding CLDN6 on the surface of a cell that expresses CLDN6 binds to the E1A and/or the E2B regions the sequence corresponding to the E1A and/or E2B regions of these loops that are depicted in SEQ ID NOS: 9 and 10.

In embodiments of the invention the polypeptide/polypeptide construct comprising, or consisting of, a domain, which comprises a paratope (i.e., an antigen-binding domain, more particularly an epitope-binding structure), which binds to CLDN6, wherein optionally the domain comprising a paratope (i.e., an antigen-binding (epitope-binding) structure) of the polypeptides/polypeptide constructs of the invention is capable of binding CLDN6 on the surface of a cell that expresses CLDN6 binds to the E1A and/or the E2B regions of CLDN6 (SEQ ID NO: 1) does not bind to amino acids 138-150 of CLDN6 as depicted in SEQ ID NO: 1. In further embodiments of the invention the polypeptide/polypeptide construct comprising, or consisting of, a domain, which binds to CLDN6 binds to the E1A and/or the E2B regions of CLDN6 (SEQ ID NO: 1) does not bind to amino acids 138-150 of CLDN6 as depicted in SEQ ID NO: 1.

Accordingly, the present invention provides polypeptides/polypeptide constructs comprising, or consisting of, a domain, which comprises a paratope (i.e. an antigen-binding domain, more particularly an epitope-binding structure), which binds to an epitope region comprising amino acids of the extracellular loop 1 (ECL1) of CLDN6, preferably one comprising amino acids 29-39 of SEQ ID NO: 1 and/or comprising amino acids of the extracellular loop 2 (ECL2) of CLDN6 corresponding to amino acids 151-160 of SEQ ID NO: 1 on the surface of a target cell. Thus, the present invention provides polypeptides/polypeptide constructs comprising, or consisting of, a domain, which binds to an epitope region comprising amino acids of the extracellular loop 1 (ECL1) of CLDN6, preferably one comprising amino acids 29-39 of SEQ ID NO: 1 and/or comprising amino acids of the extracellular loop 2 (ECL2) of CLDN6 corresponding to amino acids 151-160 of SEQ ID NO: 1 on the surface of a target cell.

Accordingly, the present invention provides polypeptides/polypeptide constructs as defined in any one of the preceding paragraphs comprising another domain, which comprises a paratope (i.e. an antigen-binding structure (epitope-binding structure)) that recognizes and/or binds to an extracellular epitope of the CD3ε chain (preferably, the human and the Macaca CD3ε chain), and a domain which extends the half-life (HLE domain) of the polypeptide after administration to an individual, which optionally comprises two polypeptide monomers, each comprising a hinge, a CH2 domain and a CH3 domain. Thus, the present invention provides polypeptides/polypeptide constructs as defined in any one of the preceding paragraphs comprising another domain, which recognizes and/or binds to an extracellular epitope of the CD3ε chain (preferably, the human and the Macaca CD3ε chain), and a domain which extends the half-life (HLE domain) of the polypeptide after administration to an individual, which optionally comprises two polypeptide monomers, each comprising a hinge, a CH2 domain and a CH3 domain.

According to the invention, polypeptides/polypeptide constructs are provided, wherein a domain of the construct binds immunoselectively to an epitope of CLDN6 recognized and/or bound by a paratope (an antigen-binding or epitope-binding structure) comprised in any one of the sequences referred to in in a) to s) below, a) to d), n) and s) being preferred, a) to c), e) and s) being much preferred):
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256,
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270, and
t) a VH region comprising a CDR-H1 depicted in SEQ ID NO: 680, a CDR-H2 depicted in any one of SEQ ID NOs: 681, 682 or 683, and a CDR-H3 depicted in any one of SEQ ID NOs: 684, 685, 686 or 687, and a VL region comprising a CDR-L1 depicted in any one of SEQ ID NOs: 688 or 689, a CDR-L2 depicted in SEQ ID NO: 690, and a CDR-L3 as depicted in any one of SEQ ID NOs: 691, 692, 693 or 694, and any possible combination to the herein described CDR of the heavy and light chains.

The constructs of the preceding paragraph accordingly preferably comprise at least one domain comprising a paratope binding CLDN6 as defined in sections (a) to (s), optionally further comprising a domain comprising a domain to CD3, e.g., the constructs of the preceding paragraph accordingly preferably binding CLDN6 and having the VL and or VH regions comprising the CDRs as defined in sections (a) to (s), optionally further comprising a domain comprising a domain to CD3.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18. Therefore, according to the invention, polypeptides/polypeptide constructs are provided comprising a domain that binds (immunoselectively) to an epitope recognized by a domain comprising a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32. In other words, according to the invention, polypeptides/polypeptide constructs are provided comprising a domain that binds (immunoselectively) to an epitope recognized by a domain comprising a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46. Therefore, according to the invention, polypeptides/polypeptide constructs are provided comprising a domain that binds (immunoselectively) to an epitope recognized by a domain comprising a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74. Therefore, according to the invention, polypeptides/polypeptide constructs are provided comprising a domain that binds (immunoselectively) to an epitope recognized by a domain comprising a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200. Therefore, according to the invention, polypeptides/polypeptide constructs are provided comprising a domain that binds (immunoselectively) to an epitope recognized by a domain comprising a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200.

According to the invention, polypeptides/polypeptide constructs are provided comprising a domain comprising a paratope (an antigen-binding (epitope-binding) structure)) that binds (immunoselectively) to an epitope recognized by a domain comprising a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241, and a CDR-L3 as depicted in SEQ ID NO: 242. Therefore, according to the invention, polypeptides/polypeptide constructs are provided comprising a domain that binds (immunoselectively) to an epitope recognized by a domain comprising a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241, and a CDR-L3 as depicted in SEQ ID NO: 242.

According to the invention, polypeptides/polypeptide constructs are provided, wherein
(i) a domain comprises a paratope (an antigen-binding (epitope-binding) structure) that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a) to s) below, and/or
(ii) a domain comprises a paratope (an antigen-binding (epitope-binding) structure) that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a) to s) below; and/or
(iii) a domain comprises a paratope (an antigen-binding (epitope-binding) structure) that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a) to s) below; and/or
(iv) a domain comprises a paratope (an antigen-binding (epitope-binding) structure) that binds (immunoselectively) to the same epitope of CLDN6 as an antibody or polypeptide construct comprising a paratope which binds to CLDN6 on the surface of a target cell and which comprises any one of the sequences referred to in a) to s) below:
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256, and
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.

Therefore, according to the invention, polypeptides/polypeptide constructs are provided, wherein
(i) a domain that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a) to s) below, and/or
(ii) a domain that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a) to s) below; and/or
(iii) a domain that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a) to s) below; and/or
(iv) a domain that binds (immunoselectively) to the same epitope of CLDN6 as an antibody or polypeptide construct comprising a paratope which binds to CLDN6 on the surface of a target cell and which comprises any one of the sequences referred to in a) to s) below:
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
1) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256, and
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.

According to the invention, polypeptides/polypeptide constructs are provided, wherein:
(i) a domain comprises a paratope (an antigen-binding (epitope-binding) structure)) that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a-1) to s-1) below, and/or
(ii) a domain comprises a paratope (an antigen-binding (epitope-binding) structure)) that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a-1) to s-1) below; and/or
(iii) a domain comprises a paratope (an antigen-binding (epitope-binding) structure)) that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a-1) to s-1) below; and/or
(iv) a domain comprises a paratope (an antigen-binding (epitope-binding) structure)) that binds (immunoselectively) to the same epitope of CLDN6 as an antibody or polypeptide construct comprising a paratope which binds to CLDN6 on the surface of a target cell and which comprises any one of the sequences referred to in a-1) to s-1) below:
a-1) a VH region as depicted in SEQ ID NO: 11, and/or a VL region as depicted in SEQ ID NO: 12;
b-1) a VH region as depicted in SEQ ID NO: 25, and/or a VL region as depicted in SEQ ID NO: 26;
c-1) a VH region as depicted in SEQ ID NO: 39, and/or a VL region as depicted in SEQ ID NO: 40;
d-1) a VH region as depicted in SEQ ID NO: 53, and/or a VL region as depicted in SEQ ID NO: 54;
e-1) a VH region as depicted in SEQ ID NO: 67, and/or a VL region as depicted in SEQ ID NO: 68;
f-1) a VH region as depicted in SEQ ID NO: 81, and/or a VL region as depicted in SEQ ID NO: 82;
g-1) a VH region as depicted in SEQ ID NO: 95, and/or a VL region as depicted in SEQ ID NO: 96;
h-1) a VH region as depicted in SEQ ID NO: 109, and/or a VL region as depicted in SEQ ID NO: 110;
i-1) a VH region as depicted in SEQ ID NO: 123, and/or a VL region as depicted in SEQ ID NO: 124;
j-1) a VH region as depicted in SEQ ID NO: 137, and/or a VL region as depicted in SEQ ID NO: 138;
k-1) a VH region as depicted in SEQ ID NO: 151, and/or a VL region as depicted in SEQ ID NO: 152;
l-1) a VH region as depicted in SEQ ID NO: 165, and/or a VL region as depicted in SEQ ID NO: 166;
m-1) a VH region as depicted in SEQ ID NO: 179, and/or a VL region as depicted in SEQ ID NO: 180;
n-1) a VH region as depicted in SEQ ID NO: 193, and/or a VL region as depicted in SEQ ID NO: 194;
o-1) a VH region as depicted in SEQ ID NO: 207, and/or a VL region as depicted in SEQ ID NO: 208;
p-1) a VH region as depicted in SEQ ID NO: 221, and/or a VL region as depicted in SEQ ID NO: 222;
q-1) a VH region as depicted in SEQ ID NO: 235, and/or a VL region as depicted in SEQ ID NO: 236;
r-1) a VH region as depicted in SEQ ID NO: 249, and/or a VL region as depicted in SEQ ID NO: 250; and
s-1) a VH region as depicted in SEQ ID NO: 263, and/or a VL region as depicted in SEQ ID NO: 264.

Therefore, according to the invention, polypeptides/polypeptide constructs are provided, wherein:
(i) a domain that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a-1) to s-1) below, and/or
(ii) a domain that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a-1) to s-1) below; and/or
(iii) a domain that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a-1) to s-1) below; and/or
(iv) a domain that binds (immunoselectively) to the same epitope of CLDN6 as an antibody or polypeptide construct comprising a paratope which binds to CLDN6 on the surface of a target cell and which comprises any one of the sequences referred to in a-1) to s-1) below:
a-1) a VH region as depicted in SEQ ID NO: 11, and/or a VL region as depicted in SEQ ID NO: 12;
b-1) a VH region as depicted in SEQ ID NO: 25, and/or a VL region as depicted in SEQ ID NO: 26;
c-1) a VH region as depicted in SEQ ID NO: 39, and/or a VL region as depicted in SEQ ID NO: 40;
d-1) a VH region as depicted in SEQ ID NO: 53, and/or a VL region as depicted in SEQ ID NO: 54;
e-1) a VH region as depicted in SEQ ID NO: 67, and/or a VL region as depicted in SEQ ID NO: 68;
f-1) a VH region as depicted in SEQ ID NO: 81, and/or a VL region as depicted in SEQ ID NO: 82;
g-1) a VH region as depicted in SEQ ID NO: 95, and/or a VL region as depicted in SEQ ID NO: 96;
h-1) a VH region as depicted in SEQ ID NO: 109, and/or a VL region as depicted in SEQ ID NO: 110;
i-1) a VH region as depicted in SEQ ID NO: 123, and/or a VL region as depicted in SEQ ID NO: 124;
j-1) a VH region as depicted in SEQ ID NO: 137, and/or a VL region as depicted in SEQ ID NO: 138;
k-1) a VH region as depicted in SEQ ID NO: 151, and/or a VL region as depicted in SEQ ID NO: 152;
l-1) a VH region as depicted in SEQ ID NO: 165, and/or a VL region as depicted in SEQ ID NO: 166;
m-1) a VH region as depicted in SEQ ID NO: 179, and/or a VL region as depicted in SEQ ID NO: 180;
n-1) a VH region as depicted in SEQ ID NO: 193, and/or a VL region as depicted in SEQ ID NO: 194;
o-1) a VH region as depicted in SEQ ID NO: 207, and/or a VL region as depicted in SEQ ID NO: 208;
p-1) a VH region as depicted in SEQ ID NO: 221, and/or a VL region as depicted in SEQ ID NO: 222;
q-1) a VH region as depicted in SEQ ID NO: 235, and/or a VL region as depicted in SEQ ID NO: 236;
r-1) a VH region as depicted in SEQ ID NO: 249, and/or a VL region as depicted in SEQ ID NO: 250; and
s-1) a VH region as depicted in SEQ ID NO: 263, and/or a VL region as depicted in SEQ ID NO: 264.

According to the invention, polypeptides/polypeptide constructs are provided that compete for binding with a polypeptide construct comprising, or consisting of, a domain:
(i) a domain comprises a paratope (an antigen-binding (epitope-binding) structure)) that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a-1) to s-1) below, and/or
(ii) a domain comprises a paratope (an antigen-binding (epitope-binding) structure)) that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a-1) to s-1) below; and/or
(iii) a domain comprises a paratope (an antigen-binding (epitope-binding) structure)) that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a-1) to s-1) below; and/or
(iv) a domain comprises a paratope (an antigen-binding (epitope-binding) structure)) that binds (immunoselectively) to the same epitope of CLDN6 as an antibody or polypeptide construct comprising a paratope which binds to CLDN6 on the surface of a target cell and which comprises any one of the sequences referred to in a-1) to s-1) below:
a-1) a VH region as depicted in SEQ ID NO: 11, and/or a VL region as depicted in SEQ ID NO: 12;
b-1) a VH region as depicted in SEQ ID NO: 25, and/or a VL region as depicted in SEQ ID NO: 26;
c-1) a VH region as depicted in SEQ ID NO: 39, and/or a VL region as depicted in SEQ ID NO: 40;
d-1) a VH region as depicted in SEQ ID NO: 53, and/or a VL region as depicted in SEQ ID NO: 54;
e-1) a VH region as depicted in SEQ ID NO: 67, and/or a VL region as depicted in SEQ ID NO: 68;
f-1) a VH region as depicted in SEQ ID NO: 81, and/or a VL region as depicted in SEQ ID NO: 82;
g-1) a VH region as depicted in SEQ ID NO: 95, and/or a VL region as depicted in SEQ ID NO: 96;
h-1) a VH region as depicted in SEQ ID NO: 109, and/or a VL region as depicted in SEQ ID NO: 110;
i-1) a VH region as depicted in SEQ ID NO: 123, and/or a VL region as depicted in SEQ ID NO: 124;
j-1) a VH region as depicted in SEQ ID NO: 137, and/or a VL region as depicted in SEQ ID NO: 138;
k-1) a VH region as depicted in SEQ ID NO: 151, and/or a VL region as depicted in SEQ ID NO: 152;
l-1) a VH region as depicted in SEQ ID NO: 165, and/or a VL region as depicted in SEQ ID NO: 166;
m-1) a VH region as depicted in SEQ ID NO: 179, and/or a VL region as depicted in SEQ ID NO: 180;
n-1) a VH region as depicted in SEQ ID NO: 193, and/or a VL region as depicted in SEQ ID NO: 194;
o-1) a VH region as depicted in SEQ ID NO: 207, and/or a VL region as depicted in SEQ ID NO: 208;
p-1) a VH region as depicted in SEQ ID NO: 221, and/or a VL region as depicted in SEQ ID NO: 222;
q-1) a VH region as depicted in SEQ ID NO: 235, and/or a VL region as depicted in SEQ ID NO: 236;
r-1) a VH region as depicted in SEQ ID NO: 249, and/or a VL region as depicted in SEQ ID NO: 250; and
s-1) a VH region as depicted in SEQ ID NO: 263, and/or a VL region as depicted in SEQ ID NO: 264.

Therefore, according to the invention, polypeptides/polypeptide constructs are provided that compete for binding with a polypeptide construct comprising, or consisting of, a domain:
(i) a domain that binds (immunoselectively) to an epitope region that comprises amino acids of the first extracellular loop of CLDN6 (as depticed in SEQ ID NO: 1), also referred to as extracellular loop 1 (ECL1); said epitope region being depicted in SEQ ID NO: 9, and optionally comprises any one of the sequences referred to in a-1) to s-1) below, and/or
(ii) a domain that binds (immunoselectively) to an epitope region that comprises amino acids of the second extracellular loop of CLDN6 (as depticed in SEQ ID NO: 1), also referred to as extracellular loop 2 (ECL2); said epitope region being depicted in SEQ ID NO:10, and optionally comprises any one of the sequences referred to in a-1) to s-1) below; and/or (iii) a domain that binds (immunoselectively) to an epitope region comprising amino acids of ECL1 and ECL2 of CLDN6, preferably those comprising amino acids of the epitope region comprising SEQ ID NOs: 9 and 10, and which optionally comprises any one of the structures referred to in a-1) to s-1) below; and/or
(iv) a domain that binds (immunoselectively) to the same epitope of CLDN6 as an antibody or polypeptide construct comprising a paratope which binds to CLDN6 on the surface of a target cell and which comprises any one of the sequences referred to in a-1) to s-1) below:
a-1) a VH region as depicted in SEQ ID NO: 11, and/or a VL region as depicted in SEQ ID NO: 12;
b-1) a VH region as depicted in SEQ ID NO: 25, and/or a VL region as depicted in SEQ ID NO: 26;
c-1) a VH region as depicted in SEQ ID NO: 39, and/or a VL region as depicted in SEQ ID NO: 40;
d-1) a VH region as depicted in SEQ ID NO: 53, and/or a VL region as depicted in SEQ ID NO: 54;
e-1) a VH region as depicted in SEQ ID NO: 67, and/or a VL region as depicted in SEQ ID NO: 68;
f-1) a VH region as depicted in SEQ ID NO: 81, and/or a VL region as depicted in SEQ ID NO: 82;
g-1) a VH region as depicted in SEQ ID NO: 95, and/or a VL region as depicted in SEQ ID NO: 96;
h-1) a VH region as depicted in SEQ ID NO: 109, and/or a VL region as depicted in SEQ ID NO: 110;
i-1) a VH region as depicted in SEQ ID NO: 123, and/or a VL region as depicted in SEQ ID NO: 124;
j-1) a VH region as depicted in SEQ ID NO: 137, and/or a VL region as depicted in SEQ ID NO: 138;
k-1) a VH region as depicted in SEQ ID NO: 151, and/or a VL region as depicted in SEQ ID NO: 152;
l-1) a VH region as depicted in SEQ ID NO: 165, and/or a VL region as depicted in SEQ ID NO: 166;
m-1) a VH region as depicted in SEQ ID NO: 179, and/or a VL region as depicted in SEQ ID NO: 180;
n-1) a VH region as depicted in SEQ ID NO: 193, and/or a VL region as depicted in SEQ ID NO: 194;
o-1) a VH region as depicted in SEQ ID NO: 207, and/or a VL region as depicted in SEQ ID NO: 208;
p-1) a VH region as depicted in SEQ ID NO: 221, and/or a VL region as depicted in SEQ ID NO: 222;
q-1) a VH region as depicted in SEQ ID NO: 235, and/or a VL region as depicted in SEQ ID NO: 236;
r-1) a VH region as depicted in SEQ ID NO: 249, and/or a VL region as depicted in SEQ ID NO: 250; and
s-1) a VH region as depicted in SEQ ID NO: 263, and/or a VL region as depicted in SEQ ID NO: 264.

Further, the polypeptide construct of the invention competes for binding with a construct comprising a domain which selectively binds to CLDN6 on the surface of a target cell and which comprises any one of the group of sequences referred to in in a) to s) below, a) to d), n) and s) being preferred, a) to c), e) and s) being particularly preferred), and, a polypeptide construct of the invention competes for binding with a construct comprising a domain comprising a paratope (i.e., an antigen-binding (epitope-binding) structure) which selectively binds to CLDN6 on the surface of a target cell and which comprises any one of the group of sequences referred to in in a) to s) below, a) to d), n) and s) being preferred, a) to c), e) and s) being particularly preferred):
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256, and
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.

Further, the polypeptide construct of the invention binds to or competes for binding with an antibody or a polypeptide construct comprising a paratope (i.e., an antigen-binding or epitope-binding structure), which (immunoselectively) binds to CLDN6 on the surface of a target cell and which comprises any one of the group of sequences, and a polypeptide construct of the invention binds to or competes for binding with an antibody or a polypeptide construct which (immunoselectively) binds to CLDN6 on the surface of a target cell and which comprises any one of the group of sequences:
a-1) a VH region as depicted in SEQ ID NO: 11, and/or a VL region as depicted in SEQ ID NO: 12;
b-1) a VH region as depicted in SEQ ID NO: 25, and/or a VL region as depicted in SEQ ID NO: 26;
c-1) a VH region as depicted in SEQ ID NO: 39, and/or a VL region as depicted in SEQ ID NO: 40;
d-1) a VH region as depicted in SEQ ID NO: 53, and/or a VL region as depicted in SEQ ID NO: 54;
e-1) a VH region as depicted in SEQ ID NO: 67, and/or a VL region as depicted in SEQ ID NO: 68;
f-1) a VH region as depicted in SEQ ID NO: 81, and/or a VL region as depicted in SEQ ID NO: 82;
g-1) a VH region as depicted in SEQ ID NO: 95, and/or a VL region as depicted in SEQ ID NO: 96;
h-1) a VH region as depicted in SEQ ID NO: 109, and/or a VL region as depicted in SEQ ID NO: 110;
i-1) a VH region as depicted in SEQ ID NO: 123, and/or a VL region as depicted in SEQ ID NO: 124;
j-1) a VH region as depicted in SEQ ID NO: 137, and/or a VL region as depicted in SEQ ID NO: 138;
k-1) a VH region as depicted in SEQ ID NO: 151, and/or a VL region as depicted in SEQ ID NO: 152;
l-1) a VH region as depicted in SEQ ID NO: 165, and/or a VL region as depicted in SEQ ID NO: 166;
m-1) a VH region as depicted in SEQ ID NO: 179, and/or a VL region as depicted in SEQ ID NO: 180;
n-1) a VH region as depicted in SEQ ID NO: 193, and/or a VL region as depicted in SEQ ID NO: 194;
o-1) a VH region as depicted in SEQ ID NO: 207, and/or a VL region as depicted in SEQ ID NO: 208;
p-1) a VH region as depicted in SEQ ID NO: 221, and/or a VL region as depicted in SEQ ID NO: 222;
q-1) a VH region as depicted in SEQ ID NO: 235, and/or a VL region as depicted in SEQ ID NO: 236;
r-1) a VH region as depicted in SEQ ID NO: 249, and/or a VL region as depicted in SEQ ID NO: 250; and
s-1) a VH region as depicted in SEQ ID NO: 263, and/or a VL region as depicted in SEQ ID NO: 264.

The invention relates to polypeptides/polypeptide constructs according to any one of the preceding paragraphs, wherein the paratope (i.e., the antigen-binding (epitope-binding) structure) binding to CLDN6 is comprised by a pair of VH and VL regions, and polypeptides/polypeptide constructs according to any one of the preceding paragraphs, wherein the domain binding to CLDN6 is comprised by a pair of VH and VL regions, comprising amino acid sequences depicted in comprising amino acid sequences depicted in SEQ ID NOs: 11+12, SEQ ID NO: 25+26, SEQ ID NO: 39+40, SEQ ID NO: 53+54, SEQ ID NO: 67+68, SEQ ID NO: 81+82, SEQ ID NO: 95+96, SEQ ID NO: 109+110, SEQ ID NO: 123+124, SEQ ID NO: 137+138, SEQ ID NO: 151+152, SEQ ID NO: 165+166, SEQ ID NO: 179+180, SEQ ID NO: 193+194, SEQ ID NO: 207+208, SEQ ID NO: 221+222, SEQ ID NO: 235+236, SEQ ID NO: 249+250, or SEQ ID NO: 263+264, or which compete with the polypeptide construct binding to CLDN6.

The invention relates to polypeptides/polypeptide constructs according to any one of the preceding paragraphs comprising or consisting of an amino acid sequence as depicted in SEQ ID NO: 19, SEQ ID NO: 22, SEQ ID NO: 33, SEQ ID NO: 36, SEQ ID NO: 47, SEQ ID NO: 50, SEQ ID NO: 61, SEQ ID NO: 64, SEQ ID NO: 75, SEQ ID NO: 78, SEQ ID NO: 89, SEQ ID NO: 92, SEQ ID NO: 103, SEQ ID NO: 106, SEQ ID NO: 117, SEQ ID NO: 120, SEQ ID NO: 131, SEQ ID NO: 134, SEQ ID NO: 145, SEQ ID NO: 148, SEQ ID NO: 159, SEQ ID NO: 162, SEQ ID NO: 173, SEQ ID NO: 176, SEQ ID NO: 187, SEQ ID NO: 190, SEQ ID NO: 201, SEQ ID NO: 204, SEQ ID NO: 215, SEQ ID NO: 218, SEQ ID NO: 229, SEQ ID NO: 232, SEQ ID NO: 243, SEQ ID NO: 246, SEQ ID NO: 257, or SEQ ID NO: 260, SEQ ID NO: 271 or SEQ ID NO: 274, or which compete with the polypeptide construct binding to CLDN6.

The invention relates to polypeptides/polypeptide constructs according to any one of the preceding paragraphs comprising or consisting of an amino acid sequence selected from the group of those depicted in:
SEQ ID NO: 19, SEQ ID NO: 20, SEQ ID NO: 21, SEQ ID NO: 22, SEQ ID NO: 23, and SEQ ID NO: 24,
SEQ ID NO: 33, SEQ ID NO: 34, SEQ ID NO: 35, SEQ ID NO: 36, SEQ ID NO: 37, and SEQ ID NO: 38,
SEQ ID NO: 47, SEQ ID NO: 48, SEQ ID NO: 49, SEQ ID NO: 50, SEQ ID NO: 51, and SEQ ID NO: 52,
SEQ ID NO: 61, SEQ ID NO: 62, SEQ ID NO: 63, SEQ ID NO: 64, SEQ ID NO: 65, and SEQ ID NO: 66,
SEQ ID NO: 75, SEQ ID NO: 76, SEQ ID NO: 77, SEQ ID NO: 78, SEQ ID NO: 79, and SEQ ID NO: 80,
SEQ ID NO: 89, SEQ ID NO: 90, SEQ ID NO: 91, SEQ ID NO: 92, SEQ ID NO: 93, and SEQ ID NO: 94,
SEQ ID NO: 103, SEQ ID NO: 104, SEQ ID NO: 105, SEQ ID NO: 106, SEQ ID NO: 107, and SEQ ID NO: 108,
SEQ ID NO: 117, SEQ ID NO: 118, SEQ ID NO: 119, SEQ ID NO: 120, SEQ ID NO: 121, and SEQ ID NO: 122,
SEQ ID NO: 131, SEQ ID NO: 132, SEQ ID NO: 133, SEQ ID NO: 134, SEQ ID NO: 135, and SEQ ID NO: 136,
SEQ ID NO: 145, SEQ ID NO: 146, SEQ ID NO: 147, SEQ ID NO: 148, SEQ ID NO: 149, and SEQ ID NO: 150,
SEQ ID NO: 159, SEQ ID NO: 160, SEQ ID NO: 161, SEQ ID NO: 162, SEQ ID NO: 163, and SEQ ID NO: 164,
SEQ ID NO: 173, SEQ ID NO: 174, SEQ ID NO: 175, SEQ ID NO: 176, SEQ ID NO: 177, and SEQ ID NO: 178,
SEQ ID NO: 187, SEQ ID NO: 188, SEQ ID NO: 189, SEQ ID NO: 190, SEQ ID NO: 191, and SEQ ID NO: 192,
SEQ ID NO: 201, SEQ ID NO: 202, SEQ ID NO: 203, SEQ ID NO: 204, SEQ ID NO: 205, and SEQ ID NO: 206,
SEQ ID NO: 215, SEQ ID NO: 216, SEQ ID NO: 217, SEQ ID NO: 218, SEQ ID NO: 219, and SEQ ID NO: 220,
SEQ ID NO: 229, SEQ ID NO: 230, SEQ ID NO: 231, SEQ ID NO: 232, SEQ ID NO: 233, and SEQ ID NO: 234,
SEQ ID NO: 243, SEQ ID NO: 244, SEQ ID NO: 245, SEQ ID NO: 246, SEQ ID NO: 247, and SEQ ID NO: 248,
SEQ ID NO: 257, SEQ ID NO: 258, SEQ ID NO: 259, SEQ ID NO: 260, SEQ ID NO: 261, and SEQ ID NO: 262, and
SEQ ID NO: 271, SEQ ID NO: 272, SEQ ID NO: 273, SEQ ID NO: 274, SEQ ID NO: 275, and SEQ ID NO: 276, or a polypeptide construct which competes with the binding to CLDN6.

The invention relates to polypeptides/polypeptide constructs according to any one of the preceding paragraphs comprising or consisting of the amino acid depicted in SEQ ID NO: 21.

The invention relates to polypeptides/polypeptide constructs according to any one of the preceding paragraphs comprising or consisting of the amino acid depicted in SEQ ID NO: 24.

The invention relates to polypeptides/polypeptide constructs according to any one of the preceding paragraphs comprising or consisting of the amino acid depicted in SEQ ID NO: 35.

The invention relates to polypeptides/polypeptide constructs according to any one of the preceding paragraphs comprising or consisting of the amino acid depicted in SEQ ID NO: 38.

The invention relates to polypeptides/polypeptide constructs according to any one of the preceding paragraphs comprising or consisting of the amino acid depicted in SEQ ID NO: 49.

The invention relates to polypeptides/polypeptide constructs according to any one of the preceding paragraphs comprising or consisting of the amino acid depicted in SEQ ID NO: 52.

The invention relates to polypeptides/polypeptide constructs according to any one of the preceding paragraphs comprising or consisting of the amino acid depicted in SEQ ID NO: 63.

The invention relates to polypeptides/polypeptide constructs according to any one of the preceding paragraphs comprising or consisting of the amino acid depicted in SEQ ID NO: 66.

The invention relates to polypeptides/polypeptide constructs according to any one of the preceding paragraphs comprising or consisting of the amino acid depicted in SEQ ID NO: 77.

The invention relates to polypeptides/polypeptide constructs according to any one of the preceding paragraphs comprising or consisting of the amino acid depicted in SEQ ID NO: 80.

The invention relates to polypeptides/polypeptide constructs according to any one of the preceding paragraphs comprising or consisting of the amino acid depicted in SEQ ID NO: 234.

The invention relates to polypeptides/polypeptide constructs according to any one of the preceding paragraphs comprising or consisting of the amino acid depicted in SEQ ID NO: 276.

The polypeptide/polypeptide construct according to any one of the preceding embodiments, wherein the polypeptide/polypeptide construct induces at least 100 fold, at least 250 fold, at least 500 fold lower cytotoxicity, or at least 1000 fold lower T cell-dependent cytotoxicity as determined in an in vitro assay using a cell that expresses a mutant of wild-type CLDN6 as depicted in SEQ ID NO: 1 that comprises at least one or more of the following mutations M29X, wherein X is preferably L, R145X, wherein X is preferably Q, and/or Q156X, wherein X is preferably L, as compared with the T cell-dependent cytotoxicity measured in the in vitro assay using a cell that expresses CLDN6 as depicted in SEQ ID NO: 1.

According to the invention, polypeptides/polypeptide constructs are provided,
wherein a domain (comprising a paratope, i.e., an antigen-binding (epitope-binding) structure)) of the polypeptide construct of the invention is capable of binding and discriminating CLDN6 on the surface of a cell that expresses CLDN6 as depicted in SEQ ID NO: 1 and a CLDN6 mutant on the surface of a cell that expresses said CLDN6 mutant, wherein said CLDN6 mutant comprises the sequence depicted in SEQ ID NO: 1, in which at least one of residues 31, 38, and 39 is replaced by another amino acid residue, particularly, wherein residue 31 is R and/or residue 38 is S and/or residue 39 is N, and/or wherein at least one of residues 31, 38, and 39 is replaced by another amino acid residue, particularly, wherein residue 156 is not Q,
wherein optionally a domain (comprising a paratope (i.e., an antigen-binding (epitope-binding) structure)) of the polypeptide construct of the invention binds CD3 (particularly human or non-human primate CD3),
wherein said polypeptides/polypeptide constructs are capable of engaging, activating T cells and inducing T cell-dependent cytotoxicity when it (a paratope (i.e., an antigen-binding (epitope-binding) structure)) binds to CLDN6 on the surface of a cell that expresses CLDN6 and when a further antigen-binding (epitope-binding) domain comprises a paratope that binds to CD3, and
wherein a domain (comprising a paratope (i.e., an antigen-binding (epitope-binding) structure)) that binds CLDN6 comprises a heavy chain CDR3 region comprising the sequence: X1LIVX2APX3 (SEQ ID NO. 667), wherein X1 is either A or N; X2 is either V or E; and X3 is either V or A,
wherein optionally the polypeptide construct does not selectively bind to CLDN1, CLDN2, CLDN3, CLDN4, CLDN9, and/or CLDN18.1,
wherein preferably the polypeptides/polypeptide constructs binds to the E1A and/or the E2B regions of CLDN6 (SEQ ID NO: 1) as depicted in SEQ ID NOs: 9 and 10, and wherein preferably the polypeptides/polypeptide constructs do not bind to an epitope comprising amino acids 138-150 of CLDN6 (SEQ ID NO: 1).

According to the invention, polypeptides/polypeptide constructs are provided, wherein a domain (comprising a paratope (i.e., an antigen-binding (epitope-binding) structure)) of the polypeptide construct of the invention is capable of binding and discriminating CLDN6 on the surface of a cell that expresses CLDN6 as depicted in SEQ ID NO: 1 and a CLDN6 mutant on the surface of a cell that expresses said CLDN6 mutant, wherein said CLDN6 mutant comprises the sequence as depicted in SEQ ID NO: 1 in which at least one of residues 31, 38, and 39 is replaced by another amino acid residue, particularly, wherein residue 31 is R and/or residue 38 is S and/or residue 39 is N,
wherein optionally a domain (comprising a paratope (i.e., an antigen-binding (epitope-binding) structure)) of the construct of the invention binds CD3 (particularly human or non-human primate CD3),
further wherein said polypeptides/polypeptide constructs are capable of engaging, activating T cells and inducing T cell-dependent cytotoxicity when it (e.g., a paratope (i.e., an antigen-binding (epitope-binding) structure)) binds to CLDN6 on the surface of a cell that expresses CLDN6 and when a further antigen-binding (epitope-binding) domain (comprises a paratope) that binds to CD3, and
wherein the domain (comprising a paratope (i.e., an antigen-binding (epitope-binding) structure)) that binds CLDN6 comprises a heavy chain CDR3 region comprising the sequence: DX1LIVX2APX3T (SEQ ID NO. 668), wherein X1 is either A or N; X2 is either V or E; and X3 is either V or A,
wherein optionally the domain (comprises a paratope (i.e., an antigen-binding (epitope-binding) structure)) that does not immunospecifically or immunoselectively bind to CLDN1, CLDN2, CLDN3, CLDN4, CLDN9, and/or CLDN18.1,
wherein optionally the domain (comprising a paratope (i.e., an antigen-binding (epitope-binding) structure)) of the polypeptide construct of the invention is capable of binding and discriminating CLDN6 on the surface of a cell that expresses CLDN6 binds to the E1A and/or the E2B regions of CLDN6 (SEQ ID NO: 1).

According to the invention, polypeptides/polypeptide constructs are provided, wherein a domain of said polypeptides/polypeptide constructs (comprise a paratope (i.e., an antigen-binding (epitope-binding) structure)) that is capable of binding and discriminating CLDN6 on the surface of a cell that expresses CLDN6 as depicted in SEQ ID NO: 1 and a CLDN6 mutant on the surface of a cell that expresses said CLDN6 mutant, wherein said CLDN6 mutant comprises the sequence as depicted in SEQ ID NO: 1, in which at least one of residues 31, 38, and 39 is replaced by another amino acid residue, particularly, wherein residue 31 is R and/or residue 38 is S and/or residue 39 is N, wherein optionally a domain (comprising a paratope (i.e., an antigen-binding (epitope-binding) structure)) of the construct of the invention binds CD3 (particularly human or non-human primate CD3), further wherein said polypeptides/polypeptide constructs are capable of engaging, activating T cells and inducing T cell-dependent cytotoxicity when it (e.g., a paratope (i.e., an antigen-binding (epitope-binding) structure)) binds to CLDN6 on the surface of a cell that expresses CLDN6 and when a further antigen-binding (epitope-binding) domain comprises a paratope that binds to CD3, and wherein the domain (comprising a paratope (i.e., an antigen-binding (epitope-binding) structure)) that binds CLDN6 comprises a heavy chain CDR3 region comprising the sequence: DX₁LIVX₂APX₃TRDYYYYGMDV (SEQ ID NO. 669), wherein X₁ is either A or N; X₂ is either V or E; and X₃ is either V or A, wherein optionally the domain (comprising a paratope (i.e., an antigen-binding (epitope-binding) structure)) that binds CLDN6 does not (immunoselectively or immunoselectively) bind to CLDN1, CLDN2, CLDN3, CLDN4, CLDN9, CLDN18.1 and/or CLDN18.2, wherein optionally the domain (comprising a paratope (i.e., an antigen-binding (epitope-binding) structure)) of the polypeptide construct of the invention is capable of binding and discriminating CLDN6 on the surface of a cell that expresses CLDN6 binds to the E1A and/or the E2B regions of CLDN6 (SEQ ID NO: 1). In embodiments the polypeptide/polypeptide constructs are capable of binding CLDN6 on the surface of a cell that expresses CLDN6 binds to the E1A and/or the E2B regions of CLDN6 (SEQ ID NO: 1) and do not bind to amino acids 138-150 of CLDN6 as depicted in SEQ ID NO: 1.

According to the invention, polypeptides/polypeptide constructs are provided,
wherein a domain of said polypeptides/polypeptide constructs (comprise a paratope (i.e., an antigen-binding (epitope-binding) structure)) that is capable of binding and discriminating CLDN6 on the surface of a cell that expresses CLDN6 as depicted in SEQ ID NO: 1 and a CLDN6 mutant on the surface of a cell that expresses said CLDN6 mutant, wherein said CLDN6 mutant comprises the sequence as depicted in SEQ ID NO: 1, in which at least one of residues 31, 38, and 39 is replaced by another amino acid residue, particularly, wherein residue 31 is R and/or residue 38 is S and/or residue 39 is N,
wherein optionally a domain (comprising a paratope (i.e., an antigen-binding (epitope-binding) structure)) of the construct of the invention binds CD3 (particularly human or non-human primate CD3),
further wherein said polypeptides/polypeptide constructs are capable of engaging, activating T cells and inducing T cell-dependent cytotoxicity when it (through a paratope (i.e., an antigen-binding (epitope-binding) structure)) binds to CLDN6 on the surface of a cell that expresses CLDN6 and when a further antigen-binding (epitope-binding) domain (comprises a paratope) that binds to CD3,
wherein the domain (comprising a paratope (i.e., an antigen-binding (epitope-binding) structure)) that is capable of binding and discriminating CLDN6 on the surface of a cell that expresses CLDN6 as depicted in SEQ ID NO: 1 comprises a heavy chain fragment comprising the heavy chain CDR3 region comprising a sequence that is selected from the group of sequences that are depicted in any one of SEQ ID Nos: 15, 23, 31, 39, 47, 55, 63, 71, 79, 87, 95, 103, 111, 119, 127, 135, 143, and 151, particularly from the group comprising sequences depicted in SEQ ID Nos: 15, 23, 31, and 47, very particularly the heavy chain CDR3 region comprises or consists of SEQ ID NO: 15;
wherein optionally the polypeptide construct does not selectively bind to CLDN2 (SEQ ID NO: 5), CLDN3 (SEQ ID NO: 6), CLDN4 (SEQ ID NO: 7), CLDN9 (SEQ ID NO: 8), CLDN18.1 (SEQ ID NO: 2) and/or CLDN18.2 (SEQ ID NO: 3), and/or
wherein the construct binds to the E1A and/or the E2B regions of CLDN6 (SEQ ID NO: 1), and do not bind to amino acids 138-150 of CLDN6 as depicted in SEQ ID NO: 1.

According to the invention, polypeptides/polypeptide constructs are provided that are comprising a domain which binds to human CLDN6 (SEQ ID NO: 1), and a domain which binds to human CD3, and a domain extending the half-life of the polypeptide as defined throughout the description and the claims, wherein the domain which binds to CLDN6 comprises a variable light (VL) chain domain that comprises a CDR1 region as depicted in the following sequence RASQSVX₁SX₂YLA (SEQ ID NO: 695), wherein X₁ is selected from S and R, preferably S, and wherein X₂ is selected from S and T, preferably S; and/or a CDR3 region as depicted in the following sequence QQYX₁X2SPX₃T (SEQ ID NO: 696) wherein X₁ is selected from G, D, and Q, preferably G, and wherein X₂ is selected from S, A and T, preferably S, and X₃ is selected from L and I, preferably L. In one particular embodiment, the polypeptides/polypeptide constructs have a VL chain comprising a CDR1 region as depicted in SEQ ID NO: 16 and a CDR3 region as depicted in SEQ ID NO: 18, further preferably in combination with a VL CDR2 region depicted in SEQ ID NO: 17, further particularly in combination with CDR1, CDR2, CDR3 region of the variable heavy (VH) chain domain as depicted in SEQ ID NOs: 13, 14, and/or 15; These polypeptides/polypeptides bind to CLDN6 regions depicted in SEQ ID NO: 9 and/or 10 as determined in domain swapping experiments (cf. the Examples section). It was found out that polypeptides or polypeptide constructs of the present invention are particularly well-suited to distinguish between CLDN6 and CLDN9 and preferably bind to and in vitro effectively kill CLDN6 cells, e.g. CHO cells transformed with nucleic acids encoding either CLDN6 or CLDN9. Not only is the cytotoxic activity better, but the polypeptides or polypeptide constructs also show a surprisingly high protein stability as determined in a DLS ° C. aggregation thermostability test at 1 mg/ml when they have the above captioned CDRs. These characteristics are important in polypeptides and/or polypeptides that are used in immune-oncology (T-cell engaging) therapeutic methods and for the preparation and storaging of pharmaceutical formulations.

According to the invention, polypeptides/polypeptide constructs are provided, wherein a domain (comprising a paratope (i.e., an antigen-binding (epitope-binding) structure)) binds to CLDN6 as defined in any one of the sections above, which further comprise a domain (comprising a paratope (i.e., an antigen-binding (epitope-binding) structure)) that binds to CD3, particularly to CD3-binding paratopes as disclosed, for example, in WO2019/133961, which only show cross-species specificity for the human and the Macaca, or Callithrix jacchus, Saguinus oedipus or Saimiri sciureus CD3ε chain, but also, due to recognizing this specific epitope (instead of previously described epitopes of CD3 binders in bispecific T cell engaging molecules), do not demonstrate unspecific activation of T cells to the same degree as observed for the previous generation of T cell engaging antibodies. The sequences of the CD3-binding domains/paratopes that can be used in context with the antibodies and constructs of the present invention are described below in the respective paragraphs.

Advantageously, targeting the epitope of CLDN6, which is recognized by the constructs of the present invention (see also the Examples section), provides the following benefits:
(1) Immunospecificity/immunoselectivity of the CLDN6×CD3 constructs over CLDN9 (Examples 1 and 5), and
(2) An unexpectedly high cytotoxic potency for the CLDN6×CD3 constructs (Examples 4, 6, and 7).

According to the invention, the polypeptides/polypeptide constructs of the present invention comprising a domain (comprising a paratope (i.e., an antigen-binding (epitope-binding) structure)) antigen-binding (epitope-binding) specifically and selectively binding to CD3, which is normally expressed on T cells.

Examples of a CD3c extracellular domain bound by the present domains/paratopes are shown in SEQ ID NOs: 442 and 443, respectively. Further, examples of CD3ε-binding domain/paratope amino acids, scFv's comprising the same, VH and VL chains are shown in SEQ ID NOs: 444 to 562 as well as in and particularly in SEQ ID NOs: 670 to 678.

The present invention relates also to polypeptides according to any of the preceding paragraphs, wherein a binding domain binding to an extracellular epitope of the human CD3ε chain comprising or consisting of a VH region linked to a VL region, wherein
i) the VH region comprises:
a CDR-H1 sequence of X1YAX2N, where X1 is K, V, S, G, R, T, or I; and X2 is M or I;
a CDR-H2 sequence of RIRSKYNNYATYYADX1VK X2, where X1 is S or Q; and X2 is D, G, K, S, or E; and
a CDR-H3 sequence of HX1NFGNSYX2SX3X4AY, where X1 is G, R, or A; X2 is I, L, V, or T; X3 is Y, W or F; and X4 is W, F or Y; and
ii) wherein the VL region comprises:
a CDR-L1 sequence of X1SSTGAVTX2X3X4YX5N, where X1 is G, R, or A; X2 is S or T; X3 is G or S; X4 is N or Y; and X5 is P or A;
a CDR-L2 sequence of X1TX2X3X4X5X6; where X1 is G or A; X2 is K, D, or N; X3 is F, M or K; X4 is L or R; X5 is A, P, or V; and X6 is P or S; and
a CDR-L3 sequence of X1LWYSNX2WV, where X1 is V, A, or T; and X2 is R or L; and
iii) wherein the CDR sequence of i) and/or ii) comprise one or more amino acid substitutions selected from X24V or X24F in CDR-H1;
D15 (preferably E), X116A in CDR-H2;
H1 (preferably A or N), X12E, F4 (preferably I), and/or N6 (preferably S or T) in CDR-H3; and
W93 (preferably Y) in CDR-L3.

The present invention relates to compounds which may have linkers, half-life extending peptides, and other structural moieties as disclosed in SEQ ID NOs: 563 to 575, and in SEQ ID NOs: 576 to 666, respectively. Details on functions of these structures are found in the Sequence table following the Examples section.

It is envisaged that polypeptides/polypeptide constructs in accordance with the present invention that the domain (comprising a paratope) binding to CD3 on the surface of a T cell comprise a VL region selected from the group consisting of VL regions as depicted in the respective SEQ ID numbers 444 to 562 and 677 exemplified in the sequence listing, particularly in SEQ ID numbers 507-512, and 534-541 and 677.

In another embodiment polypeptides/polypeptide constructs in accordance with the present invention that the domain (comprising a paratope) binding to CD3 on the surface of a T cell comprise a VL region as depicted in SEQ ID NO: 677.

It is also envisaged that polypeptides/polypeptide constructs in accordance with the present invention that the domain (comprising a paratope) binding to CD3 on the surface of a T cell comprise a VH region selected from the group consisting of VH regions as depicted in the respective SEQ ID numbers 444 to 562 and 676 exemplified in the sequence listing, particularly in SEQ ID numbers 513-533 and 676.

In another embodiment polypeptides/polypeptide constructs in accordance with the present invention that the domain (comprising a paratope) binding to CD3 on the surface of a T cell comprise a VH region as depicted in SEQ ID NO: 676.

More preferably, the polypeptides/polypeptide constructs in accordance with the present invention comprising the domain (comprising a paratope) binding to CD3 on the surface of a T cell comprise a VL region and a VH region selected from the group consisting of VL regions and VH regions as depicted in the respective SEQ ID numbers exemplified in the sequence listing, particularly, in the following pairs of VL regions and and VH regions, particularly in SEQ ID numbers 507+514, 508+519, 509+521, 510+525, 511+528, 512+532, 534+513, 535+515, 536+516, 537+517, 538+518, 539+520, 540+522, and 541+523, and very particularly the pair of VL regions and and VH regions, particularly in SEQ ID numbers 676+677.

A preferred embodiment of the above described polypeptides/polypeptide constructs in accordance with the present invention is characterized by a domain (comprising a paratope) which binds to CD3 on the surface of a T cell comprising an amino acid sequence selected from the group consisting of SEQ ID NOs: 542-562 and SEQ ID NO: 678.

A particular embodiment of the above described polypeptides/polypeptide constructs in accordance with the present invention is characterized by a domain (comprising a paratope) which binds to CD3 on the surface of a T cell comprising an amino acid sequence depicted in SEQ ID NO: 678.

A particular embodiment of the above described polypeptides/polypeptide constructs in accordance with the present invention is characterized by a domain (comprising a paratope) which binds to CD3 on the surface of a T cell comprising an amino acid sequence depicted in SEQ ID NO: 678, and wherein the domain (comprising the paratope (i.e., the antigen-binding (epitope-binding) structure)) binding to CLDN6 is comprised by, or which competes with the binding to CLDN6, with a pair of VH and VL regions comprising amino acid sequences depicted in SEQ ID NOs: 11+12, SEQ ID NO: 25+26, SEQ ID NO: 39+40, SEQ ID NO: 53+54, SEQ ID NO: 67+68, SEQ ID NO: 81+82, SEQ ID NO: 95+96, SEQ ID NO: 109+110, SEQ ID NO: 123+124, SEQ ID NO: 137+138, SEQ ID NO: 151+152, SEQ ID NO: 165+166, SEQ ID NO: 179+180, SEQ ID NO: 193+194, SEQ ID NO: 207+208, SEQ ID NO: 221+222, SEQ ID NO: 235+236, SEQ ID NO: 249+250, or SEQ ID NO: 263+264.

A particular embodiment of the above described polypeptides/polypeptide constructs in accordance with the present invention is characterized by a domain (comprising a paratope) which binds to CD3 on the surface of a T cell comprising an amino acid sequence depicted in SEQ ID NO: 678, and wherein the domain (comprising the paratope (i.e., the antigen-binding (epitope-binding) structure)) binding to CLDN6 is comprised by, or which competes with the binding to CLDN6, comprises amino acid sequences depicted in SEQ ID NOs: SEQ ID NO: 19, SEQ ID NO: 22, SEQ ID NO: 33, SEQ ID NO: 36, SEQ ID NO: 47, SEQ ID NO: 50, SEQ ID NO: 61, SEQ ID NO: 64, SEQ ID NO: 75, SEQ ID NO: 78, SEQ ID NO: 89, SEQ ID NO: 92, SEQ ID NO: 103, SEQ ID NO: 106, SEQ ID NO: 117, SEQ ID NO: 120, SEQ ID NO: 131, SEQ ID NO: 134, SEQ ID NO: 145, SEQ ID NO: 148, SEQ ID NO: 159, SEQ ID NO: 162, SEQ ID NO: 173, SEQ ID NO: 176, SEQ ID NO: 187, SEQ ID NO: 190, SEQ ID NO: 201, SEQ ID NO: 204, SEQ ID NO: 215, SEQ ID NO: 218, SEQ ID NO: 229, SEQ ID NO: 232, SEQ ID NO: 243, SEQ ID NO: 246, SEQ ID NO: 257, or SEQ ID NO: 260, SEQ ID NO: 271 or SEQ ID NO: 274.

A particular embodiment of the above described polypeptides/polypeptide constructs in accordance with the present invention is characterized by a domain (comprising a paratope) which binds to CD3 on the surface of a T cell comprising an amino acid sequence depicted in SEQ ID NO: 678, and wherein the domain (comprising the paratope (i.e., the antigen-binding (epitope-binding) structure)) binding to CLDN6 is depicted in a SEQ ID number selected from the group comprising SEQ ID NOs: 19, 22, 33, 36, 47, 50, 75, 78, 201, and 204, particularly SEQ ID NOs: 19 and 22, very particularly SEQ ID NO: 22.

Another particular embodiment of the above described polypeptides/polypeptide constructs in accordance with the present invention is characterized by a domain (comprising a paratope) which binds to CD3 on the surface of a T cell comprising an amino acid sequence depicted in SEQ ID NO: 678, and wherein the domain (comprising the paratope (i.e., the antigen-binding (epitope-binding) structure)) binding to CLDN6 is depicted in SEQ ID NO: 22.

A very particular embodiment of the above described polypeptides/polypeptide constructs in accordance with the present invention is characterized by a domain (comprising a paratope) which binds to CD3 on the surface of a T cell, said domain comprising VH CDR sequences HCDR1, HCDR2, and/or HCDR3, depicted in SEQ ID NOs: 670, 671, and/or 672, and/or wherein the domain (comprising a paratope) which binds to CD3 on the surface of a T cell comprises VL CDR sequences LCDR1, LCDR2, and/or LCDR3 is depicted in SEQ ID NOs: 673, 674, and/or 675, and wherein the domain (comprising the paratope (i.e., the antigen-binding (epitope-binding) structure)) binding to CLDN6 is depicted in any one of the sequences shown in SEQ ID NO: 22, 36, 50, 78, and 204.

Another very particular embodiment of the above described polypeptides/polypeptide constructs in accordance with the present invention is characterized by a domain (comprising a paratope) which binds to CD3 on the surface of a T cell, wherein said domain comprising VH CDR sequences HCDR1, HCDR2, and/or HCDR3, depicted in SEQ ID NOs: 670, 671, and/or 672, and/or wherein the domain comprises VL CDR sequences LCDR1, LCDR2, and/or LCDR3 is depicted in SEQ ID NOs: 673, 674, and/or 675, and wherein said domain (comprising the paratope (i.e., the antigen-binding (epitope-binding) structure)) binding to CLDN6 comprises VH CDR sequences HCDR1, HCDR2, and/or HCDR3 as depicted in SEQ ID NOs: 13, 14, and/or 15, and/or wherein the domain (comprising a paratope) comprises VL CDR sequences LCDR1, LCDR2, and/or LCDR3 as depicted in any one of the sequences shown in SEQ ID NOs: 16, 17, and/or 18.

Yet another very particular embodiment of the above described polypeptides/polypeptide constructs in accordance with the present invention is characterized by a domain (comprising a paratope) which binds to CD3 on the surface of a T cell, wherein said domain comprising VH CDR sequences HCDR1, HCDR2, and/or HCDR3 as depicted in any one of the sequences shown in SEQ ID NOs: 670, 671, and/or 672, and/or wherein the domain comprises VL CDR sequences LCDR1, LCDR2, and/or LCDR3 as depicted in any one of the sequences shown in SEQ ID NOs: 673, 674, and/or 675, and wherein said domain (comprising the paratope (i.e., the antigen-binding (epitope-binding) structure)) binding to CLDN6 comprises VH CDR sequences HCDR1, HCDR2, and/or HCDR3 as depicted in any one of the sequences shown in SEQ ID NOs: 27, 28, and/or 29, and/or wherein the domain (comprising a paratope) comprises VL CDR sequences LCDR1, LCDR2, and/or LCDR3 as depicted in any one of the sequences shown in SEQ ID NOs: 30, 31, and/or 32.

Still another very particular embodiment of the above described polypeptides/polypeptide constructs in accordance with the present invention is characterized by a domain (comprising a paratope) which binds to CD3 on the surface of a T cell, wherein said domain comprising VH CDR sequences HCDR1, HCDR2, and/or HCDR3 as depicted in any one of the sequences shown in SEQ ID NOs: 670, 671, and/or 672, and/or wherein the domain comprises VL CDR sequences LCDR1, LCDR2, and/or LCDR3 as depicted in any one of the sequences shown in SEQ ID NOs: 673, 674, and/or 675, and wherein said domain (comprising the paratope (i.e., the antigen-binding (epitope-binding) structure)) binding to CLDN6 comprises VH CDR sequences HCDR1, HCDR2, and/or HCDR3 as depicted in any one of the sequences shown in SEQ ID NOs: 41, 42, and/or 42, and/or wherein the domain (comprising a paratope) comprises VL CDR sequences LCDR1, LCDR2, and/or LCDR3 as depicted in any one of the sequences shown in SEQ ID NOs: 44, 45, and/or 46.

Still another very particular embodiment of the above described polypeptides/polypeptide constructs in accordance with the present invention is characterized by a domain (comprising a paratope) which binds to CD3 on the surface of a T cell, wherein said domain comprising VH CDR sequences HCDR1, HCDR2, and/or HCDR3 as depicted in any one of the sequences shown in SEQ ID NOs: 670, 671, and/or 672, and/or wherein the domain comprises VL CDR sequences LCDR1, LCDR2, and/or LCDR3 as depicted in any one of the sequences shown in SEQ ID NOs: 673, 674, and/or 675, and wherein said domain (comprising the paratope (i.e., the antigen-binding (epitope-binding) structure)) binding to CLDN6 comprises VH CDR sequences HCDR1, HCDR2, and/or HCDR3 as depicted in any one of the sequences shown in SEQ ID NOs: 69, 70, and/or 71, and/or wherein the domain (comprising a paratope) comprises VL CDR sequences LCDR1, LCDR2, and/or LCDR3 as depicted in any one of the sequences shown in SEQ ID NOs: 72, 73, and/or 74.

Still another very particular embodiment of the above described polypeptides/polypeptide constructs in accordance with the present invention is characterized by a domain (comprising a paratope) which binds to CD3 on the surface of a T cell, wherein said domain comprising VH CDR sequences HCDR1, HCDR2, and/or HCDR3 as depicted in any one of the sequences shown in SEQ ID NOs: 670, 671, and/or 672, and/or wherein the domain comprises VL CDR sequences LCDR1, LCDR2, and/or LCDR3 as depicted in any one of the sequences shown in SEQ ID NOs: 673, 674, and/or 675, and wherein said domain (comprising the paratope (i.e., the antigen-binding (epitope-binding) structure)) binding to CLDN6 comprises VH CDR sequences HCDR1, HCDR2, and/or HCDR3 as depicted in any one of the sequences shown in SEQ ID NOs: 195, 196, and/or 197, and/or wherein the domain (comprising a paratope) comprises VL CDR sequences LCDR1, LCDR2, and/or LCDR3 as depicted in any one of the sequences shown in SEQ ID NOs: 198, 199, and/or 200.

### Nucleic Acids, Host Cells and Processes of Producing the Compounds of the Invention

In a second aspect, it is further envisaged in the context of the present invention to provide a polynucleotide encoding a polypeptide construct of the present invention as depicted in any one of the preceding sections.

It is also envisaged in the context of the present invention to provide a vector comprising a polynucleotide of the present invention.

Further, the present invention to provides host cells transformed or transfected with the polynucleotide or with the vector of the present invention.

It is also envisaged in the context of the present invention to provide a process to produce a polypeptide construct of the present invention, said process comprising culturing a host cell of the present invention under conditions allowing the expression of the construct and recovering the produced polypeptide construct from the culture.

### Pharmaceutical Compositions of the Invention

In a further aspect, the present invention provides pharmaceutical compositions comprising a polypeptide compound of the present invention or polypeptide compounds produced according to the process of the present invention.

Within said aspect, is also envisaged in the context of the present invention that the pharmaceutical composition is stable for at least four weeks at about −20° C.

### Therapeutic Uses/Methods of the Invention

It is further envisaged in the context of the present invention to provide the polypeptide compounds and pharmaceutical compositions of the present invention or polypeptide compounds and pharmaceutical compositions comprising such polypeptide compounds that are produced according to processes of the present invention, for the use as a medicament, particularly for the use in the prevention, treatment or amelioration of a disease selected from a proliferative disease, a tumorous disease, cancer or an immunological disorder.

It is further envisaged in the context of the present invention to provide a method for the treatment or amelioration of a proliferative disease, a tumorous disease, cancer, or an immunological disorder, comprising the step of administering to a subject in need thereof a polypeptide compound or pharmaceutical composition of the present invention, wherein optionally, the compound is produced according to the process of the present invention.

Preferably the disease is selected from the group comprising various types of cancer expressing CLDN6 selected from the group consisting of urinary bladder cancer, ovarian cancer, in particular ovarian adenocarcinoma and ovarian teratocarcinoma, lung cancer, including small cell lung cancer (SCLC) and non-small cell lung cancer (NSCLC), in particular squamous cell lung carcinoma and adenocarcinoma, gastric cancer, breast cancer, hepatic cancer, pancreatic cancer, skin cancer, in particular basal cell carcinoma and squamous cell carcinoma, malignant melanoma, head and neck cancer, in particular malignant pleomorphic adenoma, sarcoma, in particular synovial sarcoma and carcinosarcoma, bile duct cancer, cancer of the urinary bladder, in particular transitional cell carcinoma and papillary carcinoma, kidney cancer, in particular renal cell carcinoma including clear cell renal cell carcinoma and papillary renal cell carcinoma, colon cancer, small bowel cancer, including cancer of the ileum, in particular small bowel adenocarcinoma and adenocarcinoma of the ileum, testicular embryonal carcinoma, placental choriocarcinoma, cervical cancer, testicular cancer, in particular testicular seminoma, testicular teratoma and embryonic testicular cancer, uterine cancer, germ cell tumors such as a teratocarcinoma or an embryonal carcinoma, in particular germ cell tumors of the testis, and the metastatic forms thereof, very particularly testicular germ cell cancer, ovarian cancer, particularly ovarian serous cystadenocarcinoma, and uterine cancer such as uterine corpus endometrial carcinoma, lung cancer, including small cell lung cancer (SCLC) and non-small cell lung cancer (NSCLC), such as lung adenocarcinoma, triple negative breast cancer, stomach cancer, cholangiocarcinoma, esophagus cancer, Wilms tumor, rhabdoid tumor, particularly, ovarian cancer, uterine cancer, and/or lung cancer, and more particularly ovarian serous cystadenocarcinoma, uterine carcinosarcoma, uterine corpus endometrial carcinoma, and/or in particular squamous cell lung carcinoma and lung adenocarcinoma.

Also provided are uses of the herein described compounds for the preparation of medicaments for the treatment or prevention or amelioration of a neoplastic disease, particularly, ovarian cancer, uterine cancer, and/or lung cancer.

It is envisaged in the context of the present invention to provide a method for the treatment or amelioration of gastrointestinal cancer, comprising the step of administering to a subject in need thereof a construct directed against CLDN6 and CD3.

It is also envisaged in the context of the present invention to provide polypeptides/polypeptide constructs directed against CLDN6 and CD3 for the use as a medicament, particularly for the use in the treatment or amelioration of, e.g., ovarian cancer, uterine cancer, lung cancer, particularly ovarian cancer, in particular ovarian adenocarcinoma and ovarian teratocarcinoma, lung cancer, including small cell lung cancer (SCLC) and non-small cell lung cancer (NSCLC), in particular squamous cell lung carcinoma and adenocarcinoma.

### Kits of the Invention

In another aspect, it is also envisaged in the context of the present invention to provide a kit comprising a polypeptide construct of the present invention or produced according to the process of the present invention, a polynucleotide of the present invention, a vector of the present invention, and/or a host cell of the present invention.

### Definitions of Terms According to the Invention

The term “polypeptide construct” (alternatively referred to simply as “compound”) refers to an antigen-binding (or epitope-binding) molecule comprising domains themselves comprising paratopes. In the context of the present invention, a polypeptide construct is understood as an organic polymer which comprises at least one continuous, unbranched amino acid chain that naturally is not existing, but was engineered. An example of a polypeptide construct that is a single polypeptide is a BiTE® molecule that comprises a core structure comprising at least one functional target binding domain together with at least one complete functional CD3 binding domain on a single polypeptide chain, wherein these domains are linked directly by flexible peptide (a “linker”) without any further inserted domain unlike Xmabs that comprise the target binder and the CD3 binder on different polypeptide chains. In the context of the present invention, such a polypeptide construct comprising more than one amino acid chain is likewise envisaged. It is preferred that the term “polypeptide” is used in connection with single chain forms of the compounds of the present invention, whereas “polypeptide construct” may preferably be more adequate to describe also polypeptides that comprise more than one polypeptide chain, for example two, three or four polypeptide chains. Additionally, the term “polypeptide construct” is also suitable to describe compounds of the invention that comprise one or more non-amino acid-based constituents, e.g. human serum albumin, etc. (HSA). An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The polypeptides comprise structural and/or functional features based on the structure and/or function of an antibody, e.g., of a full-length immunoglobulin molecule. A polypeptide construct, hence, specifically and, preferably, selectively or immunospecifically binds to its target or antigen, more precisely to an epitope of said target or target antigen, and/or it comprises the heavy chain variable region (VH) and/or the light chain variable region (VL) naturally found in an antibody, or comprises domains derived therefrom. Accordingly, the constructs may alternatively be regarded as comprising paratope-structured (i.e., paratope-structure forming) and epitope-binding structures, such as those found in natural antibodies or fragments thereof. A polypeptide construct according to the invention comprises the minimum structural requirements of an antibody which allow for immunospecific target binding, i.e., a paratope that recognizes immunospecifically or immunoselectively an epitope on a target antigen. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. A polypeptide construct may hence be characterized by the presence of three or six CDRs in either one or both binding domains, and the skilled person knows where (in which order) those CDRs are located within the paratopic binding structures. The term “antigen-binding structure”, as used herein, refers to any polypeptide that comprises an antigen-binding structure or any molecule that has binding activity to an antigen. The peptide and protein are not limited to those derived from a living organism, and for example, they may be a polypeptide produced from an artificially designed sequence. They may also be any of a naturally occurring polypeptide, synthetic polypeptide, recombinant polypeptide, and such. As the antigen-binding structure in accordance with the present invention bind specifically to parts of an antigen, i.e., they bind specifically to an epitope, the antigen (epitope)-binding structure may also be defined as “paratopic structure”. Accordingly, the polypeptides/polypeptide constructs according to the invention may also be defined as a domain comprising a paratope that are preferably immunospecifically or immunoselectively binding to a target antigen/target epitope and a further paratopic domain, preferably immunospecifically or immunoselectively, binding to a further target antigen/target epitope of the CD3 molecule as defined herein. Therefore, whenever the present description refers to a domain of a contruct or molecule of the present invention, the construct comprises at least one paratopic structure (or paratope) binding CLDN6 as defined herein, particularly according to any one of the appended claims, and a further paratopic structure binding CD3 as defined herein.

The term “antibody” as used in accordance with the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Polypeptides/polypeptide constructs” of the present invention may also comprise the structure of a full-length immunoglobulin as it occurs naturally. For example, they may comprise (at least) two full-length antibody heavy chains and two full-length antibody light chains. However, given that the polypeptides/polypeptide constructs according to the invention comprise one domain comprising a paratope binding to CLDN6 and another domain comprising a paratope binding to CD3, they do not occur naturally, and they are markedly different in their function from naturally occurring products. A polypeptide or polypeptide construct of the invention is hence an artificial “hybrid” molecule comprising distinct binding domains with different specificities and/or selectivities.

As indicated above, the polypeptides of the invention may comprise more than one polypeptide chain, i.e. polypeptides comprising two or more polypeptide chains are also subject to the present invention, particularly polypeptides forming a three-dimensional protein-like structure that allows for the immunospecific binding to CLDN6 and CD3. Therefore, the definition of the term “polypeptide construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and constructs derived therefrom are described inter alia in Harlow and Lane, Antibodies: A laboratory manual, CSHL Press (1988); Kontermann and Dubel, Antibody Engineering, Springer, 2nd ed. 2010; and Little, Recombinant Antibodies for Immunotherapy, Cambridge University Press 2009

“Polypeptides/polypeptide constructs” of the present invention may also comprise fragments of full-length antibodies, such as VH, VHH, VL, (s)dAb, Fv, light chain (VL-CL), Fd (VH-CH1), heavy chain, Fab, Fab′, F(ab′)2 or “rIgG” (“half antibody” consisting of a heavy chain and a light chain). Polypeptides/polypeptide constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL, that selectively and, preferably, specifically binds to an antigen or target independently of other variable regions or domains. Further possible formats of the polypeptides/polypeptide constructs according to the invention are cross bodies, maxi bodies, hetero Fc constructs, mono Fc constructs and scFc constructs. Examples for those formats will be described herein below.

Furthermore, the definition of the term “polypeptide construct” includes bivalent and polyvalent/multivalent polypeptides/polypeptide constructs as well as bispecific and polyspecific/multispecific polypeptides/polypeptide constructs, which selectively and, preferably, specifically bind to two, three or more antigenic structures (epitopes), through distinct binding domains. A polypeptide construct can have more binding valences than specificities, e.g. in a case where it has two binding domains for one target (CLDN6) and one binding domain for another target (CD3), or vice versa, in which case the polypeptide construct is trivalent and bispecific. In general, the term “bispecific” includes the meaning that a polypeptide construct binds to (at least) two different antigens, such as CLDN6 and CD3.

The terms “paratope”, “antigen-binding domain”, “epitope-binding domain”, “binding domain” or “domain which binds to . . . ” characterize, in connection with the present invention, a domain of the construct which selectively and, preferably, specifically or immunospecifically binds to/interacts with/recognizes an epitope on the target or antigen (here: CLDN6 in the case of the first domain, and CD3 in the case of the second domain). The terms “binding domain” or “domain which binds to . . . ” or “domain” as far as it relates to the herein described “constructs” characterizes in connection with the present invention, a domain of the construct which immunospecifically binds to/interacts with/recognizes an epitope (i.e. interacts selectively with certain amino acids) on the target or antigen. The structure and function of the first domain (binding to a target antigen), and preferably also the structure and/or function of the second domain (binding to CD3), is/are based on the structure and/or function of an antibody, e.g. of a full-length immunoglobulin polypeptide. The “binding domain” or “domain which binds to . . . ” may hence comprise the minimum structural requirements of an antibody which allow for immunospecific target binding. This minimum structural requirement of the first domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. It is envisaged that the second domain also comprises this minimum structural requirement of an antibody which allow for the immunospecific target binding. More preferably, the second domain also comprises at least three light chain CDRs (i.e. CDR1, CDR2 and CDR3 of the VL region) and/or three heavy chain CDRs (i.e. CDR1, CDR2 and CDR3 of the VH region), preferably all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain. The terms “paratope”, “antigen-binding structure” and “epitope-binding structure, as used herein, refer also to a portion of an antibody (or a molecule according to the invention), which comprises a region that specifically binds and is complementary to the whole or a portion of an antigen or a part thereof, i.e. an antibody can only bind to a particular portion of the antigen. The particular portion is called “epitope”. An antigen-binding domain can be provided from one or more antibody variable domains. Preferably, the antigen-binding domains contain antibody variable region that comprising both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”. A “paratope” may also be characterized by specific amino acids that interact chemically with specific amino acids on the side of the epitope (antigen/target).

Examples for the format of a “domain which binds to”, “domain comprising a paratope” (or “binding domain”, “antigen-binding structure”, “epitope-binding structure”) include, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′)2 or “r IgG” (“half antibody”)), antibody variants or derivatives such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” (selected from formats such as (VH-VL-CH3)2, (scFv-CH3)2, ((scFv)2-CH3+CH3)), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)2); (3) an antibody fragment or variant comprising VH and CH1 (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of constructs or binding domains according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272. In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a polypeptide according to the present invention is provided with a paratope comprising a set of 6 complementarity-determining regions (CDR loops) with three of each being comprised within the antibody-derived VH and VL sequence, respectively.

It is envisaged for the compounds, particularly for the constructs of the present invention that a) the construct is a single chain polypeptide or a single chain construct, b) the first domain is in the format of an scFv, c) the second domain is in the format of an scFv, d) the first and the second domain are connected via a linker, preferably a peptide linker, more preferably a glycine/serine linker, and/or e) the construct comprises a domain providing an extended serum half-life, such as an Fc-based domain, or human serum albumin (HSA). In the latter case, it is preferred embodiment, wherein the term “polypeptide construct” makes clear that it comprises more than a single peptide chain.

The constructs of the present invention are preferably “in vitro generated constructs” and/or “recombinant constructs”. In the context of the present invention, the term “in vitro generated” refers to a construct according to the above definition where all or part of the binding domain or of a variable region (e.g., at least one CDR) is generated in a non-immune cell selection, e.g., in an in vitro phage display, on a protein chip or in any other method in which candidate amino acid sequences can be tested for their ability to bind to an antigen. This term thus preferably excludes sequences generated solely by genomic rearrangement in an immune cell in an animal. It is envisaged that the first and/or second domain of the construct is produced by or obtainable by phage display or library screening methods rather than by grafting CDR sequences from a pre-existing (monoclonal) antibody into a scaffold. A “recombinant construct” is a construct generated or produced using (inter alia) recombinant DNA technology or genetic engineering.

The constructs of the present invention are envisaged to be monoclonal. As used herein, polypeptides or constructs that are denominated “monoclonal” (mAb) are obtained from a population of substantially homogeneous antibodies/constructs, i.e., the individual antibodies/constructs comprised in the population are identical (in particular with respect to their amino acid sequence) except for possible naturally occurring mutations and/or post-translational modifications (e.g., isomerizations, amidations) that may be present in minor amounts. Monoclonal antibodies/constructs are highly specific, being directed against a single epitope within the antigen, in contrast to polyclonal antibody preparations which typically include different antibodies directed against different determinants (or epitopes). In addition to their specificity, monoclonal antibodies are advantageous in that they are synthesized by the hybridoma culture, hence uncontaminated by other immunoglobulins. The modifier “monoclonal” indicates the character of the antibody/construct as being obtained from a substantially homogeneous population of antibodies and is not to be construed as requiring production of the antibody by any specific method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that selectively and, preferably, specifically or immunospecifically binds to a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore™ system can be used to increase the efficiency of phage antibodies/constructs which bind to an epitope of a target antigen (Schier, Human Antibodies Hybridomas 7 (1996), 97-105; Malmborg, J. Immunol. Methods 183 (1995), 7-13).

Another exemplary method of making constructs or binding domains includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., Xenomouse™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified constructs or binding domains include humanized variants of non-human antibodies/constructs, “affinity matured” constructs or binding domains (see, e.g. Hawkins et al. J. Mol. Biol. 254, 889-896 (1992) and Lowman et al., Biochemistry 30, 10832-10837 (1991)) and antibody variants or mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, constructs or binding domains with affinities in the low nanomolar range.

A preferred type of an amino acid substitutional variation of the constructs or binding domains of the invention involves substituting one or more residues within the hypervariable region of a parent antibody structure (e.g. a humanized or human antibody structure). Generally, the resulting variant(s) selected for further development will have improved biological properties relative to the parent antibody structure from which they are generated. A convenient way for generating such substitutional variants involves affinity maturation using phage display. Briefly, several sites of the hypervariable region (e. g. 6-7 sites) are mutated to generate all possible amino acid substitutions at each site. The variants thus generated are displayed in a monovalent fashion from filamentous phage particles as fusions to the gene III product of M13 packaged within each particle. The phage-displayed variants are then screened for their biological activity (e.g. binding affinity) as disclosed herein. To identify candidate hypervariable region sites contributing significantly to antigen binding (candidates for modification), alanine scanning mutagenesis can also be performed. Alternatively, or additionally, it may be beneficial to analyze a crystal structure of the complex between the antigen and the construct or the binding domain to identify contact points between the binding domain and its specific antigen. Such contact residues and neighbouring residues are candidates for substitution according to the techniques elaborated herein. Once such variants are generated, the panel of variants is subjected to screening as described herein and antibodies, their antigen-binding fragments, constructs or binding domains with superior properties in one or more relevant assays may be selected for further development.

The constructs and binding domains of the present invention specifically include “chimeric” versions in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments or variants of such antibodies, so long as they exhibit the desired biological activity (U.S. Pat. No. 4,816,567; Morrison et al., Proc. Natl. Acad. Sci. USA, 81: 6851-6855 (1984)). Chimeric constructs or binding domains of interest herein include “primitized” constructs comprising variable domain antigen-binding sequences derived from a non-human primate (e.g., Old World Monkey, Ape etc.) and human constant region sequences. A variety of approaches for making chimeric antibodies or constructs have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, polypeptide construct, antibody fragment, antibody variant or binding domain may also be modified by specific deletion of human T cell epitopes (a method called “deimmunization”) using methods disclosed for example in WO 98/52976 or WO 00/34317. Briefly, the heavy and light chain variable regions of an antibody, construct or binding domain can be analyzed for peptides that bind to MHC class II; these peptides represent potential T cell epitopes (as defined e.g. in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains or variable regions, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. MoI. Biol. 227:776-798; Cook, G. P. et al. (1995) Immunol. Today Vol. 16 (5): 237-242; and Tomlinson et al. (1995) EMBO J. 14: 14:4628-4638. The V BASE directory (www2.mrc-lmb.cam.ac.uk/vbase/list2.php) provides a comprehensive directory of human immunoglobulin variable region sequences (compiled by Tomlinson, LA. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, variants or fragments thereof, constructs and binding domains are based on immunoglobulins of mostly human sequences, which contain (a) minimal sequence(s) derived from non-human immunoglobulin. For the most part, humanized antibodies, variants or fragments thereof, constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, constructs and binding domains as used herein may also comprise residues which are found neither in the recipient antibody nor the donor antibody. These modifications are made to further refine and optimize antibody performance. The humanized antibodies, variants or fragments thereof, constructs and binding domains may also comprise at least a portion of an immunoglobulin constant region (such as Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies, variants or fragments thereof, constructs and binding domains can be generated by replacing sequences of the (Fv) variable region that are not directly involved in antigen binding with equivalent sequences from human (Fv) variable regions. Exemplary methods for generating such molecules are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. These methods include isolating, manipulating, and expressing the nucleic acid sequences that encode all or part of immunoglobulin (Fv) variable regions from at least one of a heavy or light chain. Such nucleic acids may be obtained from a hybridoma producing an antibody against a predetermined target, as described above, as well as from other sources. The recombinant DNA encoding the humanized antibody, variant or fragment thereof, construct or binding domain can then be cloned into an appropriate expression vector.

Humanized antibodies, variants or fragments thereof, constructs and binding domains may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized molecules described herein (U.S. Pat. No. 5,225,539). All the CDRs of a given human sequence may be replaced with at least a portion of a non-human CDR, or only some of the CDRs may be replaced with non-human CDRs. It is only necessary to replace the number of CDRs required for binding of the humanized molecule to a predetermined antigen.

A humanized antibody, variant or fragment thereof, construct or binding domain can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

Human anti-mouse antibody (HAMA) responses have led the industry to prepare chimeric or otherwise humanized antibodies/constructs. It is however expected that certain human anti-chimeric antibody (HACA) responses will be observed, particularly in chronic or multi-dose utilizations of an antibody or construct. Thus, it would be desirable to provide constructs comprising a human binding domain against CLDN6 and/or a human binding domain against CD3, to vitiate concerns and/or effects of HAMA or HACA response.

Therefore, according to one embodiment, the polypeptide construct, one binding domain and/or another binding domain are “human”. The term “human antibody”, “human construct” and “human binding domain” includes antibodies, constructs and binding domains, respectively, having antibody-derived regions such as variable and constant regions or domains which correspond substantially to human germline immunoglobulin sequences known in the art, including, for example, those described by Kabat et al. (1991) (loc. cit.). The human constructs or binding domains of the invention may include amino acid residues not encoded by human germline immunoglobulin sequences (e.g., mutations introduced by random or site-specific mutagenesis in vitro or by somatic mutation in vivo), for example in the CDRs, and particularly in CDR3. The human constructs or binding domains can have at least one, two, three, four, five, or more positions replaced with an amino acid residue that is not encoded by the human germline immunoglobulin sequence. The definition of human antibodies, constructs and binding domains as used herein also contemplates fully human antibodies, constructs and binding domains which include only non-artificially and/or genetically altered human sequences of antibodies as those can be derived by using technologies or systems such as the Xenomouse.

Polypeptides/polypeptide constructs comprising at least one human binding domain avoid some of the problems associated with antibodies or constructs that possess non-human such as rodent (e.g. murine, rat, hamster or rabbit) variable and/or constant regions. The presence of such rodent derived proteins can lead to the rapid clearance of the antibodies or constructs or can lead to the generation of an immune response against the antibody or construct by a patient. To avoid the use of rodent-derived constructs, humanized or fully huma constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or constructs derived therefrom are expected to minimize the immunogenic and allergic responses intrinsic to mouse or mouse-derivatized mAbs and thus to increase the efficacy and safety of the administered antibodies/constructs. The use of fully human antibodies or constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse model is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. No. 5,591,669 and U.S. Pat. No. 6,023.010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the constructs of the invention are “isolated” or “substantially pure” constructs. “Isolated” or “substantially pure”, when used to describe the constructs disclosed herein, means a construct that has been identified, separated and/or recovered from a component of its production environment. Preferably, the construct is free or substantially free of association with all other components from its production environment. Contaminant components of its production environment, such as that resulting from recombinant transfected cells, are materials that could interfere with diagnostic or therapeutic uses for the construct, and may include enzymes, hormones, and other proteinaceous or non-proteinaceous compounds. It is understood that the isolated or substantially pure construct may constitute from 5% to 99.9% by weight of the total protein/polypeptide content in a given sample, depending on the circumstances. The desired construct may be produced at a significantly higher concentration using an inducible promoter or high expression promoter. The definition includes the production of a construct in a wide variety of organisms and/or host cells that are known in the art. In certain embodiments, the construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie Blue or, preferably, silver staining. Usually, however, an isolated construct will be prepared by at least one purification step.

According to one embodiment, the entire construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins based on size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule, as mentioned above. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The terms “selectively” and, “preferably, selectively”, “(specifically or immunospecifically) binds to”, “(specifically or immunospecifically) recognizes”, or “(specifically or immunospecifically) reacts with” mean in accordance with this invention that a construct or a binding domain selectively interacts or (immuno-)specifically interacts with a given epitope on the target molecule (antigen), here: CLDN6 and CD3, respectively. This selective interaction or association occurs more frequently, more rapidly, with greater duration, with greater affinity, or with some combination of these parameters, to an epitope on the specific target (here CLDN6) than to alternative substances (non-target molecules, e.g., here CLDN4, CLDN9, CLDN3, etc.). Because of the sequence similarity between homologous proteins in different species, a construct or a binding domain that selectively and/or immunspecifically binds to its target (such as a human target) may, however, cross-react with homologous target molecules from different species (such as, from non-human primates). The terms “selectively binds to”, “specific/immunospecific binding”, etc. can hence include the binding of a construct or binding domain to epitopes or structurally related epitopes in more than one species. In the context of the present invention, a polypeptide of the present invention binds to its respective target structure in a particular manner. Preferably, a polypeptide according to the present invention comprises one paratope per binding domain which specifically or immunospecifically binds to”, “(specifically or immunospecifically) recognizes”, or “(specifically or immunospecifically) reacts with” its respective target structure. This means in accordance with this invention that a polypeptide or a binding domain thereof interacts or (immuno-)specifically interacts with a given epitope on the target molecule (antigen) and CD3, respectively. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, an antibody construct or a binding domain that immunspecifically binds to its target (such as a human target) may, however, cross-react with homologous target molecules from different species (such as, from non-human primates). The term “specific/immunospecific binding” can hence include the binding of an antibody construct or binding domain to epitopes and/or structurally related epitopes in more than one species. The term “(immuno-) selectively binds does exclude the binding to structurally related epitopes.

In the context of the present invention, the term “epitope” refers to the part or region of the antigen that is selectively recognized/immunospecifically recognized by the binding structure, i.e. the paratope. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved because of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

A method for CLDN6 epitope mapping is described in the following: A pre-defined region (a contiguous amino acid stretch) within the extracellular loops of human CLDN6 protein is exchanged/replaced with a corresponding region of a CLDN6 paralogue (such as human CLDN4 or human CLDN18.2, but other paralogues are also conceivable, so long as the binding domain is not cross-reactive with the paralogue used). These human CLDN6/paralogue chimeras are expressed on the surface of host cells (such as CHO cells). Binding of the antibody or construct can be tested via FACS analysis. When the binding of the antibody or construct to the chimeric molecule is entirely abolished, or when a significant binding decrease is observed, it can be concluded that the region of human CLDN6 which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to human (wild-type) CLDN6, whereby binding to human CLDN6 is set to be 100%. Alternatively, the above described epitope mapping analysis can be modified by introducing one or several point mutations into the sequence of CLDN6, specifically in the sequences of the extracellular loop 1 or loop 2, more specifically in the sequence corresponding to the E1A and/or E2B regions of these loops that are depicted in SEQ ID NOS: 9 and 10. These point mutations can e.g. reflect the differences between CLDN6 and its closely related paralogue CLDN4.

A further method to determine the contribution of a specific residue of a target antigen to the recognition by a construct or binding domain is alanine scanning (see e.g. Morrison K L & Weiss G A. Curr Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired.

The interaction between the binding domain and the epitope of the target antigen implies that a binding domain exhibits appreciable or significant affinity for the epitope/the target antigen (here: CLDN6 and CD3, respectively) and, generally, does not exhibit significant affinity for proteins or antigens other than the target antigen (here: CLDN6/CD3)—notwithstanding the above discussed cross-reactivity with homologous targets e.g. from other species, or with CLDN9 of the same species, particularly human CLDN6 and CLDN9. “Significant affinity” includes binding with an affinity (dissociation constant, KD) of ≤10-6 M. Preferably, binding is considered specific when the binding affinity is ≤10-7 M, ≤10-8 M, ≤10-9 M, ≤10-10 M, or even ≤10-11 M, or ≤10-12 M. Whether a binding domain (immuno-)specifically reacts with or binds to a target can be tested readily e.g. by comparing the affinity of said binding domain to its desired target protein or antigen with the affinity of said binding domain to non-target proteins or antigens (here: proteins other than CLDN6 or CD3, respectively). Preferably, a construct of the invention does not significantly bind to proteins or antigens other than CLDN6 or CD3, respectively (i.e., the first domain does not bind to proteins other than CLDN6 and the second domain does not bind to proteins other than CD3)—unless any further binding domain(s) directed against a further target is/are deliberately introduced into the construct of the invention, in which case the binding of that binding domain to its specific target is also provided by the present invention.

It is envisaged that the affinity of the first domain for CLDN6 (e.g. human CLDN6) is ≤100 nM, ≤90 nM, ≤80 nM, ≤70 nM, ≤60 nM, ≤50 nM, ≤40 nM, ≤30 nM, or ≤20 nM. These values are preferably measured in a cell-based assay, such as a Scatchard assay. Other methods of determining the affinity are also well-known. It is furthermore envisaged that the affinity of the second domain for CD3 (e.g. human CD3) is ≤100 nM, ≤90 nM, ≤80 nM, ≤70 nM, ≤60 nM, ≤50 nM, ≤40 nM, ≤30 nM, ≤20 nM, or ≤10 nM. These values are preferably measured in a surface plasmon resonance assay, such as a Biacore assay.

The term “does not significantly bind” and “does not selectively bind” mean that a construct or binding domain of the present invention does not bind to a protein or antigen other than CLDN6 or CD3, when said protein or antigen is expressed on the surface of a cell. The construct hence shows reactivity of 30%, preferably 20%, more preferably 10%, particularly preferably ≤9%, 8%, ≤7%, 6%, ≤5%, ≤4%, ≤3%, 2%, or ≤1% with proteins or antigens other than CLDN6 or CD3 (when said proteins or antigens are expressed on the surface of a cell), whereby binding to CLDN6 or CD3, respectively, is set to be 100%. The “reactivity” can e.g. be expressed in an affinity value (see above).

It is envisaged that the construct of the invention (and more specifically the domain comprising a paratope that binds to CLDN6) does not bind or does not significantly bind to CLDN6 paralogues, more specifically to human CLDN6 paralogues and/or to macaque/cyno CLDN6 paralogues. It is also envisaged that the construct does not bind or does not significantly bind to (human or macaque/cyno) CLDN6 paralogues on the surface of a target cell. The CLDN6 paralogues include—but are not limited to CLDN1, CLDN2, CLDN3, CLDN4, CLDN18.1, CLDN18.2, and particularly CLDN9. According to one embodiment, the human paralogues of CLDN6 have sequences as depicted in SEQ ID NOs: 2-8. It is hence envisaged that the first domain of the construct of the invention does not bind or does not significantly selectively bind to CLDN1, CLDN2, CLDN3, CLDN4, CLDN18.1, CLDN18.2, and/or CLDN9 (on the surface of a cell). It is envisaged that the constructs of the present invention substantially do not bind to CLDN9, which is expressed on various organs. Selective binding to CLDN6, and essentially no binding to CLDN9, avoids potential adverse events that could arise by off-target binding.

One domain comprising a paratope (antigen-binding (epitope-binding) structure) of the polypeptide construct of the invention binds specifically and/or selectively to CLDN6 on the surface of a target cell. The “target cell” can be any prokaryotic or eukaryotic cell expressing CLDN6 on its surface; preferably the target cell is a cell that is part of the human or animal body, such as a specific CLDN6 expressing cancer cell or tumor cell or a cell of a CLDN6 positive neoplasm or an artificially generated CLDN6 expressing cell (the latter may be used, for example, in assays ex vivo). It is understood that the term “on the surface”, in the context of the present invention, means that the first antigen-binding domain of the construct selectively and, preferably, specifically binds to an epitope comprised within the first CLDN6 extracellular loop (CLDN6 ECL1), within the second CLDN6 extracellular loop (CLDN6 ECL2), particularly it binds to an epitope that is formed by a combination of both loops. It is hence envisaged that the domain comprising a paratope (antigen-binding (epitope-binding) structure) of the construct of the invention binds to an epitope formed by one or both extracellular loops of CLDN6, preferably of human CLDN6. The extracellular loop can be the first loop and/or the second loop. It is also particularly envisaged that both loops contribute to the binding. In this case, it is possible that one loop (such as the first loop) represents the main binding partner for the construct, and the other loop (such as the second loop) contributes to the binding, e.g. as a stabilizing partner, but is not essential for the binding. The domain comprising a paratope (antigen-binding (epitope-binding) structure) according to the invention may hence bind to CLDN6 when it is expressed by naturally expressing cells or cell lines (such as human cancer lines OVCAR-3, OAW28, LCLC97TM1, and NCI-H1435), and/or by cells or cell lines transformed or (stably/transiently) transfected with CLDN6. In one embodiment, the domain comprising a paratope (antigen-binding (epitope-binding) structure) that binds to CLDN6 when CLDN6 is used as a target molecule in a cell-based binding assay such as Scatchard. It is furthermore envisaged that the construct/its first domain binds to human CLDN6 on the surface of a target cell. A preferred amino acid sequence for human CLDN6 is depicted in SEQ ID NO: 1.

It is envisaged that the polypeptide construct according to the invention (and, more specifically, the domain comprising a paratope (antigen-binding (epitope-binding) structure) binding CLDN6 of said construct) binds to the first extracellular loop (ECL1, loop 1) of CLDN6. This does not necessarily exclude that the second extracellular loop also contributes, albeit to a different, e.g. a lesser, extent, to the paratope-eptiope interaction site. The term “CLDN6 ECL” (ECL=extracellular loop) refers to those parts of of CLDN6 which are essentially free of the transmembrane and cytoplasmic domains of CLDN6. It is understood that the transmembrane domains identified for the CLDN6-binding polypeptide of the present invention are identified pursuant to criteria routinely employed in the art for identifying that type of hydrophobic domain. The exact boundaries of a transmembrane domain may vary but most likely by no more than about 5 amino acids at either end of the domain explicitly mentioned herein. A preferred human CLDN6 ECL1 is shown in SEQ ID NO: 9, and a preferred human CLDN6 ECL2 is shown in SEQ ID NO: 10. In one very specific embodiment the construct according to the invention (and, more specifically, the first domain of said construct) binds to the the E1A domain of the first extracellular loop (ECL1, loop 1) and to the E2B domain of the second extracellular loop (ECL2, loop 2) of CLDN6, preferably of human CLDN6, which is advantageously expressed on the surface of a cancer cell or a cell that has been induced to express CLDN6, e.g. human CLDN6, by transformation or transfection, and do not bind to amino acids 138-150 of CLDN6 as depicted in SEQ ID NO: 1.

The present invention furthermore provides that the domain comprising a paratope (antigen-binding (epitope-binding) structure) of the construct of the invention, preferably selectively, binds to the same epitope of CLDN6 as an antibody or construct comprising a domain which binds to CLDN6 on the surface of a target cell and which comprises:
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256, or
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270, or
a-1) a VH region as depicted in SEQ ID NO: 11, and a VL region as depicted in SEQ ID NO: 12;
b-1) a VH region as depicted in SEQ ID NO: 19, and a VL region as depicted in SEQ ID NO: 20;
c-1) a VH region as depicted in SEQ ID NO: 27, and a VL region as depicted in SEQ ID NO: 28;
d-1) a VH region as depicted in SEQ ID NO: 35, and a VL region as depicted in SEQ ID NO: 36;
e-1) a VH region as depicted in SEQ ID NO: 43, and a VL region as depicted in SEQ ID NO: 44;
f-1) a VH region as depicted in SEQ ID NO: 51, and a VL region as depicted in SEQ ID NO: 52;
g-1) a VH region as depicted in SEQ ID NO: 59, and a VL region as depicted in SEQ ID NO: 60;
h-1) a VH region as depicted in SEQ ID NO: 67, and a VL region as depicted in SEQ ID NO: 68;
i-1) a VH region as depicted in SEQ ID NO: 75, and a VL region as depicted in SEQ ID NO: 76;
j-1) a VH region as depicted in SEQ ID NO: 83, and a VL region as depicted in SEQ ID NO: 84;
k-1) a VH region as depicted in SEQ ID NO: 91, and a VL region as depicted in SEQ ID NO: 92;
l-1) a VH region as depicted in SEQ ID NO: 99, and a VL region as depicted in SEQ ID NO: 100;
m-1) a VH region as depicted in SEQ ID NO: 107, and a VL region as depicted in SEQ ID NO: 108;
n-1) a VH region as depicted in SEQ ID NO: 115, and a VL region as depicted in SEQ ID NO: 116;
o-1) a VH region as depicted in SEQ ID NO: 123, and a VL region as depicted in SEQ ID NO: 124;
p-1) a VH region as depicted in SEQ ID NO: 131, and a VL region as depicted in SEQ ID NO: 132;
q-1) a VH region as depicted in SEQ ID NO: 139, and a VL region as depicted in SEQ ID NO: 140; or
r-1) a VH region as depicted in SEQ ID NO: 147, and a VL region as depicted in SEQ ID NO: 148, or
s-1) a VH region as depicted in SEQ ID NO: 263, and/or a VL region as depicted in SEQ ID NO: 264.

In further embodiments, the polypeptide constructs of the present invention comprise a domain binding CLDN6, which comprises any of the CDR regions depicted in SEQ ID NOs: 680 to 694, e.g., heavy chain CDR1 depicted in SEQ ID NO: 680, or heavy chain CDR2 depicted in SEQ ID NO: 681, or heavy chain CDR2 depicted in SEQ ID NO: 682, or heavy chain CDR2 depicted in SEQ ID NO: 683, or heavy chain CDR3 depicted in SEQ ID NO: 684, or heavy chain CDR3 depicted in SEQ ID NO: 685, or heavy chain CDR3 depicted in SEQ ID NO: 686, or heavy chain CDR3 depicted in SEQ ID NO: 687, and/or light chain CDR1 depicted in SEQ ID NO: 688, light chain CDR1 depicted in SEQ ID NO: 689, light chain CDR2 depicted in SEQ ID NO: 690, light chain CDR3 depicted in SEQ ID NO: 691, light chain CDR3 depicted in SEQ ID NO: 692, light chain CDR3 depicted in SEQ ID NO: 693, and light chain CDR3 depicted in SEQ ID NO: 694, or any combinations thereof comprising heavy chain sequences as depicted in SEQ ID NOs: 680 to 687 and/or combinations thereof comprising light chain sequences as depicted in SEQ ID NOs: 688 to 694. A person of skill in the art knows that a binding domain binding CLDN6 may comprise only one of a HCDR1, HCDR2, HCDR3, LCDR1, LCDR2, and LCDR3, which may be present in the combinations according to the invention.

Other anti-CLDN6 binders were also analyzed for their CLDN6 binding specificities during epitope mapping (see Example 2). These CLDN6×CD3 constructs were found to have a different epitope specificity, and at the same time were shown to have a significantly inferior cytotoxic potential compared with the constructs of the invention. In Example 4, it was demonstrated that the constructs of the present invention display EC50 values in the two-digit picomolar range, while the comparative constructs displayed EC50 values in the three-digit up to the five-digit picomolar range, despite having similar affinities for CLDN6. Constructs displaying cytotoxic activity of the latter range might not be potent enough for a therapeutic use in directing a patient's immune system, more specifically the T cells' cytotoxic activity, against cancer cells. On the other hand, constructs according to the invention present with a very favorable epitope-activity relationship, hence supporting potent construct mediated cytotoxic activity.

Whether or not an antibody, polypeptide construct or a domain comprising a paratope (antigen-binding (epitope-binding) structure) binds to the same epitope of CLDN6 on the surface of a target cell as another given antibody, construct or binding domain can be measured by different analyses as described herein, e.g. by epitope mapping with chimeric or mutated CLDN6 molecules, as described herein above or in Examples 1 and 2. Other methods of determining epitopes are described herein, such as alanine scanning.

Whether or not an antibody or polypeptide construct or a domain comprising a paratope (antigen-binding (epitope-binding) structure) competes for binding to an antigen (such as CLDN6) on the surface of a target cell with another given antibody or construct can be measured in a competition assay such as a competitive ELISA. Avidin-coupled microparticles (beads) can also be used. Similarly, to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. Preferably a cell-based competition assay is used, using either cells that naturally express CLDN6 or cells that were stably or transiently transformed with CLDN6. The term “competes for binding”, in the present context, means that competition occurs between the two tested antibodies of at least 20%, at least 30%, at least 40%, at least 50%, at least 60%, at least 70%, at least 80% or at least 90%, as determined by any one of the assays disclosed above, preferably the cell-based assay. The same analysis can of course be applied for other targets such as CD3.

Competitive antibody/polypeptide construct binding assays include assays determining the competitive binding of two antibodies/constructs to a cell surface bound antigen. Common methods aim to detect binding of two antibodies/constructs, A and B, to the same antigen on the surface of a cell may include steps of:
a) blocking of the cell surface antigen by pre-incubation of cells with antibody/polypeptide construct A followed by a sub-maximal addition of labeled antibody/polypeptide construct B and detecting the binding of B compared with binding in the absence of A;
b) titration (i.e. adding different amounts) of antibody/polypeptide construct A in the presence of sub-maximal amounts of labeled antibody/polypeptide construct B and detecting the effect on binding of B; or
c) co-titration of A and B, wherein both antibodies/polypeptides/polypeptide constructs are incubated together at maximal concentration and detecting whether the total binding equals or exceeds that of either A or B alone, i.e. a method which cannot be affected by the order of addition or relative amounts of the antibodies/constructs.

When two antibodies/polypeptides/polypeptide constructs A and B compete for a cell surface bound antigen, the antibodies will very often compete in blocking assays independently from the order of the addition of the antibodies. In other words, competition is detected if the assay is carried out in either direction. However, this is not always the case, and under certain circumstances the order of the addition of the antibodies or the direction of the assay may influence the signal generated. This may be due to differences in affinities or avidities of the potentially competing antibodies/constructs. If the order of the addition has a significant effect on the signal generated, it is concluded that the two antibodies/constructs do compete if competition is detected in at least one order.

In the context of the present invention, the term “variable” refers to those portions of antibody or immunoglobulin domains that exhibit variability in their sequence and that are involved in determining the specificity and binding affinity of a particular antibody (i.e., the “variable region(s)”). Usually, the pairing of a heavy chain variable region (VH) and a light chain variable region (VL) together forms a single antigen-binding site.

Variability is not evenly distributed throughout the variable regions of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable regions are called the “framework” (FR) regions and provide a scaffold for the six CDRs in three-dimensional space to form an antigen-binding surface. The variable regions of naturally occurring antibody heavy and light chains each comprise four FR regions (FR1, FR2, FR3, and FR4), largely adopting a f-sheet configuration. Together with the CDRs, they form the following sequence within a variable heavy or light chain: FR1-CDR1-FR2-CDR2-FR3-CDR3-FR4. The hypervariable regions in each chain are held together by the framework regions and, usually together with the hypervariable regions from the other chain, contribute to the formation of the antigen-binding site (see Kabat et al., loc. cit.). As used herein, the polypeptides/polypeptide constructs of the invention may have modifications in the framework region. These modifications may be substitutions of one or more amino residue of the herein disclosed sequences by other amino acid residues. Further modifications that are possible are deletions, inversions, additions of amino acid residues if these modifications do not prevent the selective binding of the polypeptides/polypeptide constructs to CLDN6 and/or the as long as these modifications do not prevent the selective binding of the polypeptides/polypeptide constructs to CLDN6-expressing target cells and the constructs' ability to engage and activate T cells and induce T cell-mediated cytotoxicity. Accordingly, the modifications of the framework regions of the herein disclosed constructs still allow an at least 100%, at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 65%, at least 60%, at least 55%, at least 50%, at least 45%, at least 40%, at least 35%, at least 30%, at least 25%, at least 20%, at least 15%, at least 10%, at least 5% ability to engage T cells and induce T cell-mediated cytotoxicity when compared with a non-framework-modified antibody. Modifications in the framework regions may also be associated with a higher activity than the non-modified constructs. It is also contemplated to modify the framework regions of the constructs of the present invention for purposes such as increasing the solubility of the construct in a given medium, to increase the stability of the construct, and the like.

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of antibodies (or constructs or binding domain) with an antigen and hence contribute to the functional activity of an antibody molecule: they are the main determinants of antigen specificity.

The exact definition of CDR boundaries and lengths is subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Corresponding loops between antibodies may, therefore, have very similar three-dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable region in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate class sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibodies or constructs/binding domains, the heavy chain CDR3 appears to constitute the major area of contact between the antigen and the antibody. In vitro selection schemes in which CDR3 alone is varied can be used to vary the binding properties of an antibody or construct/binding domain or determine which residues contribute to the binding of an antigen. Hence, CDR3 is typically the greatest source of molecular diversity within the antibody binding site. CDR-H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The heavy chain constant (CH) domain most proximal to VH is usually designated as CH1. The constant (“C”) domains are not directly involved in antigen binding, but exhibit various effector functions, such as antibody-dependent cell-mediated cytotoxicity (ADCC) and complement activation (complement dependent cytotoxicity, CDC). The Fc region of an antibody is the “tail” region of a classical antibody that interacts with cell surface receptors called Fc receptors and some proteins of the complement system. In IgG, IgA and IgD antibody isotypes, the Fc region is composed of two identical protein fragments, derived from the second and third constant domains (CH2 and CH3) of the antibody's two heavy chains. IgM and IgE Fc regions contain three heavy chain constant domains (CH2, CH3 and CH4) in each polypeptide chain. The Fc regions also contains part of the so-called “hinge” region held together by one or more disulfides and noncovalent interactions. The Fc region of a naturally occurring IgG bears a highly conserved N-glycosylation site. Glycosylation of the Fc fragment is essential for Fc receptor-mediated activity.

ADCC is a mechanism of cell-mediated immune defense whereby an effector cell of the immune system actively lyses a target cell, whose membrane-surface antigens have been bound by specific antibodies. ADCC requires an immune effector cell which classically is known to be a natural killer (NK) cell that typically interacts with IgG antibodies. However, ADCC can also be mediated by macrophages, neutrophils and eosinophils. Naturally occurring ADCC involves activation of effector cells expressing Fc receptors by antibodies expressing an Fc portion. For example, the most common Fc receptor on the surface of an NK cell is calles CD16 or FcγRIII. Once the Fc receptor binds to the Fc region of IgG, the NK cell releases cytotoxic factors that cause the death of the target cell. Likewise, the Fc receptor (FceRI) of an eosinophil will recognize IgE. In CDC, in contrast, the molecule “C1q” of the complement system binds to the antibody Fc region, and this binding triggers the complement cascade which leads to the formation of the membrane attack complex (MAC) at the surface of the target cell, as a result of the classical pathway complement activation. In therapeutic antibodies, both ADCC and CDC can be modulated by Fc isotype engineering, Fc genetic mutations, or Fc glycosylation profile modifications. As used herein, the polypeptides/polypeptide constructs of the present invention are not inducing ADCC as commonly understood. Instead, the polypeptides can engage T cells and induce T cell-mediated cytotoxicity, e.g. by secretion of perforin and/or inducing apoptosis.

The sequence of antibody genes after assembly and somatic mutation is highly varied, and these varied genes are estimated to encode 1010 different antibody molecules (Immunoglobulin Genes, 2nd ed., eds. Jonio et al., Academic Press, San Diego, CA, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

It is envisaged that the polypeptide construct of the invention has a cysteine clamp within the first domain. This cysteine clamp may be introduced to improve stability of the construct. See e.g. US 2016/0193295.

In one embodiment of the invention, the CLDN6-binding paratope (antigen-binding (epitope-binding) structure) of one domain of the construct of the invention comprises a VH region having an amino acid sequence as depicted in SEQ ID NO: 11, SEQ ID NO: 25, SEQ ID NO: 39, SEQ ID NO: 67, or SEQ ID NO: 193.

In a further embodiment, the CLDN6-specific paratope, i.e. the antigen-binding (epitope-binding) domain of the construct of the invention comprises a VL region having an amino acid sequence as depicted in SEQ ID NO: 12, SEQ ID NO: 26, SEQ ID NO: 40, SEQ ID NO: 68, or SEQ ID NO: 194.

In another embodiment, the CLDN6-specific paratope, i.e. the antigen-binding (epitope-binding) domain of the construct of the invention comprises a VH region and a VL region having an amino acid sequence as depicted in SEQ ID NOs: 11+12 (VH+VL), SEQ ID NOs: 25+26, SEQ ID NOs: 39+40, SEQ ID NOs: 67+68, or SEQ ID NOs: 193+194 (VH+VL).

In yet a further embodiment, the CLDN6-specific paratope, i.e. the antigen-binding (epitope-binding) domain of the construct of the invention comprises a polypeptide having an amino acid sequence as depicted in SEQ ID NO: 19, SEQ ID NO: 22, SEQ ID NO: 33, SEQ ID NO: 36, SEQ ID NO: 47, SEQ ID NO: 50, SEQ ID NO: 76, SEQ ID NO: 78, SEQ ID NO: 201, or SEQ ID NO: 204, particularly SEQ ID NOs: 19 and 22.

As described herein above, the invention provides an embodiment wherein the polypeptide construct is in a format selected from the group consisting of (scFv)2, scFv-single domain mAb, diabodies and oligomers of any of the aforementioned formats. The term “is in a format” does not exclude that the construct can be further modified, e.g. by attachment or fusion to other moieties, as described herein. According to one embodiment of the polypeptide construct of the present invention, the domains comprising the herein described paratopes are in the format of an scFv. In an scFv, the VH region and the and VL region are arranged in the order VH-VL or VL-VH (from N- to C-terminus). It is envisaged that the VH and the VL regions of the domains comprising the herein described paratopes are connected via a linker, preferably a peptide linker. According to one embodiment of the domains comprising the herein described paratopes, the VH-region is positioned N-terminally of the linker, and the VL-region is positioned C-terminally of the linker. In other words, in one embodiment of the domains comprising the herein described paratopes, the scFv comprises from the N-terminus to the C-terminus: VH-linker-VL. It is furthermore envisaged that the domains comprising the herein described paratopes of the construct are connected via a linker, preferably a peptide linker. The construct may e.g. comprise the domains in the order (from N-terminus to C-terminus) one domain—linker—second further domain. The inverse order (further domain—linker—first domain) is also possible.

The linkers are preferably peptide linkers, more preferably short peptide linkers. In accordance with the present invention, a “peptide linker” comprises an amino acid sequence which connects the amino acid sequences of one domain with another (variable and/or binding) domain (e.g. a variable domain or a binding domain) of the construct. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the construct of the invention. Examples of useful peptide linkers are shown in SEQ ID NOs: 563-575 and SEQ ID NO: 679. In the present context, a “short” linker has between 2 and 50 amino acids, preferably between 3 and 35, between 4 and 30, between 5 and 25, between 6 and 20, or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13, and the linker between the two binding domains may have a length between 3 and 10 amino acids, preferably between 4 and 8. It is further envisaged that the peptide linkers are glycine/serine linkers, such as those depicted in SEQ ID NOs: 563-575 and SEQ ID NO: 679. Most of the amino acids in glycine/serine linkers are selected from glycine and serine.

If a linker is used, this linker is preferably of a length and sequence to ensure that each of the first and second domains can, independently from one another, retain their differential binding specificities. For peptide linkers which connect the at least two binding domains (or the two variable regions forming one binding domain) in the construct, those peptide linkers are envisaged which comprise only a few amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A “single amino acid” linker in the context of said “peptide linker” is Gly. Another embodiment of a peptide linker is characterized by the amino acid sequence Gly-Gly-Gly-Gly-Ser, i.e. Gly4Ser (SEQ ID NO: 563), or polymers thereof, i.e. (Gly4Ser)x, where x is an integer of 1 or greater (e.g. 2 or 3). Usable linkers are depicted in SEQ ID NOs: 563-575 and SEQ ID NO: 679. The characteristics of said peptide linkers are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which do not promote any secondary structures are preferred. The linkage of said domains to each other can be provided, e.g., by genetic engineering. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

According to one embodiment of the invention, the polypeptide construct of the invention is a “single chain construct”. It is also envisaged that either the first or the second or both binding domains may be in the format of a “single chain Fv” (scFv). Although the two domains of the Fv fragment, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brdhl, Immunol., (2001), 166, 2420-2426, Kipriyanov, J. Mol. Biol., (1999), 293, 41-56. Techniques described for producing single chain constructs (see, inter alia, U.S. Pat. No. 4,946,778, Kontermann and Dübel (2010), loc. cit. and Little (2009), loc. cit.) can be adapted to produce single chain constructs selectively and, preferably, specifically recognizing (an) elected target(s).

Bivalent (also called divalent) or bispecific single-chain variable fragments (bi-scFvs or di-scFvs) having the format (scFv)2 can be engineered by linking two scFv molecules (e.g. with linkers as described hereinbefore). The linking can be done by producing a single polypeptide chain with two VH regions and two VL regions, yielding tandem scFvs (see e.g. Kufer, P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. In this case, the VH and th VL of a binding domain (binding either to the target antigen CLDN6 or to CD3) are not directly connected via a peptide linker. Thus, the VH of the CD3 binding domain may e.g. be fused to the VL of the CLDN6 binding domain via a peptide linker, and the VH of the CLDN6 binding domain is fused to the VL of the CD3 binding domain via such peptide linker. This type is known as diabodies (see e.g. Hollinger, Philipp et al., (July 1993) Proceedings of the National Academy of Sciences of the United States of America 90 (14): 6444-8).

Constructs denominated “single domain constructs” (or occasionally “antibody constructs”) comprise one (monomeric) antibody variable region which can bind selectively to a specific antigen, independently of other variable regions. The first single domain antibodies were engineered from heavy chain antibodies found in camelids, and these are called VHH fragments. Cartilaginous fishes also have heavy chain antibodies (IgNAR) from which single domain antibodies called VNAR fragments can be obtained. An alternative approach is to split the dimeric variable regions from common immunoglobulins into monomers, hence obtaining VH or VL as a single domain Ab. Although most research into single domain antibodies is currently based on heavy chain variable regions, nanobodies derived from light chains were also shown to bind specifically to target epitopes. Examples of single domain antibodies are called sdAb, nanobodies or single variable domain antibodies. A (single domain mAb)2 is hence a monoclonal construct composed of (at least) two single domain monoclonal constructs, which are individually selected from the group comprising VH, VL, VHH and VNAR. The linker is preferably in the form of a peptide linker. Similarly, an “scFv-single domain mAb” is a monoclonal construct composed of at least one single domain antibody as described above and one scFv molecule as described above. Again, the linker is preferably in the form of a peptide linker.

It is also envisaged that the polypeptide construct of the invention has, in addition to its function to bind to the target molecules CLDN6 and CD3, a further function. In this format, the construct may be a trifunctional or multifunctional construct by targeting target cells through CLDN6 binding, mediating cytotoxic T cell activity through CD3 binding and providing a further function such as means or domains to enhance or extend serum half-life, a fully functional or modified Fc constant domain mediating cytotoxicity through recruitment of effector cells, a label (fluorescent etc.), a therapeutic agent such as a toxin or radionuclide, etc.

Examples for means or domains to extend serum half-life of the polypeptides/polypeptide constructs of the invention include peptides, proteins or domains of proteins, which are fused or otherwise attached to the polypeptides/polypeptide constructs. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the constructs of the present invention. The concept of attaching larger domains of proteins or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may e.g. be optimized/modified to allow the desired pairing of dimers or mulimers, to abolish Fc receptor binding (e.g. to avoid ADCC or CDC) or for other reasons. A further concept known in the art to extend the half-life of substances or molecules in the human body is the pegylation of those molecules (such as the constructs of the present invention).

In one embodiment, the polypeptides/polypeptide constructs according to the invention are linked (e.g. via peptide bond) with a fusion partner (such as a protein, polypeptide or peptide), e.g. for extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. Exemplary sequences of these fusion partners are depticed in SEQ ID NOs: 576-637. In general, the fusion partners may be linked to the N-terminus or to the C-terminus of the constructs according to the invention, either directly (e.g. via peptide bond) or through a peptide linker such as (GGGGS)n (wherein “n” is an integer of 2 or greater, e.g. 2 or 3 or 4). Suitable peptide linkers are discussed above and are shown in SEQ ID NOs: 563-575.

Hence, it is envisaged that a polypeptide construct according to the present invention comprises a polypeptide comprising in the following order from N-terminus to C-terminus:
a) VL (comprising part of the CLDN6 binding paratope)—(G4S)3—VH (comprising part of the CLDN6 binding paratope)—Peptide linker (SG4S)—VH (comprising part of the CD3 binding paratope)—(G4S)3—VL (comprising part of the CD3 binding paratope)—Peptide linker (G4)—Fc monomer (part of the HLE domain)—(G4S)6—Fc monomer (part of the HLE domain); or
b) VH (comprising part of the CLDN6 binding paratope)—(G4S)3—VL (comprising part of the CLDN6 binding paratope)—Peptide linker (SG4S)—VH (comprising part of the CD3 binding paratope)—(G4S)3—VL (comprising part of the CD3 binding paratope)—Peptide linker (G4)—Fc monomer (part of the HLE domain)—(G4S)6—Fc monomer (part of the HLE domain).

Hence, it is envisaged that a polypeptide construct according to the present invention comprises:
(a) a polypeptide comprising in the following order from N-terminus to C-teminus:
   a polypeptide having an amino acid sequence selected from the group consisting of SEQ ID NO: 12, SEQ ID NO: 26, SEQ ID NO: 40, SEQ ID NO: 68, and SEQ ID NO: 194;
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID NO: 11, SEQ ID NO: 25, SEQ ID NO: 39, SEQ ID NO: 67, and SEQ ID NO: 193;
(b) a polypeptide comprising in the following order from N-terminus to C-teminus:
   a polypeptide having an amino acid sequence selected from the group consisting of SEQ ID NO: 11, SEQ ID NO: 25, SEQ ID NO: 39, SEQ ID NO: 67, and SEQ ID NO: 193
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID SEQ ID NO: 12, SEQ ID NO: 26, SEQ ID NO: 40, SEQ ID NO: 68, and SEQ ID NO: 194;
(c) a polypeptide comprising in the following order from N-terminus to C-teminus:
   a polypeptide having an amino acid sequence selected from the group consisting of SEQ ID NO: 12 and SEQ ID NO: 194, particularly SEQ ID NO: 12;
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID NO: 11 and SEQ ID NO: 193, particularly SEQ ID NO: 11;
(d) a polypeptide comprising in the following order from N-terminus to C-teminus:
   a polypeptide having an amino acid sequence selected from the group consisting of SEQ ID NO: 25, SEQ ID NO: 39, and SEQ ID NO: 67,
   a peptide linker having an amino acid sequence selected from the group consisting of SEQ ID NO: 563, which can be replaced by any one of SEQ ID NOs: 564-575; and 679; and
   a polypeptide having an amino acid sequence selected from the group consisting of SEQ ID NO: 26, SEQ ID NO: 40, and SEQ ID NO: 68;
(e) a polypeptide comprising in the following order from N-terminus to C-teminus:
   a polypeptide having an amino acid sequence selected from the group consisting of SEQ ID NO: 12, SEQ ID NO: 26, SEQ ID NO: 40, SEQ ID NO: 68, and SEQ ID NO: 194;
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID NO: 11, SEQ ID NO: 25, SEQ ID NO: 39, SEQ ID NO: 67, and SEQ ID NO: 193;
(f) a polypeptide comprising in the following order from N-terminus to C-teminus:
   a polypeptide having an amino acid sequence selected from the group consisting of SEQ ID NO: 12, SEQ ID NO: 26, SEQ ID NO: 40, SEQ ID NO: 68, and SEQ ID NO: 194;
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID NO: 11, SEQ ID NO: 25, SEQ ID NO: 39, SEQ ID NO: 67, and SEQ ID NO: 193;
   a peptide linker having an amino acid sequence selected from the group consisting of SEQ ID NO: 565, which can be replaced by any one of SEQ ID NOs: 563, 564, 566-575; or 679; and
   a polypeptide having an amino acid sequence selected from the group consisting of SEQ ID NO: 19, 22, 33, 36, 47, 50, 75, 78, 201, and 204, particularly 19, and 22;
(g) a polypeptide comprising in the following order from N-terminus to C-teminus:
   a polypeptide having an amino acid sequence selected from the group consisting of SEQ ID NO: 11, SEQ ID NO: 25, SEQ ID NO: 39, SEQ ID NO: 67, and SEQ ID NO: 193
   a peptide linker having an amino acid sequence selected from the group consisting of SEQ ID NO: 563, which can be replaced by any one of SEQ ID NOs: 564-575; or 679; and
   a polypeptide having an amino acid sequence selected from the group consisting of SEQ ID SEQ ID NO: 12, SEQ ID NO: 26, SEQ ID NO: 40, SEQ ID NO: 68, and SEQ ID NO: 194
   a peptide linker having an amino acid sequence selected from the group consisting of SEQ ID NO: 565, which can be replaced by any one of SEQ ID NOs: 563, 564, 566-575; or 679; and
   a polypeptide having an amino acid sequence selected from the group consisting of SEQ ID NO: 19, 22, 33, 36, 47, 50, 75, 78, 201, and 204, particularly 19, and 22.

According to another embodiment, the construct of the invention comprises (in addition to the domains comprising the herein described paratopes binding CLDN6 and CD3) an additional domain which comprises two polypeptide monomers, each comprising a hinge, a CH2 and a CH3 domain, wherein said two polypeptide monomers are fused to each other via a peptide linker. It is envisaged that said third domain comprises in N-terminal to C-terminal order: hinge-CH2-CH3-linker-hinge-CH2-CH3. Amino acid sequences that can be used for said third domain are depicted in SEQ ID NOs: 581-637. Each of said polypeptide monomers can have an amino acid sequence that is selected from the group consisting of SEQ ID NOs: 630-637, or that is at least 90% identical to those sequences.

One preferred polypeptide monomer is depicted in SEQ ID NO: 622, and a preferred third domain is depicted in SEQ ID NO: 630.

In another embodiment, the first and second domains of the construct of the invention are fused to the third domain via a peptide linker which is for example selected from the group consisting of SEQ ID NO: 563, 564, 565, 566, 567, 568, 569, 570, 571, 572, 573, 574, and 575 and SEQ ID NO: 679.

In line with the present invention, a “hinge” is an IgG hinge region. This region can be identified by analogy using the Kabat numbering, see e.g. Kabat positions 223-243. In line with the above, the minimal requirement for a “hinge” are the amino acid residues corresponding to the IgG1 sequence stretch of D231 to P243 according to the Kabat numbering. The terms “CH2” and “CH3” refer to the immunoglobulin heavy chain constant regions 2 and 3. These regions can as well be identified by analogy using the Kabat numbering, see e.g. Kabat positions 244-360 for CH2 and Kabat positions 361-478 for CH3. Is is understood that there is some variation between the immunoglobulins in terms of their IgG1 Fc region, IgG2 Fc region, IgG3 Fc region, IgG4 Fc region, IgM Fc region, IgA Fc region, IgD Fc region and IgE Fc region (see, e.g., Padlan, Molecular Immunology, 31(3), 169-217 (1993)). The term Fc region refers to the last two heavy chain constant regions of IgA, IgD, and IgG, and the last three heavy chain constant regions of IgE and IgM. The Fc region can also include the flexible hinge N-terminal to these domains. For IgA and IgM, the Fc region may include the J chain. For IgG, the Fc region comprises immunoglobulin domains CH2 and CH3 and the hinge between the first two domains and CH2. Although the boundaries of the Fc region of an immunoglobulin may vary, an example for a human IgG heavy chain Fc portion comprising a functional hinge, CH2 and CH3 domain can be defined e.g. to comprise residues D231 (of the hinge domain) to P476 (of the C-terminus of the CH3 domain), or D231 to L476, respectively, for IgG4, wherein the numbering is according to Kabat.

The polypeptide construct of the invention may hence comprise in an N- to C-terminal order:
(a) one domain comprising a paratope (antigen-binding (epitope-binding) structure) binding an epitope of CLDN6;
(b) a peptide linker having an amino acid sequence selected from the group consisting of SEQ ID NOs: 563-575, particularly 563, 568, 570 to 575, more particularly SEQ ID NO: 570;
(c) another domain comprising a paratope (antigen-binding (epitope-binding) structure) binding an epitope of CD3;
(d) a peptide linker having an amino acid sequence selected from the group consisting of SEQ ID NOs: 563-575 and SEQ ID NO: 679, particularly SEQ ID NO: 679;
(e) one polypeptide monomer of a half-life extending domain (comprising a hinge, a CH2 and a CH3 domain), having an amino acid sequence selected from the group consisting of SEQ ID NOs: 630-637, particularly SEQ ID NO: 630;
(f) a peptide linker having an amino acid sequence selected from the group consisting of SEQ ID NOs: 563-575, particularly SEQ ID NO: 573; and
(g) another polypeptide monomer of the half-life extending domain (comprising a hinge, a CH2 and a CH3 domain), having an amino acid sequence selected from the group consisting of SEQ ID NOs: 630-637, particularly SEQ ID NO: 630.

It is also envisaged that the polypeptide construct of the invention comprises in an N- to C-terminal order:
one domain comprising a paratope (antigen-binding (epitope-binding) structure) binding an epitope of CLDN6 having an amino acid sequence selected from the group consisting of SEQ ID NO: 19, SEQ ID NO: 22, SEQ ID NO: 33, SEQ ID NO: 36, SEQ ID NO: 47, SEQ ID NO: 50, SEQ ID NO: 75, SEQ ID NO: 78, SEQ ID NO: 201, SEQ ID NO: 204, particularly SEQ ID NO: 19, SEQ ID NO: 22, SEQ ID NO: 33, SEQ ID NO: 36, SEQ ID NO: 47, more particularly SEQ ID NO: 19, SEQ ID NO: 22; wherein the peptide linker comprised within those sequences and having SEQ ID NO: 570 can be replaced by any one of SEQ ID NOs: 563-575;
a peptide linker having an amino acid sequence selected from the group consisting of SEQ ID NOs: 563, 565, 566, 569, 570, particularly SEQ ID NO: 565;
another domain comprising a paratope (antigen-binding (epitope-binding) structure) binding an epitope of CD3 having an amino acid sequence selected from the group consisting of SEQ ID NOs: 542-562 and 678; wherein a peptide linker comprised within those sequences is selected from amino acids having SEQ ID NO: 563-575 and 679;
a peptide linker having an amino acid sequence selected from the group consisting of SEQ ID NOs: 563-575; and
an additional domain comprising one or more amino acid sequences selected from the group consisting of SEQ ID NOs: 630-637, particularly SEQ ID NO: 630, and a peptide linker having an amino acid sequence selected from the group consisting of SEQ ID NOs: 563-575, particularly SEQ ID NO: 573.

Hence, in one embodiment, the polypeptide construct of the present invention comprises or consists of a polypeptide having an amino acid sequence selected from the group of those SEQ ID NO: 21, 24, 35, 38, 49, 52, 63, 66, 77, 80, 91, 94, 105, 108, 119, 122, 133, 136, 147, 150, 161, 164, 175, 178, 189, 192, 203, 206, 217, 220, 231, 234, 245, 148, 259, 262, 273, 276, 287, 290, 301, 304, 315, 318, 329, 332, 343, 346, 357, 360, 371, 374, 385, 388, 399, 402, 413, 416, 427, and 430, particularly, 21, 24, 35, 38, 49, 52, 63, 66, 77, 80, 91, 94, more particularly, 21, 24, 35, 38, 49, 52, 77, and 80, and particularly, 21, 35, 49, and 77.

Covalent modifications of the polypeptides/polypeptide constructs are also included within the scope of this invention, and are generally, but not always, done post-translationally. For example, several types of covalent modifications of the construct are introduced into the molecule by reacting specific amino acid residues of the construct with an organic derivatizing agent that can react with selected side chains or with the N- or C-terminal residues. Derivatization with bifunctional agents is useful for crosslinking the constructs of the present invention to a water-insoluble support matrix or surface for use in a variety of methods. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the constructs included within the scope of this invention comprises altering the glycosylation pattern of the protein. As is known in the art, glycosylation patterns can depend on both the sequence of the protein (e.g., the presence or absence of specific glycosylation amino acid residues, discussed below), or the host cell or organism in which the protein is produced. Specific expression systems are discussed below. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of a construct may be altered through changes at the DNA level, particularly by mutating the DNA encoding the polypeptide at preselected bases such that codons are generated that will translate into the desired amino acids.

Another means of increasing the number of carbohydrate moieties on the construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved using a variety of endo- and exo-glycosidases as described by Thotakura et al., 1987, Meth. Enzymol. 138:350. Glycosylation at potential glycosylation sites may be prevented using the compound tunicamycin as described by Duskin et al., 1982, J. Biol. Chem. 257:3105. Tunicamycin blocks the formation of protein-N-glycoside linkages.

Other modifications of the construct are also contemplated herein. For example, another type of covalent modification of the construct comprises linking the construct to various non-proteinaceous polymers, including polyols, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the construct, e.g. to facilitate the addition of polymers such as polyethylene glycol (PEG).

In some embodiments, the covalent modification of the constructs of the invention comprises the addition of one or more labels. The labelling group may be coupled to the construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to: a) isotopic labels, which may be radioactive or heavy isotopes, such as radioisotopes or radionuclides (e.g., ³H, ¹⁴C, ¹⁵N, 35S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.).

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank® Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78.

The polypeptide construct of the invention may also comprise additional domains, which are e.g. helpful in the isolation of the molecule or relate to an adapted pharmacokinetic profile of the molecule. Domains helpful for the isolation of a construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed constructs characterized by the identified CDRs may comprise a His-tag domain, which is generally known as a repeat of consecutive His residues in the amino acid sequence of a molecule, e.g. of five His residues (SEQ ID NO: 638), or of six His residues (hexa-histidine, SEQ ID NO: 639). The His-tag may be located e.g. at the N- or C-terminus of the construct. In one embodiment, a hexa-histidine tag (HHHHHH) is linked via peptide bond to the C-terminus of the construct according to the invention.

It is also envisaged that the polypeptide construct of the present invention comprises or consists of a polypeptide which has an amino acid sequence selected from the group consisting of those depicted in SEQ ID NOs: 22 and 24, and which is linked at its N-terminus or at its C-terminus with a protein purification tag, preferably via a peptide bond (amide bond). The linking of the protein purification tag at the C-terminus of the polypeptide is preferred. It is envisaged that the protein purification tag is a short peptide. For example, the length of the short peptide may be 2-30 amino acids, 4-25 amino acids, 5-20 amino acids or 6-19 amino acids. Examples of protein purification tags include, but are not limited to, AU1 epitope (e.g. as depicted in SEQ ID NO: 644), AU5 epitope (e.g. as depicted in SEQ ID NO: 645), T7-tag (e.g. as depicted in SEQ ID NO: 646), V5-tag (e.g. as depicted in SEQ ID NO: 647), B-tag (e.g. as depicted in SEQ ID NO: 648), E2 epitope (e.g. as depicted in SEQ ID NO: 649), FLAG epitope/FLAG tag (e.g. as depicted in SEQ ID NO: 650), Glu-Glu tag (e.g. as depicted in SEQ ID NOs: 651 or 652), HA tag, Histidine affinity tag (e.g. as depicted in SEQ ID NO: 653), HSV epitope (e.g. as depicted in SEQ ID NO: 654), KT3 epitope (e.g. as depicted in SEQ ID NO: 655), Myc epitope (e.g. as depicted in SEQ ID NO: 656), polyarginine tag (5-6 Arg residues), polyaspartate tag (5-16 Asp residues), polyhistidine tag (2-10 His residues, usually 6 His residues, see e.g. SEQ ID NO: 639), polyphenylalanine tag (usually 11 Phe residues), S1 tag (e.g. as depicted in SEQ ID NO: 659), S-tag (e.g. as depicted in SEQ ID NO: 660), Strep-tag (e.g. as depicted in SEQ ID NOs: 661 or 662), universal tag (e.g. as depicted in SEQ ID NO: 663), VSV-G (e.g. as depicted in SEQ ID NO: 664), Protein C (e.g. as depicted in SEQ ID NO: 665), and Protein A. A histidine tag is preferred, especially a 6×His tag (SEQ ID NO: 639). Is it hence further envisaged that the construct of the present invention consists of a polypeptide which has an amino acid sequence selected from the group consisting of those depicted in SEQ ID NOs: 22 and 24, and which is linked at its C-terminus with a 6×His tag via a peptide bond.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The polypeptide construct of the invention comprises a domain which binds to CD3 on the surface of a T cell. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to for the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide which in humans is encoded by the CD3 epsilon gene which resides on chromosome 11. In the context of the present invention, CD3 is understood as a protein complex and T cell co-receptor that is involved in activating both the cytotoxic T cell (CD8+ naive T cells) and T helper cells (CD4+ naive T cells). It is typically composed of four distinct chains. Especially in mammals, the complex contains a CD3γ chain, a CD36 chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The redirected lysis of target cells via the recruitment of T cells by a construct which binds to CD3 on the T cell and to a target protein on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see e.g. WO 2007/042261.

Cytotoxicity mediated by CLDN6×CD3 constructs can be measured in various ways. The “half maximal effective concentration” (EC50) is commonly used as a measure of potency of a biologically active molecule such as a construct of the present invention. It may be expressed in molar units. In the present case of measuring cytotoxicity, the EC50 value refers to the concentration of a construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). An EC50 value may typically be expected to be lower when stimulated/enriched CD8+ T cells are used as effector cells, compared with unstimulated PBMC. If the target cells are of macaque origin or express or are transfected with macaque CLDN6, the effector cells should also be of macaque origin, such as a macaque T cell line, e.g. 4119LnPx. The target cells should express CLDN6, such as human or macaque CLDN6, on the cell surface. Preferably the target cells should express at least the extracellular loop(s) of CLDN6, such as CLDN6 loop 1 and/or loop 2, on the cell surface. Target cells can be a cell line (such as CHO) which is stably or transiently transfected with CLDN6, e.g. human or macaque CLDN6. Alternatively, the target cells can be a CLDN6 positive natural expresser cell line, such as the human cancer lines. Usually EC50 values are expected to be lower when using target cells that express higher levels of CLDN6 on the cell surface compared with target cells having a lower target expression rate.

The effector to target cell (E:T) ratio in a cytotoxicity assay is usually about 10:1, but can also vary. Cytotoxic activity of CLDN6×CD3 constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by CLDN6×CD3 constructs of the present invention is measured in a cell-based cytotoxicity assay. It may also be measured in a 51-chromium release assay. It is envisaged that the EC50 value of the constructs of the invention is ≤300 pM, ≤280 pM, ≤260 pM, ≤250 pM, ≤240 pM, ≤220 pM, 200 pM, ≤180 pM, ≤160 pM, 150 pM, ≤140 pM, ≤120 pM, ≤100 pM, ≤90 pM, ≤80 pM, ≤70 pM, ≤60 pM, ≤50 pM, ≤40 pM, 30 pM, ≤20 pM, ≤15 pM, ≤10 pM, or ≤5 pM.

The above given EC50 values can be measured in different assays and under different conditions. For example, when human PBMCs are used as effector cells and CLDN6 transfected cells such as CHO cells are used as target cells, it is envisaged that the EC50 value of the CLDN6×CD3 construct is ≤500 pM, 400 pM, 300 pM, 280 pM, 260 pM, ≤250 pM, 240 pM, 220 pM, ≤200 pM, 180 pM, 160 pM, ≤150 pM, 140 pM, ≤120 pM, 100 pM, 90 pM, ≤80 pM, ≤70 pM, 60 pM, ≤50 pM, 40 pM, ≤30 pM, ≤20 pM, ≤15 pM, 10 pM, or ≤5 pM. When human PBMCs are used as effector cells and when the target cells are a CLDN6 positive cell line such as, it is envisaged that the EC50 value of the CLDN6×CD3 construct is 300 pM, 280 pM, 260 pM, ≤250 pM, 240 pM, 220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, 40 pM, ≤30 pM, ≤20 pM, ≤15 pM, 10 pM, or ≤5 pM.

According to one embodiment, the CLDN6×CD3 polypeptides/polypeptide constructs of the present invention do not induce/mediate lysis or do not essentially induce/mediate lysis of cells that do not express CLDN6 on their surface (CLDN6 negative cells), such as CHO cells. The term “do not induce lysis”, “do not essentially induce lysis”, “do not mediate lysis” or “do not essentially mediate lysis” means that a construct of the present invention does not induce or mediate lysis of more than 30%, preferably not more than 20%, more preferably not more than 10%, particularly preferably not more than 9%, 8%, 7%, 6% or 5% of CLDN6 negative cells, whereby lysis of CLDN6 expressing target cells (such as cells transformed or transfected with CLDN6 or a natural expresser cell line such as the human cancer lines) is set to be 100%. This usually applies for concentrations of the construct of up to 500 nM. Cell lysis measurement is a routine technique. Moreover, the present specification teaches specific instructions how to measure cell lysis.

The difference in cytotoxic activity between the monomeric and the dimeric isoform of individual CLDN6×CD3 polypeptides/polypeptide constructs is referred to as “potency gap”. This potency gap can e.g. be calculated as ratio between EC50 values of the molecule's monomeric and dimeric form. In one method to determine this gap, an 18 hour 51-chromium release assay or a 48 h FACS-based cytotoxicity assay is carried out as described hereinbelow with purified construct monomer and dimer. Effector cells are stimulated enriched human CD8+ T cells or unstimulated human PBMC. Target cells are hu CLDN6 transfected CHO cells. Effector to target cell (E:T) ratio is 10:1. Potency gaps of the CLDN6×CD3 constructs of the present invention are preferably ≤5, more preferably ≤4, even more preferably ≤3, even more preferably ≤2 and most preferably ≤1.

The domains of the polypeptide construct of the invention is/are preferably cross-species specific for members of the mammalian order of primates, such as macaques. Cross-species specific CD3 binding domains are, for example, described in WO 2008/119567. According to one embodiment, the domain, in addition to binding to human CD3, will also bind to CD3 of primates including (but not limited to) new world primates (such as Callithrix jacchus, Saguinus Oedipus or Saimiri sciureus), old world primates (such as baboons and macaques), gibbons, orangutans and non-human homininae. It is envisaged that the domain which binds to human CD3 on the surface of a T cell also binds at least to macaque CD3. A preferred macaque is Macaca fascicularis. Macaca mulatta (Rhesus) is also envisaged. One construct of the invention comprises a domain which binds to human CLDN6 on the surface of a target cell and another domain which binds to human CD3 on the surface of a T cell and at least macaque CD3.

In one embodiment, the affinity gap of the constructs according to the invention for binding macaque CD3 versus human CD3 [KD ma CD3: KD hu CD3](as determined e.g. by BiaCore or by Scatchard analysis) is between 0.01 and 100, preferably between 0.1 and 10, more preferably between 0.2 and 5, more preferably between 0.3 and 4, even more preferably between 0.5 and 3 or between 0.5 and 2.5, and most preferably between 0.5 and 1.

One domain of the construct of the invention binds to CD3. More preferably, it binds to CD3 on the surface of a T cell. It is furthermore envisaged that said domain binds to human CD3, preferably to human CD3 on the surface of a T cell. It is also envisaged that said domain binds to CD3 epsilon. More preferably, it binds to human CD3 epsilon, e.g. to human CD3 epsilon on the surface of a T cell. A preferred amino acid sequence for the extracellular domain of human CD3 epsilon is depicted in SEQ ID NO: 442.

In one embodiment of the present invention, said domain of the construct binds to human CD3 epsilon (or human CD3 epsilon on the surface of a T cell) and to Callithrix jacchus or Saimiri sciureus CD3 epsilon. It is also envisaged that said domain binds to an extracellular epitope of CD3 epsilon, preferably to an extracellular epitope of human CD3 epsilon. It is also envisaged that said domain binds to an extracellular epitope of the human and the Macaca CD3 epsilon chain. One preferred epitope of CD3 epsilon is comprised within amino acid residues 1-27 of the human CD3 epsilon extracellular domain (see SEQ ID NO: 443). Even more particularly, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or bispecific constructs directed against (human) CD3 or selectively and, preferably, specifically against CD3 epsilon are known in the art, and their CDRs, VH and VL sequences can serve as a basis for the binding domain of the polypeptide construct of the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific constructs directed against a (cancer) target and CD3 are also being developed and (pre-)clinically tested, and their CD3 binding domain (CDRs, VH, VL) may serve as a basis for the second binding domain of the construct of the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, R06958688), AFM11, and MGD006 (S80880). Other examples of CD3 binding domains are disclosed e.g. in U.S. Pat. No. 7,994,289 B2, U.S. Pat. No. 7,728,114 B2, U.S. Pat. No. 7,381,803 B1, U.S. Pat. No. 6,706,265 B1.

It is envisaged for the polypeptide construct used in accordance with the present invention that the domain which binds to CD3 on the surface of a T cell comprises a VL region comprising CDR-L1, CDR-L2 and CDR-L3, wherein the sequence of CDR-L1 is depicted in SEQ ID NO: 673, the sequence of CDR-L2 is depicted in SEQ ID NO: 674, and the sequence of CDR-L3 is depicted in SEQ ID NO: 675; or a VL region comprising CDR-L1, CDR-L2 and CDR-L3, CDR-L1 sequences as depicted in SEQ ID NO: 673, CDR-L2 as depicted in SEQ ID NO: 674, and CDR-L3 as depicted in SEQ ID NO: 675, wherein one or more of the CDRs have at least one amino acid residue modification.

It is also envisaged for the polypeptide construct used in accordance with the present invention that the domain which binds to CD3 on the surface of a T cell comprises a VH region comprising CDR-H1, CDR-H2 and CDR-H3 wherein the sequence of CDR-H1 as depicted in SEQ ID NO: 670, the sequence of CDR-H2 as depicted in SEQ ID NO:671, and the sequence of CDR-H3 as depicted in SEQ ID NO: 672; or a VH region comprising CDR-H1, CDR-H2 and CDR-H3 wherein the sequence of CDR-H1 as depicted in SEQ ID NO: 670, the sequence of CDR-H2 as depicted in SEQ ID NO:671, and the sequence of CDR-H3 as depicted in SEQ ID NO: 672, wherein one or more of the CDRs have at least one amino acid residue modification.

It is furthermore envisaged for the polypeptide construct used in accordance with the present invention that the domain which binds to CD3 comprises a VL region comprising CDR-L1, CDR-L2 and CDR-L3 and a VH region comprising CDR-H1, CDR-H2 and CDR-H3, wherein the sequence of the CDR-L1 is depicted in SEQ ID NO: 673, the sequence of the CDR-L2 is depicted in SEQ ID NO: 674, and the sequence of the CDR-L3 is depicted in SEQ ID NO: 675, wherein the sequence of the CDR-H1 as depicted in SEQ ID NO: 670, the sequence of the CDR-H2 as depicted in SEQ ID NO:671, and the sequence of the CDR-H3 as depicted in SEQ ID NO: 672; or a domain which binds to CD3 comprises a VL region comprising CDR-L1, CDR-L2 and CDR-L3 and a VH region comprising CDR-H1, CDR-H2 and CDR-H3, wherein the sequence of the CDR-L1 is depicted in SEQ ID NO: 673, the sequence of the CDR-L2 is depicted in SEQ ID NO: 674, and the sequence of the CDR-L3 is depicted in SEQ ID NO: 675, wherein the sequence of the CDR-H1 as depicted in SEQ ID NO: 670, the sequence of the CDR-H2 as depicted in SEQ ID NO:671, and the sequence of the CDR-H3 as depicted in SEQ ID NO: 672, wherein one or more of the CDRs have at least one amino acid residue modification.

It is envisaged for the polypeptide construct used in accordance with the present invention that the domain which binds to CD3 on the surface of a T cell comprises a VL region depicted in SEQ ID NO: 677, or wherein the VL region comprises at least one amino acid residue modification.

It is envisaged for the polypeptide construct used in accordance with the present invention that the domain which binds to CD3 on the surface of a T cell comprises a VH region depicted in SEQ ID NO: 676, or wherein the VH region comprises at least one amino acid residue modification.

More preferably, the polypeptide construct used in accordance with the present invention is characterized by the domain which binds to CD3 on the surface of a T cell comprising a VL region and a VH region selected from the group consisting of (a) a VL region as depicted in SEQ ID NO: 677 and a VH region as depicted in SEQ ID NO: 676; or wherein the VL region or the VH region comprises at least one amino acid residue modification.

A preferred embodiment of the above described polypeptide construct used in accordance with the present invention is characterized by the domain which binds to CD3 on the surface of a T cell comprising an amino acid sequence depicted in SEQ ID NO: 678, or wherein the domain which binds to CD3 on the surface of a T cell comprising an amino acid sequence depicted in SEQ ID NO: 678 comprises at least one amino acid residue modification.

It is envisaged for the polypeptide construct used in accordance with the present invention that the domain which binds to CD3 on the surface of a T cell comprises a VL region (e.g. the VL region depicted in SEQ ID NO: 540) or a VH region (e.g. the VL region depicted in SEQ ID NO: 522 or 533), or CDRs that are depicted in SEQ ID NO: 444 to 506, particularly CDRs depicted in SEQ ID NO: 480 to 482 and 504 to 506, or an scFv as depicted, for example, in SEQ ID NO: 551 or 562, or in any one SEQ ID NOs: 542-561.

Amino acid sequence modifications of the polypeptides/polypeptide constructs described herein are also contemplated. For example, it may be desirable to improve the binding affinity and/or other biological properties of the polypeptide construct. Amino acid sequence variants of the polypeptides/polypeptide constructs are prepared by peptide synthesis or by introducing appropriate nucleotide changes into the nucleic acid molecule encoding the polypeptides/polypeptide constructs. All below described amino acid sequence modifications should result in a polypeptide construct which retains the desired biological activity of the unmodified parental molecule (such as binding to CLDN6 and to CD3, inducing cytotoxicity against CLDN6 positive target cells).

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (GIn or Q); glutamic acid (GIu or E); glycine (GIy or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. There are basically four different classes of amino acids determined by different side chains: (1) non-polar and neutral (uncharged): Ala, Gly, Ile, Leu, Met, Phe, Pro, Val; (2) polar and neutral (uncharged): Asn, Cys (being only slightly polar), Gln, Ser, Thr, Trp (being only slightly polar), Tyr; (3) acidic and polar (negatively charged): Asp and Glu; (4) basic and polar (positively charged): Arg, His, Lys.

Hydrophobic amino acids can be divided according to whether they have aliphatic or aromatic side chains. Phe and Trp (being very hydrophobic), Tyr and His (being less hydrophobic) are classified as aromatic amino acids. Strictly speaking, aliphatic means that the side chain contains only hydrogen and carbon atoms. By this strict definition, the amino acids with aliphatic side chains are alanine, isoleucine, leucine (also norleucine), proline and valine. Alanine's side chain, being very short, means that it is not particularly hydrophobic, and proline has an unusual geometry that gives it special roles in proteins. It is often convenient to consider methionine in the same category as isoleucine, leucine and valine, although it also contains a sulphur atom. The unifying theme is that these amino acids contain largely non-reactive and flexible side chains. The amino acids alanine, cysteine, glycine, proline, serine and threonine are often grouped together because they are all small. Gly and Pro may influence chain orientation.

Amino acid modifications include, for example, deletions of residues from, insertions of residues into, and/or substitutions of residues within the amino acid sequences of the polypeptides/polypeptide constructs. Any combination of deletion, insertion, and/or substitution is made to arrive at a final construct, provided that the final construct possesses the desired characteristics, e.g. the biological activity of the unmodified parental molecule (such as binding to CLDN6 and to CD3, inducing cytotoxicity against CLDN6 positive target cells). The amino acid changes may also alter post-translational processes of the constructs, such as changing the number or position of glycosylation sites.

For example, 1, 2, 3, 4, 5, or 6 amino acids may be inserted, deleted and/or substituted in each of the CDRs (of course, dependent on their respective length), while 1, 2, 3, 4, 5, 6, 7, 8, 9, 10, 11, 12, 13, 14, 15, 16, 17, 18, 19, 20, or 25 amino acids may be inserted, deleted and/or substituted in each of the framework regions (FRs). Amino acid sequence insertions also include N-terminal and/or C-terminal additions of amino acids ranging in length from e.g. 1, 2, 3, 4, 5, 6, 7, 8, 9 or 10 residues to polypeptides containing more than 10, e.g. one hundred or more residues, as well as intra-sequence insertions of single or multiple amino acid residues. An insertional variant of the construct of the invention includes the fusion of a polypeptide which increases or extends the serum half-life of the construct to the N-terminus or to the C-terminus of the construct. It is also conceivable that such insertion occurs within the construct, e.g. between the first and the second domain.

The sites of greatest interest for amino acid modifications, particularly for amino acid substitutions, include the the hypervariable regions, particularly the individual CDRs of the heavy and/or light chain, but FR alterations in the heavy and/or light chain are also contemplated. The substitutions can be conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR, respectively. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for the identification of certain residues or regions within the constructs that are preferred locations for mutagenesis is called “alanine scanning mutagenesis” and is described e.g. in Cunningham B. C. and Wells J. A. (Science. 1989 Jun. 2; 244(4908):1081-5). Here, a residue or group of residues within the construct is/are identified (e.g. charged residues such as Arg, His, Lys, Asp, and Glu) and replaced by a neutral or non-polar amino acid (most preferably alanine or polyalanine) to affect the interaction of the respective amino acid(s) with the epitope of the target protein. Alanine scanning is a technique used to determine the contribution of a specific residue to the stability or function of given protein. Alanine is used because of its non-bulky, chemically inert, methyl functional group that nevertheless mimics the secondary structure preferences that many of the other amino acids possess. Sometimes bulky amino acids such as valine or leucine can be used in cases where conservation of the size of mutated residues is needed. This technique can also be useful to determine whether the side chain of a specific residue plays a significant role in bioactivity. Alanine scanning is usually accomplished by site-directed mutagenesis or randomly by creating a PCR library. Furthermore, computational methods to estimate thermodynamic parameters based on theoretical alanine substitutions have been developed. The data can be tested by IR, NMR Spectroscopy, mathematical methods, bioassays, etc.

Those amino acid locations demonstrating functional sensitivity to the substitutions (as determined e.g. by alanine scanning) can then be refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning, or random mutagenesis may be conducted at a target codon or region, and the expressed construct variants are screened for the optimal combination of desired activity. Techniques for making substitution mutations at predetermined sites in the DNA having a known sequence are well known, for example, M13 primer mutagenesis and PCR mutagenesis. Screening of the mutants is done e.g. using assays of antigen (e.g. CLDN6 or CD3) binding activity and/or of cytotoxic activity.

Generally, if amino acids are substituted in one or more or all the CDRs of the heavy and/or light chain, it is envisaged that the then-obtained “substituted” sequence is at least 60% or 65%, more preferably 70% or 75%, even more preferably 80% or 85%, and particularly preferably 90% or 95% identical/homologous to the “original” or “parental” CDR sequence. This means that the degree of identity/homology between the original and the substituted sequence depends on the length of the CDR. For example, a CDR having 5 amino acids in total and comprising one amino acid substitution is 80% identical to the “original” or “parental” CDR sequence, while a CDR having 10 amino acids in total and comprising one amino acid substitution is 90% identical to the “original” or “parental” CDR sequence. Accordingly, the substituted CDRs of the construct of the invention may have different degrees of identity to their original sequences, e.g., CDRL1 may have 80%, while CDRL3 may have 90% of homology. The same considerations apply to the framework regions and to the entire VH and VL regions.

A “variant CDR” is a CDR with a specific sequence homology, similarity, or identity to the parent CDR of the invention, and shares biological function with the parent CDR, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. Generally, the amino acid homology, similarity, or identity between individual variant CDRs is at least 60% to the parent sequences depicted herein, and more typically with increasing homologies, similarities or identities of at least 65% or 70%, preferably at least 75% or 80%, more preferably at least 85%, 90%, 91%, 92%, 93%, 94%, and most preferably 95%, 96%, 97%, 98%, 99%, and almost 100%. The same applies to “variant VH” and “variant VL”. According to one embodiment, the sequence variations within a “variant VH” and/or a “variant VL” do not extend to the CDRs. The present invention is hence directed to a construct as defined herein, comprising VH and VL sequences having a certain sequence homology (see above) to the specific sequences as defined herein (the “parental” VH and VL), wherein the CDR sequences are 100% identical to the specific CDR sequences as defined herein (the “parental” CDRs).

Preferred substitutions (or replacements) are conservative substitutions. However, any substitution (including non-conservative substitutions or one or more from the “exemplary substitutions” listed in Table 1, below) is envisaged, as long as the construct retains its capacity to bind to CLDN6 via the first domain and to CD3 or CD3 epsilon via the second domain, and/or provided its CDRs, FRs, VH and/or VL sequences have a degree of identity to the original or parental sequence of at least 60% or 65%, more preferably at least 70% or 75%, even more preferably at least 80% or 85%, and particularly preferably at least 90% or 95%.

A conservative replacement (also called a conservative mutation or a conservative substitution) is an amino acid replacement that changes a given amino acid to a different amino acid with similar biochemical properties (e.g. charge, hydrophobicity, size). Conservative replacements in proteins often have a smaller effect on protein function than non-conservative replacements. Conservative substitutions are shown in Table 1. Exemplary conservative substitutions are shown as “exemplary substitutions”. If such substitutions result in a change in biological activity, then more substantial changes, as further described herein with reference to amino acid classes, may be introduced and the products screened for a desired characteristic.

TABLE 1
Amino acid substitutions (aa = amino acid)

| Original aa | Conservative substitutions | Exemplary Substitutions |
|---|---|---|
| Ala (A) | Small aa | Gly, Ser, Thr |
| Arg (R) | Polar aa, particularly Lys | Lys, Gln, Asn |
| Asn (N) | Polar aa, particularly Asp | Asp, Gln, His, Lys, Arg |
| Asp (D) | Glu or other polar aa, particularly Asn | Glu, Asn |
| Cys (C) | Small aa | Ser, Ala |
| Gln (Q) | Polar aa, particularly Glu | Glu, Asn |
| Glu (E) | Asp or other polar aa, particularly Gln | Asp, Gln |
| Gly (G) | Small aa, such as Ala | Ala |
| His (H) | | Asn, Gln, Arg, Lys, Tyr |
| Ile (I) | Hydrophobic, particularly aliphatic aa | Ala, Val, Met, Leu, Phe |
| Leu (L) | Hydrophobic, particularly aliphatic aa | Norleucine, Ile, Ala, Val, Met |
| Lys (K) | Polar aa, particularly Arg | Arg, Gln, Asn |
| Met (M) | Hydrophobic, particularly aliphatic aa | Leu, Ala, Ile, Val, Phe |
| Phe (F) | Aromatic or hydrophobic aa, particularly | Tyr, Trp, Leu, Val, Ile, Ala |
| | Tyr | |
| Pro (P) | Small aa | Ala |
| Ser (S) | Polar or small aa, particularly Thr | Thr |
| Thr (T) | Polar aa, particularly Ser | Ser |
| Trp (W) | Aromatic aa | Tyr, Phe |
| Tyr (Y) | Aromatic aa, particularly Phe | Phe, Trp, Thr, Ser |
| Val (V) | Hydrophobic, particularly aliphatic aa | Leu, Ile, Ala, Met, Phe |

Substantial modifications in the biological properties of the construct of the present invention are accomplished by selecting substitutions that differ significantly in their effect on maintaining (a) the structure of the polypeptide backbone around the substitution, for example, as a sheet or helical conformation, (b) the charge or hydrophobicity of the molecule at the target site, or (c) the bulk of the side chain. Non-conservative substitutions will usually entail exchanging a member of one of the above defined amino acid classes (such as polar, neutral, acidic, basic, aliphatic, aromatic, small . . . ) for another class. Any cysteine residue not involved in maintaining the proper conformation of the construct may be substituted, generally with serine, to improve the oxidative stability of the construct.

Sequence identity, homology and/or similarity of amino acid sequences is determined by using standard techniques known in the art, including, but not limited to, the local sequence identity algorithm of Smith and Waterman, 1981, Adv. Appl. Math. 2:482, the sequence identity alignment algorithm of Needleman and Wunsch (J Mol Biol. 1970 March; 48(3):443-53), the search for similarity method of Pearson and Lipman (Proc Natl Acad Sci USA. 1988 April; 85(8):2444-8), computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (Nucleic Acids Res. 1984 Jan. 11; 12(1 Pt 1):387-95), preferably using the default settings, or by inspection. It is envisaged that percent identity is calculated by FastDB based upon the following parameters: mismatch penalty of 1; gap penalty of 1; gap size penalty of 0.33; and joining penalty of 30. See also “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle (J Mol Evol. 1987; 25(4):351-60); the method is similar to that described by Higgins and Sharp (Comput Appl Biosci. 1989 April; 5(2):151-3). Useful PILEUP parameters include a default gap weight of 3.00, a default gap length weight of 0.10, and weighted end gaps.

Another example of a useful algorithm is the BLAST algorithm, described in: Altschul et al. (J Mol Biol. 1990 Oct. 5; 215(3):403-10); Altschul et al., (Nucleic Acids Res. 1997 Sep. 1; 25(17):3389-402); and Karlin and Altschul (Proc Natl Acad Sci USA. 1993 Jun. 15; 90(12):5873-7). A particularly useful BLAST program is the WU-Blast-2 program which was obtained from Altschul et al., (Methods Enzymol. 1996; 266:460-80). WU-Blast-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the specific sequence and composition of the respective database against which the sequence of interest is being searched; however, the values may be adjusted to increase sensitivity.

An additional useful algorithm is gapped BLAST as reported by Altschul et al. (Nucleic Acids Res. 1997 Sep. 1; 25(17):3389-402). Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

In line herewith, the term “percent (%) nucleic acid sequence identity/homology/similarity” with respect to the nucleic acid sequence encoding the constructs identified herein is defined as the percentage of nucleotide residues in a candidate sequence that are identical with the nucleotide residues in the coding sequence of the construct. One method to align two sequences and thereby determine their homology uses the BLASTN module of WU-Blast2 set to the default parameters, with overlap span and overlap fraction set to 1 and 0.125, respectively. Generally, the nucleic acid sequence homology, similarity, or identity between the nucleotide sequences encoding individual variant CDRs and the nucleotide sequences depicted herein are at least 60%, and more typically with increasing homologies, similarities or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Again, the same applies to nucleic acid sequence encoding the “variant VH” and/or “variant VL”.

In one embodiment, the percentage of identity to human germline of the polypeptides/polypeptide constructs according to the invention, or of the domains comprising paratopes (antigen-binding (epitope-binding) structures; (binding domains)) of these constructs, is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang W. Y. and Foote J. (Methods. 2005 May; 36(1):3-10) demonstrate that the reduction of non-human portions of drug constructs leads to a decrease of risk of inducing anti-drug antibodies in the patients during treatment. By comparing an exhaustive number of clinically evaluated antibody drugs and the respective immunogenicity data, the trend is shown that humanization of the variable regions of antibodies/constructs makes the protein less immunogenic (average 5.1% of patients) than antibodies/constructs carrying unaltered non-human variable regions (average 23.59% of patients). Higher degrees of identity to human sequences are hence desirable for protein therapeutics based on variable regions and in the form of polypeptides/polypeptide constructs. To determine the germline identity, the V-regions of VL can be aligned with the amino acid sequences of human germline V segments and J segments (http://www2.mrc-lmb.cam.ac.uk/vbase/) using Vector NTI software and the amino acid sequence calculated by dividing the identical amino acid residues by the total number of amino acid residues of the VL in percent. The same can be done for the VH segments (http://www2.mrc-lmb.cam.ac.uk/vbase/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the polypeptides/polypeptide constructs of the present invention exhibit high monomer yields under standard research scale conditions, e.g., in a standard two-step purification process. It is envisaged that the monomer yield of the constructs according to the invention is ≥0.25 mg/L supernatant (SN), preferably ≥0.5 mg/L SN, more preferably ≥1 mg/L SN, even more preferably ≥2 mg/L SN and most preferably ≥3 mg/L SN. The yield of the construct denominated “CL-1×12C-6His” was shown to be 4.1 mg/L supernatant, and the yield of the construct denominated “CL-1×12C-scFc” was shown to be 36.5 mg/L supernatant.

Likewise, the yield of the dimeric polypeptide construct isoforms and hence the monomer percentage (i.e., monomer: (monomer+dimer)) of the constructs can be determined. The productivity of monomeric and dimeric constructs and the calculated monomer percentage can e.g. be obtained in the SEC purification step of culture supernatant from standardized research-scale production in roller bottles. According to one embodiment, the monomer percentage of the constructs of the invention is ≥80%, more preferably ≥85%, even more preferably ≥90%, and most preferably ≥95%.

According to one embodiment, the polypeptides/polypeptide constructs of the invention have a plasma stability (ratio of EC50 with plasma to EC50 w/o plasma) of ≤5 or ≤4, more preferably ≤3.5 or ≤3, even more preferably ≤2.5 or ≤2, and most preferably ≤1.5 or ≤1. The plasma stability of a construct can be tested by incubation of the purified construct in human plasma at 37° C. for 24 to 96 hours, e.g. at a concentration of 2-20 μg/ml, followed by EC50 determination in an 18 h 51-chromium release or in a 48 h FACS cytotoxicity assay (assays e.g. as described in the Examples section). The effector cells in the cytotoxicity assay can be stimulated enriched human CD8 positive T cells (preferred) or unstimulated human PBMC. Target cells can e.g. be CHO cells transfected with human CLDN6. The effector to target cell (E:T) ratio can be 10:1. The starting concentration of the constructs in the cytotoxicity assay can be 0.01-0.1 μg/ml. The human plasma pool used for this purpose is derived from the blood of healthy donors collected by EDTA coated syringes. Cellular components are removed by centrifugation and the upper plasma phase is collected and subsequently pooled. As control, non-incubated constructs are diluted immediately prior to the cytotoxicity assay in appropriate medium such as RPMI-1640. The plasma stability is calculated as ratio of EC50 (after plasma incubation) to EC50 (control/no incubation).

It is furthermore envisaged that the monomer to dimer conversion of the constructs of the invention is low. The conversion can be measured under different conditions and analyzed by high performance size exclusion chromatography. See Example 8. For example, incubation of the monomeric isoforms of the constructs can be carried out for 7 days at 37° C. in generic formulation buffer and at concentrations of e.g. 100 μg/ml or 250 μg/ml in an incubator, followed by high performance SEC to determine the percentage of initially monomeric construct which had been converted into dimeric construct. Under these conditions, it is envisaged that the polypeptides/polypeptide constructs of the invention show a dimer percentage that is ≤8%, preferably ≤6%, more preferably ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is likewise envisaged that the polypeptides/polypeptide constructs of the present invention present with very low dimer conversion after several freeze/thaw cycles. For example, the construct monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric construct which had been converted into dimeric construct. It is envisaged that the dimer percentages of the constructs are 8%, preferably 6%, more preferably ≤5%, more preferably ≤4%, even more preferably ≤3%, even more preferably 2.5%, even more preferably 2%, even more preferably 1.5%, and most preferably 1% or 0.5% or even 0%, for example after three freeze/thaw cycles.

According to one embodiment, the polypeptides/polypeptide constructs of the present invention show a favorable thermostability with aggregation temperatures ≥45° C. or ≥46° C., more preferably ≥47° C. or ≥48° C., even more preferably ≥49° C. or ≥50° C., and most preferably ≥51° C. The thermostability parameter can be determined in terms of antibody aggregation temperature as follows: Antibody solution at a concentration 250 μg/ml is transferred into a single use cuvette and placed in a dynamic light scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by differential scanning calorimetry (DSC) to determine intrinsic biophysical protein stabilities of the constructs. These experiments can be performed using a MicroCal LLC VP-DSC device. The energy uptake of a sample containing a construct is recorded from 20° C. to 90° C. compared to a sample containing only the formulation buffer. The constructs are adjusted to a final concentration of 250 μg/ml e.g. in SEC running buffer. For recording of the respective melting curve, the overall sample temperature is increased stepwise. Energy uptake of the sample and the formulation buffer reference is recorded at each temperature. The difference in energy uptake Cp (kcal/mole/° C.) of the sample minus the reference is plotted against the respective temperature. The melting temperature is defined as the temperature at the first maximum of energy uptake.

The polypeptides/polypeptide constructs of the invention are also envisaged to have a turbidity of ≤0.2 or ≤0.15, preferably of ≤0.10 or ≤0.08, more preferably of ≤0.06 or ≤0.05, and most preferably of ≤0.04 or ≤0.03. The turbidity can be measured by OD340 at a concentration of the construct of 2.5 mg/ml and 16 h incubation at 5° C.

Changes in the potency of a target×CD3 construct as a function of preincubation of the construct on the target cells in the absence of T cells can be measured. If a construct is internalized, it is expected to undergo lysosomal degradation. The effective concentration is hence expected to decrease over time, and thus the apparent potency should decrease as well. The effect has been observed with some targets, for which this is a known phenomenon. Constructs of the invention are envisaged to not be internalized or to not undergo significant internalization by the target cell. The rate of internalization can be assayed e.g. as described in the following: T cells are counted and diluted to a concentration of 1×105/ml in assay media. Target positive target cells are counted and plated e.g. at 2500 cells per well (cpw). The construct is diluted serially 1:2, e.g. at a starting concentration of 100 nM. The construct is added to the culture assay plates to allow for 0 hours, 1 hour or 2 hours of incubation prior to addition of the T cells. Then the T cells are plated at 25000 cpw (E:T=10:1), and the assay is incubated for 48 hours at 37° C. Target cell survival is analyzed e.g. with the Steady-Glo® system (25 μl/well). Preferably, the internalization rate (e.g. measured as a decrease in cytotoxicity) is ≤20% after a 2-hour (pre-)incubation of the construct with the target cell, more preferably ≤15%, even more preferably ≤10%, and most preferably ≤5%.

It is furthermore envisaged for a polypeptide construct of the invention that shed or soluble target does not significantly impair its efficacy or biologic activity. This can be measured, e.g. in a cytotoxicity assay where soluble target is added at increasing concentrations to the assay, e.g. at 0 nM-0.3 nM-0.7 nM-1 nM-3 nM-7 nM-12 nM. An exemplary E:T value is 10:1. The EC50 value of the tested construct should not be significantly increased in the presence of soluble target.

The EC50 values of the polypeptides/polypeptide constructs of the invention may be compared in in vitro cytotoxicity assays using CLDN6-expressing cells (e.g., CHO cells expressing the CLDN6, which are used as targets) and CLDN9-expressing cells (e.g., CHO cells expressing the CLDN9, which are used as targets), the latter serving as negative controls. The selectivity and specificity of the polypeptides/polypeptide constructs of the invention may be determined using T cells (e.g., PBMCs) as effector cells and the above CHO cells as targets. The cytotoxic effect of the polypeptides/polypeptide constructs of the invention may be determined. According to the invention, the polypeptides/polypeptide constructs described herein are at least 500 fold, at least 1000 fold, at least 2000 fold, and preferably at least 3000 fold more effective towards CLDN6-positive target cells than towards CLDN9-positive target cells, wherein the targets are preferably of the same cellular origin, e.g. CHO-cells, which are transfected or transformed with and expressing the genes encoding CLDN6 and CLDN9, respectively. Of course, it is possible to use other cell type expressing CLDN6, and a control cell that does not express CLDN6, but CLDN9 or CLDN4 or no CLDN-family member at all. These cells may be cell lines naturally expressing the molecules of interest or they may have been genetically modified to express CLDN6 and/or other CLDN-molecules, the latter being the controls. These cells may be used in methods of determining the T cell-dependent cytotoxicity associated with the present polypeptides/polypeptide constructs.

In a further embodiment, the polypeptide construct according to the invention is stable at acidic pH. The more tolerant the construct behaves at unphysiologic pH such as pH 5.5 (a pH which is required to run e.g. a cation exchange chromatography), the higher is the recovery of the construct eluted from an ion exchange column relative to the total amount of loaded protein. Recovery of the construct from an ion (e.g., cation) exchange column at pH 5.5 is preferably ≥30%, more preferably ≥40%, more preferably ≥50%, even more preferably ≥60%, even more preferably ≥70%, even more preferably ≥80%, and most preferably ≥95%. The percentage represents the area under the curve (=AUC) of the main peak.

It is furthermore envisaged that the polypeptides/polypeptide constructs of the present invention exhibit therapeutic efficacy, which manifests as anti-tumor activity or tumor growth inhibition. This can e.g. be assessed in a study as disclosed in Example 13 or 14. In one embodiment, the tumor growth inhibition of the construct of the invention T/C [%] is ≤70, ≤60, ≤50, ≤40, ≤30, ≤20, ≤10, ≤5, ≤4, ≤3, or ≤2. Modification or adjustment of certain parameters of these studies (such as the number of injected tumor cells, the site of injection, the number of transplanted human T cells, the number of constructs to be administered, and the timelines) is also envisaged, while still arriving at a meaningful and reproducible result.

The invention further provides a polynucleotide/nucleic acid molecule encoding a polypeptide construct of the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide of the present invention can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule of the invention, capable of expressing the construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, often this difference is in the second or third position. For instance, codons GAA and GAG both specify glutamic acid and exhibit redundancy; but, neither specifies any other amino acid nor thus demonstrate ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. to increase protein expression. It is hence envisaged that the polynucleotides/nucleic acid molecules of the present disclosure are codon optimized. Nevertheless, the polynucleotide/nucleic acid molecule encoding a construct of the invention may be designed using any codon that encodes the desired amino acid.

According to one embodiment, the polynucleotide/nucleic acid molecule of the present invention encoding the polypeptide construct of the invention is in the form of one single molecule or in the form of two or more separate molecules. If the construct of the present invention is a single chain construct, the polynucleotide/nucleic acid molecule encoding such construct will most likely also be in the form of one single molecule. However, it is also envisaged that different components of the polypeptide construct (such as the different domains, e.g. the paratope (antigen-binding (epitope-binding) structure)-comprising domain which binds to CLDN6, the paratope (antigen-binding (epitope-binding) structure)-comprising domain which binds to CD3, and/or further domains such as antibody constant domains) are located on separate polypeptide chains, in which case the polynucleotide/nucleic acid molecule is most likely in the form of two or more separate molecules.

The same applies for the vector comprising a polynucleotide/nucleic acid molecule of the present invention. If the construct of the present invention is a single chain construct, one vector may comprise the polynucleotide which encodes the construct in one single location (as one single open reading frame, ORF). One vector may also comprise two or more polynucleotides/nucleic acid molecules at separate locations (with individual ORFs), each one of them encoding a different component of the construct of the invention. It is envisaged that the vector comprising the polynucleotide/nucleic acid molecule of the present invention is in the form of one single vector or two or more separate vectors. In one embodiment, and for the purpose of expressing the construct in a host cell, the host cell of the invention should comprise the polynucleotide/nucleic acid molecule encoding the construct or the vector comprising such polynucleotide/nucleic acid molecule in their entirety, meaning that all components of the construct—whether encoded as one single molecule or in separate molecules/locations—will assemble after translation and form together the biologically active construct of the invention.

The invention also provides a vector comprising a polynucleotide/nucleic acid molecule of the invention. A vector is a nucleic acid molecule used as a vehicle to transfer (foreign) genetic material into a cell, usually to ensure the replication and/or expression of the genetic material. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a specific host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be achieved by artificial means. For transformation to happen, cells or bacteria must be in a state of competence, which might occur as a time-limited response to environmental conditions such as starvation and cell density and can also be artificially induced.

Moreover, the invention provides a host cell transformed or transfected with the polynucleotide/nucleic acid molecule of the invention or with the vector of the invention.

As used herein, the terms “host cell” or “recipient cell” are intended to include any individual cell or cell culture that can be or has been recipient of vectors, exogenous nucleic acid molecules and/or polynucleotides encoding the construct of the present invention; and/or recipients of the construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell but is still included within the scope of the term as used herein. Suitable host cells include prokaryotic or eukaryotic cells and include—but are not limited to—bacteria (such as E. coli), yeast cells, fungi cells, plant cells, and animal cells such as insect cells and mammalian cells, e.g., hamster, murine, rat, macaque or human.

In addition to prokaryotes, eukaryotic microbes such as filamentous fungi or yeast are suitable cloning or expression hosts for the construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silkmoth) have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

In a further embodiment, the invention provides a process for producing a construct of the invention, said process comprising culturing a host cell of the invention under conditions allowing the expression of the construct of the invention and recovering the produced construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a CO2 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures, so they can be grown to a higher density than adherent conditions would allow.

The term “expression” includes any step involved in the production of a construct of the invention including, but not limited to, transcription, post-transcriptional modification, translation, folding, post-translational modification, targeting to specific subcellular or extracellular locations, and secretion. The term “recovering” refers to a series of processes intended to isolate the construct from the cell culture. The “recovering” or “purification” process may separate the protein and non-protein parts of the cell culture, and finally separate the desired construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the construct can be produced intracellularly, in the periplasmic space, or directly secreted into the medium. If the construct is produced intracellularly, as a first step, the particulate debris, either host cells or lysed fragments, are removed, for example, by centrifugation or ultrafiltration. The construct of the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner that is like the process for purifying a construct expressed in mammalian cells and secreted into the medium. Carter et al. (Biotechnology (NY) 1992 February; 10(2):163-7) describe a procedure for isolating antibodies which are secreted to the periplasmic space of E. coli.

Where the construct is secreted into the medium, supernatants from such expression systems are generally first concentrated using a commercially available protein concentration filter, for example, an ultrafiltration unit.

The construct of the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the construct to be recovered.

A protease inhibitor may be included in any of the foregoing steps to inhibit proteolysis, and antibiotics may be included to prevent the growth of contaminants.

Moreover, the invention provides a pharmaceutical composition or formulation comprising a construct of the invention or a construct produced according to the process of the invention.

As used herein, the term “pharmaceutical composition” relates to a composition which is suitable for administration to a patient, preferably a human patient. The particularly preferred pharmaceutical composition of this invention comprises one or a plurality of the construct(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The compositions may comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the construct of the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may contain formulation materials for modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company). In such embodiments, suitable formulation materials may include, but are not limited to:
amino acids
antimicrobials such as antibacterial and antifungal agents
antioxidants
buffers, buffer systems and buffering agents that are used to maintain the composition at physiological pH or at a slightly lower pH, typically within a range of from about 5 to about 8 or 9
non-aqueous solvents, vegetable oils, and injectable organic esters
aqueous carriers including water, alcoholic/aqueous solutions, emulsions or suspensions, including saline and buffered media
biodegradable polymers such as polyesters
bulking agents
chelating agents
isotonic and absorption delaying agents
complexing agents
fillers
carbohydrates
(low molecular weight) proteins, polypeptides or proteinaceous carriers, preferably of human origin
coloring and flavouring agents
sulfur containing reducing agents
diluting agents
emulsifying agents
hydrophilic polymers
salt-forming counter-ions
preservatives
metal complexes
solvents and co-solvents
sugars and sugar alcohols
suspending agents
surfactants or wetting agents
stability enhancing agents
tonicity enhancing agents
parenteral delivery vehicles
intravenous delivery vehicles

It is common knowledge that the different constituents of the pharmaceutical composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In the context of the present invention, a pharmaceutical composition may comprise:
(a) a construct as described herein,
(b) at least one buffer agent,
(c) at least one saccharide, and
(d) at least one surfactant;
wherein the pH of the pharmaceutical composition is in the range of 3.5 to 6.

In the composition described above, the first domain preferably has an isoelectric point (pI) in the range of 4 to 9.5; the second domain has a pI in the range of 8 to 10, preferably 8.5 to 9.0; and the construct optionally comprises a third domain comprising two polypeptide monomers, each comprising a hinge, a CH2 domain and a CH3 domain, wherein said two polypeptide monomers are fused to each other via a peptide linker;

In the composition described above, it is further envisaged that the at least one buffer agent is present at a concentration range of 5 to 200 mM, more preferably at a concentration range of 10 to 50 mM. It is also envisaged that the at least one saccharide is selected from the group consisting of monosaccharide, disaccharide, cyclic polysaccharide, sugar alcohol, linear branched dextran or linear non-branched dextran. It is also envisaged that the disacchade is selected from the group consisting of sucrose, trehalose and mannitol, sorbitol, and combinations thereof. It is further envisaged that the sugar alcohol is sorbitol. It is also envisaged that the at least one saccharide is present at a concentration in the range of 1 to 15% (m/V), preferably in a concentration range of 9 to 12% (m/V). It is further envisaged that the construct is present in a concentration range of 0.1 to 8 mg/ml, preferably of 0.2-2.5 mg/ml, more preferably of 0.25-1.0 mg/ml.

According to one embodiment of the composition described above, the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged that the at least one surfactant is present at a concentration in the range of 0.004 to 0.5% (m/V), preferably in the range of 0.001 to 0.01% (m/V). It is envisaged that the pH of the composition is in the range of 4.0 to 5.0, preferably 4.2. It is also envisaged that the pharmaceutical composition has an osmolarity in the range of 150 to 500 mOsm. It is further envisaged that the pharmaceutical composition further comprises an excipient selected from the group consisting of one or more polyol(s) and one or more amino acid(s). It is envisaged in the context of the present invention that said one or more excipient is present in the concentration range of 0.1 to 15% (w/N).

The present invention also provides a pharmaceutical composition comprising (a) the construct as described herein, preferably in a concentration range of 0.1 to 8 mg/ml, preferably of 0.2-2.5 mg/ml, more preferably of 0.25-1.0 mg/ml; (b) 10 mM glutamate or acetate; (c) 9% (m/V) sucrose or 6% (m/V) sucrose and 6% (m/V) hydroxypropyl-o-cyclodextrin; (d) 0.01% (m/V) polysorbate 80; wherein the pH of the liquid pharmaceutical composition is 4.2.

It is envisaged that the composition of the invention might comprise, in addition to the construct of the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions, drugs modulating the inflammatory response, drugs acting on the circulatory system and/or agents such as cytokines known in the art. It is also envisaged that the polypeptide construct of the present invention is applied in a co-therapy, i.e., in combination with another anti-cancer medicament.

In this context, it is envisaged that the pharmaceutical composition of the invention (which comprises a construct comprising a domain which binds to CLDN6 on the surface of a target cell and another domain which binds to CD3 on the surface of a T cell, as described in more detail herein above) furthermore comprises an agent, preferably an antibody or construct, which binds to a protein of the immune checkpoint pathway (such as PD-1 or CTLA-4) or to a co-stimulatory immune checkpoint receptor (such as 4-1BB). The present invention also refers to a combination of a polypeptide construct according to the invention (which comprises a polypeptide construct comprising a domain comprising a paratope (antigen-binding (epitope-binding) structure) which binds to CLDN6 on the surface of a target cell and another domain comprising a paratope (antigen-binding (epitope-binding) structure) which binds to CD3 on the surface of a T cell, as described in more detail herein above) and an agent, preferably an antibody or polypeptide construct, which binds to a protein of the immune checkpoint pathway (such as PD-1 or CTLA-4) or to a co-stimulatory immune checkpoint receptor (such as 4-1BB). Due to the nature of the at least two ingredients of the combination, namely their pharmaceutical activity, the combination can also be referred to as a therapeutic combination. In some embodiments, the combination can be in the form of a pharmaceutical composition or of a kit. According to one embodiment, the pharmaceutical composition or the combination comprises a construct of the invention and an antibody or construct which binds to PD-1. Anti-PD-1 binding proteins useful for this purpose are e.g. described in detail in PCT/US2019/013205 incorporated herein by reference.

In certain embodiments, the optimal pharmaceutical composition is determined depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the construct of the invention. In certain embodiments, the primary vehicle or carrier in a pharmaceutical composition may be either aqueous or non-aqueous in nature. For example, a suitable vehicle or carrier may be water for injection or physiological saline solution, possibly supplemented with other materials common in compositions for parenteral administration. In certain embodiments, the compositions comprising the construct of the invention may be prepared for storage by mixing the selected composition having the desired degree of purity with optional formulation agents (Remington's Pharmaceutical Sciences, supra) in the form of a lyophilized cake or an aqueous solution. Further, in certain embodiments, the construct of the invention may be formulated as a lyophilizate using appropriate excipients.

When parenteral administration is contemplated, the therapeutic compositions for use in this invention may be provided in the form of a pyrogen-free, parenterally acceptable aqueous solution comprising the desired construct of the invention in a pharmaceutically acceptable vehicle. A particularly suitable vehicle for parenteral injection is sterile distilled water in which the construct of the invention is formulated as a sterile, isotonic solution, properly preserved. In certain embodiments, the preparation can involve the formulation of the desired molecule with an agent that may provide controlled or sustained release of the product which can be delivered via depot injection, or that may promote sustained duration in the circulation. In certain embodiments, implantable drug delivery devices may be used to introduce the desired construct.

Additional pharmaceutical compositions will be evident to those skilled in the art, including formulations involving the construct of the invention in sustained or controlled delivery formulations. Techniques for formulating a variety of sustained- or controlled-delivery means are known to those skilled in the art. The construct may also be entrapped in microcapsules prepared, for example, by coacervation techniques or by interfacial polymerization, in colloidal drug delivery systems, or in macroemulsions. Such techniques are disclosed in Remington's Pharmaceutical Sciences, supra.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions are generally placed into a container having a sterile access port, for example, an intravenous solution bag or vial having a stopper pierceable by a hypodermic injection needle.

Another aspect of the invention includes self-buffering formulations comprising the construct of the invention, which can be used as pharmaceutical compositions, as described in international patent application WO 2006/138181. A variety of publications are available on protein stabilization and formulation materials and methods useful in this regard, such as Arawaka T. et al., Pharm Res. 1991 March; 8(3):285-91; Kendrick et al., “Physical stabilization of proteins in aqueous solution” in: Rational Design of Stable Protein Formulations: Theory and Practice, Carpenter and Manning, eds. Pharmaceutical Biotechnology. 13: 61-84 (2002), and Randolph and Jones, Pharm Biotechnol. 2002; 13:159-75, see particularly the parts pertinent to excipients and processes for self-buffering protein formulations, especially as to protein pharmaceutical products and processes for veterinary and/or human medical uses.

Salts may be used in accordance with certain embodiments of the invention, e.g. to adjust the ionic strength and/or the isotonicity of a composition or formulation and/or to improve the solubility and/or physical stability of a construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, particularly the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. Several categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the construct of the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the construct of the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption. Surfactants also are commonly used to control protein conformational stability. The use of surfactants in this regard is protein specific, since one specific surfactant will typically stabilize some proteins and destabilize others.

Certain embodiments of the formulation or composition comprising the construct of the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants for use in therapeutic protein formulations in accordance with the present invention can be water-soluble and maintain their activity throughout the shelf life of the product (the composition comprising the construct). Antioxidants can also damage proteins and should hence—among other things—be selected in a way to eliminate or sufficiently reduce the possibility of antioxidants damaging the construct or other proteins in the formulation.

Certain embodiments of the formulation or composition comprising the construct of the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by in vitro cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of formulation of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to, white blood cell counts, differentials, fluorescence activated cell sorting, bone marrow aspiration. In addition, various disease specific clinical chemistry parameters and other established standard methods may be used. Furthermore, computer-aided tomography, X-ray, nuclear magnetic resonance tomography, positron-emission tomography scanning, lymph node biopsies/histologies and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a specific drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance (e.g., a construct of the invention) in blood plasma/serum to reach one-half of its steady-state value (“serum half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver. “Volume of distribution” (VD) means the degree to which a drug is distributed in body tissue rather than the blood plasma, a higher VD indicating a greater amount of tissue distribution. The retention of a drug can occur throughout the various compartments of the body, such as intracellular and extracellular spaces, tissues and organs, etc. “Degree of blood serum binding” means the propensity of a drug to interact with and bind to blood serum proteins, such as albumin, leading to a reduction or loss of biological activity of the drug.

Pharmacokinetic parameters also include bioavailability, lag time (T lag), Tmax, absorption rates, and/or Cmax for a given amount of drug administered. “Bioavailability” refers to the fraction of an administered dose of a drug/substance that reaches the systemic circulation (the blood compartment). When a medication is administered intravenously, its bioavailability is considered to be 100%. However, when a medication is administered via other routes (such as orally), its bioavailability generally decreases. “Lag time” means the time delay between the administration of the drug and its detection and measurability in blood or plasma. Cmax is the maximum plasma concentration that a drug achieves after its administration (and before the administration of a second dose). Tmax is the time at which Cmax is reached. The time to reach a blood or tissue concentration of the drug which is required for its biological effect is influenced by all parameters. Pharmacokinetic parameters of constructs exhibiting cross-species specificity may be determined in preclinical animal testing in non-chimpanzee primates as outlined above and set forth e.g. in Schlereth et al. (supra).

One embodiment provides the construct of the invention (or the construct produced according to the process of the invention), for the use as a medicament, particularly for the use in the prevention, treatment or amelioration of a disease, preferably a neoplasm. Another embodiment provides the use of the construct of the invention (or of the construct produced according to the process of the invention) in the manufacture of a medicament for the prevention, treatment or amelioration of a disease, preferably a neoplasm. It is also envisaged to provide a method for the prevention, treatment or amelioration of a disease, preferably a neoplasm, comprising the step of administering to a subject in need thereof the construct of the present invention (or the construct produced according to the process of the present invention). The terms “subject in need”, “patient” or those “in need of treatment” include those already with the disease, as well as those in which the disease is to be prevented. The terms also include human and other mammalian subjects that receive either prophylactic or therapeutic treatment.

The polypeptides/polypeptide constructs of the invention and the formulations/pharmaceutical compositions described herein are useful in the treatment, amelioration and/or prevention of the medical condition as described herein in a patient in need thereof. The term “treatment” refers to both therapeutic treatment and prophylactic or preventative measures. Treatment includes the application or administration of the polypeptides/polypeptide constructs/pharmaceutical composition to the body, to an isolated tissue, or to a cell from a patient or a subject in need who has a disease/disorder as described herein, a symptom of such disease/disorder, or a predisposition toward such disease/disorder, with the purpose to cure, heal, alleviate, relieve, alter, remedy, ameliorate, improve, or affect the disease, the symptom of the disease, or the predisposition toward the disease. The term “amelioration” as used herein refers to any improvement of the disease state of a patient, by the administration of a polypeptide construct according to the invention to such patient or subject in need thereof. Such an improvement may be a slowing down or stopping of the progression of the disease of the patient, and/or as a decrease in severity of disease symptoms, an increase in frequency or duration of disease symptom-free periods or a prevention of impairment or disability due to the disease. The term “prevention” as used herein means the avoidance of the occurrence or of the re-occurrence of a disease as specified herein, by the administration of a construct according to the invention to a subject in need thereof.

The term “disease” refers to any condition that would benefit from treatment with the construct or the pharmaceutical composition described herein. This includes chronic and acute disorders or diseases including those pathological conditions that predispose the mammal to the disease in question. The disease is preferably a neoplasm, cancer or tumor. The disease, neoplasm, cancer or tumor is preferably CLDN6 positive, i.e. it is characterized by expression or overexpression of CLDN6. An overexpression of CLDN6 means that there is an increase by at least 10%, in particular at least 25%, at least 50%, at least 100%, at least 250%, at least 500%, at least 750%, at least 1000% or even more. Expression is only found in a diseased tissue, while expression in a corresponding healthy tissue is not or significantly not detectable. According to the invention, diseases associated with cells expressing CLDN6 include cancer diseases. Furthermore, according to the invention, cancer diseases preferably are those wherein the cancer cells express CLDN6.

A “neoplasm” is an abnormal growth of tissue, usually but not always forming a mass. When also forming a mass, it is commonly referred to as a “tumor”. Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. Most cancers develop at their primary site but then go on to metastasize or spread to other parts (e.g. tissues and organs) of the body. These further tumors are “secondary tumors”. Most cancers continue to be called after their primary site, even after they have spread to other parts of the body.

Lymphomas and leukemias are lymphoid neoplasms. For the purposes of the present invention, they are also encompassed by the terms “tumor” and “cancer”. For the purposes of the present invention, the terms “neoplasm”, “tumor” and “cancer” may be used interchangeably, and they comprise both primary tumors/cancers and secondary tumors/cancers (or “metastases”) as well as mass-forming neoplasms (tumors) and lymphoid neoplasms (such as lymphomas and leukemias), and minimal residual disease (MRD).

The term “minimal residual disease” (MRD) refers to the evidence for the presence of small numbers of residual cancer cells that remain in the patient after cancer treatment, e.g. when the patient is in remission (no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a cancer are envisaged to also encompass “prevention, treatment or amelioration of MRD”, whether the MRD was detected or not.

In one embodiment of the invention, the neoplasm, cancer or tumor is selected from the group including, but not limited to, (or consisting of) germ cell cancer, ovarian cancer and lung cancer.

According to one embodiment of the invention, the ovarian cancer is ovarian epithelial cancer selected from the group comprising mucinous endometrioid, clear cell and undifferentiated ovarian cancer, ovarian stromal tumors including include granulosa cell tumors, granulosa-theca tumors and Sertoli-Leydig tumors, ovarian germ cell tumors comprising Teratomas, dysgerminoma ovarian germ cell cancer, endodermal sinus tumor (yolk sac tumor) and choriocarcinoma tumors, from Ovarian sarcomas, Krukenberg tumors, or Ovarian cysts. According to another embodiment, the ovarian cancer is recurrent or relapsed ovarian cancer, or an ovarian cancer that is refractory to plantinum and/or standard chemotherapy treatments. Treatment efficacy can be determined by measuring CA-125. CA-125 is a protein found in the blood. High amounts of CA-125 may indicate ovarian, fallopian tube cancer, and decreasing amounts may indicate efficacy of the selected treatment. Hereditary factors that may predispose the development of ovarian cancer are mutations on one of two genes called breast cancer gene 1 (BRCA1) and breast cancer gene 2 (BRCA2). Women with the BRCA1 mutation have a 35 to 70 percent higher risk of ovarian cancer. Women with the BRCA2 mutation have a 10 to 30 percent higher risk (www.cancercenter.com/cancer-types/ovarian-cancer/risk-factors). However, most women who are diagnosed with ovarian cancer do not have these mutations.

According to a further embodiment of the invention, the lung cancer is non-small cell lung cancer, which may be further selected from the group comprising squamous cell carcinoma, large cell carcinoma, and adenocarcinoma. Subsets of adenocarcinoma can be defined by specific mutations in genes encoding components of the epidermal growth factor receptor (EGFR) and downstream mitogen-activated protein kinases (MAPK) and phosphatidylinositol 3-kinases (PI3K) signaling pathways. Genetic abnormalities of potential relevance to treatment decisions include translocations involving the anaplastic lymphoma kinase (ALK)-tyrosine kinase receptor, which are sensitive to ALK inhibitors, and amplification of MET (mesenchymal epithelial transition factor), which encodes the hepatocyte growth factor receptor. MET amplification has been associated with secondary resistance to EGFR tyrosine kinase inhibitors.

The construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration. Formulations and compositions thus may be designed in accordance with the invention for delivery by any suitable route of administration. In the context of the present invention, the routes of administration include, but are not limited to topical routes, enteral routes and parenteral routes.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical compositions and the construct of this invention are particularly useful for parenteral administration, e.g., intravenous delivery, for example by injection or infusion. Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

The compositions of the present invention can be administered to the subject at a suitable dose which can be determined e.g. in dose escalating studies. As set forth above, the construct of the invention exhibiting cross-species specificity as described herein can also be advantageously used in in preclinical testing in non-chimpanzee primates. The dosage regimen will be determined by the attending physician and clinical factors. As is well known in the medical art, dosages for any one patient depend upon many factors, including the patient's size, body surface area, age, the specific compound to be administered, sex, time and route of administration, general health, and other drugs being administered concurrently.

An “effective dose” is an amount of a therapeutic agent that is sufficient to achieve or at least partially achieve a desired effect. A “therapeutically effective dose” is an amount that is sufficient to cure or at least partially arrest the disease and its complications, signs and symptoms in a patient suffering from the disease. Amounts or doses effective for this use will depend on the disease to be treated (the indication), the delivered construct, the therapeutic context and objectives, the severity of the disease, prior therapy, the patient's clinical history and response to the therapeutic agent, the route of administration, the size (body weight, body surface) and/or condition (the age and general health) of the patient, and the general state of the patient's own immune system. The proper dose can be adjusted according to the judgment of the attending physician, to obtain the optimal therapeutic effect.

A therapeutically effective amount of a construct of the invention preferably results in a decrease in severity of disease symptoms, an increase in frequency or duration of disease symptom-free periods or a prevention of impairment or disability due to the disease. In the treatment of CLDN6-expressing tumors, a therapeutically effective amount of the construct of the invention preferably inhibits tumor cell growth by at least about 20%, at least about 40%, at least about 50%, at least about 60%, at least about 70%, at least about 80%, or at least about 90% relative to untreated patients. The ability of a compound to inhibit tumor growth may also be evaluated in an animal model predictive of efficacy in human tumors.

In a further embodiment, the invention provides a kit comprising a construct of the invention, a construct produced according to the process of the invention, a polynucleotide of the invention, a vector of the invention, and/or a host cell of the invention. In the context of the present invention, the term “kit” means two or more components—one of which corresponding to the construct, the pharmaceutical composition, the polynucleotide, the vector or the host cell of the invention—packaged together in a container, recipient or otherwise. A kit can hence be described as a set of products and/or utensils that are sufficient to achieve a certain goal, which can be marketed as a single unit.

It is envisaged that a further component of the kit of the invention is an agent, preferably an antibody or construct, which binds to a protein of the immune checkpoint pathway (such as PD-1 or CTLA-4) or to a co-stimulatory immune checkpoint receptor (such as 4-1BB). These agents are described in more detail herein above. According to one embodiment, the kit comprises a construct of the invention and an antibody or construct which binds to PD-1. Anti-PD-1 binding proteins useful for this purpose are e.g. described in detail in PCT/US2019/013205. In certain embodiment, the kit allows for for the simultaneous and/or sequential administration of the components.

The kit may comprise one or more recipients (such as vials, ampoules, containers, syringes, bottles, bags) of any appropriate shape, size and material (preferably waterproof, e.g. plastic or glass) containing the construct or the pharmaceutical composition of the present invention in an appropriate dosage for administration (see above). The kit may additionally contain directions for use (e.g. in the form of a leaflet or instruction manual), means for administering the construct or the pharmaceutical composition of the present invention such as a syringe, pump, infuser or the like, means for reconstituting the construct of the invention and/or means for diluting the construct of the invention.

The invention also provides kits for a single-dose administration unit. The kit of the invention may also contain a first recipient comprising a dried/lyophilized construct or pharmaceutical composition and a second recipient comprising an aqueous formulation. In certain embodiments of this invention, kits containing single-chambered and multi-chambered pre-filled syringes are provided.

The present invention refers to the following items:
i) A polypeptide or polypeptide construct comprising or consisting of
a domain (comprising a paratope) which binds to human CLDN6 (SEQ ID NO: 1) on the surface of a target cell, and
a domain (comprising a paratope) which binds to human CD3, and
a domain extending the half-life of the polypeptide.
ii) The polypeptide or polypeptide construct according to item i), wherein said polypeptide construct is T-cell activating construct.
iii) The polypeptide or polypeptide construct according to any one of item i) and ii), wherein said polypeptide construct is a T-cell activating polypeptide as determined by a T cell activation assay selected from the group comprising determining the expression quantity of CD69, determining the expression quantity of CD25, determining the quantity of secreted IL-2, and determining the cytolytic activity of the T cells.
iv) The polypeptide or polypeptide construct according to any one of items i) to iii), wherein the domain which extends the half-life of the polypeptide comprises, or consists of, two polypeptide monomers, each comprising a hinge, a CH2 domain and a CH3 domain.
v) The polypeptide or polypeptide construct according to any one of items i) to iv), wherein the antigen-binding (epitope-binding) domain binding CLDN6 comprises, or consists of, a paratope that binds to an epitope within human CLDN6 which corresponds to amino acids 29-81 of SEQ ID NO. 1 (UniProt entry P56747).
vi) The polypeptide or polypeptide construct according to any one of items i) to v), wherein the first antigen-binding (epitope-binding) domain binding CLDN6 comprises, or consists of, a paratope that binds to an epitope within human CLDN6 which corresponds to amino acids 138-160 of SEQ ID NO. 1 (UniProt entry P56747).
vii) The polypeptide or polypeptide construct according to any one of items i) to vi), wherein said domain comprising, or consisting of, a paratope which binds to CLDN6 binds to amino acids 29-39 of SEQ ID NO: 1 in the extracellular loop 1 (ECL1) of CLDN6 as depicted in SEQ ID NO: 9 and/or to amino acids 151-160 of SEQ ID NO: 1 in the extracellular loop 2 (ECL2) of CLDN6 on the surface of a target cell as depicted in SEQ ID NO: 10; It is clear that a binder does not require a direct chemical interaction within the sequences depicted in SEQ ID NOs: 9 and 10, but that at least one or more amino acids in these sequences are in direct contact, e.g. through hydrogen bonds, with one and generally more amino acids of the binding domain. General principles of interactions between a binding domain (i.e. the paratope) and the target domain (i.e the epitope) are known in the art (cf. Janeway et al. Immunobiology, 9th Ed., 2016).
viii) The polypeptide or polypeptide construct according to any one of items i) to vii), wherein said domain comprising, or consisting of, a paratope binding to CD3 binds to an extracellular epitope of the human and the Macaca CD3ε chain.
ix) The polypeptide or polypeptide construct according to any one of items i) to viii), wherein the paratope that binds to CLDN6 binds to the same epitope on CLDN6 as a polypeptide construct or an antibody or derivative or fragment thereof that comprise a domain comprising a paratope binding to CLDN6 on the surface of a target cell, wherein the paratope comprises complementarity determining regions CDR-H1, CDR-H2, and CDR-H3 of a variable heavy (VH) chain and/or complementarity determining regions CDR-L1, CDR-L2, and CDR-L3 of a variable light (VL) chain selected from the groups depicted in a) to s) below, a) to d), n) and s) being preferred, a) to c), e) and s) being very preferred):
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising, or consisting of, a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising, or consisting of, a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising, or consisting of, a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising, or consisting of, a CDR-L1 depicted in SEQ ID NO: 254, a CDR-L2 depicted in SEQ ID NO: 255 and a CDR-L3 as depicted in SEQ ID NO: 256,
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising, or consisting of, a CDR-L1 depicted in SEQ ID NO: 268, a CDR-L2 depicted in SEQ ID NO: 269, and a CDR-L3 as depicted in SEQ ID NO: 270.
x) The polypeptide or polypeptide construct according to any one of items i) to ix), wherein the domain (comprising, or consisting of, the paratope) binding to human CD3 epsilon also binds to Callithrix jacchus or Saimiri sciureus CD3 epsilon.
xi) The polypeptide or polypeptide construct according to any one of items i) to x), wherein
a) the polypeptide or construct is a single chain construct,
b) the domain (comprising a paratope) binding to CLDN6 is in the format of an scFv,
c) the domain (comprising a paratope) binding to CD3 is in the format of an scFv,
d) the domains (comprising the paratopes) are connected via a linker, and/or
e) the polypeptide or polypeptide construct comprises a domain providing an extended serum half-life.
xii) The polypeptide or polypeptide construct according to any one of items i) to xi), wherein the domain (comprising, or consisting of, the paratope) binding to CLDN6 does not bind to CLDN1, CLDN2, CLDN3, CLDN4, CLDN9, and/or CLDN18.1/CLDN18.2.
xiii) The polypeptide or polypeptide construct according to any one of the preceding items, wherein the domain (comprising, or consisting of, a paratope) binding to CLDN6 comprises a VH region comprising a CDR-H1, a CDR-H2 and a CDR-H3 and a VL region comprising a CDR-L1, a CDR-L2 and a CDR-L3 selected from the groups depicted in in a) to s) below, a) to d), n) and s) being preferred, a) to c), e) and s) being very preferred):
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256,
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.
xiv) The polypeptide or polypeptide construct according to any one of the preceding items, wherein the domain comprising, or consisting of, a paratope binding to CLDN6 comprises a VH region having an amino acid sequence selected from the group comprising the sequences depicted in SEQ ID NO: 11, SEQ ID NO: 25, SEQ ID NO: 39, SEQ ID NO: 53, SEQ ID NO: 67, SEQ ID NO: 81, SEQ ID NO: 95, SEQ ID NO: 109, SEQ ID NO: 123, SEQ ID NO: 137, SEQ ID NO: 151, SEQ ID NO: 165, SEQ ID NO: 179, SEQ ID NO: 193, SEQ ID NO: 207, SEQ ID NO: 221, SEQ ID NO: 235, SEQ ID NO: 249, or SEQ ID NO: 263,
wherein said VH region amino acid sequence may have one or more modifications of one or several amino acid residues in the framework and/or hypervariable regions, provided said domain comprising said modified VH region selectively binds to CLDN6, and
optionally wherein said domain is part of a polypeptide or polypeptide construct that activates T cells and maintains the ability to induce T cell-dependent cytotoxicity,
further, optionally, wherein said domain is part of a polypeptide or polypeptide construct that activates T cells and maintains the ability to induce T cell-dependent cytotoxicity with more than 1000 fold efficacy than in an identical cell type which expresses CLDN9, but does not express CLDN6, and
still further optionally, wherein said domain is part of a polypeptide or polypeptide construct that is not capable of activating T cells and inducing T cell-dependent cytotoxicity in CLDN6-negative cells of the same cell type, preferably when tested in an in vitro cytotoxicity assay.
xv) The polypeptide or polypeptide construct according to any one of the preceding items, wherein the domain (comprising, or consisting of, a) paratope binding to CLDN6 comprises a VL region having an amino acid sequence selected from the group comprising the sequences depicted in SEQ ID NO: 12, SEQ ID NO: 26, SEQ ID NO: 40, SEQ ID NO: 54, SEQ ID NO: 68, SEQ ID NO: 82, SEQ ID NO: 96, SEQ ID NO: 110, SEQ ID NO: 124, SEQ ID NO: 138, SEQ ID NO: 152, SEQ ID NO: 166, SEQ ID NO: 180, SEQ ID NO: 194, SEQ ID NO: 208, SEQ ID NO: 222, SEQ ID NO: 236, SEQ ID NO: 250, or SEQ ID NO: 264,
wherein said VL region amino acid sequence may have one or more modifications of one or several amino acid residues in the framework and/or hypervariable regions, provided said domain comprising said modified VL region selectively binds to CLDN6, and
optionally wherein said domain is part of a polypeptide or polypeptide construct that activates T cells and maintains the ability to induce T cell-dependent cytotoxicity in target cells,
further, optionally, wherein said domain is part of a polypeptide or polypeptide construct that activates T cells and maintains the ability to induce T cell-dependent cytotoxicity with more than 500 fold efficacy than in control cells which do not express CLDN6, wherein these cells optionally express CLDN9, but
still further optionally, wherein said domain is part of a polypeptide or polypeptide construct that is not capable of activating T cells and inducing T cell-dependent cytotoxicity in CLDN6-negative cells of the same cell type, preferably when tested in a in vitro cytotoxicity assay.
xvi) The polypeptide or polypeptide construct according to any one of the preceding items, wherein the domain (comprising, or consisting of, a paratope) binding to CLDN6 comprises a pair of a VH region and a VL region having amino acid sequences as depicted in SEQ ID NOs: 11+12, SEQ ID NO: 25+26, SEQ ID NO: 39+40, SEQ ID NO: 53+54, SEQ ID NO: 67+68, SEQ ID NO: 81+82, SEQ ID NO: 95+96, SEQ ID NO: 109+110, SEQ ID NO: 123+124, SEQ ID NO: 137+138, SEQ ID NO: 151+152, SEQ ID NO: 165+166, SEQ ID NO: 179+180, SEQ ID NO: 193+194, SEQ ID NO: 207+208, SEQ ID NO: 221+222, SEQ ID NO: 235+236, SEQ ID NO: 249+250, or SEQ ID NO: 263+264.
xvii) The polypeptide or polypeptide construct according to any one of the preceding items, wherein the domain (comprising, or consisting of, a paratope) binding to CLDN6 comprises an amino acid sequence as depicted in SEQ ID NO: 19, SEQ ID NO: 22, SEQ ID NO: 33, SEQ ID NO: 36, SEQ ID NO: 47, SEQ ID NO: 50, SEQ ID NO: 61, SEQ ID NO: 64, SEQ ID NO: 75, SEQ ID NO: 78, SEQ ID NO: 89, SEQ ID NO: 92, SEQ ID NO: 103, SEQ ID NO: 106, SEQ ID NO: 117, SEQ ID NO: 120, SEQ ID NO: 131, SEQ ID NO: 134, SEQ ID NO: 145, SEQ ID NO: 148, SEQ ID NO: 159, SEQ ID NO: 162, SEQ ID NO: 173, SEQ ID NO: 176, SEQ ID NO: 187, SEQ ID NO: 190, SEQ ID NO: 201, SEQ ID NO: 204, SEQ ID NO: 215, SEQ ID NO: 218, SEQ ID NO: 229, SEQ ID NO: 232, SEQ ID NO: 243, SEQ ID NO: 246, SEQ ID NO: 257, or SEQ ID NO: 260, SEQ ID NO: 271 or SEQ ID NO: 274.
xviii) The polypeptide or polypeptide construct according to any one of the preceding items, comprising or consisting of a polypeptide having an amino acid sequence selected from the group of those depicted in SEQ ID NO: 19, SEQ ID NO: 20, SEQ ID NO: 21, SEQ ID NO: 22, SEQ ID NO: 23, and SEQ ID NO: 24, SEQ ID NO: 33, SEQ ID NO: 34, SEQ ID NO: 35, SEQ ID NO: 36, SEQ ID NO: 37, and SEQ ID NO: 38, SEQ ID NO: 47, SEQ ID NO: 48, SEQ ID NO: 49, SEQ ID NO: 50, SEQ ID NO: 51, and SEQ ID NO: 52, SEQ ID NO: 61, SEQ ID NO: 62, SEQ ID NO: 63, SEQ ID NO: 64, SEQ ID NO: 65, and SEQ ID NO: 66, SEQ ID NO: 75, SEQ ID NO: 76, SEQ ID NO: 77, SEQ ID NO: 78, SEQ ID NO: 79, and SEQ ID NO: 80, SEQ ID NO: 89, SEQ ID NO: 90, SEQ ID NO: 91, SEQ ID NO: 92, SEQ ID NO: 93, and SEQ ID NO: 94, SEQ ID NO: 103, SEQ ID NO: 104, SEQ ID NO: 105, SEQ ID NO: 106, SEQ ID NO: 107, and SEQ ID NO: 108, SEQ ID NO: 117, SEQ ID NO: 118, SEQ ID NO: 119, SEQ ID NO: 120, SEQ ID NO: 121, and SEQ ID NO: 122, SEQ ID NO: 131, SEQ ID NO: 132, SEQ ID NO: 133, SEQ ID NO: 134, SEQ ID NO: 135, and SEQ ID NO: 136, SEQ ID NO: 145, SEQ ID NO: 146, SEQ ID NO: 147, SEQ ID NO: 148, SEQ ID NO: 149, and SEQ ID NO: 150, SEQ ID NO: 159, SEQ ID NO: 160, SEQ ID NO: 161, SEQ ID NO: 162, SEQ ID NO: 163, and SEQ ID NO: 164, SEQ ID NO: 173, SEQ ID NO: 174, SEQ ID NO: 175, SEQ ID NO: 176, SEQ ID NO: 177, and SEQ ID NO: 178, SEQ ID NO: 187, SEQ ID NO: 188, SEQ ID NO: 189, SEQ ID NO: 190, SEQ ID NO: 191, and SEQ ID NO: 192, SEQ ID NO: 201, SEQ ID NO: 202, SEQ ID NO: 203, SEQ ID NO: 204, SEQ ID NO: 205, and SEQ ID NO: 206, SEQ ID NO: 215, SEQ ID NO: 216, SEQ ID NO: 217, SEQ ID NO: 218, SEQ ID NO: 219, and SEQ ID NO: 220, SEQ ID NO: 229, SEQ ID NO: 230, SEQ ID NO: 231, SEQ ID NO: 232, SEQ ID NO: 233, and SEQ ID NO: 234, SEQ ID NO: 243, SEQ ID NO: 244, SEQ ID NO: 245, SEQ ID NO: 246, SEQ ID NO: 247, and SEQ ID NO: 248, SEQ ID NO: 257, SEQ ID NO: 258, SEQ ID NO: 259, SEQ ID NO: 260, SEQ ID NO: 261, and SEQ ID NO: 262, SEQ ID NO: 271, SEQ ID NO: 272, SEQ ID NO: 273, SEQ ID NO: 274, SEQ ID NO: 275, and SEQ ID NO: 276, or from polypeptides/polypeptide constructs having an amino acid having at least 90, 91, 92, 93, 94, 95, 96, 97, 98 or 99% identity to said sequences.
xix) The polypeptide or polypeptide construct according to any one of the preceding items, wherein the domain comprising, or consisting of, a paratope binding to CLDN6 induces at least 100 fold, at least 250 fold, at least 500 fold lower cytotoxicity, or at least 1000 fold lower T cell-dependent cytotoxicity as determined in an in vitro assay using a cell that expresses a mutant of wild-type CLDN6 as depicted in SEQ ID NO: 1 that comprises at least one or more of the following mutations M29X, wherein X is preferably L, R145X, wherein X is preferably Q, and/or Q156X, wherein X is preferably L, as compared with the T cell-dependent cytotoxicity measured in the in vitro assay using a cell that expresses CLDN6 as depicted in SEQ ID NO: 1.
xx) The polypeptide or polypeptide construct according to any one of the preceding items, wherein the domain comprising, or consisting of, a paratope binding to CLDN6 induces at least 100 fold, at least 250 fold, at least 500 fold lower cytotoxicity, or at least 1000 fold lower T cell-dependent cytotoxicity as determined in an in vitro assay using a cell that expresses a mutant of wild-type CLDN6 as depicted in SEQ ID NO: 1 that comprises at least one or more of the following mutations M29X, wherein X is preferably L, R145X, wherein X is preferably Q, and/or Q156X, wherein X is preferably L, as compared with the T cell-dependent cytotoxicity measured in the in vitro assay using a cell that expresses CLDN6 as depicted in SEQ ID NO: 1, wherein said construct is capable of activating T cells and inducing T cell-dependent cytotoxicity in target cells expressing CLDN6, and wherein said construct has a heavy chain CDR3 sequence comprising: X1LIVX2APX3 (SEQ ID NO. 667), wherein X1 is either A or N; X2 is either V or E; and X3 is either V or A.
xxi) The polypeptide or polypeptide construct according to one of the preceding items, wherein the construct is a single chain construct.
xxii) The polypeptide or polypeptide construct according to one of the preceding items, wherein said half-life extending domain comprising, or consisting of, two polypeptide monomers comprises a hinge, a CH2 domain and a CH3 domain comprising in an amino to carboxyl order: hinge-CH2-CH3-linker-hinge-CH2-CH3.
xxiii) The polypeptide or polypeptide construct according to one of the preceding items, wherein the CH2 domain comprises an intra domain cysteine disulfide bridge.
xxiv) The polypeptide or polypeptide construct according to one of the preceding items, wherein
(a) the antigen-binding (epitope-binding) domain comprising the paratope binding CLDN6 comprises, or consists of, two antibody variable domains and the antigen-binding (epitope-binding) domain comprising the paratope binding CD3 comprises, or consists of, two antibody variable domains;
(b) the antigen-binding (epitope-binding) domain comprising the paratope binding CLDN6 comprises, or consists of, one antibody variable domain and the antigen-binding (epitope-binding) domain comprising the paratope binding CD3 comprises, or consists of, two antibody variable domains;
(c) the antigen-binding (epitope-binding) domain comprising the paratope binding CLDN6 comprises, or consists of, two antibody variable domains and the antigen-binding (epitope-binding) domain comprising the paratope binding CD3 comprises, or consists of, one antibody variable domain; or
(d) the antigen-binding (epitope-binding) domain comprising the paratope binding CLDN6 comprises, or consists of, one antibody variable domain and the antigen-binding (epitope-binding) domain comprising the paratope binding CD3 comprises, or consists of, one antibody variable domain.
xxv) The polypeptide or polypeptide construct according to one of the preceding items, wherein the antigen-binding (epitope-binding) comprising, or consisting of, the paratope binding CLDN6 and the antigen-binding (epitope-binding) domain comprising, or consisting of, the paratope binding CD3 are fused to another domain via a peptide linker.
xxvi) The polypeptide or polypeptide construct according to any one of the preceding items, wherein the polypeptide or polypeptide construct comprises, or consists of, in an amino to carboxyl order, or in a carboxyl to amino order:
(a) an antigen-binding (epitope-binding) domain (comprising a paratope) binding to CLDN6;
(b) a peptide linker preferably having an amino acid sequence selected from the group consisting of SEQ ID NOs: 563-575;
(c) an antigen-binding (epitope-binding) domain (comprising a paratope) binding to CD3.
xxvii) The polypeptide or polypeptide construct according to item xxvi), wherein the polypeptide or polypeptide construct further comprises in an amino to carboxyl order, or in a carboxyl to amino order, or between the antigen-binding (epitope-binding) domain comprising a paratope binding to CLDN6 and the antigen-binding (epitope-binding) domain (comprising a paratope) binding to CD3:
(a) a peptide linker having an amino acid sequence selected from the group consisting of SEQ ID NOs: 563-575;
(b) the first polypeptide monomer of a third domain;
(c) a peptide linker having an amino acid sequence selected from the group consisting of SEQ ID NOs: 563-575; and
(d) the second polypeptide monomer of said third domain.
xxviii) The polypeptide or polypeptide construct according to one of the preceding items, wherein the construct is depicted in any one of the sequences depicted in SEQ ID NO: 21, 24, 35, 38, 49, 52, 63, 66, 77, 80, 91, 94, 105, 108, 119, 122, 133, 136, 147, 150, 161, 164, 175, 178, 189, 192, 203, 206, 217, 220, 231, 234, 245, 148, 259, 262, 273, 276, 287, 290, 301, 304, 315, 318, 329, 332, 343, 346, 357, 360, 371, 374, 385, 388, 399, 402, 413, 416, 427, and 430, particularly, 21, 24, 35, 38, 49, 52, 63, 66, 77, 80, 91, 94, more particularly, 21, 24, 35, 38, 49, 52, 77, and 80, and even more particularly, 21, 35, 49, and 77.
xxix) The polypeptide or polypeptide construct according to one of the preceding items, wherein the construct comprises a domain (comprising a paratope) binding to CD3 comprising a VH domain comprising at least one, two, or all of the following CDR sequences as depicted in SEQ ID NO: 670, 671, and/or 672.
xxx) The polypeptide or polypeptide construct according to one of the preceding items, wherein the construct comprises a domain (comprising a paratope) binding to CD3 comprising a VL domain comprising at least one, two, or all of the following CDR sequences as depicted in SEQ ID NO: 673, 674, and/or 675.
xxxi) The polypeptide or polypeptide construct according to one of the preceding items, wherein the construct comprises a domain (comprising a paratope) binding to CD3 comprising a VH and a VL domain comprising at least one, two, or all of the following CDR sequences as depicted in SEQ ID NO: 670, 671, 672, 673, 674, and/or 675.
xxxii) The polypeptide or polypeptide construct according to one of the preceding items, wherein the construct comprises a domain (comprising a paratope) binding to CD3 comprising a VH domain as depicted in SEQ ID NO: 676.
xxxiii) The polypeptide construct according to one of the preceding items, wherein the construct comprises a domain (comprising a paratope) binding to CD3 comprising a VL domain as depicted in SEQ ID NO: 677.
xxxiv) The polypeptide or polypeptide construct according to one of the preceding items, wherein the construct comprises a domain (comprising a paratope) binding to CD3 comprising a VH domain as depicted in SEQ ID NO: 676 and a VL domain as depicted in SEQ ID NO: 677.
xxxv) The polypeptide or polypeptide construct according to one of the preceding items, wherein the construct comprises a domain (comprising a paratope) binding to CD3 comprising a scFv domain as depicted in SEQ ID NO: 678.
xxxvi) A polynucleotide encoding a polypeptide or polypeptide construct as defined in any one of the preceding items.
xxxvii) A vector comprising a polynucleotide as defined in item xxxvi).
xxxviii)A host cell transformed or transfected with the polynucleotide as defined in item xxxvi) or with the vector as defined in item xxxvii).
xil) A process for producing a polypeptide or polypeptide construct as defined in any one of items i) to xxxv), said process comprising culturing a host cell as defined in item xxxviii) under conditions allowing the expression of said polypeptide construct and recovering the produced polypeptide or polypeptide construct from the culture.
xl) A pharmaceutical composition comprising a polypeptide or polypeptide construct as defined in any one of items i) to xxxv), or that is produced according to the process of item xxxix).
xli) The polypeptide or polypeptide construct according to any one of items i) to xxxv) or that is produced according to the process of item xxxix) for the use as a medicament, particularly for the use in the prevention, treatment or amelioration of a disease, preferably a neoplasm.
xlii) The polypeptide or polypeptide construct according to item xli) for the use as a medicament, particularly for the use in the prevention, treatment or amelioration of a disease, wherein the disease or neoplasm is selected from the group consisting of germ cell cancer, particularly ovarian cancer, in particular ovarian adenocarcinoma and ovarian teratocarcinoma, uterine cancer, and lung cancer, including small cell lung cancer (SCLC) and non-small cell lung cancer (NSCLC), in particular squamous cell lung carcinoma and adenocarcinoma.
xliii) The polypeptide or polypeptide construct according to item xli), wherein the lung cancer is non-small cell lung cancer (NSCLC), in particular squamous cell lung carcinoma and adenocarcinoma.
xliv) A kit comprising a polypeptide or polypeptide construct as defined in any one of items i) to xxxv), a polypeptide or polypeptide construct produced according to the process of item xil), a polynucleotide as defined in item xxxvi), a vector as defined in item xxxvii), and/or a host cell as defined in item xxxviii).
xlv) A method for the treatment or amelioration of a proliferative disease, a tumorous disease, cancer, or an immunological disorder, comprising the step of administering to a subject in need thereof the polypeptide or polypeptide construct according to any one of items i) to xxxv), or produced according to the process of item xil), wherein the disease preferably is selected from the group consisting of germ cell cancer, ovarian cancer, in particular ovarian adenocarcinoma and ovarian teratocarcinoma, uterine cancer, more particularly from ovarian serous cystadenocarcinoma, uterine carcinosarcoma, uterine corpus endometrial carcinoma, and lung cancer, including small cell lung cancer (SCLC) and non-small cell lung cancer (NSCLC), in particular squamous cell lung carcinoma and adenocarcinoma.
xlvi) A polypeptide or a polypeptide construct comprising
   a domain which binds to human CLDN6 (SEQ ID NO: 1), and
   a domain which binds to human CD3, and
   a domain extending the half-life of the polypeptide.
xlvii) The polypeptide or polypeptide construct according to item xlvi), wherein said polypeptide construct is a T-cell activating construct.
xlviii) The polypeptide or polypeptide construct according to any one of item xlvi) and xlvii), wherein said polypeptide construct is a T-cell activating polypeptide as determined in a T cell activation assay selected from the group comprising determining the expression quantity of CD69, determining the expression quantity of CD25, determining the quantity of secreted IL-2, and determining the cytolytic activity of the T cells.
xlix) The polypeptide or polypeptide construct according to any one of item xlvi) to xlviii), wherein the domain which extends the half-life of the polypeptide comprises two polypeptide monomers, each comprising a hinge, a CH2 domain and a CH3 domain.
xlx) The polypeptide or polypeptide construct according to any one of item xlvi) and xlix), wherein the antigen-binding domain binding CLDN6 binds to an epitope within human CLDN6 which corresponds to amino acids 29-81 of SEQ ID NO. 1 (UniProt entry P56747).
xlxi) The polypeptide or polypeptide construct according to any one of item xlvi) and xlx), wherein the first antigen-binding domain binding CLDN6 binds to an epitope within human CLDN6 which corresponds to amino acids 138-160 of SEQ ID NO. 1 (UniProt entry P56747).
xlxii) The polypeptide or polypeptide construct according to any one of item xlvi) and xlxi), wherein said domain binds to CLDN6 amino acids 29-39 of SEQ ID NO: 1 in the extracellular loop 1 (ECL1) of CLDN6 which is depicted in SEQ ID NO: 9 and/or by amino acids 151-160 of SEQ ID NO: 1 in the extracellular loop 2 (ECL2) of CLDN6 as depicted in SEQ ID NO: 10.
xlxiii) The polypeptide or polypeptide construct according to any one of item xlvi) and xlxii), wherein said domain binding to CD3 binds to an extracellular epitope of the human and the Macaca CD3ε chain.
xlxiv) The polypeptide or polypeptide construct according to any one of item xlvi) and xlxiii), wherein the domain that binds to CLDN6 binds to the same epitope on CLDN6 as a polypeptide construct or an antibody or derivative or fragment thereof that comprise a domain binding to CLDN6, wherein the domain comprises complementarity determining regions CDR-H1, CDR-H2, and CDR-H3 of a variable heavy (VH) chain and/or complementarity determining regions CDR-L1, CDR-L2, and CDR-L3 of a variable light (VL) chain selected from the groups depicted in a) to s) below, a) to d), n) and s) being preferred, a) to c), e) and s) being very preferred):
a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256,
s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.
xlxv) The polypeptide or polypeptide construct according to any one of item xlvi) and xlxiv), wherein the domain binding to human CD3 epsilon also binds to Callithrix jacchus or Saimiri sciureus CD3 epsilon.
xlxvi) The polypeptide or polypeptide construct according to any one of item xlvi) and xlxv), wherein
   a) the polypeptide is a single chain construct,
   b) the domain binding to CLDN6 is in the format of an scFv,
   c) the domain binding to CD3 is in the format of an scFv,
   d) the domains are connected via a linker, and/or
   e) the polypeptide or polypeptide construct comprises a domain providing an extended serum half-life.
xlxvii) The polypeptide or polypeptide construct according to any one of item xlvi) and xlxvi), wherein the domain binding to CLDN6 does not bind to CLDN1, CLDN2, CLDN3, CLDN4, CLDN9, and/or CLDN18.1/CLDN18.2.
xlxviii) The polypeptide or polypeptide construct according to any one of item xlvi) and xlxvii), wherein the domain binding to CLDN6 comprises a VH region comprising a CDR-H1, a CDR-H2 and a CDR-H3 and a VL region comprising a CDR-L1, a CDR-L2 and a CDR-L3 selected from the groups depicted in in a) to s) below, a) to d), n) and s) being preferred, a) to c), e) and s) being very preferred:
   a) a VH region comprising a CDR-H1 depicted in SEQ ID NO: 13, a CDR-H2 depicted in SEQ ID NO: 14, and a CDR-H3 depicted in SEQ ID NO: 15, and a VL region comprising a CDR-L1 depicted in SEQ ID NO: 16, a CDR-L2 depicted in SEQ ID NO: 17 and a CDR-L3 depicted in SEQ ID NO: 18;
   b) a VH region comprising a CDR-H1 depicted in SEQ ID NO: 27, a CDR-H2 depicted in SEQ ID NO: 28, and a CDR-H3 depicted in SEQ ID NO: 29, and a VL region comprising a CDR-L1 depicted in SEQ ID NO: 30, a CDR-L2 depicted in SEQ ID NO: 31 and a CDR-L3 depicted in SEQ ID NO: 32;
   c) a VH region comprising a CDR-H1 depicted in SEQ ID NO: 41, a CDR-H2 depicted in SEQ ID NO: 42, and a CDR-H3 depicted in SEQ ID NO: 43, and a VL region comprising a CDR-L1 depicted in SEQ ID NO: 44, a CDR-L2 depicted in SEQ ID NO: 45 and a CDR-L3 depicted in SEQ ID NO: 46;
   d) a VH region comprising a CDR-H1 depicted in SEQ ID NO: 55, a CDR-H2 depicted in SEQ ID NO: 56, and a CDR-H3 depicted in SEQ ID NO: 57, and a VL region comprising a CDR-L1 depicted in SEQ ID NO: 58, a CDR-L2 depicted in SEQ ID NO: 59 and a CDR-L3 depicted in SEQ ID NO: 60;
   e) a VH region comprising a CDR-H1 depicted in SEQ ID NO: 69, a CDR-H2 depicted in SEQ ID NO: 70, and a CDR-H3 depicted in SEQ ID NO: 71, and a VL region comprising a CDR-L1 depicted in SEQ ID NO: 72, a CDR-L2 depicted in SEQ ID NO: 73 and a CDR-L3 depicted in SEQ ID NO: 74;
   f) a VH region comprising a CDR-H1 depicted in SEQ ID NO: 83, a CDR-H2 depicted in SEQ ID NO: 84, and a CDR-H3 depicted in SEQ ID NO: 85, and a VL region comprising a CDR-L1 depicted in SEQ ID NO: 86, a CDR-L2 depicted in SEQ ID NO: 87 and a CDR-L3 depicted in SEQ ID NO: 88;
   g) a VH region comprising a CDR-H1 depicted in SEQ ID NO: 97, a CDR-H2 depicted in SEQ ID NO: 98, and a CDR-H3 depicted in SEQ ID NO: 99, and a VL region comprising a CDR-L1 depicted in SEQ ID NO: 100, a CDR-L2 depicted in SEQ ID NO: 101 and a CDR-L3 depicted in SEQ ID NO: 102;
   h) a VH region comprising a CDR-H1 depicted in SEQ ID NO: 111, a CDR-H2 depicted in SEQ ID NO: 112, and a CDR-H3 depicted in SEQ ID NO: 113, and a VL region comprising a CDR-L1 depicted in SEQ ID NO: 114, a CDR-L2 depicted in SEQ ID NO: 115 and a CDR-L3 depicted in SEQ ID NO: 116;
   i) a VH region comprising a CDR-H1 depicted in SEQ ID NO: 125, a CDR-H2 depicted in SEQ ID NO: 126, and a CDR-H3 depicted in SEQ ID NO: 127, and a VL region comprising a CDR-L1 depicted in SEQ ID NO: 128, a CDR-L2 depicted in SEQ ID NO: 129 and a CDR-L3 depicted in SEQ ID NO: 130;
   j) a VH region comprising a CDR-H1 depicted in SEQ ID NO: 139, a CDR-H2 depicted in SEQ ID NO: 140, and a CDR-H3 depicted in SEQ ID NO: 141, and a VL region comprising a CDR-L1 depicted in SEQ ID NO: 142, a CDR-L2 depicted in SEQ ID NO: 143 and a CDR-L3 depicted in SEQ ID NO: 144;
   k) a VH region comprising a CDR-H1 depicted in SEQ ID NO: 153, a CDR-H2 depicted in SEQ ID NO: 154, and a CDR-H3 depicted in SEQ ID NO: 155, and a VL region comprising a CDR-L1 depicted in SEQ ID NO: 156, a CDR-L2 depicted in SEQ ID NO: 157 and a CDR-L3 depicted in SEQ ID NO: 158;
   l) a VH region comprising a CDR-H1 depicted in SEQ ID NO: 167, a CDR-H2 depicted in SEQ ID NO: 168, and a CDR-H3 depicted in SEQ ID NO: 169, and a VL region comprising a CDR-L1 depicted in SEQ ID NO: 170, a CDR-L2 depicted in SEQ ID NO: 171 and a CDR-L3 depicted in SEQ ID NO: 172;
   m) a VH region comprising a CDR-H1 depicted in SEQ ID NO: 181, a CDR-H2 depicted in SEQ ID NO: 182, and a CDR-H3 depicted in SEQ ID NO: 183, and a VL region comprising a CDR-L1 depicted in SEQ ID NO: 184, a CDR-L2 depicted in SEQ ID NO: 185 and a CDR-L3 depicted in SEQ ID NO: 186;
   n) a VH region comprising a CDR-H1 depicted in SEQ ID NO: 195, a CDR-H2 depicted in SEQ ID NO: 196, and a CDR-H3 depicted in SEQ ID NO: 197, and a VL region comprising a CDR-L1 depicted in SEQ ID NO: 198, a CDR-L2 depicted in SEQ ID NO: 199 and a CDR-L3 depicted in SEQ ID NO: 200;
   o) a VH region comprising a CDR-H1 depicted in SEQ ID NO: 209, a CDR-H2 depicted in SEQ ID NO: 210, and a CDR-H3 depicted in SEQ ID NO: 211, and a VL region comprising a CDR-L1 depicted in SEQ ID NO: 212, a CDR-L2 depicted in SEQ ID NO: 213 and a CDR-L3 depicted in SEQ ID NO: 214;
   p) a VH region comprising a CDR-H1 depicted in SEQ ID NO: 223, a CDR-H2 depicted in SEQ ID NO: 224, and a CDR-H3 depicted in SEQ ID NO: 225, and a VL region comprising a CDR-L1 depicted in SEQ ID NO: 226, a CDR-L2 depicted in SEQ ID NO: 227 and a CDR-L3 depicted in SEQ ID NO: 228;
   q) a VH region comprising a CDR-H1 depicted in SEQ ID NO: 237, a CDR-H2 depicted in SEQ ID NO: 238, and a CDR-H3 depicted in SEQ ID NO: 239, and a VL region comprising a CDR-L1 depicted in SEQ ID NO: 240, a CDR-L2 depicted in SEQ ID NO: 241 and a CDR-L3 depicted in SEQ ID NO: 242;
   r) a VH region comprising a CDR-H1 depicted in SEQ ID NO: 251, a CDR-H2 depicted in SEQ ID NO: 252, and a CDR-H3 depicted in SEQ ID NO: 253, and a VL region comprising a CDR-L1 depicted in SEQ ID NO: 254, a CDR-L2 depicted in SEQ ID NO: 255 and a CDR-L3 as depicted in SEQ ID NO: 256,
   s) a VH region comprising a CDR-H1 depicted in SEQ ID NO: 265, a CDR-H2 depicted in SEQ ID NO: 266, and a CDR-H3 depicted in SEQ ID NO: 267, and a VL region comprising a CDR-L1 depicted in SEQ ID NO: 268, a CDR-L2 depicted in SEQ ID NO: 269, and a CDR-L3 as depicted in SEQ ID NO: 270.
xlxix) The polypeptide or polypeptide construct according to any one of the preceding items, wherein the domain binding to CLDN6 comprises a VH region having an amino acid sequence selected from the group comprising the sequences depicted in SEQ ID NO: 11, SEQ ID NO: 25, SEQ ID NO: 39, SEQ ID NO: 53, SEQ ID NO: 67, SEQ ID NO: 81, SEQ ID NO: 95, SEQ ID NO: 109, SEQ ID NO: 123, SEQ ID NO: 137, SEQ ID NO: 151, SEQ ID NO: 165, SEQ ID NO: 179, SEQ ID NO: 193, SEQ ID NO: 207, SEQ ID NO: 221, SEQ ID NO: 235, SEQ ID NO: 249, or SEQ ID NO: 263,
   wherein said VH region amino acid sequence may have one or more modifications of one or several amino acid residues in the framework and/or hypervariable regions, provided said domain comprising said modified VH region selectively binds to CLDN6, and
   optionally wherein said domain is part of a polypeptide or polypeptide construct that activates T cells and maintains the ability to induce T cell-dependent cytotoxicity,
   further, optionally, wherein said domain is part of a polypeptide or polypeptide construct that activates T cells and maintains the ability to induce T cell-dependent cytotoxicity with more than 1000 fold efficacy than in an identical cell type which expresses CLDN9, but does not express CLDN6, and
   still further optionally, wherein said domain is part of a polypeptide or polypeptide construct that is not capable of activating T cells and inducing T cell-dependent cytotoxicity in CLDN6-negative cells of the same cell type, preferably when tested in an in vitro cytotoxicity assay.
xlxix) The polypeptide or polypeptide construct according to any one of the preceding items, wherein the domain binding to CLDN6 comprises a VL region having an amino acid sequence selected from the group comprising the sequences depicted in SEQ ID NO: 12, SEQ ID NO: 26, SEQ ID NO: 40, SEQ ID NO: 54, SEQ ID NO: 68, SEQ ID NO: 82, SEQ ID NO: 96, SEQ ID NO: 110, SEQ ID NO: 124, SEQ ID NO: 138, SEQ ID NO: 152, SEQ ID NO: 166, SEQ ID NO: 180, SEQ ID NO: 194, SEQ ID NO: 208, SEQ ID NO: 222, SEQ ID NO: 236, SEQ ID NO: 250, or SEQ ID NO: 264,
   wherein said VL region amino acid sequence may have one or more modifications of one or several amino acid residues in the framework and/or hypervariable regions, provided said domain comprising said modified VL region selectively binds to CLDN6, and
   optionally wherein said domain is part of a polypeptide or polypeptide construct that activates T cells and maintains the ability to induce T cell-dependent cytotoxicity in target cells,
   further, optionally, wherein said domain is part of a polypeptide or polypeptide construct that activates T cells and maintains the ability to induce T cell-dependent cytotoxicity with more than 500 fold efficacy than in control cells which do not express CLDN6, wherein these cells optionally express CLDN9, but
   still further optionally, wherein said domain is part of a polypeptide or polypeptide construct that is not capable of activating T cells and inducing T cell-dependent cytotoxicity in CLDN6-negative cells of the same cell type, preferably when tested in a in vitro cytotoxicity assay.
xlxx) The polypeptide or polypeptide construct according to any one of the preceding items, wherein the domain binding to CLDN6 comprises a pair of a VH region and a VL region having amino acid sequences as depicted in SEQ ID NOs: 11+12, SEQ ID NO: 25+26, SEQ ID NO: 39+40, SEQ ID NO: 53+54, SEQ ID NO: 67+68, SEQ ID NO: 81+82, SEQ ID NO: 95+96, SEQ ID NO: 109+110, SEQ ID NO: 123+124, SEQ ID NO: 137+138, SEQ ID NO: 151+152, SEQ ID NO: 165+166, SEQ ID NO: 179+180, SEQ ID NO: 193+194, SEQ ID NO: 207+208, SEQ ID NO: 221+222, SEQ ID NO: 235+236, SEQ ID NO: 249+250, or SEQ ID NO: 263+264.
xlxxi) The polypeptide or polypeptide construct according to any one of the preceding items, wherein the domain binding to CLDN6 comprises an amino acid sequence as depicted in SEQ ID NO: 19, SEQ ID NO: 22, SEQ ID NO: 33, SEQ ID NO: 36, SEQ ID NO: 47, SEQ ID NO: 50, SEQ ID NO: 61, SEQ ID NO: 64, SEQ ID NO: 75, SEQ ID NO: 78, SEQ ID NO: 89, SEQ ID NO: 92, SEQ ID NO: 103, SEQ ID NO: 106, SEQ ID NO: 117, SEQ ID NO: 120, SEQ ID NO: 131, SEQ ID NO: 134, SEQ ID NO: 145, SEQ ID NO: 148, SEQ ID NO: 159, SEQ ID NO: 162, SEQ ID NO: 173, SEQ ID NO: 176, SEQ ID NO: 187, SEQ ID NO: 190, SEQ ID NO: 201, SEQ ID NO: 204, SEQ ID NO: 215, SEQ ID NO: 218, SEQ ID NO: 229, SEQ ID NO: 232, SEQ ID NO: 243, SEQ ID NO: 246, SEQ ID NO: 257, or SEQ ID NO: 260, SEQ ID NO: 271 or SEQ ID NO: 274.
xlxxii) The polypeptide or polypeptide construct according to any one of the preceding items, comprising a polypeptide having an amino acid sequence selected from the group of those depicted in SEQ ID NO: 19, SEQ ID NO: 20, SEQ ID NO: 21, SEQ ID NO: 22, SEQ ID NO: 23, and SEQ ID NO: 24, SEQ ID NO: 33, SEQ ID NO: 34, SEQ ID NO: 35, SEQ ID NO: 36, SEQ ID NO: 37, and SEQ ID NO: 38, SEQ ID NO: 47, SEQ ID NO: 48, SEQ ID NO: 49, SEQ ID NO: 50, SEQ ID NO: 51, and SEQ ID NO: 52, SEQ ID NO: 61, SEQ ID NO: 62, SEQ ID NO: 63, SEQ ID NO: 64, SEQ ID NO: 65, and SEQ ID NO: 66, SEQ ID NO: 75, SEQ ID NO: 76, SEQ ID NO: 77, SEQ ID NO: 78, SEQ ID NO: 79, and SEQ ID NO: 80, SEQ ID NO: 89, SEQ ID NO: 90, SEQ ID NO: 91, SEQ ID NO: 92, SEQ ID NO: 93, and SEQ ID NO: 94, SEQ ID NO: 103, SEQ ID NO: 104, SEQ ID NO: 105, SEQ ID NO: 106, SEQ ID NO: 107, and SEQ ID NO: 108, SEQ ID NO: 117, SEQ ID NO: 118, SEQ ID NO: 119, SEQ ID NO: 120, SEQ ID NO: 121, and SEQ ID NO: 122, SEQ ID NO: 131, SEQ ID NO: 132, SEQ ID NO: 133, SEQ ID NO: 134, SEQ ID NO: 135, and SEQ ID NO: 136, SEQ ID NO: 145, SEQ ID NO: 146, SEQ ID NO: 147, SEQ ID NO: 148, SEQ ID NO: 149, and SEQ ID NO: 150, SEQ ID NO: 159, SEQ ID NO: 160, SEQ ID NO: 161, SEQ ID NO: 162, SEQ ID NO: 163, and SEQ ID NO: 164, SEQ ID NO: 173, SEQ ID NO: 174, SEQ ID NO: 175, SEQ ID NO: 176, SEQ ID NO: 177, and SEQ ID NO: 178, SEQ ID NO: 187, SEQ ID NO: 188, SEQ ID NO: 189, SEQ ID NO: 190, SEQ ID NO: 191, and SEQ ID NO: 192, SEQ ID NO: 201, SEQ ID NO: 202, SEQ ID NO: 203, SEQ ID NO: 204, SEQ ID NO: 205, and SEQ ID NO: 206, SEQ ID NO: 215, SEQ ID NO: 216, SEQ ID NO: 217, SEQ ID NO: 218, SEQ ID NO: 219, and SEQ ID NO: 220, SEQ ID NO: 229, SEQ ID NO: 230, SEQ ID NO: 231, SEQ ID NO: 232, SEQ ID NO: 233, and SEQ ID NO: 234, SEQ ID NO: 243, SEQ ID NO: 244, SEQ ID NO: 245, SEQ ID NO: 246, SEQ ID NO: 247, and SEQ ID NO: 248, SEQ ID NO: 257, SEQ ID NO: 258, SEQ ID NO: 259, SEQ ID NO: 260, SEQ ID NO: 261, and SEQ ID NO: 262, SEQ ID NO: 271, SEQ ID NO: 272, SEQ ID NO: 273, SEQ ID NO: 274, SEQ ID NO: 275, and SEQ ID NO: 276, or from polypeptides/polypeptide constructs having an amino acid having at least 90, 91, 92, 93, 94, 95, 96, 97, 98 or 99% identity to said sequences.
xlxxiii) The polypeptide or polypeptide construct according to any one of the preceding items, wherein the domain binding to CLDN6 induces at least 100 fold, at least 250 fold, at least 500 fold lower cytotoxicity as determined in an in vitro assay using a cell that expresses a mutant of wild-type CLDN6 as depicted in SEQ ID NO: 1 that comprises at least one or more of the following mutations M29X, wherein X is preferably L, R145X, wherein X is preferably Q, and/or Q156X, wherein X is preferably L, as compared with the cytotoxicity measured in the in vitro assay using a cell that expresses CLDN6 as depicted in SEQ ID NO: 1.
xlxxiv) The polypeptide or polypeptide construct according to any one of the preceding items, wherein the domain binding to CLDN6 induces at least 100 fold, at least 250 fold, at least 500 fold lower cytotoxicity as determined in an in vitro assay using a cell that expresses a mutant of wild-type CLDN6 as depicted in SEQ ID NO: 1 that comprises at least one or more of the following mutations M29X, wherein X is preferably L, R145X, wherein X is preferably Q, and/or Q156X, wherein X is preferably L, as compared with the cytotoxicity measured in the in vitro assay using a cell that expresses CLDN6 as depicted in SEQ ID NO: 1, wherein said construct is capable of activating T cells and inducing cytotoxicity in target cells expressing CLDN6, and wherein said construct has a heavy chain CDR3 sequence comprising: X1LIVX2APX3 (SEQ ID NO. 667), wherein X1 is either A or N; X2 is either V or E; and X3 is either V or A.
xlxxv) The polypeptide or polypeptide construct according to any one of the preceding items, wherein the construct is a single chain construct.
xlxxvi) The polypeptide or polypeptide construct according to any one of the preceding items, wherein said half-life extending domain comprising two polypeptide monomers comprises a hinge, a CH2 domain and a CH3 domain comprising in an amino to carboxyl order:
   hinge-CH2-CH3-linker-hinge-CH2-CH3.
xlxxvii) The polypeptide or polypeptide construct according to any one of the preceding items, wherein the CH2 domain comprises an intra domain cysteine disulfide bridge.
xlxxviii) The polypeptide or polypeptide construct according to any one of the preceding items, wherein
   (i) the antigen-binding (epitope-binding) domain binding CLDN6 comprises two antibody variable domains and the antigen-binding (epitope-binding) domain binding CD3 comprises two antibody variable domains;
   (ii) the antigen-binding (epitope-binding) domain binding CLDN6 comprises one antibody variable domain and the antigen-binding (epitope-binding) domain binding CD3 comprises two antibody variable domains;
   (iii) the antigen-binding (epitope-binding) domain binding CLDN6 comprises two antibody variable domains and the antigen-binding (epitope-binding) domain binding CD3 comprises one antibody variable domain; or
   (iv) the antigen-binding (epitope-binding) domain binding CLDN6 comprises one antibody variable domain and the antigen-binding (epitope-binding) domain binding CD3 comprises one antibody variable domain.
xlxxix) The polypeptide or polypeptide construct according to any one of the preceding items, wherein the antigen-binding (epitope-binding) binding CLDN6 and the antigen-binding (epitope-binding) domain binding CD3 are fused to another domain via a peptide linker.
xlxxx) The polypeptide or polypeptide construct according to any one of the preceding items, wherein the polypeptide or polypeptide construct comprises in an amino to carboxyl order, or in a carboxyl to amino order:
   (a) an antigen-binding (epitope-binding) domain binding to CLDN6;
   (b) a peptide linker, particularly a peptide linker having an amino acid sequence selected from the group consisting of SEQ ID NOs: 563-575;
   (c) an antigen-binding (epitope-binding) domain binding to CD3.
xlxxxi) The polypeptide or polypeptide construct according to any one of the preceding items, wherein the polypeptide or polypeptide construct further comprises in an amino to carboxyl order, or in a carboxyl to amino order, or between the antigen-binding (epitope-binding) domain binding to CLDN6 and the antigen-binding (epitope-binding) domain binding to CD3:
   (a) a peptide linker having an amino acid sequence selected from the group consisting of SEQ ID NOs: 563-575;
   (b) the first polypeptide monomer of a third domain;
   (c) a peptide linker having an amino acid sequence selected from the group consisting of SEQ ID NOs: 563-575; and
   (d) the second polypeptide monomer of said third domain.
xlxxxii) The polypeptide or polypeptide construct according to any one of the preceding items, wherein the construct is depicted in any one of the sequences depicted in SEQ ID NO: 21, 24, 35, 38, 49, 52, 63, 66, 77, 80, 91, 94, 105, 108, 119, 122, 133, 136, 147, 150, 161, 164, 175, 178, 189, 192, 203, 206, 217, 220, 231, 234, 245, 148, 259, 262, 273, 276, 287, 290, 301, 304, 315, 318, 329, 332, 343, 346, 357, 360, 371, 374, 385, 388, 399, 402, 413, 416, 427, and 430, particularly, 21, 24, 35, 38, 49, 52, 63, 66, 77, 80, 91, 94, more particularly, 21, 24, 35, 38, 49, 52, 77, and 80, and even more particularly, 21, 35, 49, and 77.
xlxxxiii) The polypeptide or polypeptide construct according to any one of the preceding items, wherein the construct comprises a domain binding to CD3 comprising a VH domain comprising at least one, two, or all of the following CDR sequences as depicted in SEQ ID NO: 670, 671, and/or 672.
xlxxxiv) The polypeptide or polypeptide construct according to any one of the preceding items, wherein the construct comprises a domain binding to CD3 comprising a VL domain comprising at least one, two, or all of the following CDR sequences as depicted in SEQ ID NO: 673, 674, and/or 675.
xlxxxv) The polypeptide or polypeptide construct according to any one of the preceding items, wherein the construct comprises a domain binding to CD3 comprising a VH and a VL domain comprising at least one, two, or all of the following CDR sequences as depicted in SEQ ID NO: 670, 671, 672, 673, 674, and/or 675.
xlxxxvi) The polypeptide or polypeptide construct according to any one of the preceding items, wherein the construct comprises a domain binding to CD3 comprising a VH domain as depicted in SEQ ID NO: 676.
xlxxxvii) The polypeptide or polypeptide construct according to any one of the preceding items, wherein the construct comprises a domain binding to CD3 comprising a VL domain as depicted in SEQ ID NO: 677.
xlxxxviii) The polypeptide or polypeptide construct according to any one of the preceding items, wherein the construct comprises a domain binding to CD3 comprising a VH domain as depicted in SEQ ID NO: 676 and a VL domain as depicted in SEQ ID NO: 677.
xlxxxix) The polypeptide or polypeptide construct according to any one of the preceding items, wherein the construct comprises a domain binding to CD3 comprising a scFv domain as depicted in SEQ ID NO: 678.
xc) A polynucleotide encoding a polypeptide or polypeptide construct as defined in any one of the preceding items.
ixc) A vector comprising a polynucleotide as defined in item xc).
iiivc) A host cell transformed or transfected with the polynucleotide as defined in item xc) or with the vector as defined in item ixc).
iivc) A process for producing a polypeptide or polypeptide construct as defined in any one of the preceding items, said process comprising culturing a host cell as defined in item iiivc) under conditions allowing the expression of said polypeptide construct and recovering the produced polypeptide or polypeptide construct from the culture.
ivc) A pharmaceutical composition comprising a polypeptide or polypeptide construct as defined in any one of preceding items, or that is produced according to the process of claim iivc).
vc) The polypeptide or polypeptide construct according to any one of the preceding items or that is produced according to the process of claim iivc) for the use as a medicament, particularly for the use in the prevention, treatment or amelioration of a disease, preferably a neoplasm.
vic) The polypeptide or polypeptide construct according to claim vc) for the use as a medicament, particularly for the use in the prevention, treatment or amelioration of a disease, wherein the disease or neoplasm is selected from the group consisting of germ cell cancer, ovarian cancer, in particular ovarian adenocarcinoma and ovarian teratocarcinoma, uterine cancer, and lung cancer, including small cell lung cancer (SCLC) and non-small cell lung cancer (NSCLC), in particular squamous cell lung carcinoma and adenocarcinoma.
viic) The polypeptide or polypeptide construct according to claim vc), wherein the lung cancer is non-small cell lung cancer (NSCLC), in particular squamous cell lung carcinoma and adenocarcinoma.
viiic) A kit comprising a polypeptide or polypeptide construct as defined in any one of the preceding items, a polypeptide or polypeptide construct produced according to the process of claim vc, a polynucleotide, a vector, and/or a host cell as defined in the above items.
ic) A method for the treatment or amelioration of a proliferative disease, a tumorous disease, cancer, or an immunological disorder, comprising the step of administering to a subject in need thereof the polypeptide or polypeptide construct according to any one of above items, or produced according to the process according to the above item, wherein the disease preferably is selected from the group consisting of germ cell cancer, ovarian cancer, in particular ovarian adenocarcinoma and ovarian teratocarcinoma, uterine cancer, more particularly from ovarian serous cystadenocarcinoma, uterine carcinosarcoma, uterine corpus endometrial carcinoma, and lung cancer, including small cell lung cancer (SCLC) and non-small cell lung cancer (NSCLC), in particular squamous cell lung carcinoma and adenocarcinoma.

Whenever the term “construct” is used in the figures, said term refers to the used polypeptide/polypeptide constructs of the invention or controls thereof as indicated.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

The above description and the below examples provide exemplary arrangements, but the present invention is not limited to the specific methodologies, techniques, protocols, material, reagents, substances, etc., described herein and as such can vary. The terminology used herein serves to describe specific embodiments only. The terminology used herein does not intend to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### FIGURES

FIG. 1 shows the results of the epitope mapping analysis. The topmost row provides structure representations of either CLDN4 (dashed graph) and CLDN6 (continuous graphs) and chimeric proteins, indicating the various regions of the CLDN6 that have been replaced by the respective CLDN4 regions. The second and third rows from the above show the results of FACS analyses using isotype controls and anti-CLDN4-Ab and anti-CLDN6-Ab (5 μg/ml Isotype control (R&D IC003P)+5 μg/ml anti-CLDN4-ab (clone 382321 R&D MAB4219). The two left FACS results show that cells that neither express CLDN4 nor CLDN6 are not recognized by either of the antibodies immunospecifically detecting CLDN-4 or CLDN-6. The four rows at the bottom show the results of FACS analyses using four different polypeptides/polypeptide constructs of the invention as primary binders (5 μg/ml CLDN6-polypeptides/polypeptide constructs) or PBS with 2% FCS as controls 4 lead candidates: SEQ ID NO: 343, SEQ ID NO. 35, SEQ ID NO. 21, and SEQ ID NO: 105 (from top to bottom). As secondary binders, 1:50 anti-hu Fcy-PE (Jacks.Imm.Res. 109-116-98) is used. The E1A and E2B regions are particularly important for the binding of some polypeptides/polypeptide constructs of the invention to CLDN6. On the ordinate axis (x-axis) the numbers 0, 20, 40, 60, 80, and 100 are depicted. On the abscissa (y-axis) the values 10¹, 10², 10³, 10⁴, and 10⁵ are indicated.

FIG. 2 shows the results of experiments of human pan T cells that were incubated with target cells in a 10:1 ratio and polypeptides/polypeptide constructs in the concentrations indicated. After 48 hours, cell viability was measured with a Cell Titer-glossary and percent cytotoxicity was calculated. Graphs show representative data for duplicate samples (>2 independent experiments were run). Data were analyzed with GraphPad Prism. Similarly, experiments were conducted with a polypeptide construct based on a prior art antibody (A3E-20; disclosed in WO 2009/087978). The CLDN6 constructs in SEQ ID NOs: 21, 24, 217, 147, 119, and 90 are more potent than the A3E-20-based polypeptide construct; CLDN6×CD3 polypeptides/polypeptide constructs according to the invention have >3000-fold selectivity for CLDN6 as compared to CLDN9. FIG. 2A=PA-1 cells; FIG. 2B=CHO-CLDN6; FIG. 2C=CHO-CLDN9.

FIG. 3 shows that equivalent activity of constructs selectively binding CLDN6 having different CD3 binding moieties CLDN6XI2C and CLDN6XI2E molecules according to the invention (SEQ ID NOs.: 21 and 24); CLDN6-dependent cytotoxic activity. Human pan T cells were incubated with target cells in a 10:1 ratio and polypeptide construct in the concentrations indicated. After 48 hours, cell viability was measured with a Cell Titer-gloassay and percent cytotoxicity was calculated. Graphs show representative data for duplicate samples (>2 independent experiments were run). Data were analyzed with GraphPad Prism. The CLDN6xI2C molecule and CLDN6xI2E molecule molecules according to the invention (SEQ ID NOs.: 21 and 24) have equivalent cytotoxic activity in vitro; both molecules show specificity for killing of CLDN6-expressing cells (FIGS. 3 A) and B)).

FIG. 4 shows the results of human PBMCs that were incubated with different target cells in a 5:1 ratio and polypeptides/polypeptide constructs in the concentrations indicated. After 48 hours, cell viability was measured with a flow-cytometry-based assay and percent cytotoxicity was calculated. Graphs show representative data from three PBMC donors (>2 independent experiments were run). Data were analyzed with GraphPad Prism. Black lines, CLDN6xI2C (SEQ ID NO: 24); gray lines, CLDN6xI2E (SEQ ID NO: 21). The numbers (#150, #156, #158) refer to three different donors of human PBMCs FIGS. 4A) to 4F)). The cell lines used are indicated above the graphs (FIG. 4A: COV-362, FIG. 4B: LCLC-97TIM1, FIG. 4C: NCI-H322, FIG. 4D: NCI-H1435, FIG. 4E: OV-90, and FIG. 4F: OVCAR-3).

FIG. 5 shows the results of a different concentration ranges of CLDN6 HLE BiTE (I2C) (SEQ ID NO.: 24) that were incubated with CHO-CLDN6 and CHO-CLDN9 cells. CHO-CLDN6 and CHO-On cell binding was assessed by flow cytometry. CHO-CLDN6 (left): dashed=mouse isotype control (BD Biosciences), solid=anti-CLDN6 antibody (Creative Biolabs, TAB-510LC, based on AE3-20 clone); CHO-CLDN9 (right): dashed=rat isotype control (BD Biosciences), solid=anti-CLDN9 antibody (Abcam, ab187116, YD4E9 clone); Secondary antibody (BV421 channel, BD Biosciences)

FIGS. 6 to 8 show that CLDN6 HLE polypeptides/polypeptide constructs have cytotoxic activity against different types of target cells that show low level CLDN6 expression by IHC. FIG. 6A: CHO-CLDN6: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIG. 6B: OVCAR-3: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIG. 6C: COV-362: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIG. 6D: NCI-H322: dashed=mouse isotype control (BD Biosciences), solid=anti-CLDN6 antibody(Creative Biolabs, TAB-510LC, based on AE3-20 clone); Secondary antibody (BV421 channel, BD Biosciences); FIGS. 7A and 7B: PA-1: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIGS. 7C and 7D: OVCAR-3: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIGS. 7E and 7F: OAW28: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIGS. 7G and 7H: LCLC-97TM1: dashed=mouse isotype control (BD Biosciences), solid=anti-CLDN6 antibody (Creative Biolabs, TAB-510LC, based on AE3-20 clone); Secondary (APC channel, Jackson ImmunoResearch); FIGS. 7I and 7J: NCI-H1435: dashed=mouse isotype control (BD Biosciences), solid=anti-CLDN6 antibody (Creative Biolabs, TAB-510LC, based on AE3-20 clone); Secondary (APC channel, Jackson ImmunoResearch).

FIG. 9—Antitumor efficacy against established OVCAR-3 xenograph tumors

FIG. 10 shows that hallmarks of engagement characteristic of T cell-activating polypeptides/polypeptide constructs were observed in an exploratory toxicology study.

FIG. 11 demonstrates that a CLDN6 HLE polypeptide construct according to the invention (SEQ ID NOs.:24) has extended half-life in non-human primates.

### EXAMPLE 1

### CLDN6 Binder Generation

### Hybridoma Generation

Pooled lymphocytes from spleen and lymph nodes are enriched for IgG+B-cells followed by electrocell fusion with mouse P3 myeloma cells. Fused hybridoma cells are cultured for 2-5 days in hyaluronic acid (HA) selection medium and then plated onto 96-well culture plates and cultured for 2 weeks to generate exhausted supernatant (ESN).

### Screening and Sequence Analysis

In primary screening, 837 human CLDN6 on-cell binding hybridomas are identified using Fluorescent Microvolume Assay Technology (FMAT). A second late primary screening via Flow Cytometry also confirms 776 of these hybridomas that bind to 293T transiently expressing cynomolgus monkey CLDN6. To further characterize the 837 CLDN6 binding hybridomas, the ESN are tested by antibody-dependent cellular cytotoxicity (ADCC) activity on PA-1 cells for a shortlist of 288 hybridomas with best killing activity. Most of these hybridomas show ADCC killing of OVCAR3 cells expressing CLDN6. The ESN of these hybridomas are further characterized for cross-reactive binding to 293 T cells transiently expressing human CLDN3, CLDN4, CLDN8 or CLDN9 on the cell surface.

In total, 20 hybridoma lines identified based on the criteria of ADCC killing in PA-1 and OVCAR3 cell lines show no cross-reactivity to CLDN3, CLDN4 and CLDN8. These hybridomas are sequenced followed by recombinant mAb generation and scaling up. These antibodies included, inter alia, three clones that showed qualifying sequence and binding properties and were selected for conversion into scFv and polypeptides/polypeptide constructs.

### Anti-CLDN6 Polypeptide Construct Generation

Assembly of anti-CLDN6 targeting polypeptides/polypeptide constructs binders is carried out by gene synthesis. In more detail: The VH of the anti-CLDN6 heavy chain and VL of the anti-CLDN6 light chain DNA are derived from hybridoma clones. Amino acid position 44 in VH and position 100 in VL (Kabat numbering) are changed to cysteine, which results in an additional disulfide bond stabilizing the target binder. A linker, e.g. consisting of three repeats of four glycines and a serine (G4S1)3-linker may be inserted between VL and VH creating a single chain Fragment variable (scFv). A human IgG heavy chain signal peptide containing a start codon embedded within a Kozak consensus sequence may be added to the N-terminus of the anti-CLDN6 binders. The assembled anti-CLDN6 target binders are converted into the recombinant bispecific single-chain binder format by joining with an anti-CD3ε specific scFv-binder, which is human in sequence and cross-reactive with human and macaque CD3ε. In these constructs the anti-CD3ε scFv is fused to a single chain Fc (scFc) half-life extension (HLE) moiety which has a C-terminal stop codon attached in frame. Human anti-CLDN6 binders were joined with the anti-CD3ε binder and the scFc in a mammalian expression vector. Some of the human anti-CLDN6 HLE polypeptides/polypeptide constructs have the domain arrangement VL CLDN6—(G4S1)3-VH CLDN6-S1G4S1—VHCD3—Peptide linker (G4S1)3—VLCD3—Peptide linker (G4)—Fc—(G4S1)6—Fc; others have the domain arrangement VH (CLDN6)—(G4S)3—VL (CLDN6)—Peptide linker (SG4S)—VH (CD3)—(G4S)3—VL (CD3)—Peptide linker (G4)—Fc—(G4S)6—Fc.

### Exemplary Sequence Optimization of Anti-CLDN6 Binder

Based on rational design a sequence optimization approach was carried out. Furthermore, a LC shuffling approach was performed to further improve the binding and protein properties of a selected polypeptide construct clone using a clonal hitpick repertoire of 899 CLDN6 binding clones. For this repertoire, a library of human light chain Vkappa variable regions was constructed using PCR Primers and ligated with the phagemid pComb3H5BHis (WO 99/25818; Saci-Spel digested) containing the VH of selected polypeptide construct clone CLDN6. The resulting combinatorial Ig light chain variable antibody library was then transformed into E. coli TG1, plated on agar and single clones were screened for binding to human CLDN6 and human CLDN9 in flow cytometry measurements. To this end, the periplasmic cell extracts of single colonies containing scFv molecules were incubated on CLDN6 transfected CHO-cells or CLDN9 transfected CHO cells and binding was detected by a mouse anti-Flag antibody and a R-Phycoerythrin-conjugated goat anti-mouse IgG or anti-mouse Fcγ-Alexa488 secondary antibody. Samples were measured on a FACSCanto II (BD Biosciences, Heidelberg, FRG).

In the screening approach clones were identified, combined with the rational design optimization approach described above and led to target binding polypeptides/polypeptide constructs as depicted, for example, in SEQ ID NOs: 21, 24, 35, 38, 49, 52, 63, 66, 77, and 80.

### Evaluation of CLDN6 Bispecific Constructs In Vitro Affinity

Cell-based affinity of CLDN6 bispecific constructs is determined by nonlinear regression (one site—specific binding) analysis. CHO cells expressing human CLDN6 are incubated with decreasing concentrations of CLDN6 bispecific constructs (400 nM, step 1:2, 11 steps) for 16 h at 4° C. Bound CLDN6 bispecific constructs were detected with Alexa Fluor 488-conjugated AffiniPure Fab Fragment Goat Anti-Human IgG (H+L; Jackson ImmunoResearch).

Fixed cells were stained with DRAQ5, Far-Red Fluorescent Live-Cell Permeant DNA Dye and signals were detected by Fluorescence Activated Cell Sorting (FACS). Respective equilibrium dissociation constant (Kd) values were calculated with the one site specific binding evaluation tool in the GraphPad Prism software (Graph Pad).

### Cytotoxic Activity

Human peripheral blood mononuclear cells (PBMC) are prepared by Ficoll density gradient centrifugation from enriched lymphocyte preparations (buffy coats), a side product of blood banks collecting blood for transfusions. Buffy coats were supplied by a local blood bank and PBMC were prepared on the same day of blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Platelets were removed via the supernatant upon centrifugation of PBMC at 100×g. Remaining lymphocytes mainly encompass B and T lymphocytes, NK cells and monocytes. PBMC were kept in culture at 37° C./5% CO₂ in RPMI medium (Gibco) with 10% FCS (Gibco).

### Depletion of CD14+ and CD56+ Cells

For depletion of CD14+ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded, and the cell pellet resuspended in MACS isolation buffer [80 μL/107 cells; PBS (Invitrogen, #20012-043), 0.5% (v/v) FBS (Gibco, #10270-106), 2 mM EDTA (Sigma-Aldrich, #E-6511)]. CD14 MicroBeads and CD56 MicroBeads (20 μL/107 cells) were added and incubated for 15 min at 4-8° C. The cells were washed with MACS isolation buffer (1-2 mL/107 cells). After centrifugation (see above), supernatant was discarded, and cells resuspended in MACS isolation buffer (500 μL/108 cells). CD14/CD56 negative cells were then isolated using LS Columns (Miltenyi Biotec, #130-042-401). PBMC w/o CD14+/CD56+ cells were cultured in RPMI complete medium i.e. RPMI1640 (Biochrom AG, #FG1215) supplemented with 10% FBS (Biochrom AG, #S0115), lx non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C. in an incubator until needed.

### Target Cell Labeling

For the analysis of cell lysis in flow cytometry assays, the fluorescent membrane dye DiOC18 (DiO) (Molecular Probes, #V22886) was used to label human CLDN6- or macaque CLDN6-transfected CHO cells as target cells and distinguish them from effector cells. Briefly, cells were harvested, washed once with PBS and adjusted to 106 cell/mL in PBS containing 2% (v/v) FBS and the membrane dye DiO (5 μL/106 cells). After incubation for 3 min at 37° C., cells were washed twice in complete RPMI medium and the cell number adjusted to 1.25×105 cells/mL. The vitality of cells was determined using the NC-250 cell counter (Chemometec)

### Flow Cytometry-Based Analysis

This assay was designed to quantify the lysis of cynomolgus or human CLDN6-transfected CHO cells in the presence of serial dilutions of CLDN6 bispecific constructs. Equal volumes of DiO-labeled target cells and effector cells (i.e., PBMC w/o CD14+ cells) were mixed, resulting in an E:T cell ratio of 10:1. 80 μl of this suspension were transferred to each well of a 96-well plate. 20 μL of serial dilutions of the CLDN6×CD3 bispecific constructs and a negative control bispecific (a CD3-based bispecific construct recognizing an irrelevant target antigen) or RPMI complete medium as an additional negative control were added. The bispecific antibody-mediated cytotoxic reaction proceeded for 48 hours in a 7% C02 humidified incubator. Then cells were transferred to a new 96-well plate and loss of target cell membrane integrity was monitored by adding propidium iodide (PI) at a final concentration of 1 μg/mL. Samples were measured by flow cytometry on an iQue Plus instrument and analyzed by Forecyt software (both from Intellicyt). Target cells were identified as DiO-positive cells. PI-negative target cells were classified as living target cells. Percentage of cytotoxicity was calculated according to the following formula:

$$\begin{matrix}Cytotoxicity [ \% ] = \frac{{n}_{dead target cells}}{{n}_{target cells}} \times 100 \\ n = number of events\end{matrix}$$

Using GraphPad Prism 5 software (Graph Pad Software, San Diego), the percentage of cytotoxicity was plotted against the corresponding bispecific construct concentrations. Dose response curves were analyzed with the four parametric logistic regression models for evaluation of sigmoid dose response curves with fixed hill slope and EC50 values were calculated.

### Bispecific Binding and Interspecies Cross-Reactivity

For confirmation of binding to human CLDN6 and CD3 and to cynomolgus CLDN6 and CD3, polypeptides/polypeptide constructs of the invention were tested by flow cytometry using
Chinese Hamster Ovary (CHO) cells transfected with human CLDN6, with human CLDN6 isoform (I143V), and with macaque CLDN6, respectively,
the human CLDN6 positive human cell line PA-1,
CD3-expressing human T cell leukemia cell line HPB-all (DSMZ), and
the cynomolgus CD3-expressing T cell line LnPx 4119

For confirmation of absence of binding to CHO CLDN1, -3, -4, -8, -17 bispecific constructs of the invention were tested by flow cytometry using CHO cells transfected with human CLDN1, -3, -4, -8 and -17. For flow cytometry 200,000 cells of the respective cell lines were incubated for 60 min at 4° C. with 50 l of purified bispecific construct at a concentration of 5 μg/ml. The cells were washed twice in PBS/2% FCS and then incubated with an in-house mouse antibody (2 μg/ml) specific for the CD3 binding part of the bispecific constructs for 30 min at 4° C. After washing, bound mouse antibodies were detected with a goat anti-mouse Fcγ-PE (Jackson ImmunoResearch; 1:100) for 30 min at 4° C. Samples were measured by flow cytometry. Non-transfected CHO cells (DSMZ) were used as negative control.

### Generation of CHO Cells Expressing CLDN6 Mutations

For the generation of CHO cells expressing hu-CLDN6, hu-CLDN9, hu-CLDN4 (SEQ ID NOs: 1, 8, and 7) as controls, the respective coding sequences were cloned into a plasmid designated pEF-DHFR (pEF-DHFR as described in Raum et al. Cancer Immunol Immunother 50 (2001) 141-150). All cloning procedures were carried out according to standard protocols (Sambrook, Molecular Cloning; A Laboratory Manual, 3rd edition, Cold Spring Harbour Laboratory Press, Cold Spring Harbour, New York (2001)). For each construct, a corresponding plasmid was transfected into DHFR deficient CHO cells for eukaryotic expression, as described by Kaufman R. J. (1990) Methods Enzymol. 185, 537-566. The expression of the above polypeptides/polypeptide constructs on CHO cells was verified in a FACS assay using antibodies against CLDN4, CLDN6 (R&D mouse anti-human CLDN6 monoclonal antibody MAB3656) and CLDN9 (rat anti-human CLDN9 monoclonal antibody ABIN1720917), respectively, at a concentration of 5 μg/ml. As negative control, cells were incubated with an isotype control antibody (BD 553454/R&D MAB0041/R&D MAB0061) instead of the first antibody. Bound monoclonal antibody was detected with a secondary anti-mouse/anti-rat/anti-human IgG Fc-gamma-PE (Jackson ImmunoResearch 115-116-071/112-116-071/109-116-098). The samples were measured by flow cytometry.

### EXAMPLE 2

### Epitope Mapping of Anti-CLDN6 Constructs

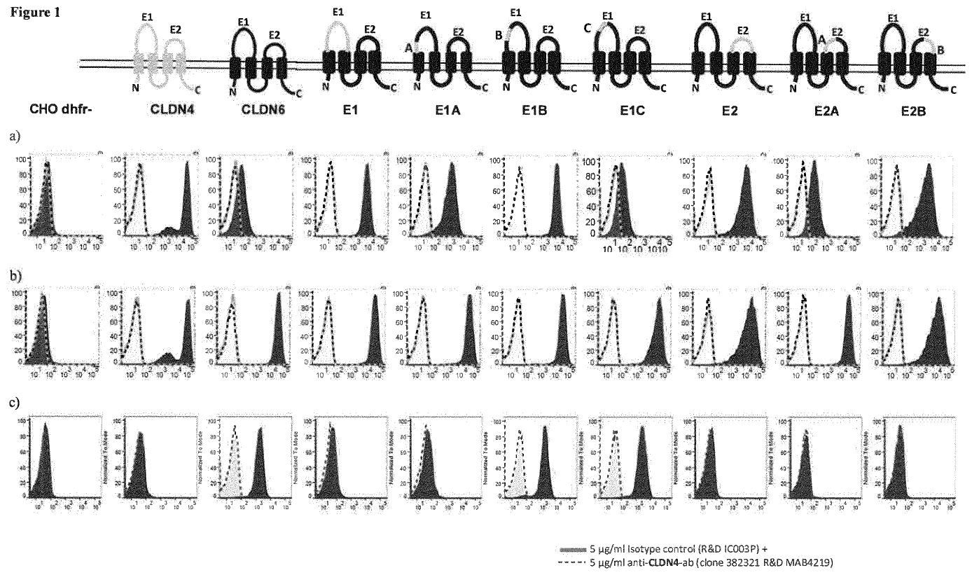

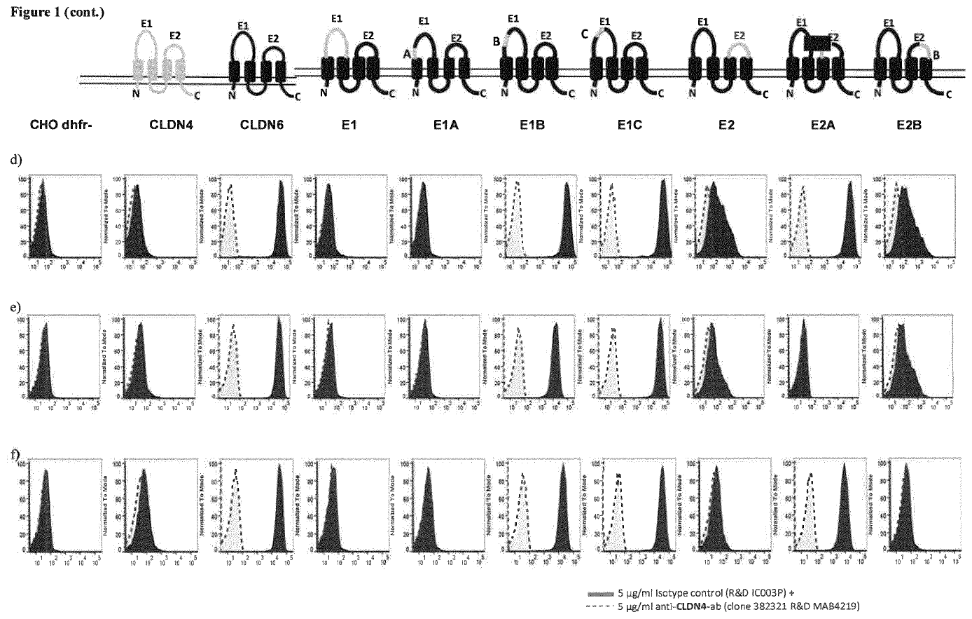

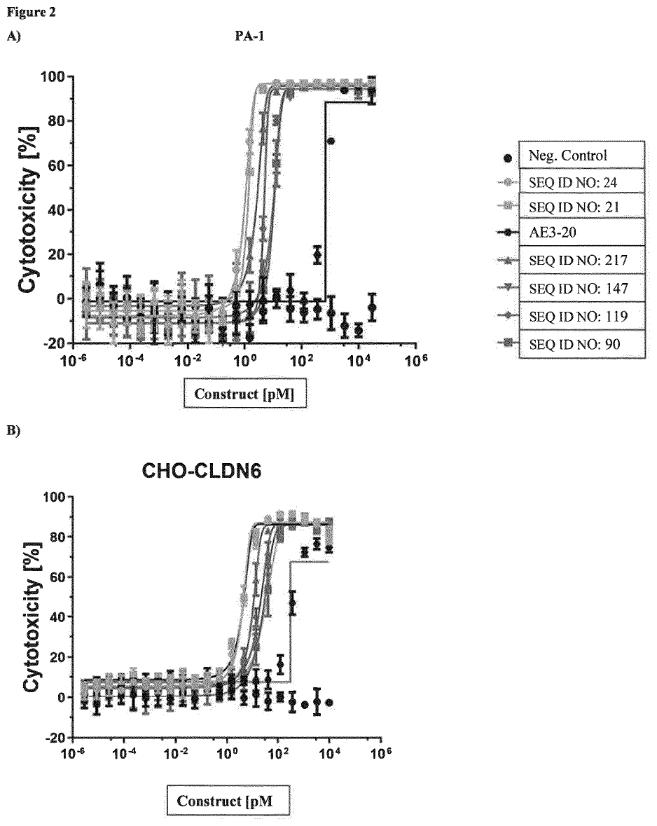

For epitope mapping, E1 (extracellular loop 1; ECL1) of CLDN6 is divided into three sub-domains (E1A, E1B, and E1C), and E2 (extracellular loop 2; ECL2) is divided into two sub-domains (E2A and E2B). The amino acid sequence of the respective epitope region (loop/domain or sub-domain) of human CLDN6 (E1, E1A, E1B, E1C, E2, E2A and E2B) is exchanged for a counterpart sequence of human CLDN4. The expression of all chimeric constructs in CHO cells is verified via FACS analysis. CHO cells transfected with the constructs described in Example 1 are stained with purified CLDN6×CD3 polypeptide construct at a concentration of 20 μg/ml. Bound constructs are detected with an anti-human IgG Fc-gamma-PE (Jackson ImmunoResearch; 1:100). Antibodies are diluted in PBS/2% FCS. As negative control, cells are incubated with PBS/2% FCS followed by the anti-human IgG Fc-gamma-PE. The samples are measured by flow cytometry. The results of the epitope mapping analysis are shown in FIG. 1. When a loss of the FACS signal is observed for cells expressing a certain CLDN6 chimera or mutation, the respective CLDN6×CD3 polypeptide construct is assumed to bind to the epitope (loop/domain/sub-domain) or to the specific amino acid that was exchanged in this CLDN6 chimeric or mutated polypeptide construct. In other words, this epitope region or amino acid is required for the binding of the CLDN6×CD3 polypeptide construct that was analyzed. In addition to the control antibodies which were used to demonstrate proper expression of the respective target, CLDN6×CD3 polypeptides/polypeptide constructs were specifically tested in the epitope mapping analysis. As shown in FIG. 1, the CLDN6×CD3 polypeptides/polypeptide constructs according to the invention (e.g., SEQ ID NOs: 21, 35, 49, 77, 203, etc.) require regions E1A and/or E2B for its selective binding to CLDN6. Consequently, and likewise, an exchange of these sub-domains with the CLDN4 counterpart sequence leads to a loss of the FACS signal.

### EXAMPLE 3

### Biacore-Based Determination of Affinity to Human and Cynomolgus CD3 and FcRn

Biacore analysis experiments were performed using recombinant human/macaque CD3-ECD (ECD=extracellular domain) fusion proteins with chicken albumin to determine target binding of the constructs of the invention.

### EXAMPLE 4

### Comparison of CLDN6×CD3 HLE Polypeptides/Polypeptide Constructs with Different CD3-Specific Paratopes and a Polypeptide Construct Based on Antibody AE3-20

A comparison of cytotoxic activity and specificity for CLDN6 of CLDN6×CD3 (Variant I2C), CLDN6×CD3 (Variant 12E) as depicted in SEQ ID NOs.: 21, 24 and other CLDN6×CD3 (Variant I2C) polypeptides/polypeptide constructs according to the invention as well as a polypeptide construct based on monoclonal antibody AE3-20 (disclosed in WO 2009/087978; depicted in SEQ ID NO: 441) was performed.

Additional comparisons of the CLDN6xI2C and CLDN6xI2E (as depicted in SEQ ID NOs.: 21 and 24) cytotoxicity in ovarian cancer cell lines and non-small cell lung cancer cell lines. For binding analysis of the AE3-20 polypeptide construct via flow cytometry CHO cells transfected with human CLDN6, hu CLDN4 or human CLDN9 were used. For flow cytometry respective CHO cells were incubated with AE3-20 polypeptide construct (5 μg/ml) or with the following monoclonal antibodies as positive control for cell surface expression: anti-CLDN6 (R&D Systems, MAB3656), anti-CLDN4 (R&D Systems, MAB4219) and anti-CLDN9 (Origene, AM26751PU-N). Binding of the AE-320 polypeptide construct or positive control antibodies was detected using mouse anti-human IgG Fc antibody conjugated to R-phycoerythrin (PE), goat anti-mouse Fc gamma-specific antibody conjugated to PE (Jackson ImmunoResearch 115-116-071) or goat anti-rat Fc gamma-specific antibody conjugated to PE (Jackson ImmunoResearch 112-116-071). As negative control respective isotype control antibodies were used.

Polypeptides that target CLDN6 epitope clusters E1A/E2B or E1A/(E2B) show unexpected higher potency compared to BiTE molecules targeting E1A/E2A+B or E2A/(E2B) (AE-320 epitope). The higher potency of polypeptides targeting CLDN6 epitope clusters E1A/E2B or E1A/(E2B) can also be observed compared to BiTE molecules targeting E2A/(E2B) (AE-3-20 epitope), although the candidates are in a similar affinity range. Surprisingly, it was noted that the epitope cluster E2A should be avoided to get sufficient potency while remaining selectivity over CLDN9. The herein investigated polypeptides, particularly those depicted in SEQ ID NOs.: 21, 24, 35, 38, 49, 52, 63, 66, 77, and 80, and more particularly SEQ ID NOs: 21 and 24 have also favourable protein properties, in particular stability in terms of favourable % monomer conversion after storage in solution at 1 mg/ml and upon repeated freeze/thaw cycles, minimal turbidity at higher protein concentration in solution over night and thermostability whilst maintaining favourable potency against CLDN6+ cell lines and very good affinity.

### Potency and Specificity of CLDN6×CD3 Constructs

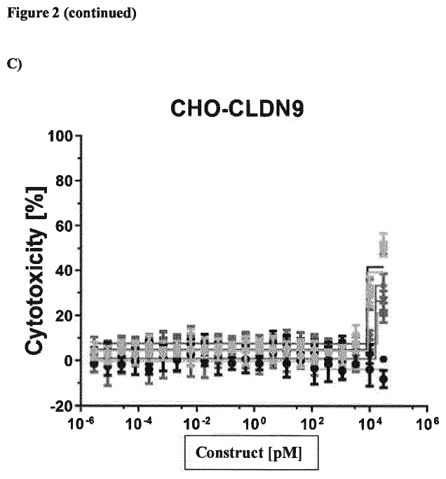

Human pan T cells (AllCells) were incubated with target cells in a 10:1 ratio and polypeptides/polypeptide constructs in the concentrations indicated in a T cell-dependent cellular cytotoxicity (TDCC) assay. After 48 hours, cell viability was measured with a Cell Titer-glo® assay (Promega) and percent cytotoxicity was calculated. Graphs in FIG. 2 show representative data for duplicate samples (>2 independent experiments were run). Data were analyzed with GraphPad Prism. Similarly, experiments were conducted with a polypeptide construct based on a prior art antibody (AE3-20; Chugai) as disclosed in WO 2009/087978. The CLDN6 polypeptides/polypeptide constructs in SEQ ID NOs: 21 and 24 are more potent than the A3E-20-based polypeptide construct; CLDN6×CD3 polypeptides/polypeptide constructs have >3000-fold selectivity for CLDN6 as compared to CLDN9.

CLDN6 HLE Polypeptides/Polypeptide Constructs—Comparison with Constructs Based on AE3-20

TDCC assay: CLDN6-expressing ovarian cancer cell line PA-1, and CHO cells transfected to stably express CLDN6 or CLDN9, were used as target cells to evaluate the in vitro cytotoxicity of CLDN6 constructs of the present invention. Cells were plated in media containing 10% fetal bovine serum in 384-well microplates (PerkinElmer), and human pan-T cells from two donors (AllCells) were added at a 10:1 ratio to the target cells. CLDN6 constructs of the present invention were added in a 22-point dose range with 60 nM as the top concentration and 5-fold dilutions. After 48 hours incubation at 37° C. in a 5% CO2 humid chamber, cell viability was assessed with a Cell Titer-glo® assay (Promega) according to the manufacturer's instructions. Luminescent signal was measured with a PerkinElmer Envision. Data were analyzed in GraphPad Prism using non-linear regression—variable slope (four parameters). The graphs show the mean values and standard deviation for dose-response curves of duplicate samples.

### Activity of Polypeptides/Polypeptide Constructs Selectively Binding CLDN6 Having Different CD3 Binding Moieties

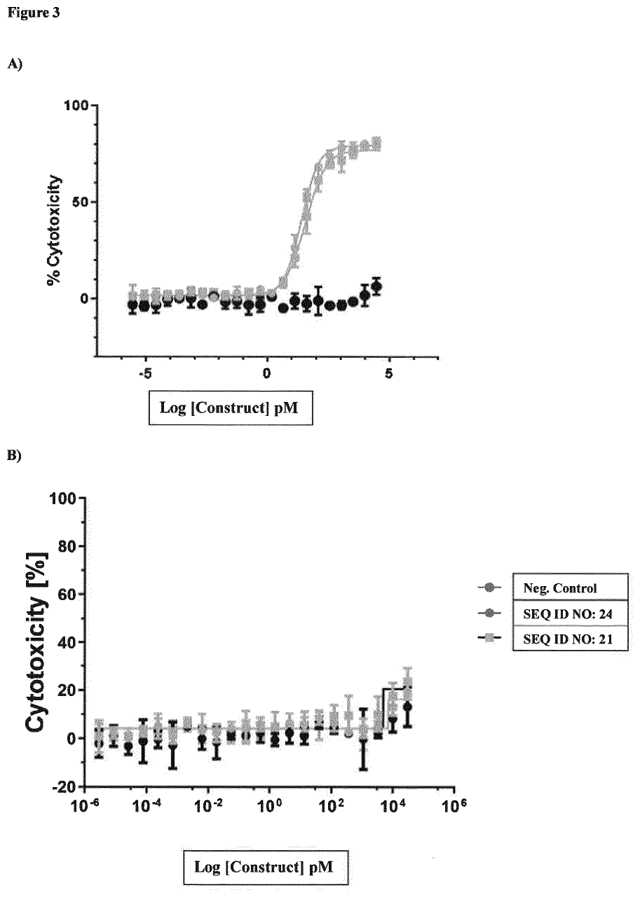

Human pan T cells were incubated with target cells in a 10:1 ratio and polypeptide construct in the concentrations indicated. After 48 hours, cell viability was measured with a Cell Titer-glo assay and percent cytotoxicity was calculated. Graphs in FIG. 3 show representative data for duplicate samples (>2 independent experiments were run). Data were analyzed with GraphPad Prism. CLDN6xI2C molecule and the CLDN6xI2E molecules according to the invention (SEQ ID NOs.: 21 and 24); CLDN6-dependent cytotoxic activity were used (FIG. 3).

The CLDN6xI2C molecule and the CLDN6xI2E molecules according to the invention (SEQ ID NOs.: 21 and 24) have equivalent cytotoxic activity in vitro and both molecules show specificity for killing of CLDN6-expressing cells. In another set of experiments, human PBMCs were incubated with target cells in a 5:1 ratio and polypeptides/polypeptide constructs in the concentrations indicated. After 48 hours, cell viability was measured with a flow-cytometry-based assay and percent cytotoxicity was calculated. Graphs show representative data from three PBMC donors (>2 independent experiments were run).

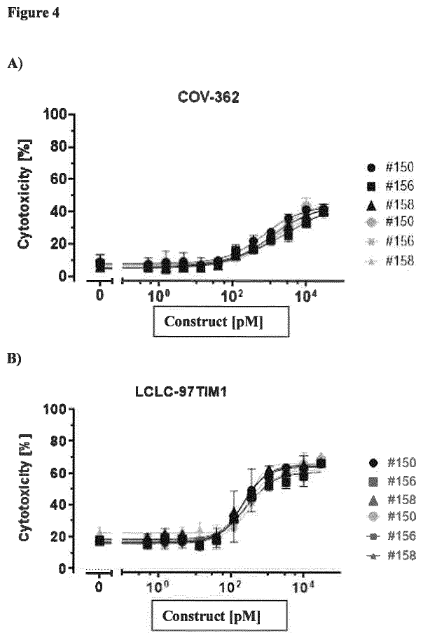

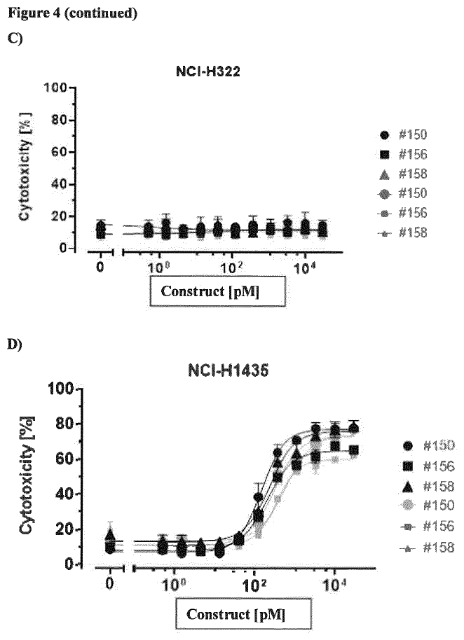

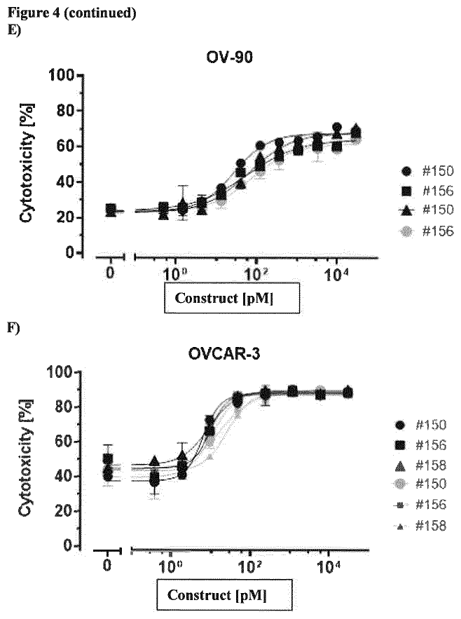

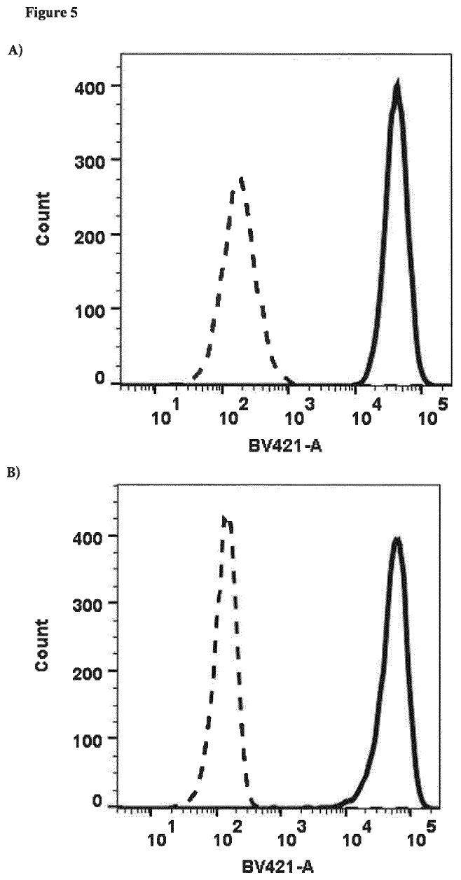

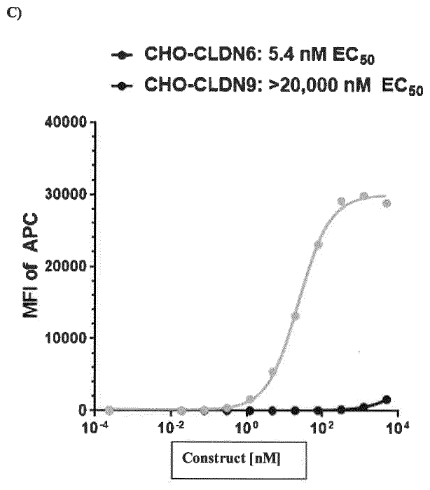

Data were analyzed with GraphPad Prism. Black lines, CLDN6xI2C (SEQ ID NO: 24); gray lines, CLDN6xI2E (SEQ ID NO: 21). The numbers (#150, #156, #158) refer to three different donors of human PBMCs FIGS. 4A) to 4F)). The cell lines used are indicated above the graphs (FIG. 4A: COV-362, FIG. 4B: LCLC-97TIM1, FIG. 4C: NCI-H322, FIG. 4D: NCI-H1435, FIG. 4E: OV-90, and FIG. 4F: OVCAR-3).

### EXAMPLE 5

### CLDN6 HLE Polypeptides/Polypeptide Constructs are Selective for CLDN6 Over CLDN9

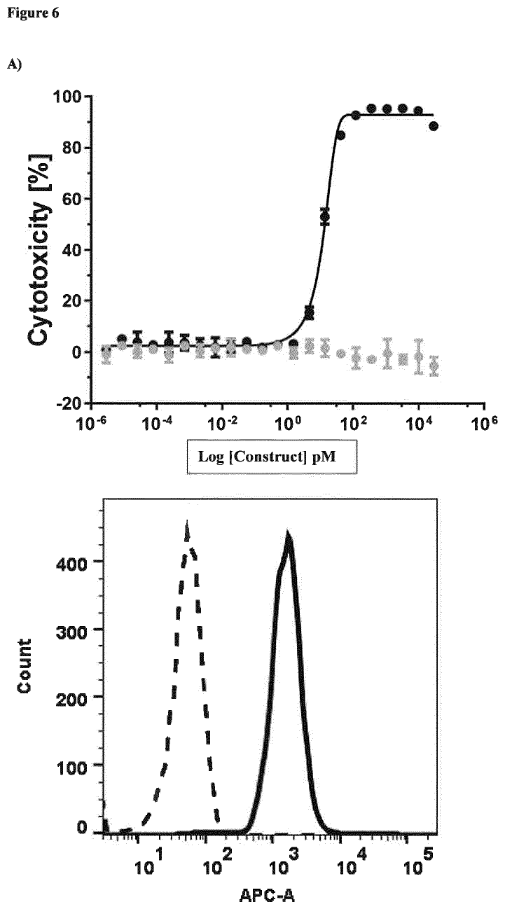

A concentration range of CLDN6 HLE BiTE (I2C) (SEQ ID NO.: 24) was incubated with CHO-CLDN6 and CHO-CLDN9 cells (FIG. 5A) and B)). CHO-CLDN6 and CHO-CLDN9 On-cell binding was assessed by flow cytometry (FIG. 5). On cell binding was assessed by flow cytometry, using a secondary antibody conjugated to APC. FIG. 5C) shows that polypeptides/polypeptide constructs of the present invention bind selectively and specifically to CLDN6, but not to CLDN9 expressed by CHO cells. Data were analyzed with FlowJo software (FlowJo, LLC). Alignment of CLDN6 and CLDN9 was conducted using Geneious software.

### EXAMPLE 6

### CLDN6 HLE Polypeptides/Polypeptide Constructs have Cytotoxic Activity Against Cells that Show Low Level CLDN6 Expression by IHC

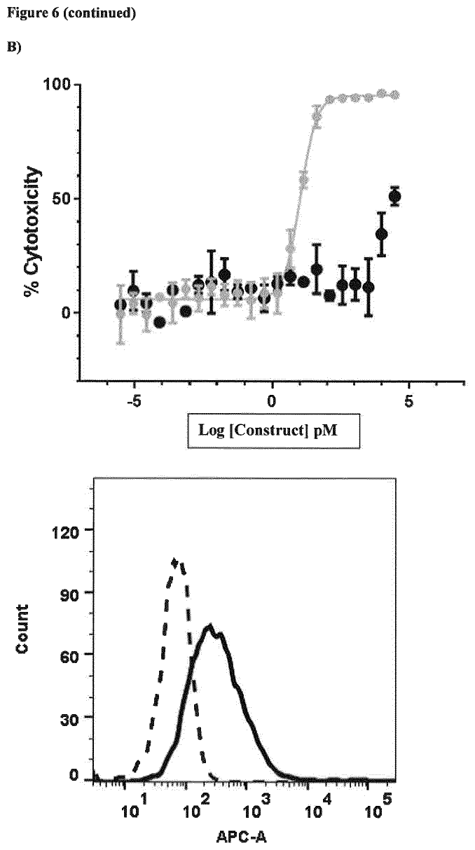

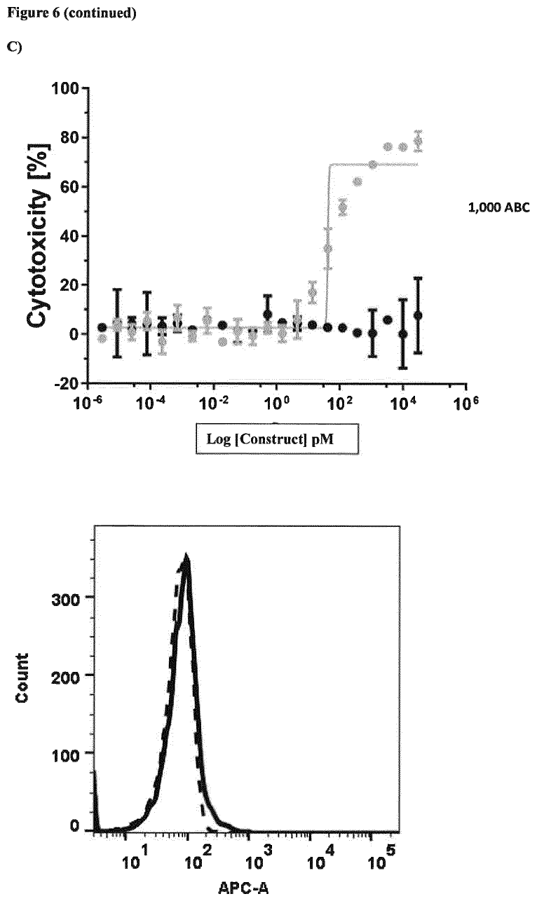

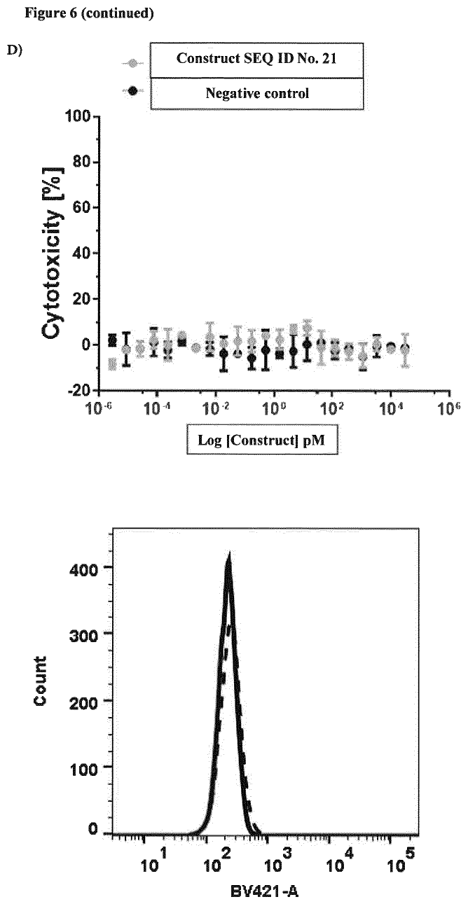

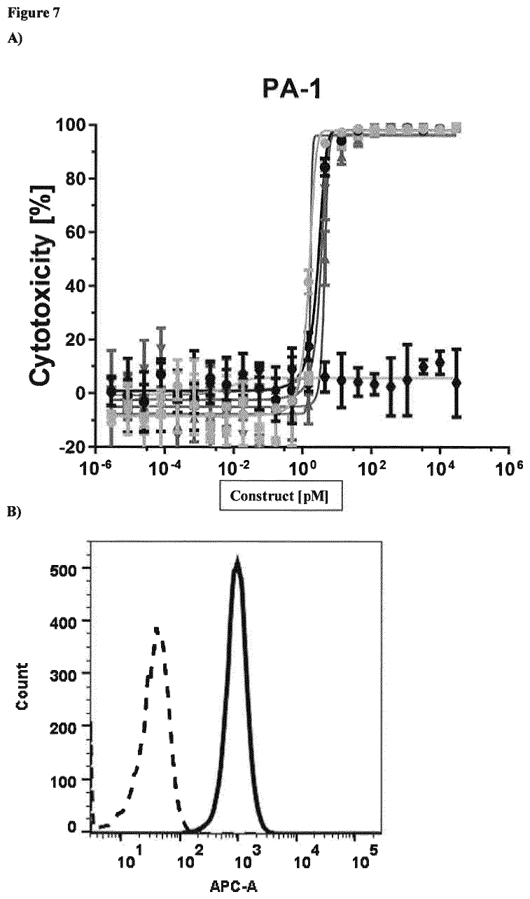

Various cancer cell lines and CHO cells expressing CLDN6 and CLDN9 were subjected to TDCC assays (FIG. 6). The antibody binding sites (ABC) EC50 values are shown in the table below. Clearly, the polypeptides/polypeptide constructs of the present invention recognize and kill cells expressing lower and higher numbers of CLDN6 sites/cell (see Table 2).

TABLE 2
TDCC EC₅₀ pM if cytotoxicity assays in various cell lines
TDCC EC₅₀ PM

| | Cell line (CLDN6 sites/cell) | | | | | | |
|---|---|---|---|---|---|---|---|
| | | | | | NCI- | CHO- | CHO- |
| | PA-1 | OVCAR-3 | OAW28 | LCLC97TM1 | H1435 | CLDN6 | CLDN9 |
| | (400,000) | (200,000) | (65,000) | (100,000) | (55,000) | (900,000) | (400,000) |
| EC₅₀ | 1.8 + 0.7 | 10.4 + 4.1 | 24.2 + 14.7 | 3.6 ± 1.0 | 1.9 ± 0.3 | 9.1 ± 10.4 | >30,000 |

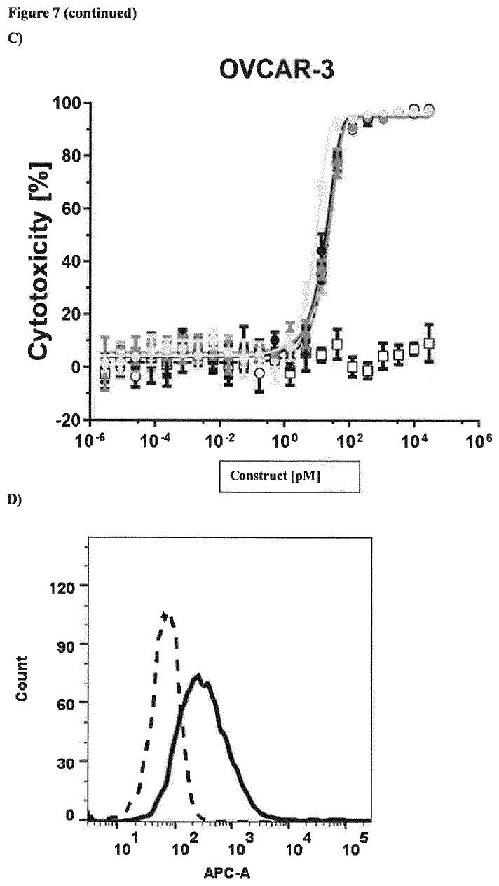

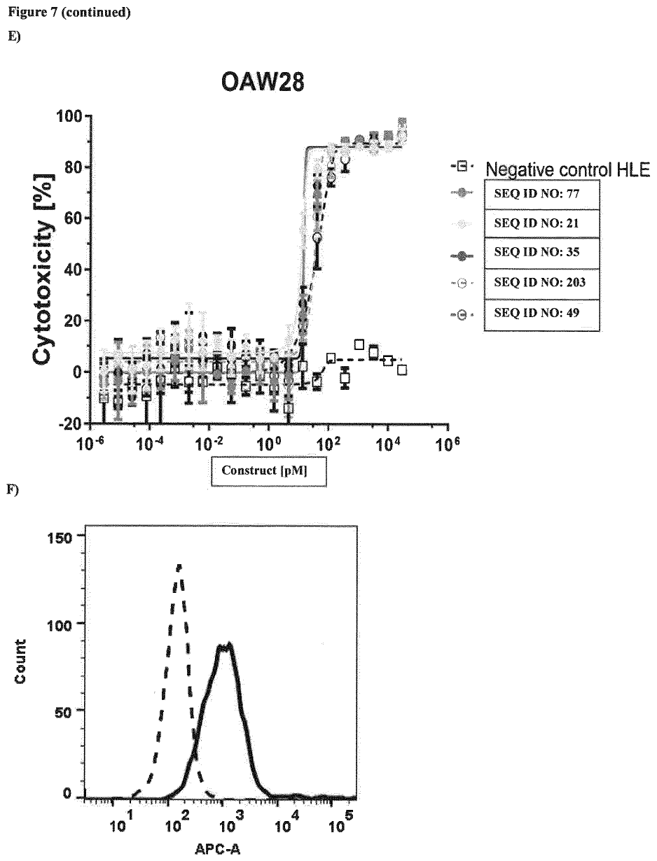

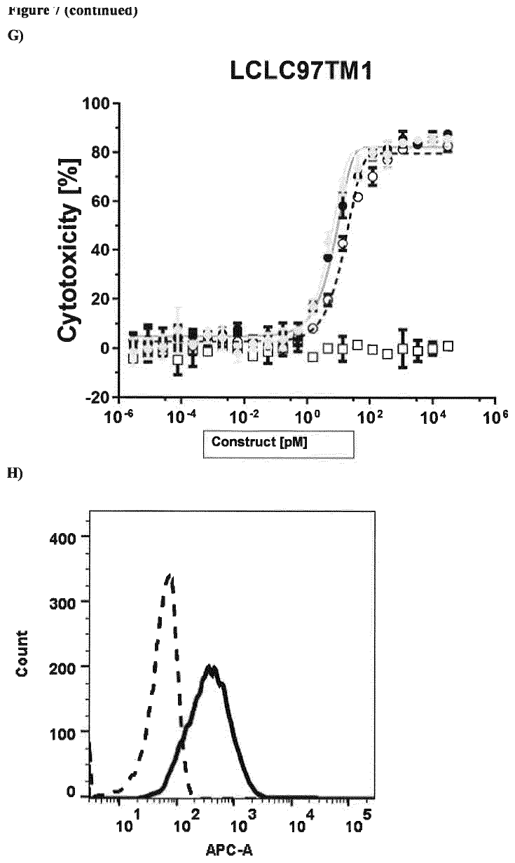

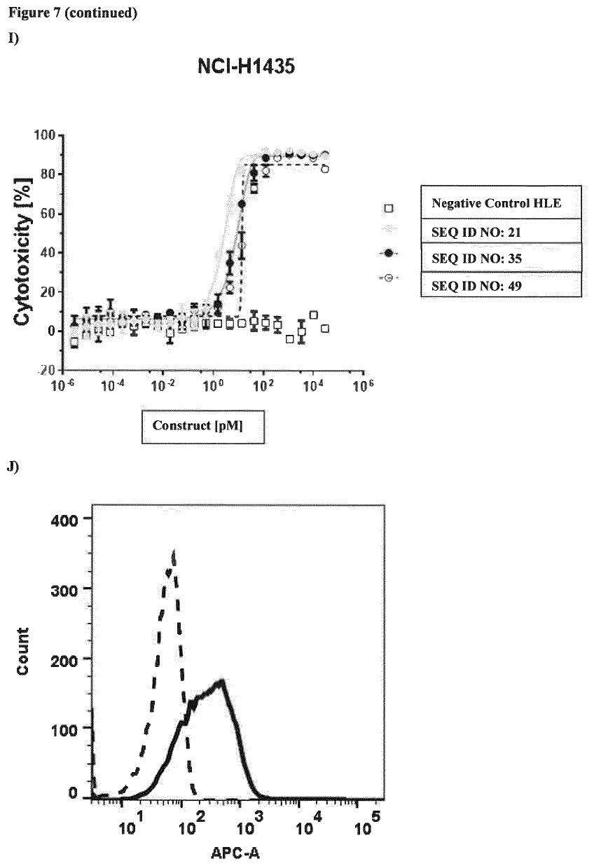

*ABC = antibody binding sites. CLDN6 cell surface expression was evaluated by flow cytometry, using a QIFIKit ® (Agilent). Cytotoxic activity was evaluated in TDCC assays. Human T cells were incubated with target cells in a 10:1 ratio and different polypeptides according to the invention (see FIG. 7E); SEQ ID NOs.: 21, 35, 49, 77, 203 at the concentrations indicated. After 48 hours, cell viability was assessed by a Cell Titer-glo ® assay (Promega). Data were analyzed in GraphPad Prism. Cytotoxicity in vitro was determined to choose polypeptides/polypeptide constructs for subsequent in vivo studies. As shown below in the table CLDN6 HLE polypeptides/polypeptide constructs (SEQ ID NOs.: 21 and 24) have potent cytotoxic activity (FIGS. 6 and 7).

### EXAMPLE 7

### CLDN6 HLE Polypeptides/Polypeptide Constructs Induce T Cell Activation

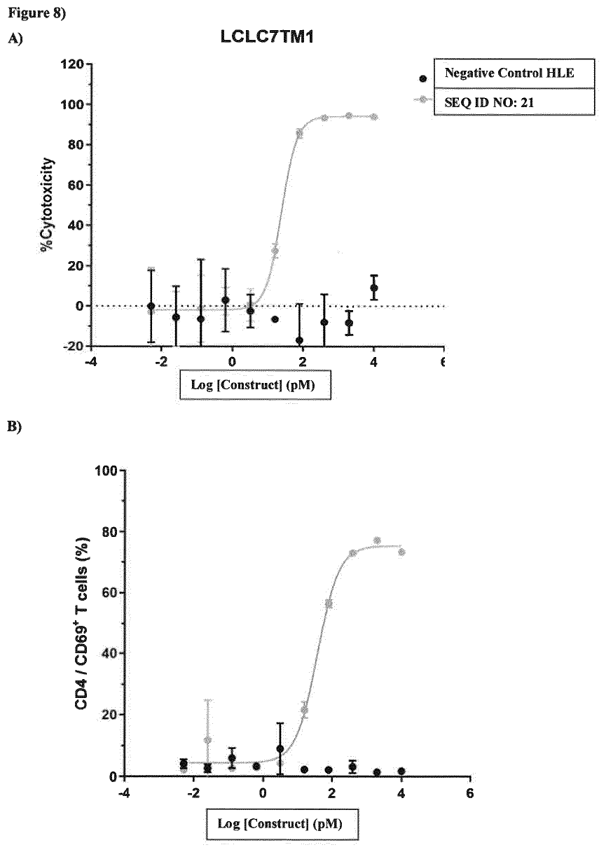

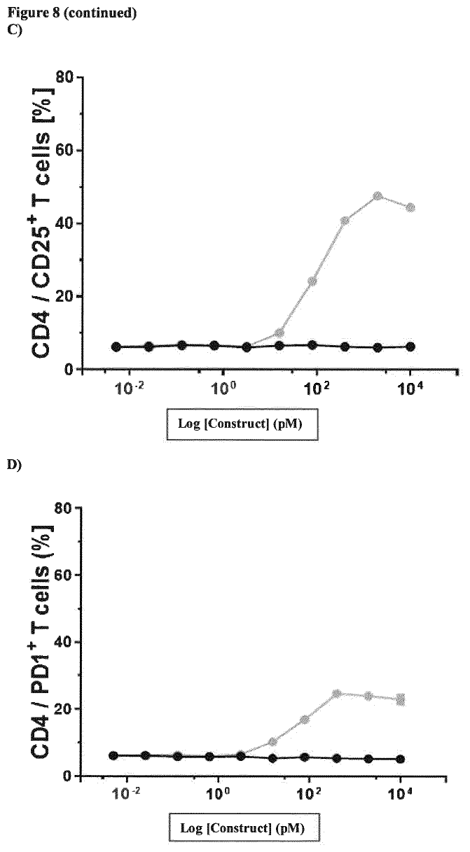

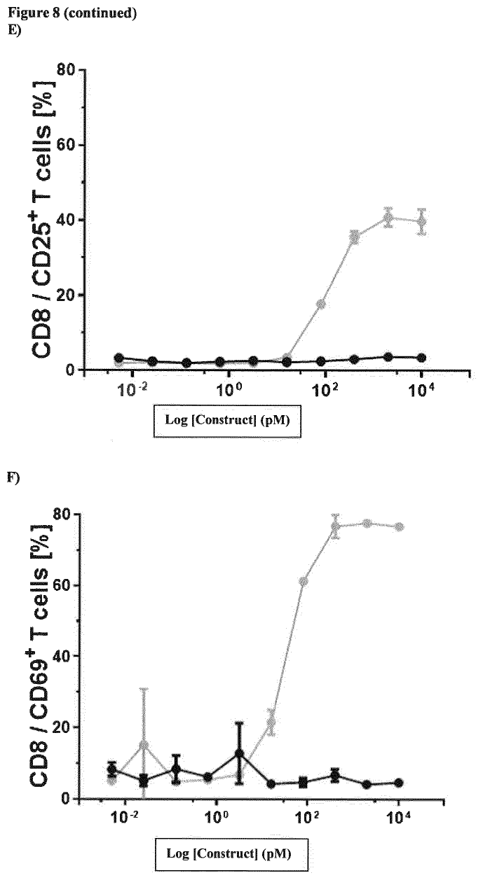

Human pan-T cells were incubated with target cells in a 10:1 ratio and an exemplary polypeptides/polypeptide constructs according to the invention (SEQ ID NO.: 21) at the indicated concentrations. After 48 hours, T cell activation (up-regulation of CD25, CD69 and PD-1) was evaluated by flow cytometry. Cell viability was assessed by a Cell Titer-glo® assay. These data showing the expression of these biomarkers demonstrate clearly that the polypeptides/polypeptide constructs induce T cell activity (FIGS. 8 A) to E)).

### EXAMPLE 8

### Antitumor Efficacy Against Established OVCAR-3 Xenograph Tumors

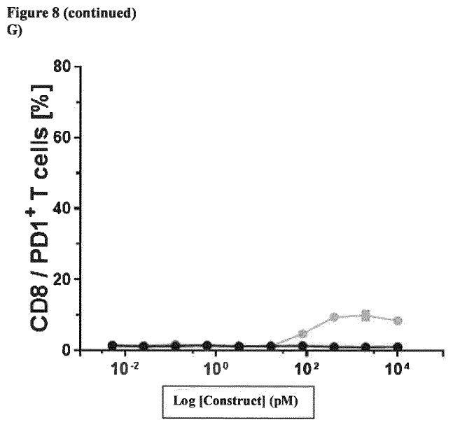

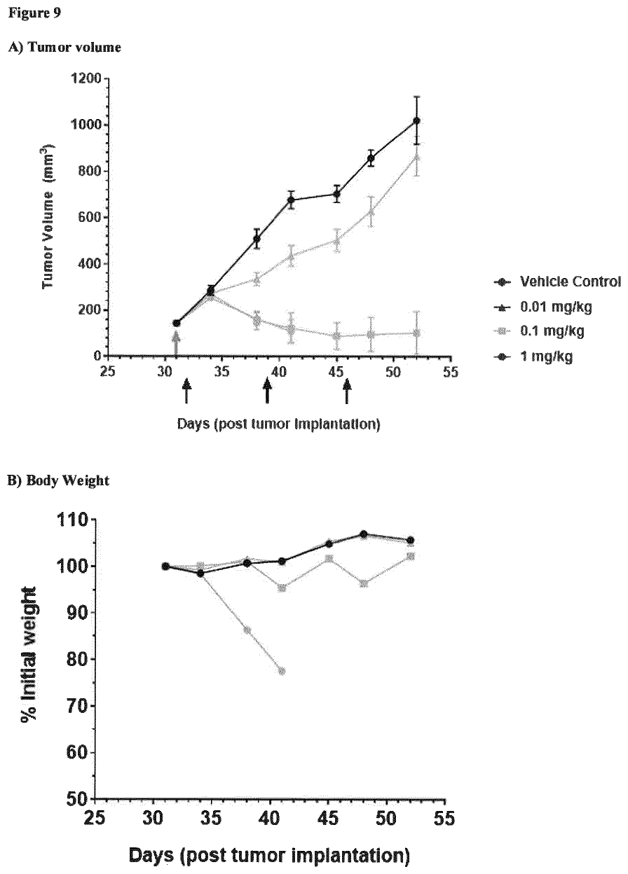

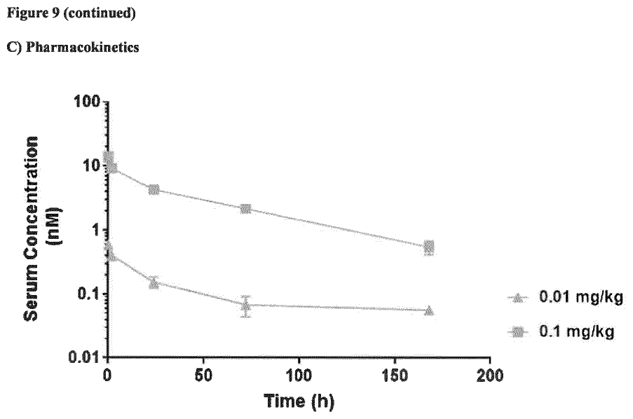

Female NSG mice (10 per group) were inoculated with 5e6 cells/mouse subcutaneously. T cells that were used were activated human pan T cells, 2e7 cells per mouse. Once tumors were allowed to form to a size of 200 mm3, and then human T cells were injected IP, mice were treated with vehicle only or with CLDN6×CD3 HLE (SEQ ID NO: 21) once weekly (FIG. 9A) to C)). FIGS. 9 A) to C) shows the results of measurements of the tumor volume over time (days at the x-axis), measurement of the body weight over time, as well as pharmacokinetic data (serum concentration over time) at different concentrations.

### EXAMPLE 9

### CLDN6×CD3 HLE was Tolerated at Doses of <100 m2/k2

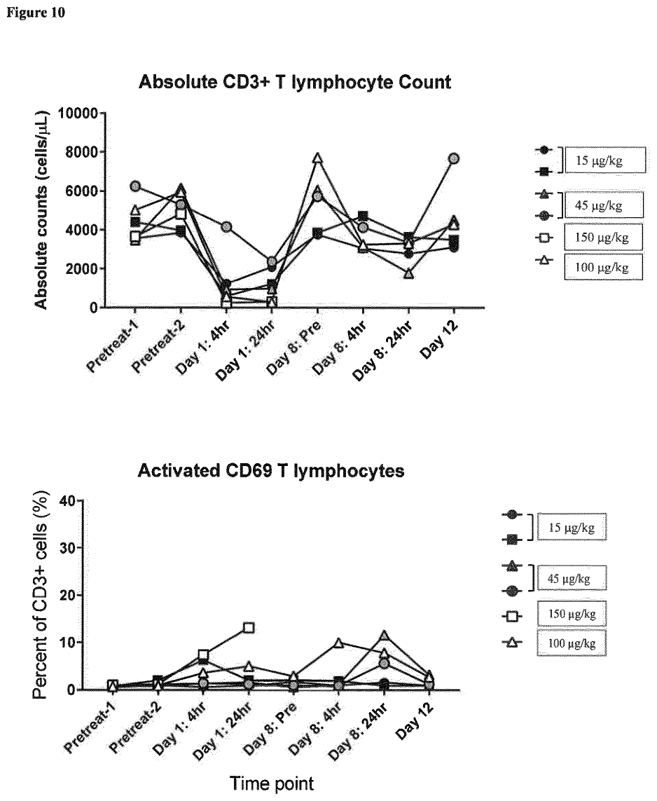

A study was performed with the objective of evaluating the tolerability of a CLDN6×CD3 HLE according to the invention (SEQ ID NO: 21) in an exploratory PK/tox study in non-human primates (NHP). To this end, flexible and staggered doses were administered to groups; 15 mg/kg, 45 mg/kg, 100 mg/kg (1 animal). The results showed that signs of T cell-dependent activation were noted at all doses. The polypeptide construct of the invention was clinically tolerated up to 100 μg/kg. The results are shown in FIG. 10. Treatment related microscopic findings (minimal to mild) were observed at doses >45 μg/kg in the liver (bile ducts) and skin (IV administration site, anus, mammary gland, dorsum and hindlimb) at ≥45 μg/kg (no findings found at all sites in all animals) and in the mucosal epithelium of esophagus and interstitial lung at ≥100 μg/kg. This study shows that CLDN6 is a safe target as a polypeptide construct.

### EXAMPLE 10

### Hallmarks of Engagement Characteristic of T Cell-Activating Polypeptides/Polypeptide Constructs were Observed in an Exploratory Toxicology Study

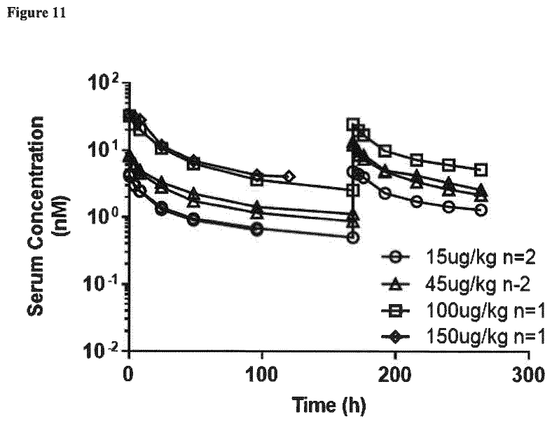

CLDN6 dose-dependent decreases in CD3+T-lymphocytes absolute counts were observed 4 hours post-dosing on Days 1 and 8. The decreases observed at Day 8 were generally not to the same magnitude as Day 1. Transient lymphocyte redistribution followed administration with a transient decrease in total T cell counts were observed in all groups. FIG. 10 indicates T cell-activating polypeptides/polypeptide constructs induce increased activity, e.g. CD69 expression which is observed in mid and high dose groups. T cell activation was assessed by flow cytometry as described above. Further, the absolute number of CD3+T lymphocyte counts and the percentage of CD3+T lymphocyte counts were analysed and it could also be shown that the transient cytokine release (IFN-γ, TNF-α, MCP-1, IL-1β, IL-1rα, IL-2, IL-5, and IL-6) is consistent with the activity of T cell-activating polypeptides/polypeptide constructs. Further, a CLDN6 HLE polypeptide construct according to the invention (SEQ ID NOs.:24) has extended half-life in non-human primates. T cell-activating polypeptides/polypeptide constructs exposure levels in non-human primate serum were evaluated in samples from the toxicology study. The construct demonstrated dose-dependent exposure and an extended half-life (8.39 days) (FIG. 11).

### EXAMPLE 11

### Cytotoxicity Studies In Vitro and In Vivo

Using the method described in Example 6, different HLE polypeptides/polypeptide constructs of the invention were analyzed for the cytotoxicity in various cell lines. As shown in table 3 the polypeptides/polypeptide constructs of the present invention are cytotoxic in various cell lines.

TABLE 3
TDCC EC₅₀ pM of cytotoxicity assays of different polypeptides
of the invention in various cell lines

| | TDCC EC50 (pM) | | | | |
|---|---|---|---|---|---|
| Polypeptide construct | PA-1 | OVCAR-3 | OAW28 | LCLC97TM1 | NCI-H1435 |
| S3N (SEQ ID NO: 77) | 3.1 + 1.2 | 25.7 + 11.4 | 35.2 + 14.2 | NT | NT |
| L4B (SEQ ID NO: 49) | 3.4 + 1.2 | 19.4 | 44.6 + 19.0 | 10.7 ± 6.3 | 11.6 ± 4.1 |
| X3S (SEQ ID NO: 35) | 2.8 + 1.3 | 19.1 + 6.6 | 30.1 + 10.8 | 5.6 ± 2.5 | 5.9 ± 2.4 |
| B6L (SEQ ID NO: 21) | 1.8 + 0.7 | 10.4 + 4.1 | 24.2 + 14.7 | 3.6 ± 1.8 | 1.9 ± 0.5 |
| A3S (SEQ ID NO: 203) | 4.7 + 1.6 | 28.1 + 12.9 | 65.3 + 37.0 | NT | NT |

### EXAMPLE 12

### Anti-Tumor Activity of AMG 794 was Assessed in a Subcutaneous Advanced-Stage NSCLC Model in Female Non-Obese Diabetic/Severe Combined Immunodeficiency (NOD/SCID) Mice

Treatment of NCI-H1435-peak-1-1 tumor-bearing mice with CLDN6×CD3 HLE (SEQ ID NO: 21) resulted in a statistically significant TGI at doses of 0.5, 0.15 and 0.05 mg/kg with p values <0.001 when tumor growth was compared with vehicle-treated mice of group 2 between days 17 and 27 using a mixed-effects model followed by Dunnett's post-hoc test. A re-growth of tumors following day 22 until end of tumor growth measurement on day 27 was observed. An increased population of target-negative cells compared to study start and a low number of human lymphocytes was detected in explanted tumors, which could explain the re-growth of tumors following day 22. The TGI achieved on day 22 at doses of 0.5, 0.15 and 0.05 mg/kg CLDN6×CD3 HLE was 91%, 67% or 82%, respectively.

| SEQ | UNIQUE | | |
|---|---|---|---|
| ID | IDENTIFIER / | NATURAL/ | |
| NO: | DESCRIPTION | ARTIFICIAL | SEQUENCE/AA |
| 1 | HUMAN | NATURAL | MASAGMQILGVVLTLLGWVNGLVSCALPMWKVTAFIGNSIVVAQVVWEGLWMSCVVQSTGQM |
| | CLDN-6 | | QCKVYDSLLALPQDLQAARALCVIALLVALFGLLVYLAGAKCTTCVEEKDSKARLVLTSGIV |
| | UNIPROT | | FVISGVLTLIPVCWTAHAIIRDFYNPLVAEAQKRELGASLYLGWAASGLLLLGGGLLCCTCP |
| | P56747 | | SGGSQGPSHYMARYSTSAPAISRGPSEYPTKNYV |
| 2 | HUMAN | NATURAL | MSTTTCQVVAFLLSILGLAGCIAATGMDMWSTQDLYDNPVTSVFQYEGLWRSCVRQSSGFTE |
| | CLDN -18.1 | | CRPYFTILGLPAMLQAVRALMIVGIVLGAIGLLVSIFALKCIRIGSMEDSAKANMTLTSGIM |
| | UNIPROT | | FIVSGLCAIAGVSVFANMLVTNFWMSTANMYTGMGGMVQTVQTRYTFGAALFVGWVAGGLTL |
| | P56856-1 | | IGGVMMCIACRGLAPEETNYKAVSYHASGHSVAYKPGGFKASTGFGSNTKNKKIYDGGARTE |
| | | | DEVQSYPSKHDYV |
| 3 | HUMAN | NATURAL | MAVTACQGLGFVVSLIGIAGIIAATCMDQWSTQDLYNNPVTAVFNYQGLWRSCVRESSGFTE |
| | CLDN -18.2 | | CRGYFTLLGLPAMLQAVRALMIVGIVLGAIGLLVSIFALKCIRIGSMEDSAKANMTLTSGIM |
| | UNIPROT | | FIVSGLCAIAGVSVFANMLVTNFWMSTANMYTGMGGMVQTVQTRYTFGAALFVGWVAGGLTL |
| | P56856-2 | | IGGVMMCIACRGLAPEETNYKAVSYHASGHSVAYKPGGFKASTGFGSNTKNKKIYDGGARTE |
| | | | DEVQSYPSKHDYV |
| 4 | CLDN1 | NATURAL | MANAGLQLLGFILAFLGWIGAIVSTALPQWRIYSYAGDNIVTAQAMYEGLWMSCVSQSTGQI |
| | UNIPROT | | QCKVFDSLLNLSSTLQATRALMVVGILLGVIAIFVATVGMKCMKCLEDDEVQKMRMAVIGGA |
| | O95832-1 | | IFLLAGLAILVATAWYGNRIVQEFYDPMTPVNARYEFGQALFTGWAAASLCLLGGALLCCSC |
| | | | PRKTTSYPTPRPYPKPAPSSGKDYV |
| 5 | CLDN2 | NATURAL | MASLGLQLVGYILGLLGLLGTLVAMLLPSWKTSSYVGASIVTAVGFSKGLWMECATHSTGIT |
| | UNIPROT | | QCDIYSTLLGLPADIQAAQAMMVTSSAISSLACIISVVGMRCTVFCQESRAKDRVAVAGGVF |
| | P57739 | | FILGGLLGFIPVAWNLHGILRDFYSPLVPDSMKFEIGEALYLGIISSLFSLIAGIILCFSCS |
| | | | SQRNRSNYYDAYQAQPLATRSSPRPGQPPKVKSEFNSYSLTGYV |
| 6 | CLDN3 | NATURAL | MSMGLEITGTALAVLGWLGTIVCCALPMWRVSAFIGSNIITSQNIWEGLWMNCVVQSTGQMQ |
| | UNIPROT | | CKVYDSLLALPQDLQAARALIVVAILLAAFGLLVALVGAQCTNCVQDDTAKAKITIVAGVLF |
| | O15551 | | LLAALLTLVPVSWSANTIIRDFYNPVVPEAQKREMGAGLYVGWAAAALQLLGGALLCCSCPP |
| | | | REKKYTATKVVYSAPRSTGPGASLGTGYDRKDYV |
| 7 | CLDN4 | NATURAL | MASMGLQVMGIALAVLGWLAVMLCCALPMWRVTAFIGSNIVTSQTIWEGLWMNCVVQSTGQM |
| | UNIPROT | | QCKVYDSLLALPQDLQAARALVIISIIVAALGVLLSVVGGKCTNCLEDESAKAKTMIVAGVV |
| | O14493 | | FLLAGLMVIVPVSWTAHNIIQDFYNPLVASGQKREMGASLYVGWAASGLLLLGGGLLCCNCP |
| | | | PRTDKPYSAKYSAARSAAASNYV |
| 8 | CLDN9 | NATURAL | MASTGLELLGMTLAVLGWLGTLVSCALPLWKVTAFIGNSIVVAQVVWEGLWMSCVVQSTGQM |
| | UNIPROT | | QCKVYDSLLALPQDLQAARALCVIALLLALLGLLVAITGAQCTTCVEDEGAKARIVLTAGVI |
| | O95484 | | LLLAGILVLIPVCWTAHAIIQDFYNPLVAEALKRELGASLYLGWAAAALLMLGGGLLCCTCP |
| | | | PPQVERPRGPRLGYSIPSRSGASGLDKRDYV |
| 9 | E1A | ARTIFICIAL | MWKVTAFIGNS |
| | HUMAN | | |
| | CLDN-6 E1A | | |
| 10 | E2B HUMAN | ARTIFICIAL | LVAEAQKREL |
| | CLDN-6 | | |
| 11 | B6L/VH | ARTIFICIAL | QVQLVQSGAEVKKPGASVKVSCKASGYTFTGYYMHWVRQAPGQCLEWMGWINPNSGETNYAQ |
| | | | KFQGRVTMTRDTSISTAYMELSRLRSDDTAVYYCARDALIVVAPVTRDYYYYGMDVWGQGTT |
| | | | VTVSS |
| 12 | B6L/VL | ARTIFICIAL | EIVLTQSPGTLSLSPGERATLSCRASQSVSSSYLAWYQQKPGQAPRLLIYGASSRATGIPDR |
| | | | FSGSGSGTDFTLTISRLEPEDFAVYYCQQYGSSPLTFGCGTKLEIK |
| 13 | B6L/CDR-H1 | ARTIFICIAL | GYYMH |
| 14 | B6L/CDR-H2 | ARTIFICIAL | WINPNSGETNYAQKFQG |
| 15 | B6L/CDR-H3 | ARTIFICIAL | DALIVVAPVTRDYYYYGMDV |
| 16 | B6L/CDR-L1 | ARTIFICIAL | RASQSVSSSYLA |
| 17 | B6L/CDR-L2 | ARTIFICIAL | GASSRAT |
| 18 | B6L/CDR-L3 | ARTIFICIAL | QQYGSSPLT |
| 19 | B6L/SCFV | ARTIFICIAL | EIVLTQSPGTLSLSPGERATLSCRASQSVSSSYLAWYQQKPGQAPRLLIYGASSRATGIPDR |
| | I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSS |
| 20 | B6L/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2E | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 21 | B6L/HLE- | ARTIFICIAL | EIVLTQSPGTLSLSPGERATLSCRASQSVSSSYLAWYQQKPGQAPRLLIYGASSRATGIPDR |
| | BITE I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSSSGGGGSLVQPGGSLKLSCAASGFTFNKYAINWVRQAPGKGLEWVARIRSKYNNYATYY |
| | | | ADAVKDRFTISRDDSKNTVYLQMNNLKTEDTAVYYCARAGNFGSSYISYWAYWGQGTLVTVS |
| | | | SGGGGSGGGGSGGGGSQTVVTQEPSLTVSPGGTVTITCGSSTGAVTSGNYPNWVQKKPGQAP |
| | | | RGLIGGTKFLAPGTPARFSGSLSGGKAALTLSGVQPEDEAEYYCVLWYSNRWVFGSGTKLTV |
| | | | LGGGGDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGK6GGGGSGGGGSGGGGS |
| | | | GGGGSGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 22 | B6L/SCFV | ARTIFICIAL | EIVLTQSPGTLSLSPGERATLSCRASQSVSSSYLAWYQQKPGQAPRLLIYGASSRATGIPDR |
| | I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSS |
| 23 | B6L/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2C | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 24 | B6L/HLE | ARTIFICIAL | EIVLTQSPGTLSLSPGERATLSCRASQSVSSSYLAWYQQKPGQAPRLLIYGASSRATGIPDR |
| | BITE I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 25 | X3S/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSDDTAVYYCVRDALIVEAPATRDYYYYGMDVWGQGTT |
| | | | VTVSS |
| 26 | X3S/VL | ARTIFICIAL | EIVLTQSPGTLSLSPGERATLSCRASQSVRSTYLAWYQQKPGQAPRLLIYGASSRATGIPDR |
| | | | FSGSGSGTDFTLTISRLEPEDFAVYYCQQYDASPITFGCGTRLEIK |
| 27 | X3S/CDR-H1 | ARTIFICIAL | GYYVH |
| 28 | X3S/CDR-H2 | ARTIFICIAL | WINPNSGATNYAQKFQG |
| 29 | X3S/CDR-H3 | ARTIFICIAL | DALIVEAPATRDYYYYGMDV |
| 30 | X3S/CDR-L1 | ARTIFICIAL | RASQSVRSTYLA |
| 31 | X3S/CDR-L2 | ARTIFICIAL | GASSRAT |
| 32 | X3S/CDR-L3 | ARTIFICIAL | QQYDASPIT |
| 33 | X3S/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSDDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIK |
| 34 | X3S/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSDDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 35 | X3S/HLE | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSDDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 36 | X3S/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSDDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIK |
| 37 | X3S/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSDDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 38 | X3S/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSDDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 39 | L4B/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSDDTAVYYCVRDRLIVEAPATRDYYYYGMDVWGQGTT |
| | | | VTVSS |
| 40 | L4B/VL | ARTIFICIAL | EIVLTQSPGTLSLSPGERATLSCRASQSVRSTYLAWYQQKPGQAPRLLIYGASSRATGIPDR |
| | | | FSGSGSGTDFTLTISRLEPEDFAVYYCQQYDASPITFGCGTRLEIK |
| 41 | L4B/CDR-H1 | ARTIFICIAL | GYYVH |
| 42 | L4B/CDR-H2 | ARTIFICIAL | WINPNSGATNYAQKFQG |
| 43 | L4B/CDR-H3 | ARTIFICIAL | DRLIVEAPATRDYYYYGMDV |
| 44 | L4B/CDR-L1 | ARTIFICIAL | RASQSVRSTYLA |
| 45 | L4B/CDR-L2 | ARTIFICIAL | GASSRAT |
| 46 | L4B/CDR-L3 | ARTIFICIAL | QQYDASPIT |
| 47 | L4B/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSDDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIK |
| 48 | L4B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSDDTAVYYCVRDRLIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 49 | L4B/HLE | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSDDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 50 | L4B/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSDDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIK |
| 51 | L4B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSDDTAVYYCVRDRLIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 52 | L4B/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSDDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIYGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 53 | I2P/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDALIVEAPATRDYYYYGMDVWGQGTT |
| | | | VTVSS |
| 54 | I2P/VL | ARTIFICIAL | EIVLTQSPGTLSLSPGERATLSCRASQSVRSTYLAWYQQKPGQAPRLLISGASSRATGIPDR |
| | | | FSGSGSGTDFTLTISRLEPEDFAVYYCQQYDASPITFGCGTRLEIKS |
| 55 | I2P/CDR-H1 | ARTIFICIAL | GYYVH |
| 56 | I2P/CDR-H2 | ARTIFICIAL | WINPNSGATNYAQKFQG |
| 57 | I2P/CDR-H3 | ARTIFICIAL | DALIVEAPATRDYYYYGMDV |
| 58 | I2P/CDR-L1 | ARTIFICIAL | RASQSVRSTYLA |
| 59 | I2P/CDR-L2 | ARTIFICIAL | GASSRAT |
| 60 | I2P/CDR-L3 | ARTIFICIAL | QQYDASPIT |
| 61 | I2P/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIKS |
| 62 | I2P/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIKSSGGGGSEVQLVESGGGLVQPGGSLKLSCAASGFTFNKYAINWVRQAPGKGLEWVARI |
| | | | RSKYNNYATYYADAVKDRFTISRDDSKNTVYLQMNNLKTEDTAVYYCARAGNFGSSYISYWA |
| | | | YWGQGTLVTVSSGGGGSGGGGSGGGGSQTVVTQEPSLTVSPGGTVTITCGSSTGAVTSGNYP |
| | | | NWVQKKPGQAPRGLIGGTKFLAPGTPARFSGSLSGGKAALTLSGVQPEDEAEYYCVLWYSNR |
| | | | WVFGSGTKLTVL |
| 63 | I2P/HLE | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIKSSGGGGSEVQLVESGGGLVQPGGSLKLSCAASGFTFNKYAINWVRQAPGKGLEWVARI |
| | | | RSKYNNYATYYADAVKDRFTISRDDSKNTVYLQMNNLKTEDTAVYYCARAGNFGSSYISYWA |
| | | | YWGQGTLVTVSSGGGGSGGGGSGGGGSQTVVTQEPSLTVSPGGTVTITCGSSTGAVTSGNYP |
| | | | NWVQKKPGQAPRGLIGGTKFLAPGTPARFSGSLSGGKAALTLSGVQPEDEAEYYCVLWYSNR |
| | | | WVFGSGTKLTVLGGGGDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKGGGGS |
| | | | GGGGSGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLNGKEY |
| | | | KCKVSNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQKSLSL |
| | | | SPGK |
| 64 | I2P/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIK |
| 65 | I2P 1 | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 66 | I2P/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 67 | S3N/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDALIVEAPATRDYYYYGMDVWGQGTT |
| | | | VTVSS |
| 68 | S3N/VL | ARTIFICIAL | EIVLTQSPGTLSLSPGERATLSCRASQSVRSTYLAWYQQKPGQAPRLLISGASSRATGIPDR |
| | | | FSGSGSGTDFTLTISRLEPEDFAVYYCQQYQTSPITFGCGTRLEIK |
| 69 | S3N/CDR-H1 | ARTIFICIAL | GYYVH |
| 70 | S3N/CDR-H2 | ARTIFICIAL | WINPNSGATNYAQKFQG |
| 71 | S3N/CDR-H3 | ARTIFICIAL | DALIVEAPATRDYYYYGMDV |
| 72 | S3N/CDR-L1 | ARTIFICIAL | RASQSVRSTYLA |
| 73 | S3N/CDR-L2 | ARTIFICIAL | GASSRAT |
| 74 | S3N/CDR-L3 | ARTIFICIAL | QQYQTSPIT |
| 75 | S3N/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIK |
| 76 | S3N/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 77 | S3N/HLE | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 78 | S3N/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIK |
| 79 | S3N/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 80 | S3N/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLISGASSRATGIPDRFSGSGSGTDFTLTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 81 | H7I/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDRLIVEAPATRDYYYYGMDVWGQGTT |
| | | | VTVSS |
| 82 | H7I/VL | ARTIFICIAL | EIVLTQSPGTLSLSPGERATLSCRASQSVRSTYLAWYQQTPGQAPRLLISGASSRATGIPDR |
| | | | FSGSGSGTDFILTISRLEPEDFAVYYCQQYDASPITFGCGTRLEIK |
| 83 | H7I/CDR-H1 | ARTIFICIAL | GYYVH |
| 84 | H7I/CDR-H2 | ARTIFICIAL | WINPNSGATNYAQKFQG |
| 85 | H7I/CDR-H3 | ARTIFICIAL | DRLIVEAPATRDYYYYGMDV |
| 86 | H7I/CDR-L1 | ARTIFICIAL | RASQSVRSTYLA |
| 87 | H7I/CDR-L2 | ARTIFICIAL | GASSRAT |
| 88 | H7I/CDR-L3 | ARTIFICIAL | QQYDASPIT |
| 89 | H7I/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIK |
| 90 | H7I/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDRLIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 91 | H7I/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 92 | H7I/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIK |
| 93 | H7I/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDRLIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 94 | H7I/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 95 | J2I/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDRLIVEAPATRDYYYYGMDVWGQGTT |
| | | | VTVSS |
| 96 | J2I/VL | ARTIFICIAL | EIVLTQSPGTLSLSPGERATLSCRASQSVRSTYLAWYQQTPGQAPRLLISGASSRATGIPDR |
| | | | FSGSGSGTDFILTISRLEPEDFAVYYCQQYQTSPITFGCGTRLEIK |
| 97 | J2I/CDR-H1 | ARTIFICIAL | GYYVH |
| 98 | J2I/CDR-H2 | ARTIFICIAL | WINPNSGATNYAQKFQG |
| 99 | J2I/CDR-H3 | ARTIFICIAL | DRLIVEAPATRDYYYYGMDV |
| 100 | J2I/CDR-L1 | ARTIFICIAL | RASQSVRSTYLA |
| 101 | J2I/CDR-L2 | ARTIFICIAL | GASSRAT |
| 102 | J2I/CDR-L3 | ARTIFICIAL | QQYQTSPIT |
| 103 | J2I/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIK |
| 104 | J2I/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDRLIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 105 | J2I/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 106 | J2I/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIK |
| 107 | J2I/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDRLIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 108 | J2I/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDRLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 109 | A4K/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDALIVEAPATRDYYYYGMDVWGQGTT |
| | | | VTVSS |
| 110 | A4K/VL | ARTIFICIAL | EIVLTQSPGTLSLSPGERATLSCRASQSVRSTYLAWYQQTPGQAPRLLISGASSRATGIPDR |
| | | | FSGSGSGTDFILTISRLEPEDFAVYYCQQYDASPITFGCGTRLEIK |
| 111 | A4K/CDR-H1 | ARTIFICIAL | GYYVH |
| 112 | A4K/CDR-H2 | ARTIFICIAL | WINPNSGATNYAQKFQG |
| 113 | A4K/CDR-H3 | ARTIFICIAL | DALIVEAPATRDYYYYGMDV |
| 114 | A4K/CDR-L1 | ARTIFICIAL | RASQSVRSTYLA |
| 115 | A4K/CDR-L2 | ARTIFICIAL | GASSRAT |
| 116 | A4K/CDR-L3 | ARTIFICIAL | QQYDASPIT |
| 117 | A4K/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIK |
| 118 | A4K/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 119 | A4K/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 120 | A4K/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIK |
| 121 | A4K/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 122 | A4K/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 123 | E5B/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDALIVEAPATRDYYYYGMDVWGQGTT |
| | | | VTVSS |
| 124 | E5B/VL | ARTIFICIAL | EIVLTQSPGTLSLSPGERATLSCRASQSVRSTYLAWYQQTPGQAPRLLISGASSRATGIPDR |
| | | | FSGSGSGTDFILTISRLEPEDFAVYYCQQYQTSPITFGCGTRLEIK |
| 125 | E5B/CDR-H1 | ARTIFICIAL | GYYVH |
| 126 | E5B/CDR-H2 | ARTIFICIAL | WINPNSGATNYAQKFQG |
| 127 | E5B/CDR-H3 | ARTIFICIAL | DALIVEAPATRDYYYYGMDV |
| 128 | E5B/CDR-L1 | ARTIFICIAL | RASQSVRSTYLA |
| 129 | E5B/CDR-L2 | ARTIFICIAL | GASSRAT |
| 130 | E5B/CDR-L3 | ARTIFICIAL | QQYQTSPIT |
| 131 | E5B 1 SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIK |
| 132 | E5B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 133 | E5B/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 134 | E5B/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIK |
| 135 | E5B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 136 | E5B/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYQTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 137 | R8B/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDALIVEAPATRDYYYYGMDVWGQGTT |
| | | | VTVSS |
| 138 | R8B/VL | ARTIFICIAL | EIVLTQSPGTLSLSPGERATLSCRASQSVRSTYLAWYQQKPGQAPRLLIIGASSRATGIPDR |
| | | | FSGSGSGTDFTLTISRLEPEDFAVYYCQQYDASPITFGCGTRLEIK |
| 139 | R8B/CDR-H1 | ARTIFICIAL | GYYVH |
| 140 | R8B/CDR-H2 | ARTIFICIAL | WINPNSGATNYAQKFQG |
| 141 | R8B/CDR-H3 | ARTIFICIAL | DALIVEAPATRDYYYYGMDV |
| 142 | R8B/CDR-L1 | ARTIFICIAL | RASQSVRSTYLA |
| 143 | R8B/CDR-L2 | ARTIFICIAL | GASSRAT |
| 144 | R8B/CDR-L3 | ARTIFICIAL | QQYDASPIT |
| 145 | R8B/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIK |
| 146 | R8B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 147 | R8B/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 148 | R8B/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIK |
| 149 | R8B/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDALIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 150 | R8B/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDALIVEAPATRDYYYYGMDVWGQGTT |
| | | | VTVSSGGGGSGGGGSGGGGSEIVLTQSPGTLSLSPGERATLSCRASQSVRSTYLAWYQQKPG |
| | | | QAPRLLIIGASSRATGIPDRFSGSGSGTDFTLTISRLEPEDFAVYYCQQYDASPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 151 | IX9/VH | ARTIFICIAL | QVQLVQSGAEVKKPGASVKVSCKASGYTFTGYYMHWVRQAPGQCLEWMGWINPNSGETNYAQ |
| | | | KFQGRVTMTRDTSISTAYMELSRLRSDDTAVYYCARDALIVVAPVTRDYYYYGMDVWGQGTT |
| | | | VTASS |
| 152 | IX9/VL | ARTIFICIAL | EIVLTQSPGTLSLSPGERATLSCRASQTVSSSYLVWYQQKPGQAPRLLIYGASSRATGIPDR |
| | | | FSGSGSGTDFTLTISRLEPEDFAVYYCQQYGGSPITFGCGTRLEIK |
| 153 | IX9/CDR-H1 | ARTIFICIAL | GYYMH |
| 154 | IX9/CDR-H2 | ARTIFICIAL | WINPNSGETNYAQKFQG |
| 155 | IX9/CDR-H3 | ARTIFICIAL | DALIVVAPVTRDYYYYGMDV |
| 156 | IX9/CDR-L1 | ARTIFICIAL | RASQTVSSSYLV |
| 157 | IX9/CDR-L2 | ARTIFICIAL | GASSRAT |
| 158 | IX9/CDR-L3 | ARTIFICIAL | QQYGGSPIT |
| 159 | IX9/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASS |
| 160 | IX9/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 161 | IX9/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 162 | IX9/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASS |
| 163 | IX9/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 164 | IX9/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 165 | G5X/VH | ARTIFICIAL | QVQLVQSGAEVKKPGASVKVSCKASGYTFTGYYMHWVRQAPGQCLEWMGWINPNSGDANYAQ |
| | | | KFQGRVTMTRDTSISTAYMELSRLRSDDTAVYYCARDALIVVAPVTRDYYYYGMDVWGQGTT |
| | | | VTVSS |
| 166 | G5X/VL | ARTIFICIAL | EIVLTQSPGTLSLSPGERATLSCRASQTVSSSYLVWYQQKPGQAPRLLIYGASSRATGIPDR |
| | | | FSGSGSGTDFTLTISRLEPEDFAVYYCQQYGGSPITFGCGTRLEIK |
| 167 | G5X/CDR-H1 | ARTIFICIAL | GYYMH |
| 168 | G5X/CDR-H2 | ARTIFICIAL | WINPNSGDANYAQKFQG |
| 169 | G5X/CDR-H3 | ARTIFICIAL | DALIVVAPVTRDYYYYGMDV |
| 170 | G5X/CDR-L1 | ARTIFICIAL | RASQTVSSSYLV |
| 171 | G5X/CDR-L2 | ARTIFICIAL | GASSRAT |
| 172 | G5X/CDR-L3 | ARTIFICIAL | QQYGGSPIT |
| 173 | G5X/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSS |
| 174 | G5X/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 175 | G5X/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 176 | G5X/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSS |
| 177 | G5X/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 178 | G5X/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 179 | O1C/VH | ARTIFICIAL | QVQLVQSGAEVKKPGASVKVSCKASGYTFTGYYMHWVRQAPGQCLEWMGWINPNSGETNYAQ |
| | | | KFQGRVTMTRDTSISTAYMELSRLRSDDTAVYYCARDNLIVVAPVTRDYYYYGMDVWGQGTT |
| | | | VTASS |
| 180 | O1C/VL | ARTIFICIAL | EIVLTQSPGTLSLSPGERATLSCRASQTVSSSYLVWYQQKPGQAPRLLIYGASSRATGIPDR |
| | | | FSGSGSGTDFTLTISRLEPEDFAVYYCQQYGGSPITFGCGTRLEIK |
| 181 | O1C/CDR-H1 | ARTIFICIAL | GYYMH |
| 182 | O1C/CDR-H2 | ARTIFICIAL | WINPNSGETNYAQKFQG |
| 183 | O1C/CDR-H3 | ARTIFICIAL | DNLIVVAPVTRDYYYYGMDV |
| 184 | O1C/CDR-L1 | ARTIFICIAL | RASQTVSSSYLV |
| 185 | O1C/CDR-L2 | ARTIFICIAL | GASSRAT |
| 186 | O1C/CDR-L3 | ARTIFICIAL | QQYGGSPIT |
| 187 | O1C/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASS |
| 188 | O1C/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 189 | O1C/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 190 | O1C/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASS |
| 191 | O1C/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASS |
| 192 | O1C/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 193 | A3S/VH | ARTIFICIAL | QVQLVQSGAEVKKPGASVKVSCKASGYTFTGYYMHWVRQAPGQCLEWMGWINPNSGETNYAQ |
| | | | KFQGRVTMTRDTSISTAYMELSRLRSDDTAVYYCARDNLIVVAPVTRDYYYYGMDVWGQGTT |
| | | | VTVSS |
| 194 | A3S/VL | ARTIFICIAL | EIVLTQSPGTLSLSPGERATLSCRASQSVSSSYLAWYQQKPGQAPRLLIYGASSRATGIPDR |
| | | | FSGSGSGTDFTLTISRLEPEDFAVYYCQQYGSSPLTFGCGTKLEIK |
| 195 | A3S/CDR-H1 | ARTIFICIAL | GYYMH |
| 196 | A3S/CDR-H2 | ARTIFICIAL | WINPNSGETNYAQKFQG |
| 197 | A3S/CDR-H3 | ARTIFICIAL | DNLIVVAPVTRDYYYYGMDV |
| 198 | A3S/CDR-L1 | ARTIFICIAL | RASQSVSSSYLA |
| 199 | A3S/CDR-L2 | ARTIFICIAL | GASSRAT |
| 200 | A3S/CDR-L3 | ARTIFICIAL | QQYGSSPLT |
| 201 | A3S/SCFV | ARTIFICIAL | EIVLTQSPGTLSLSPGERATLSCRASQSVSSSYLAWYQQKPGQAPRLLIYGASSRATGIPDR |
| | I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSS |
| 202 | A3S/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2E | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 203 | A3S/HLE- | ARTIFICIAL | EIVLTQSPGTLSLSPGERATLSCRASQSVSSSYLAWYQQKPGQAPRLLIYGASSRATGIPDR |
| | BITE I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 204 | A3S/SCFV | ARTIFICIAL | EIVLTQSPGTLSLSPGERATLSCRASQSVSSSYLAWYQQKPGQAPRLLIYGASSRATGIPDR |
| | I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSS |
| 205 | A3S/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2C | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 206 | A3S/HLE | ARTIFICIAL | EIVLTQSPGTLSLSPGERATLSCRASQSVSSSYLAWYQQKPGQAPRLLIYGASSRATGIPDR |
| | BITE I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGETNYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 207 | B2J/VH | ARTIFICIAL | QVQLVQSGAEVKKPGASVKVSCKASGYTFTGYYMHWVRQAPGQCLEWMGWINPNSGDANYAQ |
| | | | KFQGRVTMTRDTSISTAYMELSRLRSDDTAVYYCARDNLIVVAPVTRDYYYYGMDVWGQGTT |
| | | | VTVSS |
| 208 | B2J/VL | ARTIFICIAL | EIVLTQSPGTLSLSPGERATLSCRASQTVSSSYLVWYQQKPGQAPRLLIYGASSRATGIPDR |
| | | | FSGSGSGTDFTLTISRLEPEDFAVYYCQQYGGSPITFGCGTRLEIK |
| 209 | B2J/CDR-H1 | ARTIFICIAL | GYYMH |
| 210 | B2J/CDR-H2 | ARTIFICIAL | WINPNSGDANYAQKFQG |
| 211 | B2J/CDR-H3 | ARTIFICIAL | DNLIVVAPVTRDYYYYGMDV |
| 212 | B2J/CDR-L1 | ARTIFICIAL | RASQTVSSSYLV |
| 213 | B2J/CDR-L2 | ARTIFICIAL | GASSRAT |
| 214 | B2J/CDR-L3 | ARTIFICIAL | QQYGGSPIT |
| 215 | B2J/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSS |
| 216 | B2J/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 217 | B2J/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 218 | B2J/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSS |
| 219 | B2J/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLTISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 220 | B2J/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLTISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 221 | M5E/VH | ARTIFICIAL | QVQLVQSGAEVKKPGASVKVSCKASGYTFTGYYMHWVRQAPGQCLEWMGWINPNSGDANYAQ |
| | | | KFQGRVTMTRDTSISTAYMELSRLRSDDTAVYYCARDNLIVVAPVTRDYYYYGMDVWGQGTT |
| | | | VTVSS |
| 222 | M5E/VL | ARTIFICIAL | EIVLTQSPGTLSLSPGERATLSCRASQSVSSSYLAWYQQKPGQAPRLLIYGASSRATGIPDR |
| | | | FSGSGSGTDFTLTISRLEPEDFAVYYCQQYGSSPLTFGCGTKLEIK |
| 223 | M5E/CDR-H1 | ARTIFICIAL | GYYMH |
| 224 | M5E/CDR-H2 | ARTIFICIAL | WINPNSGDANYAQKFQG |
| 225 | M5E/CDR-H3 | ARTIFICIAL | DNLIVVAPVTRDYYYYGMDV |
| 226 | M5E/CDR-L1 | ARTIFICIAL | RASQSVSSSYLA |
| 227 | M5E/CDR-L2 | ARTIFICIAL | GASSRAT |
| 228 | M5E/CDR-L3 | ARTIFICIAL | QQYGSSPLT |
| 229 | M5E/SCFV | ARTIFICIAL | EIVLTQSPGTLSLSPGERATLSCRASQSVSSSYLAWYQQKPGQAPRLLIYGASSRATGIPDR |
| | I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSS |
| 230 | M5E/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2E | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 231 | M5E/HLE- | ARTIFICIAL | EIVLTQSPGTLSLSPGERATLSCRASQSVSSSYLAWYQQKPGQAPRLLIYGASSRATGIPDR |
| | BITE I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 232 | M5E/SCFV | ARTIFICIAL | EIVLTQSPGTLSLSPGERATLSCRASQSVSSSYLAWYQQKPGQAPRLLIYGASSRATGIPDR |
| | I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSS |
| 233 | M5E/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2C | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 234 | M5E/HLE | ARTIFICIAL | EIVLTQSPGTLSLSPGERATLSCRASQSVSSSYLAWYQQKPGQAPRLLIYGASSRATGIPDR |
| | BITE I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDNLIVVAPVTRDYYYYGMDVWGQGTTV |
| | | | TVSSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 235 | X3A/VH | ARTIFICIAL | QVQLVQSGAEVKKPGASVKVSCKASGYTFTGYYMHWVRQAPGQCLEWMGWINPNSGDANYAQ |
| | | | KFQGRVTMTRDTSISTAYMELSRLRSDDTAVYYCARDALIVVAPVTRDYYYYGMDVWGQGTT |
| | | | VTASS |
| 236 | X3A/VL | ARTIFICIAL | EIVLTQSPGTLSLSPGERATLSCRASQTVSSSYLVWYQQKPGQAPRLLIYGASSRATGIPDR |
| | | | FSGSGSGTDFTLIISRLEPEDFAVYYCQQYGGSPITFGCGTRLEIK |
| 237 | X3A/CDR-H1 | ARTIFICIAL | GYYMH |
| 238 | X3A/CDR-H2 | ARTIFICIAL | WINPNSGDANYAQKFQG |
| 239 | X3A/CDR-H3 | ARTIFICIAL | DALIVVAPVTRDYYYYGMDV |
| 240 | X3A/CDR-L1 | ARTIFICIAL | RASQTVSSSYLV |
| 241 | X3A/CDR-L2 | ARTIFICIAL | GASSRAT |
| 242 | X3A/CDR-L3 | ARTIFICIAL | QQYGGSPIT |
| 243 | X3A/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASS |
| 244 | X3A/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLIISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 245 | X3A/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 246 | X3A/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASS |
| 247 | X3A/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLIISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 248 | X3A/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 249 | S9W/VH | ARTIFICIAL | QVQLVQSGAEVKKPGASVKVSCKASGYTFTGYYMHWVRQAPGQCLEWMGWINPNSGDANYAQ |
| | | | KFQGRVTMTRDTSISTAYMELSRLRSDDTAVYYCARDALIVVAPVTRDYYYYGMDVWGQGTT |
| | | | VTASS |
| 250 | S9W/VL | ARTIFICIAL | EIVLTQSPGTLSLSPGERATLSCRASQSVSSSYLAWYQQKPGQAPRLLIYGASSRATGIPDR |
| | | | FSGSGSGTDFTLTISRLEPEDFAVYYCQQYGSSPLTFGCGTKLEIK |
| 251 | S9W/CDR-H1 | ARTIFICIAL | GYYMH |
| 252 | S9W/CDR-H2 | ARTIFICIAL | WINPNSGDANYAQKFQG |
| 253 | S9W/CDR-H3 | ARTIFICIAL | DALIVVAPVTRDYYYYGMDV |
| 254 | S9W/CDR-L1 | ARTIFICIAL | RASQSVSSSYLA |
| 255 | S9W/CDR-L2 | ARTIFICIAL | GASSRAT |
| 256 | S9W/CDR-L3 | ARTIFICIAL | QQYGSSPLT |
| 257 | S9W/SCFV | ARTIFICIAL | EIVLTQSPGTLSLSPGERATLSCRASQSVSSSYLAWYQQKPGQAPRLLIYGASSRATGIPDR |
| | I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASS |
| 258 | S9W/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2E | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 259 | S9W/HLE- | ARTIFICIAL | EIVLTQSPGTLSLSPGERATLSCRASQSVSSSYLAWYQQKPGQAPRLLIYGASSRATGIPDR |
| | BITE I2E | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 260 | S9W/SCFV | ARTIFICIAL | EIVLTQSPGTLSLSPGERATLSCRASQSVSSSYLAWYQQKPGQAPRLLIYGASSRATGIPDR |
| | I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASS |
| 261 | S9W/ | ARTIFICIAL | EIVLTQSPGTLSLSPGERATLSCRASQSVSSSYLAWYQQKPGQAPRLLIYGASSRATGIPDR |
| | BISPECIFIC | | FSGSGSGTDFTLTISRLEPEDFAVYYCQQYGSSPLTFGCGTKLEIKGGGGSGGGGSGGGGSQ |
| | MOL I2C | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 262 | S9W/HLE | ARTIFICIAL | EIVLTQSPGTLSLSPGERATLSCRASQSVSSSYLAWYQQKPGQAPRLLIYGASSRATGIPDR |
| | BITE I2C | | FSGSGSGTDFTLTISRLEPEDFAVYYCQQYGSSPLTFGCGTKLEIKGGGGSGGGGSGGGGSQ |
| | | | VQLVQSGAEVKKPGASVKVSCKASGYTFTGYYMHWVRQAPGQCLEWMGWINPNSGDANYAQK |
| | | | FQGRVTMTRDTSISTAYMELSRLRSDDTAVYYCARDALIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 263 | I7L/VH | ARTIFICIAL | QVQLVQSGAEVKKPGASVKVSCKASGYTFTGYYVHWVRQAPGQCLEWMGWINPNSGATNYAQ |
| | | | KFQGRVTMTRDTSISTAYMELSRLRSVDTAVYYCVRDGLIVEAPATRDYYYYGMDVWGQGTT |
| | | | VTVSS |
| 264 | I7L/VL | ARTIFICIAL | EIVLTQSPGTLSLSPGERATLSCRASQSVRSTYLAWYQQTPGQAPRLLISGASSRATGIPDR |
| | | | FSGSGSGTDFILTISRLEPEDFAVYYCQQYDTSPITFGCGTRLEIK |
| 265 | I7L/CDR-H1 | ARTIFICIAL | GYYVH |
| 266 | I7L/CDR-H2 | ARTIFICIAL | WINPNSGATNYAQKFQG |
| 267 | I7L/CDR-H3 | ARTIFICIAL | DGLIVEAPATRDYYYYGMDV |
| 268 | I7L/CDR-L1 | ARTIFICIAL | RASQSVRSTYLA |
| 269 | I7L/CDR-L2 | ARTIFICIAL | GASSRAT |
| 270 | I7L/CDR-L3 | ARTIFICIAL | QQYDTSPIT |
| 271 | I7L/SCFV | ARTIFICIAL | QVQLVQSGAEVKKPGASVKVSCKASGYTFTGYYVHWVRQAPGQCLEWMGWINPNSGATNYAQ |
| | I2E | | KFQGRVTMTRDTSISTAYMELSRLRSVDTAVYYCVRDGLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIK |
| 272 | I7L/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDGLIVEAPATRDYYYYGMDVWGQGTT |
| | MOL I2E | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 273 | I7L/HLE- | ARTIFICIAL | QVQLVQSGAEVKKPGASVKVSCKASGYTFTGYYVHWVRQAPGQCLEWMGWINPNSGATNYAQ |
| | BITE I2E | | KFQGRVTMTRDTSISTAYMELSRLRSVDTAVYYCVRDGLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIKSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 274 | I7L/SCFV | ARTIFICIAL | QVQLVQSGAEVKKPGASVKVSCKASGYTFTGYYVHWVRQAPGQCLEWMGWINPNSGATNYAQ |
| | I2C | | KFQGRVTMTRDTSISTAYMELSRLRSVDTAVYYCVRDGLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIK |
| 275 | I7L/ | ARTIFICIAL | QVQLVQSGAEVKKPGASVKVSCKASGYTFTGYYVHWVRQAPGQCLEWMGWINPNSGATNYAQ |
| | BISPECIFIC | | KFQGRVTMTRDTSISTAYMELSRLRSVDTAVYYCVRDGLIVEAPATRDYYYYGMDVWGQGTT |
| | MOL I2C | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 276 | I7L/HLE | ARTIFICIAL | QVQLVQSGAEVKKPGASVKVSCKASGYTFTGYYVHWVRQAPGQCLEWMGWINPNSGATNYAQ |
| | BITE I2C | | KFQGRVTMTRDTSISTAYMELSRLRSVDTAVYYCVRDGLIVEAPATRDYYYYGMDVWGQGTT |
| | | | VTVSSGGGGSGGGGSGGGGSEIVLTQSPGTLSLSPGERATLSCRASQSVRSTYLAWYQQTPG |
| | | | QAPRLLISGASSRATGIPDRFSGSGSGTDFILTISRLEPEDFAVYYCQQYDTSPITFGCGTR |
| | | | LEIKSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 277 | W5F/VH | ARTIFICIAL | QVQLVQSGAEVKKPGASVKVSCKASGYTFTGYYMHWVRQAPGQCLEWMGWINPNSGDTNYAQ |
| | | | KFQGRVTMTRDTSISTAYMELSRLRSDDTAVYYCARDGLIVVAPVTRDYYYYGMDVWGQGTT |
| | | | VTASS |
| 278 | W5F/VL | ARTIFICIAL | EIVLTQSPGTLSLSPGERATLSCRASQTVSSSYLVWYQQKPGQAPRLLIYGASSRATGIPDR |
| | | | FSGSGSGTDFTLIISRLEPEDFAVYYCQQYGGSPITFGCGTRLEIK |
| 279 | W5F/CDR-H1 | ARTIFICIAL | GYYMH |
| 280 | W5F/CDR-H2 | ARTIFICIAL | WINPNSGDTNYAQKFQG |
| 281 | W5F/CDR-H3 | ARTIFICIAL | DGLIVVAPVTRDYYYYGMDV |
| 282 | W5F/CDR-L1 | ARTIFICIAL | RASQTVSSSYLV |
| 283 | W5F/CDR-L2 | ARTIFICIAL | GASSRAT |
| 284 | W5F/CDR-L3 | ARTIFICIAL | QQYGGSPIT |
| 285 | W5F/SCFV | ARTIFICIAL | EIVLTQSPGTLSLSPGERATLSCRASQTVSSSYLVWYQQKPGQAPRLLIYGASSRATGIPDR |
| | I2E | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDTNYAQK |
| | | | FQGRVTMTRDTSISTAYMELSRLRSDDTAVYYCARDGLIVVAPVTRDYYYYGMDVWGQGTTV |
| | | | TASS |
| 286 | W5F/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLIISRLEPEDFAVYYCQQYGGSPITFGCGTRLEIKGGGGSGGGGSGGGGSQ |
| | MOL I2E | | VQLVQSGAEVKKPGASVKVSCKASGYTFTGYYMHWVRQAPGQCLEWMGWINPNSGDTNYAQK |
| | | | FQGRVTMTRDTSISTAYMELSRLRSDDTAVYYCARDGLIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVL |
| 287 | W5F/HLE- | ARTIFICIAL | EIVLTQSPGTLSLSPGERATLSCRASQTVSSSYLVWYQQKPGQAPRLLIYGASSRATGIPDR |
| | BITE I2E | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDTNYAQK |
| | | | FQGRVTMTRDTSISTAYMELSRLRSDDTAVYYCARDGLIVVAPVTRDYYYYGMDVWGQGTTV |
| | | | TASSSGGGGSEVQLVESGGGLVQPGGSLKLSCAASGFTFNKYAINWVRQAPGKGLEWVARIR |
| | | | SKYNNYATYYADAVKDRFTISRDDSKNTVYLQMNNLKTEDTAVYYCARAGNFGSSYISYWAY |
| | | | WGQGTLVTVSSGGGGSGGGGSGGGGSQTVVTQEPSLTVSPGGTVTITCGSSTGAVTSGNYPN |
| | | | WVQKKPGQAPRGLIGGTKFLAPGTPARFSGSLSGGKAALTLSGVQPEDEAEYYCVLWYSNRW |
| | | | VFGSGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 288 | W5F/SCFV | ARTIFICIAL | EIVLTQSPGTLSLSPGERATLSCRASQTVSSSYLVWYQQKPGQAPRLLIYGASSRATGIPDR |
| | I2C | | FSGSGSGTDFTLIISRLEPE |
| | | | DFAVYYCQQYGGSPITFGCGTRLEIKGGGGSGGGGSGGGGSQVQLVQSGAEVKKPGASVKVS |
| | | | CKASGYTFTGYYMHWVRQAPGQCLEWMGWINPNSGDTNYAQKFQGRVTMTRDTSISTAYMEL |
| | | | SRLRSDDTAVYYCARDGLIVVAPVTRDYYYYGMDVWGQGTTVTASS |
| 289 | W5F/ | ARTIFICIAL | EIVLTQSPGTLSLSPGERATLSCRASQTVSSSYLVWYQQKPGQAPRLLIYGASSRATGIPDR |
| | BISPECIFIC | | FSGSGSGTDFTLIISRLEPEDFAVYYCQQYGGSPITFGCGTRLEIKGGGGSGGGGSGGGGSQ |
| | MOL I2C | | VQLVQSGAEVKKPGASVKVSCKASGYTFTGYYMHWVRQAPGQCLEWMGWINPNSGDTNYAQK |
| | | | FQGRVTMTRDTSISTAYMELSRLRSDDTAVYYCARDGLIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVL |
| 290 | W5F/HLE | ARTIFICIAL | EIVLTQSPGTLSLSPGERATLSCRASQTVSSSYLVWYQQKPGQAPRLLIYGASSRATGIPDR |
| | BITE I2C | | FSGSGSGTDFTLIISRLEPEDFAVYYCQQYGGSPITFGCGTRLEIKGGGGSGGGGSGGGGSQ |
| | | | VQLVQSGAEVKKPGASVKVSCKASGYTFTGYYMHWVRQAPGQCLEWMGWINPNSGDTNYAQK |
| | | | FQGRVTMTRDTSISTAYMELSRLRSDDTAVYYCARDGLIVVAPVTRDYYYYGMDVWGQGTTV |
| | | | TASSSGGGGSEVQLVESGGGLVQPGGSLKLSCAASGFTFNKYAMNWVRQAPGKGLEWVARIR |
| | | | SKYNNYATYYADSVKDRFTISRDDSKNTAYLQMNNLKTEDTAVYYCVRHGNFGNSYISYWAY |
| | | | WGQGTLVTVSSGGGGSGGGGSGGGGSQTVVTQEPSLTVSPGGTVTLTCGSSTGAVTSGNYPN |
| | | | WVQQKPGQAPRGLIGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNGKEYK |
| | | | CKVSNKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKSLSLS |
| | | | PGK |
| 291 | K4Y/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDYYSSSYTLYYYYGMDVWGQGTTVTV |
| | | | SS |
| 292 | K4Y/VL | ARTIFICIAL | QSALTQPPSASGSPGQSVTISCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 293 | K4Y/CDR-H1 | ARTIFICIAL | DYHMH |
| 294 | K4Y/CDR-H2 | ARTIFICIAL | WIKPISGDANYAQKFQG |
| 295 | K4Y/CDR-H3 | ARTIFICIAL | DYYSSSYTLYYYYGMDV |
| 296 | K4Y/CDR-L1 | ARTIFICIAL | TGTSSDVGGYNYVS |
| 297 | K4Y/CDR-L2 | ARTIFICIAL | EVSKRPS |
| 298 | K4Y/CDR-L3 | ARTIFICIAL | SSYAGSNNFV |
| 299 | K4Y/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDYYSSSYTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 300 | K4Y/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSYTLYYYYGMDVWGQGTTVTV |
| | MOL I2E | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 301 | K4Y/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDYYSSSYTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 302 | K4Y/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDYYSSSYTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 303 | K4Y/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSYTLYYYYGMDVWGQGTTVTV |
| | MOL I2C | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 304 | K4Y/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDYYSSSYTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 305 | M4B/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDAYSSSWTLYYYYGMDVWGQGTTVTV |
| | | | SS |
| 306 | M4B/VL | ARTIFICIAL | QSALTQPPSASGSPGQSVTISCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 307 | M4B/CDR-H1 | ARTIFICIAL | DYHMH |
| 308 | M4B/CDR-H2 | ARTIFICIAL | WIKPISGDANYAQKFQG |
| 309 | M4B/CDR-H3 | ARTIFICIAL | DAYSSSWTLYYYYGMDV |
| 310 | M4B/CDR-L1 | ARTIFICIAL | TGTSSDVGGYNYVS |
| 311 | M4B/CDR-L2 | ARTIFICIAL | EVSKRPS |
| 312 | M4B/CDR-L3 | ARTIFICIAL | SSYAGSNNFV |
| 313 | M4B/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVI |
| 314 | M4B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDAYSSSWTLYYYYGMDVWGQGTTVTV |
| | MOL I2E | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 315 | M4B/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 316 | M4B/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 317 | M4B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDAYSSSWTLYYYYGMDVWGQGTTVTV |
| | MOL I2C | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 318 | M4B/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 319 | B4G/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDAYSSSWTLYYYYGMDVWGQGTTVTV |
| | | | SS |
| 320 | B4G/VL | ARTIFICIAL | QSALTQPPSASGSPGQSVTSYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 321 | B4G/CDR-H1 | ARTIFICIAL | DYHMH |
| 322 | B4G/CDR-H2 | ARTIFICIAL | WIKPISGDANYAQKFQG |
| 323 | B4G/CDR-H3 | ARTIFICIAL | DAYSSSWTLYYYYGMDV |
| 324 | B4G/CDR-L1 | ARTIFICIAL | TGTSSDVGGYNYVS |
| 325 | B4G/CDR-L2 | ARTIFICIAL | EVSKRPS |
| 326 | B4G/CDR-L3 | ARTIFICIAL | SSYAGSNNFV |
| 327 | B4G/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 328 | B4G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDAYSSSWTLYYYYGMDVWGQGTTVTV |
| | MOL I2E | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 329 | B4G/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 330 | B4G/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 331 | B4G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDAYSSSWTLYYYYGMDVWGQGTTVTV |
| | MOL I2C | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 332 | B4G/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDAYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 333 | U8B/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDYYSSSWTLYYYYGMDVWGQGTTVTV |
| | | | SS |
| 334 | U8B/VL | ARTIFICIAL | QSALTQPPSASGSPGQSVTSYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 335 | U8B/CDR-H1 | ARTIFICIAL | DYHMH |
| 336 | U8B/CDR-H2 | ARTIFICIAL | WIKPISGDANYAQKFQG |
| 337 | U8B/CDR-H3 | ARTIFICIAL | DYYSSSWTLYYYYGMDV |
| 338 | U8B/CDR-L1 | ARTIFICIAL | TGTSSDVGGYNYVS |
| 339 | U8B/CDR-L2 | ARTIFICIAL | EVSKRPS |
| 340 | U8B/CDR-L3 | ARTIFICIAL | SSYAGSNNFV |
| 341 | U8B/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 342 | U8B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2E | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 343 | U8B/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 344 | U8B/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 345 | U8B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2C | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 346 | U8B/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 347 | Y8G/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDGYSSSWTLYYYYGMDVWGQGTTVTV |
| | | | SS |
| 348 | Y8G/VL | ARTIFICIAL | QSALTQPPSASGSPGQSVTSYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 349 | Y8G/CDR-H1 | ARTIFICIAL | DYHMH |
| 350 | Y8G/CDR-H2 | ARTIFICIAL | WIKPISGDANYAQKFQG |
| 351 | Y8G/CDR-H3 | ARTIFICIAL | DGYSSSWTLYYYYGMDV |
| 352 | Y8G/CDR-L1 | ARTIFICIAL | TGTSSDVGGYNYVS |
| 353 | Y8G/CDR-L2 | ARTIFICIAL | EVSKRPS |
| 354 | Y8G/CDR-L3 | ARTIFICIAL | SSYAGSNNFV |
| 355 | Y8G/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 356 | Y8G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDGYSSSWTLYYYYGMDVWGQGTTVTV |
| | MOL I2E | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 357 | Y8G/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 358 | Y8G/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 359 | Y8G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDGYSSSWTLYYYYGMDVWGQGTTVTV |
| | MOL I2C | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 360 | Y8G/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 361 | G8B/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDYYSSSWTLYYYYGMDVWGQGTTVTV |
| | | | SS |
| 362 | G8B/VL | ARTIFICIAL | QSALTQPPSASGSPGQSVTIYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 363 | G8B/CDR-H1 | ARTIFICIAL | DYHMH |
| 364 | G8B/CDR-H2 | ARTIFICIAL | WIKPISGDANYAQKFQG |
| 365 | G8B/CDR-H3 | ARTIFICIAL | DYYSSSWTLYYYYGMDV |
| 366 | G8B/CDR-L1 | ARTIFICIAL | TGTSSDVGGYNYVS |
| 367 | G8B/CDR-L2 | ARTIFICIAL | EVSKRPS |
| 368 | G8B/CDR-L3 | ARTIFICIAL | SSYAGSNNFV |
| 369 | G8B/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 370 | G8B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2E | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 371 | G8B/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 372 | G8B/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 373 | G8B/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2C | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 374 | G8B/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTIYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 375 | W9B/VH | ARTIFICIAL | QVQLVQSGAEVKKPGASVKVSCKASGYTFTDYHMHWVRQAPGQCLEWMGWIKPISGDTNYAQ |
| | | | KFQGRVTMTRDTSISTAYMELSRLRSDDTAVYYCARDGYSSSWTLYYYYGMDVWGQGTTVTV |
| | | | SS |
| 376 | W9B/VL | ARTIFICIAL | QSALTQPPSASGSPGQSVTSYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 377 | W9B/CDR-H1 | ARTIFICIAL | DYHMH |
| 378 | W9B/CDR-H2 | ARTIFICIAL | WIKPISGDTNYAQKFQG |
| 379 | W9B/CDR-H3 | ARTIFICIAL | DGYSSSWTLYYYYGMDV |
| 380 | W9B/CDR-L1 | ARTIFICIAL | TGTSSDVGGYNYVS |
| 381 | W9B/CDR-L2 | ARTIFICIAL | EVSKRPS |
| 382 | W9B/CDR-L3 | ARTIFICIAL | SSYAGSNNFV |
| 383 | W9B/SCFV | ARTIFICIAL | QVQLVQSGAEVKKPGASVKVSCKASGYTFTDYHMHWVRQAPGQCLEWMGWIKPISGDTNYAQ |
| | I2E | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 384 | W9B/ | ARTIFICIAL | QVQLVQSGAEVKKPGASVKVSCKASGYTFTDYHMHWVRQAPGQCLEWMGWIKPISGDTNYAQ |
| | BISPECIFIC | | KFQGRVTMTRDTSISTAYMELSRLRSDDTAVYYCARDGYSSSWTLYYYYGMDVWGQGTTVTV |
| | MOL I2E | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 385 | W9B/HLE- | ARTIFICIAL | QVQLVQSGAEVKKPGASVKVSCKASGYTFTDYHMHWVRQAPGQCLEWMGWIKPISGDTNYAQ |
| | BITE I2E | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 386 | W9B/SCFV | ARTIFICIAL | QVQLVQSGAEVKKPGASVKVSCKASGYTFTDYHMHWVRQAPGQCLEWMGWIKPISGDTNYAQ |
| | I2C | | KFQGRVTMTRDT |
| | | | SISTAYMELSRLRSDDTAVYYCARDGYSSSWTLYYYYGMDVWGQGTTVTVSSGGGGSGGGGS |
| | | | GGGGSQSALTQPPSASGSPGQSVTSYCTGTSSDVGGYNYVSWYQQHPGKAPKLMIYEVSKRP |
| | | | SGVPDRFSGSKSGNTASLTVSGLQAEDEADYYCSSYAGSNNFVFGCGTKVTVL |
| 387 | W9B/ | ARTIFICIAL | QVQLVQSGAEVKKPGASVKVSCKASGYTFTDYHMHWVRQAPGQCLEWMGWIKPISGDTNYAQ |
| | BISPECIFIC | | KFQGRVTMTRDTSISTAYMELSRLRSDDTAVYYCARDGYSSSWTLYYYYGMDVWGQGTTVTV |
| | MOL I2C | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 388 | W9B/HLE | ARTIFICIAL | QVQLVQSGAEVKKPGASVKVSCKASGYTFTDYHMHWVRQAPGQCLEWMGWIKPISGDTNYAQ |
| | BITE I2C | | KFQGRVTMTRDTSISTAYMELSRLRSDDTAVYYCARDGYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 389 | A9G/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDYYSSSWTLYYYYGMDVWGQGTTVTV |
| | | | SS |
| 390 | A9G/VL | ARTIFICIAL | QSALTQPPSASGSPGQSVTISCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 391 | A9G/CDR-H1 | ARTIFICIAL | DYHMH |
| 392 | A9G/CDR-H2 | ARTIFICIAL | WIKPISGDANYAQKFQG |
| 393 | A9G/CDR-H3 | ARTIFICIAL | DYYSSSWTLYYYYGMDV |
| 394 | A9G/CDR-L1 | ARTIFICIAL | TGTSSDVGGYNYVS |
| 395 | A9G/CDR-L2 | ARTIFICIAL | EVSKRPS |
| 396 | A9G/CDR-L3 | ARTIFICIAL | SSYAGSNNFV |
| 397 | A9G/SCFV | ARTIFICIAL | QVQLVQSGAEVKKPGASVKVSCKASGYTFTDYHMHWVRQAPGQCLEWMGWIKPISGDANYAQ |
| | I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 398 | A9G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2E | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 399 | A9G/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 400 | A9G/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 401 | A9G/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSWTLYYYYGMDVWGQGTTVTV |
| | MOL I2C | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 402 | A9G/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 403 | D3F/VH | ARTIFICIAL | QVQLVQSGAEVKKPGASVKVSCKASGYTFTDYHMHWVRQAPGQCLEWMGWIKPISGDTNYAQ |
| | | | KFQGRVTMTRDTSISTAYMELSRLRSDDTAVYYCARDYYSSSWTLYYYYGMDVWGQGTTVTV |
| | | | SS |
| 404 | D3F/VL | ARTIFICIAL | QSALTQPPSASGSPGQSVTSYCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 405 | D3F/CDR-H1 | ARTIFICIAL | DYHMH |
| 406 | D3F/CDR-H2 | ARTIFICIAL | WIKPISGDTNYAQKFQG |
| 407 | D3F/CDR-H3 | ARTIFICIAL | DYYSSSWTLYYYYGMDV |
| 408 | D3F CDR-L1 | ARTIFICIAL | TGTSSDVGGYNYVS |
| 409 | D3F/CDR-L2 | ARTIFICIAL | EVSKRPS |
| 410 | D3F/CDR-L3 | ARTIFICIAL | SSYAGSNNFV |
| 411 | D3F/SCFV | ARTIFICIAL | QVQLVQSGAEVKKPGASVKVSCKASGYTFTDYHMHWVRQAPGQCLEWMGWIKPISGDTNYAQ |
| | I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 412 | D3F/ | ARTIFICIAL | QVQLVQSGAEVKKPGASVKVSCKASGYTFTDYHMHWVRQAPGQCLEWMGWIKPISGDTNYAQ |
| | BISPECIFIC | | KFQGRVTMTRDTSISTAYMELSRLRSDDTAVYYCARDYYSSSWTLYYYYGMDVWGQGTTVTV |
| | MOL I2E | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 413 | D3F/HLE- | ARTIFICIAL | QVQLVQSGAEVKKPGASVKVSCKASGYTFTDYHMHWVRQAPGQCLEWMGWIKPISGDTNYAQ |
| | BITE I2E | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 414 | D3F/SCFV | ARTIFICIAL | QVQLVQSGAEVKKPGASVKVSCKASGYTFTDYHMHWVRQAPGQCLEWMGWIKPISGDTNYAQ |
| | I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 415 | D3F/ | ARTIFICIAL | QVQLVQSGAEVKKPGASVKVSCKASGYTFTDYHMHWVRQAPGQCLEWMGWIKPISGDTNYAQ |
| | BISPECIFIC | | KFQGRVTMTRDTSISTAYMELSRLRSDDTAVYYCARDYYSSSWTLYYYYGMDVWGQGTTVTV |
| | MOL I2C | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 416 | D3F/HLE | ARTIFICIAL | QVQLVQSGAEVKKPGASVKVSCKASGYTFTDYHMHWVRQAPGQCLEWMGWIKPISGDTNYAQ |
| | BITE I2C | | KFQGRVTMTRDTSISTAYMELSRLRSDDTAVYYCARDYYSSSWTLYYYYGMDVWGQGTTVTV |
| | | | SSGGGGSGGGGSGGGGSQSALTQPPSASGSPGQSVTSYCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 417 | E4N/VH | ARTIFICIAL | QVQLVQSGAEVKKPGASVKVSCKASGYTFTDYHMHWVRQAPGQCLEWMGWIKPISGDANYAQ |
| | | | KFQGRVTMTRDTSISTAYMELSRLRSDDTAVYYCARDYYSSSFTLYYYYGMDVWGQGTTVTV |
| | | | SS |
| 418 | E4N/VL | ARTIFICIAL | QSALTQPPSASGSPGQSVTISCTGTSSDVGGYNYVSWYQQHPGKAPKLMIYEVSKRPSGVPD |
| | | | RFSGSKSGNTASLTVSGLQAEDEADYYCSSYAGSNNFVFGCGTKVTVL |
| 419 | E4N/CDR-H1 | ARTIFICIAL | DYHMH |
| 420 | E4N/CDR-H2 | ARTIFICIAL | WIKPISGDANYAQKFQG |
| 421 | E4N/CDR-H3 | ARTIFICIAL | DYYSSSFTLYYYYGMDV |
| 422 | E4N/CDR-L1 | ARTIFICIAL | TGTSSDVGGYNYVS |
| 423 | E4N/CDR-L2 | ARTIFICIAL | EVSKRPS |
| 424 | E4N/CDR-L3 | ARTIFICIAL | SSYAGSNNFV |
| 425 | E4N/SCFV | ARTIFICIAL | QVQLVQSGAEVKKPGASVKVSCKASGYTFTDYHMHWVRQAPGQCLEWMGWIKPISGDANYAQ |
| | I2F | | KFQGRVTMTRDTSISTAYMELSRLRSDDTAVYYCARDYYSSSFTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 426 | E4N/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSFTLYYYYGMDVWGQGTTVTV |
| | MOL I2E | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVL |
| 427 | E4N/HLE- | ARTIFICIAL | QVQLVQSGAEVKKPGASVKVSCKASGYTFTDYHMHWVRQAPGQCLEWMGWIKPISGDANYAQ |
| | BITE I2E | | KFQGRVTMTRDTSISTAYMELSRLRSDDTAVYYCARDYYSSSFTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAINWVRQAPGKGLEWVARIRS |
| | | | KYNNYATYYADAVKDRFTISRDDSKNTVYLQMNNLKTEDTAVYYCARAGNFGSSYISYWAYW |
| | | | GQGTLVTVSSGGGGSGGGGSGGGGSQTVVTQEPSLTVSPGGTVTITCGSSTGAVTSGNYPNW |
| | | | VQKKPGQAPRGLIGGTKFLAPGTPARFSGSLSGGKAALTLSGVQPEDEAEYYCVLWYSNRWV |
| | | | FGSGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 428 | E4N/SCFV | ARTIFICIAL | QVQLVQSGAEVKKPGASVKVSCKASGYTFTDYHMHWVRQAPGQCLEWMGWIKPISGDANYAQ |
| | I2C | | KFQGRVTMTRDTSISTAYMELSRLRSDDTAVYYCARDYYSSSFTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVL |
| 429 | E4N/ | ARTIFICIAL | QVQLVQSGAEVKKPGASVKVSCKASGYTFTDYHMHWVRQAPGQCLEWMGWIKPISGDANYAQ |
| | BISPECIFIC | | KFQGRVTMTRDTSISTAYMELSRLRSDDTAVYYCARDYYSSSFTLYYYYGMDVWGQGTTVTV |
| | MOL I2C | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVL |
| 430 | E4N/HLE | ARTIFICIAL | QVQLVQSGAEVKKPGASVKVSCKASGYTFTDYHMHWVRQAPGQCLEWMGWIKPISGDANYAQ |
| | BITE I2C | | KFQGRVTMTRDTSISTAYMELSRLRSDDTAVYYCARDYYSSSFTLYYYYGMDVWGQGTTVTV |
| | | | SSGGGGSGGGGSGGGGSQSALTQPPSASGSPGQSVTISCTGTSSDVGGYNYVSWYQQHPGKA |
| | | | PKLMIYEVSKRPSGVPDRFSGSKSGNTASLTVSGLQAEDEADYYCSSYAGSNNFVFGCGTKV |
| | | | TVLSGGGGSEVQLVESGGGLVQPGGSLKLSCAASGFTFNKYAMNWVRQAPGKGLEWVARIRS |
| | | | KYNNYATYYADSVKDRFTISRDDSKNTAYLQMNNLKTEDTAVYYCVRHGNFGNSYISYWAYW |
| | | | GQGTLVTVSSGGGGSGGGGSGGGGSQTVVTQEPSLTVSPGGTVTLTCGSSTGAVTSGNYPNW |
| | | | VQQKPGQAPRGLIGGTKFLAPGTPARFSGSLLGGKAALTLSGVQPEDEAEYYCVLWYSNRWV |
| | | | FGGGTKLTVLGGGGDKTHTCPPCPAPELLGGPSVFLFPPKPKDTLMISRTPEVTCVVVDVSH |
| | | | EDPEVKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSLSLSP |
| | | | GK |
| 431 | CL6 AE3-20 X | ARTIFICIAL | SYTMS |
| | I2C X SCFC/ | | |
| | HCDR1 | | |
| 432 | CL6 AE3-20/ | ARTIFICIAL | TISSGGGRTYYPDSVKG |
| | HCDR2 | | |
| 433 | CL6 AE3-20/ | ARTIFICIAL | GDYRYDGFAY |
| | HCDR3 | | |
| 434 | CL6 AE3-20/ | ARTIFICIAL | RASENIDSYLA |
| | LCDR1 | | |
| 435 | CL6 AE3-20/ | ARTIFICIAL | ASTLLVD |
| | LCDR2 | | |
| 436 | CL6 AE3-20/ | ARTIFICIAL | QHYYSIPYT |
| | LCDR3 | | |
| 437 | CL6 AE3-20/ | ARTIFICIAL | EVKLVESGGGLVKPGGSLKLSCAASGFTFNSYTMSWVRQTPAKRLEWVVTISSGGGRTYYPD |
| | VH | | SVKGRFTISRDNARNTLYLQMSSLRSEDTAMYYCIRGDYRYDGFAYWGQGTLVTVST |
| 438 | CL6 AE3-20/ | ARTIFICIAL | DIQMTQSPASLSASVGETVTITCRASENIDSYLAWYQQKQGKSPQLLVYASTLLVDGVPSRF |
| | VL | | SGSRSGTQFSLKINSLQSEDVARYYCQHYYSIPYTFGSGTKLEIK |
| 439 | CL6 AE3-20/ | ARTIFICIAL | EVKLVESGGGLVKPGGSLKLSCAASGFTFNSYTMSWVRQTPAKRLEWVVTISSGGGRTYYPD |
| | SCFV | | SVKGRFTISRDNARNTLYLQMSSLRSEDTAMYYCIRGDYRYDGFAYWGQGTLVTVSTGGGGS |
| | | | GGGGSGGGGSDIQMTQSPASLSASVGETVTITCRASENIDSYLAWYQQKQGKSPQLLVYAST |
| | | | LLVDGVPSRFSGSRSGTQFSLKINSLQSEDVARYYCQHYYSIPYTFGSGTKLEIK |
| 440 | CL6 AE3-20/ | ARTIFICIAL | EVKLVESGGGLVKPGGSLKLSCAASGFTFNSYTMSWVRQTPAKRLEWVVTISSGGGRTYYPD |
| | BISPECIFIC | | SVKGRFTISRDNARNTLYLQMSSLRSEDTAMYYCIRGDYRYDGFAYWGQGTLVTVSTGGGGS |
| | MOL. | | GGGGSGGGGSDIQMTQSPASLSASVGETVTITCRASENIDSYLAWYQQKQGKSPQLLVYAST |
| | | | LLVDGVPSRFSGSRSGTQFSLKINSLQSEDVARYYCQHYYSIPYTFGSGTKLEIKSGGGGSE |
| | | | VQLVESGGGLVQPGGSLKLSCAASGFTFNKYAMNWVRQAPGKGLEWVARIRSKYNNYATYYA |
| | | | DSVKDRFTISRDDSKNTAYLQMNNLKTEDTAVYYCVRHGNFGNSYISYWAYWGQGTLVTVSS |
| | | | GGGGSGGGGSGGGGSQTVVTQEPSLTVSPGGTVTLTCGSSTGAVTSGNYPNWVQQKPGQAPR |
| | | | GLIGGTKFLAPGTPARFSGSLLGGKAALTLSGVQPEDEAEYYCVLWYSNRWVFGGGTKLTVL |
| 441 | CL6 AE3-20/ | ARTIFICIAL | EVKLVESGGGLVKPGGSLKLSCAASGFTFNSYTMSWVRQTPAKRLEWVVTISSGGGRTYYPD |
| | HLE-BITE I2E | | SVKGRFTISRDNARNTLYLQMSSLRSEDTAMYYCIRGDYRYDGFAYWGQGTLVTVSTGGGGS |
| | | | GGGGSGGGGSDIQMTQSPASLSASVGETVTITCRASENIDSYLAWYQQKQGKSPQLLVYAST |
| | | | LLVDGVPSRFSGSRSGTQFSLKINSLQSEDVARYYCQHYYSIPYTFGSGTKLEIKSGGGGSE |
| | | | VQLVESGGGLVQPGGSLKLSCAASGFTFNKYAMNWVRQAPGKGLEWVARIRSKYNNYATYYA |
| | | | DSVKDRFTISRDDSKNTAYLQMNNLKTEDTAVYYCVRHGNFGNSYISYWAYWGQGTLVTVSS |
| | | | GGGGSGGGGSGGGGSQTVVTQEPSLTVSPGGTVTLTCGSSTGAVTSGNYPNWVQQKPGQAPR |
| | | | GLIGGTKFLAPGTPARFSGSLLGGKAALTLSGVQPEDEAEYYCVLWYSNRWVFGGGTKLTVL |
| | | | GGGGDKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 442 | Human CD38 | ARTIFICIAL | QDGNEEMGGITQTPYKVSISGTTVILTCPQYPGSEILWQHNDKNIGGDEDDKNIGSDEDHLS |
| | ECD/ | | LKEFSELEQSGYYVCYPRGSKPEDANFYLYLRARVCENCMEMD |
| 443 | Human CD3€ | ARTIFICIAL | QDGNEEMGGITQTPYKVSISGTTVILT |
| | ECD/pos. | | |
| | 1-27 | | |
| 444 | CDR-L1 of | ARTIFICIAL | GSSTGAVTSGYYPN |
| | H2C | | |
| 445 | CDR-L2 of | ARTIFICIAL | GTKFLAP |
| | H2C | | |
| 446 | CDR-L3 of | ARTIFICIAL | ALWYSNRWV |
| | H2C | | |
| 447 | CDR-L1 of | ARTIFICIAL | RSSTGAVTSGYYPN |
| | E2M | | |
| 448 | CDR-L2 of | ARTIFICIAL | ATDMRPS |
| | E2M | | |
| 449 | CDR-L3 of | ARTIFICIAL | ALWYSNRWV |
| | E2M | | |
| 450 | CDR-L1 of | ARTIFICIAL | GSSTGAVTSGNYPN |
| | F12Q | | |
| 451 | CDR-L2 of | ARTIFICIAL | GTKFLAP |
| | F12Q | | |
| 452 | CDR-L3 of | ARTIFICIAL | VLWYSNRWV |
| | F12Q | | |
| 453 | CDR-H1 of | ARTIFICIAL | IYAMN |
| | F6A | | |
| 454 | CDR-H2 of | ARTIFICIAL | RIRSKYNNYATYYADSVKS |
| | F6A | | |
| 455 | CDR-H3 of | ARTIFICIAL | HGNFGNSYVSFFAY |
| | F6A | | |
| 456 | CDR-H1 of | ARTIFICIAL | KYAMN |
| | H2C | | |
| 457 | CDR-H2 of | ARTIFICIAL | RIRSKYNNYATYYADSVKD |
| | H2C | | |
| 458 | CDR-H3 of | ARTIFICIAL | HGNFGNSYISYWAY |
| | H2C | | |
| 459 | CDR-H1 of | ARTIFICIAL | SYAMN |
| | H1E | | |
| 460 | CDR-H2 of H1E | ARTIFICIAL | RIRSKYNNYATYYADSVKG |
| 461 | CDR-H3 of | ARTIFICIAL | HGNFGNSYLSFWAY |
| | H1E | | |
| 462 | CDR-H1 of | ARTIFICIAL | RYAMN |
| | G4H | | |
| 463 | CDR-H2 of G4H | ARTIFICIAL | RIRSKYNNYATYYADSVKG |
| 464 | CDR-H3 of | ARTIFICIAL | HGNFGNSYLSYFAY |
| | G4H | | |
| 465 | CDR-H1 of | ARTIFICIAL | VYAMN |
| | A2J | | |
| 466 | CDR-H2 of | ARTIFICIAL | RIRSKYNNYATYYADSVKK |
| | A2J | | |
| 467 | CDR-H3 of | ARTIFICIAL | HGNFGNSYLSWWAY |
| | A2J | | |
| 468 | CDR-H1 of E1L | ARTIFICIAL | KYAMN |
| 469 | CDR-H2 of E1L | ARTIFICIAL | RIRSKYNNYATYYADSVKS |
| 470 | CDR-H3 of | ARTIFICIAL | HGNFGNSYTSYYAY |
| | E1L | | |
| 471 | CDR-H1 of E2M | ARTIFICIAL | GYAMN |
| 472 | CDR-H2 of E2M | ARTIFICIAL | RIRSKYNNYATYYADSVKE |
| 473 | CDR-H3 of E2M | ARTIFICIAL | HRNFGNSYLSWFAY |
| 474 | CDR-H1 of F70 | ARTIFICIAL | VYAMN |
| 475 | CDR-H2 of F70 | ARTIFICIAL | RIRSKYNNYATYYADSVKK |
| 476 | CDR-H3 of F70 | ARTIFICIAL | HGNFGNSYISWWAY |
| 477 | CDR-H1 of | ARTIFICIAL | SYAMN |
| | F12Q | | |
| 478 | CDR-H2 of | ARTIFICIAL | RIRSKYNNYATYYADSVKG |
| | F12Q | | |
| 479 | CDR-H3 of F12Q | ARTIFICIAL | HGNFGNSYVSWWAY |
| 480 | CDR-H1 of | ARTIFICIAL | KYAMN |
| | I2C | | |
| 481 | CDR-H2 of | ARTIFICIAL | RIRSKYNNYATYYADSVKD |
| | I2C | | |
| 482 | CDR-H3 of | ARTIFICIAL | HGNFGNSYISYWAY |
| | I2C | | |
| 483 | CDR-L2 of | ARTIFICIAL | GTKFLAP |
| | H2C | | |
| 484 | CDR-L3 of | ARTIFICIAL | ALWYSNRWV |
| | H2C | | |
| 485 | CDR-H1 of | ARTIFICIAL | KYAMN |
| | H2C | | |
| 486 | CDR-L1 of | ARTIFICIAL | GSSTGAVTSGYYPN |
| | H1E | | |
| 487 | CDR-L2 of | ARTIFICIAL | GTKFLAP |
| | H1E | | |
| 488 | CDR-L3 of | ARTIFICIAL | ALWYSNRWV |
| | H1E | | |
| 489 | CDR-H1 of H1E | ARTIFICIAL | SYAMN |
| 490 | CDR-H2 of H1E | ARTIFICIAL | RIRSKYNNYATYYADSVKG |
| 491 | CDR-H3 of H1E | ARTIFICIAL | HGNFGNSYLSFWAY |
| 492 | CDR-L1 of | ARTIFICIAL | GSSTGAVTSGYYPN |
| | G4H | | |
| 493 | CDR-L2 of G4H | ARTIFICIAL | GTKFLAP |
| 494 | CDR-L3 of G4H | ARTIFICIAL | ALWYSNRWV |
| 495 | CDR-L1 of | ARTIFICIAL | RSSTGAVTSGYYPN |
| | A2J | | |
| 496 | CDR-L2 of | ARTIFICIAL | ATDMRPS |
| | A2J | | |
| 497 | CDR-L3 of | ARTIFICIAL | ALWYSNRWV |
| | A2J | | |
| 498 | CDR-L1 of E1L | ARTIFICIAL | GSSTGAVTSGYYPN |
| 499 | CDR-L2 of E1L | ARTIFICIAL | GTKFLAP |
| 500 | CDR-L3 of | ARTIFICIAL | ALWYSNRWV |
| | E1L | | |
| 501 | CDR-L1 of | ARTIFICIAL | GSSTGAVTSGYYPN |
| | F70 | | |
| 502 | CDR-L2 of | ARTIFICIAL | GTKFLAP |
| | F70 | | |
| 503 | CDR-L3 of F70 | ARTIFICIAL | ALWYSNRWV |
| 504 | CDR-L1 of I2C | ARTIFICIAL | GSSTGAVTSGNYPN |
| 505 | CDR-L2 of | ARTIFICIAL | GTKFLAP |
| | I2C | | |
| 506 | CDR-L3 of | ARTIFICIAL | VLWYSNRWV |
| | I2C | | |
| 507 | VL of H2C | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 508 | VL of E2M | ARTIFICIAL | QTVVTQEPSLTVSPGGTVTLTCRSSTGAVTSGYYPNWVQQKPGQAPRGLIGATDMRPSGTPA |
| | | | RFSGSLLGGKAALTLSGVQPEDEAEYYCALWYSNRWVFGGGTKLTVL |
| 509 | VL of F12Q | ARTIFICIAL | QTVVTQEPSLTVSPGGTVTLTCGSSTGAVTSGNYPNWVQQKPGQAPRGLIGGTKFLAPGTPA |
| | | | RFSGSLLGGKAALTLSGVQPEDEAEYYCVLWYSNRWVFGGGTKLTVL |
| 510 | VL variant | ARTIFICIAL | ELVVTQEPSLTVSPGGTVTLTCGSSTGAVTSGYYPNWVQQKPGQAPRGLIGGTKFLAPGTPA |
| | of H2C | | RFSGSLLGGKAALTLSGVQPEDEAEYYCALWYSNRWVFGGGTKLTVL |
| 511 | VL variant | ARTIFICIAL | ELVVTQEPSLTVSPGGTVTLTCRSSTGAVTSGYYPNWVQQKPGQAPRGLIGATDMRPSGTPA |
| | of A2J | | RFSGSLLGGKAALTLSGVQPEDEAEYYCALWYSNRWVFGGGTKLTVL |
| 512 | VL variant | ARTIFICIAL | ELVVTQEPSLTVSPGGTVTLTCGSSTGAVTSGNYPNWVQQKPGQAPRGLIGGTKFLAPGTPA |
| | of F12Q | | RFSGSLLGGKAALTLSGVQPEDEAEYYCVLWYSNRWVFGGGTKLTVL |
| 513 | VH of F6A | ARTIFICIAL | EVQLVESGGGLVQPGGSLKLSCAASGFTFNIYAMNWVRQAPGKGLEWVARIRSKYNNYATYY |
| | | | ADSVKSRFTISRDDSKNTAYLQMNNLKTEDTAVYYCVRHGNFGNSYVSFFAYWGQGTLVTVS |
| | | | S |
| 514 | VH of H2C | ARTIFICIAL | EVQLVESGGGLVQPGGSLKLSCAASGFTFNKYAMNWVRQAPGKGLEWVARIRSKYNNYATYY |
| | | | ADSVKDRFTISRDDSKNTAYLQMNNLKTEDTAVYYCVRHGNFGNSYISYWAYWGQGTLVTVS |
| | | | S |
| 515 | VH of H1E | ARTIFICIAL | EVQLVESGGGLEQPGGSLKLSCAASGFTFNSYAMNWVRQAPGKGLEWVARIRSKYNNYATYY |
| | | | ADSVKGRFTISRDDSKNTAYLQMNNLKTEDTAVYYCVRHGNFGNSYLSFWAYWGQGTLVTVS |
| | | | S |
| 516 | VH of G4H | ARTIFICIAL | EVQLVESGGGLVQPGGSLKLSCAASGFTFNRYAMNWVRQAPGKGLEWVARIRSKYNNYATYY |
| | | | ADSVKGRFTISRDDSKNTAYLQMNNLKTEDTAVYYCVRHGNFGNSYLSYFAYWGQGTLVTVS |
| | | | S |
| 517 | VH of A2J | ARTIFICIAL | EVQLVESGGGLVQPGGSLKLSCAASGFTFNVYAMNWVRQAPGKGLEWVARIRSKYNNYATYY |
| | | | ADSVKKRFTISRDDSKNTAYLQMNNLKTEDTAVYYCVRHGNFGNSYLSWWAYWGQGTLVTVS |
| | | | S |
| 518 | VH of E1L | ARTIFICIAL | EVQLVESGGGLVQPGGSLKLSCAASGFTFNKYAMNWVRQAPGKGLEWVARIRSKYNNYATYY |
| | | | ADSVKSRFTISRDDSKNTAYLQMNNLKTEDTAVYYCVRHGNFGNSYTSYYAYWGQGTLVTVS |
| | | | S |
| 519 | VH of E2M | ARTIFICIAL | EVQLVESGGGLVQPGGSLKLSCAASGFTFNGYAMNWVRQAPGKGLEWVARIRSKYNNYATYY |
| | | | ADSVKERFTISRDDSKNTAYLQMNNLKTEDTAVYYCVRHRNFGNSYLSWFAYWGQGTLVTVS |
| | | | S |
| 520 | VH of F70 | ARTIFICIAL | EVQLVESGGGLVQPGGSLKLSCAASGFTFNVYAMNWVRQAPGKGLEWVARIRSKYNNYATYY |
| | | | ADSVKKRFTISRDDSKNTAYLQMNNLKTEDTAVYYCVRHGNFGNSYISWWAYWGQGTLVTVS |
| | | | S |
| 521 | VH of F12Q | ARTIFICIAL | EVQLVESGGGLVQPGGSLKLSCAASGFTFNSYAMNWVRQAPGKGLEWVARIRSKYNNYATYY |
| | | | ADSVKGRFTISRDDSKNTAYLQMNNLKTEDTAVYYCVRHGNFGNSYVSWWAYWGQGTLVTVS |
| | | | S |
| 522 | VH of I2C | ARTIFICIAL | EVQLVESGGGLVQPGGSLKLSCAASGFTFNKYAMNWVRQAPGKGLEWVARIRSKYNNYATYY |
| | | | ADSVKDRFTISRDDSKNTAYLQMNNLKTEDTAVYYCVRHGNFGNSYISYWAYWGQGTLVTVS |
| | | | S |
| 523 | VH of F12q | ARTIFICIAL | EVQLVESGGGLVQPGGSLRLSCAASGFTFNSYAMNWVRQAPGKGLEWVARIRSKYNNYATYY |
| | | | ADSVKGRFTISRDDSKNTAYLQMNSLKTEDTAVYYCVRHGNFGNSYVSWWAYWGQGTLVTVS |
| | | | S |
| 524 | VH variant | ARTIFICIAL | EVQLLESGGGLVQPGGSLKLSCAASGFTFNIYAMNWVRQAPGKGLEWVARIRSKYNNYATYY |
| | of F6A | | ADSVKSRFTISRDDSKNTAYLQMNNLKTEDTAVYYCVRHGNFGNSYVSFFAYWGQGTLVTVS |
| | | | S |
| 525 | VH variant | ARTIFICIAL | EVQLLESGGGLVQPGGSLKLSCAASGFTFNKYAMNWVRQAPGKGLEWVARIRSKYNNYATYY |
| | of H2C | | ADSVKDRFTISRDDSKNTAYLQMNNLKTEDTAVYYCVRHGNFGNSYISYWAYWGQGTLVTVS |
| | | | S |
| 526 | VH variant | ARTIFICIAL | EVQLLESGGGLEQPGGSLKLSCAASGFTFNSYAMNWVRQAPGKGLEWVARIRSKYNNYATYY |
| | of H1E | | ADSVKGRFTISRDDSKNTAYLQMNNLKTEDTAVYYCVRHGNFGNSYLSFWAYWGQGTLVTVS |
| | | | S |
| 527 | VH variant | ARTIFICIAL | EVQLLESGGGLVQPGGSLKLSCAASGFTFNRYAMNWVRQAPGKGLEWVARIRSKYNNYATYY |
| | of G4H | | ADSVKGRFTISRDDSKNTAYLQMNNLKTEDTAVYYCVRHGNFGNSYLSYFAYWGQGTLVTVS |
| | | | S |
| 528 | VH variant | ARTIFICIAL | EVQLLESGGGLVQPGGSLKLSCAASGFTFNVYAMNWVRQAPGKGLEWVARIRSKYNNYATYY |
| | of A2J | | ADSVKKRFTISRDDSKNTAYLQMNNLKTEDTAVYYCVRHGNFGNSYLSWWAYWGQGTLVTVS |
| | | | S |
| 529 | VH variant | ARTIFICIAL | EVQLLESGGGLVQPGGSLKLSCAASGFTFNKYAMNWVRQAPGKGLEWVARIRSKYNNYATYY |
| | of E1L | | ADSVKSRFTISRDDSKNTAYLQMNNLKTEDTAVYYCVRHGNFGNSYTSYYAYWGQGTLVTVS |
| | | | S |
| 530 | VH variant | ARTIFICIAL | EVQLLESGGGLVQPGGSLKLSCAASGFTFNGYAMNWVRQAPGKGLEWVARIRSKYNNYATYY |
| | of E2M | | ADSVKERFTISRDDSKNTAYLQMNNLKTEDTAVYYCVRHRNFGNSYLSWFAYWGQGTLVTVS |
| | | | S |
| 531 | VH variant | ARTIFICIAL | EVQLLESGGGLVQPGGSLKLSCAASGFTFNVYAMNWVRQAPGKGLEWVARIRSKYNNYATYY |
| | of F70 | | ADSVKKRFTISRDDSKNTAYLQMNNLKTEDTAVYYCVRHGNFGNSYISWWAYWGQGTLVTVS |
| | | | S |
| 532 | VH variant | ARTIFICIAL | EVQLLESGGGLVQPGGSLKLSCAASGFTFNSYAMNWVRQAPGKGLEWVARIRSKYNNYATYY |
| | of F12Q | | ADSVKGRFTISRDDSKNTAYLQMNNLKTEDTAVYYCVRHGNFGNSYVSWWAYWGQGTLVTVS |
| | | | S |
| 533 | VH variant | ARTIFICIAL | EVQLLESGGGLVQPGGSLKLSCAASGFTFNKYAMNWVRQAPGKGLEWVARIRSKYNNYATYY |
| | of I2C | | ADSVKDRFTISRDDSKNTAYLQMNNLKTEDTAVYYCVRHGNFGNSYISYWAYWGQGTLVTVS |
| | | | S |
| 534 | VL of F6A | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 535 | VL of H1E | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 536 | VL of G4H | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 537 | VL of A2J | ARTIFICIAL | QTVVTQEPSLTVSPGGTVTLTCRSSTGAVTSGYYPNWVQQKPGQAPRGLIGATDMRPSGTPA |
| | | | RFSGSLLGGKAALTLSGVQPEDEAEYYCALWYSNRWVFGGGTKLTVL |
| 538 | VL of E1L | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 539 | VL of F70 | ARTIFICIAL | QTVVTQEPSLTVSPGGTVTLTCGSSTGAVTSGYYPNWVQQKPGQAPRGLIGGTKFLAPGTPA |
| | | | RFSGSLLGGKAALTLSGVQPEDEAEYYCALWYSNRWVFGGGTKLTVL |
| 540 | VL of I2C | ARTIFICIAL | QTVVTQEPSLTVSPGGTVTLTCGSSTGAVTSGNYPNWVQQKPGQAPRGLIGGTKFLAPGTPA |
| | | | RFSGSLLGGKAALTLSGVQPEDEAEYYCVLWYSNRWVFGGGTKLTVL |
| 541 | VL of F12q | ARTIFICIAL | QTVVTQEPSLTVSPGGTVTLTCGSSTGAVTSGNYPNWVQQKPGQAPRGLIGGTKFLAPGTPA |
| | | | RFSGSLLGGKAALTLSGVQPEDEAEYYCVLWYSNRWVFGGGTKLTVL |
| 542 | ScFv of F6A | ARTIFICIAL | EVQLVESGGGLVQPGGSLKLSCAASGFTFNIYAMNWVRQAPGKGLEWVARIRSKYNNYATYY |
| | | | ADSVKSRFTISRDDSKNTAYLQMNNLKTEDTAVYYCVRHGNFGNSYVSFFAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 543 | ScFv of H2C | ARTIFICIAL | EVQLVESGGGLVQPGGSLKLSCAASGFTFNKYAMNWVRQAPGKGLEWVARIRSKYNNYATYY |
| | | | ADSVKDRFTISRDDSKNTAYLQMNNLKTEDTAVYYCVRHGNFGNSYISYWAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 544 | ScFv of H1E | ARTIFICIAL | EVQLVESGGGLEQPGGSLKLSCAASGFTFNSYAMNWVRQAPGKGLEWVARIRSKYNNYATYY |
| | | | ADSVKGRFTISRDDSKNTAYLQMNNLKTEDTAVYYCVRHGNFGNSYLSFWAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 545 | SCFv of G4H | ARTIFICIAL | EVQLVESGGGLVQPGGSLKLSCAASGFTFNRYAMNWVRQAPGKGLEWVARIRSKYNNYATYY |
| | | | ADSVKGRFTISRDDSKNTAYLQMNNLKTEDTAVYYCVRHGNFGNSYLSYFAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 546 | ScFv of A2J | ARTIFICIAL | EVQLVESGGGLVQPGGSLKLSCAASGFTFNVYAMNWVRQAPGKGLEWVARIRSKYNNYATYY |
| | | | ADSVKKRFTISRDDSKNTAYLQMNNLKTEDTAVYYCVRHGNFGNSYLSWWAYWGQGTLVTVS |
| | | | SGGGGSGGGGSGGGGSQTVVTQEPSLTVSPGGTVTLTCRSSTGAVTSGYYPNWVQQKPGQAP |
| | | | RGLIGATDMRPSGTPARFSGSLLGGKAALTLSGVQPEDEAEYYCALWYSNRWVFGGGTKLTV |
| | | | L |
| 547 | ScFv of E1L | ARTIFICIAL | EVQLVESGGGLVQPGGSLKLSCAASGFTFNKYAMNWVRQAPGKGLEWVARIRSKYNNYATYY |
| | | | ADSVKSRFTISRDDSKNTAYLQMNNLKTEDTAVYYCVRHGNFGNSYTSYYAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 548 | SCFv of E2M | ARTIFICIAL | EVQLVESGGGLVQPGGSLKLSCAASGFTFNGYAMNWVRQAPGKGLEWVARIRSKYNNYATYY |
| | | | ADSVKERFTISRDDSKNTAYLQMNNLKTEDTAVYYCVRHRNFGNSYLSWFAYWGQGTLVTVS |
| | | | SGGGGSGGGGSGGGGSQTVVTQEPSLTVSPGGTVTLTCRSSTGAVTSGYYPNWVQQKPGQAP |
| | | | RGLIGATDMRPSGTPARFSGSLLGGKAALTLSGVQPEDEAEYYCALWYSNRWVFGGGTKLTV |
| | | | L |
| 549 | ScFv of F70 | ARTIFICIAL | EVQLVESGGGLVQPGGSLKLSCAASGFTFNVYAMNWVRQAPGKGLEWVARIRSKYNNYATYY |
| | | | ADSVKKRFTISRDDSKNTAYLQMNNLKTEDTAVYYCVRHGNFGNSYISWWAYWGQGTLVTVS |
| | | | SGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 550 | ScFv of F12Q | ARTIFICIAL | EVQLVESGGGLVQPGGSLKLSCAASGFTFNSYAMNWVRQAPGKGLEWVARIRSKYNNYATYY |
| | | | ADSVKGRFTISRDDSKNTAYLQMNNLKTEDTAVYYCVRHGNFGNSYVSWWAYWGQGTLVTVS |
| | | | SGGGGSGGGGSGGGGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTV |
| | | | I |
| 551 | ScFv of I2C | ARTIFICIAL | EVQLVESGGGLVQPGGSLKLSCAASGFTFNKYAMNWVRQAPGKGLEWVARIRSKYNNYATYY |
| | | | ADSVKDRFTISRDDSKNTAYLQMNNLKTEDTAVYYCVRHGNFGNSYISYWAYWGQGTLVTVS |
| | | | SGGGGSGGGGSGGGGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTV |
| | | | L |
| 552 | ScFv of F12q | ARTIFICIAL | EVQLVESGGGLVQPGGSLRLSCAASGFTFNSYAMNWVRQAPGKGLEWVARIRSKYNNYATYY |
| | | | ADSVKGRFTISRDDSKNTAYLQMNSLKTEDTAVYYCVRHGNFGNSYVSWWAYWGQGTLVTVS |
| | | | SGGGGSGGGGSGGGGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTV |
| | | | L |
| 553 | scFv variant | ARTIFICIAL | EVQLLESGGGLVQPGGSLKLSCAASGFTFNIYAMNWVRQAPGKGLEWVARIRSKYNNYATYY |
| | of F6A | | ADSVKSRFTISRDDSKNTAYLQMNNLKTEDTAVYYCVRHGNFGNSYVSFFAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 554 | scFv variant | ARTIFICIAL | EVQLLESGGGLVQPGGSLKLSCAASGFTFNKYAMNWVRQAPGKGLEWVARIRSKYNNYATYY |
| | of H2C | | ADSVKDRFTISRDDSKNTAYLQMNNLKTEDTAVYYCVRHGNFGNSYISYWAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 555 | scFv variant | ARTIFICIAL | EVQLLESGGGLEQPGGSLKLSCAASGFTFNSYAMNWVRQAPGKGLEWVARIRSKYNNYATYY |
| | of H1E | | ADSVKGRFTISRDDSKNTAYLQMNNLKTEDTAVYYCVRHGNFGNSYLSFWAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 556 | scFv variant | ARTIFICIAL | EVQLLESGGGLVQPGGSLKLSCAASGFTFNRYAMNWVRQAPGKGLEWVARIRSKYNNYATYY |
| | of G4H | | ADSVKGRFTISRDDSKNTAYLQMNNLKTEDTAVYYCVRHGNFGNSYLSYFAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 557 | scFv variant | ARTIFICIAL | EVQLLESGGGLVQPGGSLKLSCAASGFTFNVYAMNWVRQAPGKGLEWVARIRSKYNNYATYY |
| | of A2J | | ADSVKKRFTISRDDSKNTAYLQMNNLKTEDTAVYYCVRHGNFGNSYLSWWAYWGQGTLVTVS |
| | | | SGGGGSGGGGSGGGGSELVVTQEPSLTVSPGGTVTLTCRSSTGAVTSGYYPNWVQQKPGQAP |
| | | | RGLIGATDMRPSGTPARFSGSLLGGKAALTLSGVQPEDEAEYYCALWYSNRWVFGGGTKLTV |
| | | | L |
| 558 | scFv variant | ARTIFICIAL | EVQLLESGGGLVQPGGSLKLSCAASGFTFNKYAMNWVRQAPGKGLEWVARIRSKYNNYATYY |
| | of E1L | | ADSVKSRFTISRDDSKNTAYLQMNNLKTEDTAVYYCVRHGNFGNSYTSYYAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | I |
| 559 | scFv variant | ARTIFICIAL | EVQLLESGGGLVQPGGSLKLSCAASGFTFNGYAMNWVRQAPGKGLEWVARIRSKYNNYATYY |
| | of E2M | | ADSVKERFTISRDDSKNTAYLQMNNLKTEDTAVYYCVRHRNFGNSYLSWFAYWGQGTLVTVS |
| | | | SGGGGSGGGGSGGGGSELVVTQEPSLTVSPGGTVTLTCRSSTGAVTSGYYPNWVQQKPGQAP |
| | | | RGLIGATDMRPSGTPARFSGSLLGGKAALTLSGVQPEDEAEYYCALWYSNRWVFGGGTKLTV |
| | | | L |
| 560 | scFv variant | ARTIFICIAL | EVQLLESGGGLVQPGGSLKLSCAASGFTFNVYAMNWVRQAPGKGLEWVARIRSKYNNYATYY |
| | of F70 | | ADSVKKRFTISRDDSKNTAYLQMNNLKTEDTAVYYCVRHGNFGNSYISWWAYWGQGTLVTVS |
| | | | SGGGGSGGGGSGGGGSELVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTV |
| | | | L |
| 561 | scFv variant | ARTIFICIAL | EVQLLESGGGLVQPGGSLKLSCAASGFTFNSYAMNWVRQAPGKGLEWVARIRSKYNNYATYY |
| | of F12Q | | ADSVKGRFTISRDDSKNTAYLQMNNLKTEDTAVYYCVRHGNFGNSYVSWWAYWGQGTLVTVS |
| | | | SGGGGSGGGGSGGGGSELVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTV |
| | | | L |
| 562 | scFv variant | ARTIFICIAL | EVQLLESGGGLVQPGGSLKLSCAASGFTFNKYAMNWVRQAPGKGLEWVARIRSKYNNYATYY |
| | of I2C | | ADSVKDRFTISRDDSKNTAYLQMNNLKTEDTAVYYCVRHGNFGNSYISYWAYWGQGTLVTVS |
| | | | SGGGGSGGGGSGGGGSELVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTV |
| | | | L |
| 563 | linker 2 | ARTIFICIAL | GGGGS |
| 564 | linker 3 | ARTIFICIAL | GGGGQ |
| 565 | linker 4 | ARTIFICIAL | SGGGGS |
| 566 | linker 5 | ARTIFICIAL | PGGGGS |
| 567 | linker 6 | ARTIFICIAL | PGGDGS |
| 568 | linker 7 | ARTIFICIAL | GGGGSGGGS |
| 569 | linker 8 = | ARTIFICIAL | GGGGSGGGGS |
| | (G4S) 2 | | |
| | linker | | |
| 570 | linker 9 = | ARTIFICIAL | GGGGSGGGGSGGGGS |
| | (G4S) 3 | | |
| | linker | | |
| 571 | (G4S) 4 | ARTIFICIAL | GGGGSGGGGSGGGGSGGGGS |
| | linker | | |
| 572 | (G4S) 5 | ARTIFICIAL | GGGGSGGGGSGGGGSGGGGSGGGGS |
| | linker | | |
| 573 | (G4S) 6 | ARTIFICIAL | GGGGSGGGGSGGGGSGGGGSGGGGSGGGGS |
| | linker | | |
| 574 | (G4S) 7 | ARTIFICIAL | GGGGSGGGGSGGGGSGSGGGGSGGGGSGGGGS |
| | linker | | |
| 575 | (G4S) 8 | ARTIFICIAL | GGGGSGGGGSGGGGSGGGGSGGGGSGGGGSGGGGSGGGGS |
| | linker | | |
| 576 | linear FcRn | ARTIFICIAL | QRFVTGHFGGLXPANG |
| | BP | | |
| 577 | linear FcRn | ARTIFICIAL | QRFVTGHFGGLYPANG |
| | BP-Y | | |
| 578 | linear FcRn | ARTIFICIAL | QRFVTGHFGGLHPANG |
| | BP-H | | |
| 579 | core FcRn | ARTIFICIAL | TGHFGGLHP |
| | BP-H | | |
| 580 | cyclic FcRn | ARTIFICIAL | QRFCTGHFGGLHPCNG |
| | BP-H | | |
| 581 | HALB | ARTIFICIAL | DAHKSEVAHRFKDLGEENFKALVLIAFAQYLQQCPFEDHVKLVNEVTEFAKTCVADESAENC |
| | | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGKKLVAASQAALGL |
| 582 | HALB | ARTIFICIAL | DAHKSEVAHRFKDLGEENFKALVLIAFAQYLQQCPFEDHVKLVNEVTEFAKTCVADESAENC |
| | variant 1 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAGTFTFHADICTLSEKERQIKKQTALVELVKHKPKATKEQLKAAMDDFAAFVEKC |
| | | | CKADDKETCFAEEGKKLVAASQAALGL |
| 583 | HALB | ARTIFICIAL | DAHKSEVAHRFKDLGEENFKALVLIAFAQYLQQCPFEDHVKLVNEVTEFAKTCVADESAENC |
| | variant 2 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPKLVAASQAALGL |
| 584 | HALB | ARTIFICIAL | DAHKSEVAHRFKDLGEENFKALVLIAFAQYLQQCPFEDHVKLVNEVTEFAKTCVADESAENC |
| | variant 3 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 585 | HALB | ARTIFICIAL | DAHKSEVAHRFKDLGEENFKALVLIAFAQYLQQCPFEDHVKLVNEVTEFAKTCVADESAENC |
| | variant 4 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALGVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 586 | HALB | ARTIFICIAL | DAHKSEVAHRFKDLGEENFKALVLIAFAQYLQQCPFEDHVKLVNEVTEFAKTCVADESAENC |
| | variant 5 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 587 | HALB | ARTIFICIAL | DAHKSEVAHRFKDLGEENFKALVLIAFAQYLQQCPFEDHVKLVNEVTEFAKTCVADESAENC |
| | variant 6 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPHLVAASQAALGL |
| 588 | HALB | ARTIFICIAL | DAHKSEVAHRFKDLGEENFKALVLIAFAQYLQQCPFEDHVKLVNEVTEFAKTCVADESAENC |
| | variant 7 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 589 | HALB | ARTIFICIAL | DAHKSEVAHRFKDLGEENFKALVLIAFAQYLQQCPFEDHVKLVNEVTEFAKTCVADESAENC |
| | variant 8 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPKLVAASKAALGL |
| 590 | HALB | ARTIFICIAL | DAHKSEVAHRFKDLGEENFKALVLIAFAQYLQQCPFEDHVKLVNEVTEFAKTCVADESAENC |
| | variant 9 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPKLVAASKAALGL |
| 591 | HALB | ARTIFICIAL | DAHKSEVAHRFKDLGEENFKALVLIAFAQYLQQSPFEDHVKLVNEVTEFAKTCVADESAENC |
| | variant 10 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGKKLVAASQAALGL |
| 592 | HALB | ARTIFICIAL | DAHKSEVAHRFKDLGEENFKALVLIAFAQYLQQSPFEDHVKLVNEVTEFAKTCVADESAENC |
| | variant 11 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAGTFTFHADICTLSEKERQIKKQTALVELVKHKPKATKEQLKAAMDDFAAFVEKC |
| | | | CKADDKETCFAEEGKKLVAASQAALGL |
| 593 | HALB | ARTIFICIAL | DAHKSEVAHRFKDLGEENFKALVLIAFAQYLQQSPFEDHVKLVNEVTEFAKTCVADESAENC |
| | variant 12 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPKLVAASQAALGL |
| 594 | HALB | ARTIFICIAL | DAHKSEVAHRFKDLGEENFKALVLIAFAQYLQQSPFEDHVKLVNEVTEFAKTCVADESAENC |
| | variant 13 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 595 | HALB | ARTIFICIAL | DAHKSEVAHRFKDLGEENFKALVLIAFAQYLQQSPFEDHVKLVNEVTEFAKTCVADESAENC |
| | variant 14 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALGVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 596 | HALB | ARTIFICIAL | DAHKSEVAHRFKDLGEENFKALVLIAFAQYLQQSPFEDHVKLVNEVTEFAKTCVADESAENC |
| | variant 15 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 597 | HALB | ARTIFICIAL | DAHKSEVAHRFKDLGEENFKALVLIAFAQYLQQSPFEDHVKLVNEVTEFAKTCVADESAENC |
| | variant 16 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPHLVAASQAALGL |
| 598 | HALB | ARTIFICIAL | DAHKSEVAHRFKDLGEENFKALVLIAFAQYLQQSPFEDHVKLVNEVTEFAKTCVADESAENC |
| | variant 17 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 599 | HALB | ARTIFICIAL | DAHKSEVAHRFKDLGEENFKALVLIAFAQYLQQSPFEDHVKLVNEVTEFAKTCVADESAENC |
| | variant 18 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPKLVAASKAALGL |
| 600 | HALB | ARTIFICIAL | DAHKSEVAHRFKDLGEENFKALVLIAFAQYLQQSPFEDHVKLVNEVTEFAKTCVADESAENC |
| | variant 19 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPKLVAASKAALGL |
| 601 | HALB | ARTIFICIAL | DAHKSEVAHRFKDLGEENFKALVLIAFAQYLQQAPFEDHVKLVNEVTEFAKTCVADESAENC |
| | variant 20 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGKKLVAASQAALGL |
| 602 | HALB | ARTIFICIAL | DAHKSEVAHRFKDLGEENFKALVLIAFAQYLQQAPFEDHVKLVNEVTEFAKTCVADESAENC |
| | variant 21 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAGTFTFHADICTLSEKERQIKKQTALVELVKHKPKATKEQLKAAMDDFAAFVEKC |
| | | | CKADDKETCFAEEGKKLVAASQAALGL |
| 603 | HALB | ARTIFICIAL | DAHKSEVAHRFKDLGEENFKALVLIAFAQYLQQAPFEDHVKLVNEVTEFAKTCVADESAENC |
| | variant 22 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPKLVAASQAALGL |
| 604 | HALB | ARTIFICIAL | DAHKSEVAHRFKDLGEENFKALVLIAFAQYLQQAPFEDHVKLVNEVTEFAKTCVADESAENC |
| | variant 23 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 605 | HALB | ARTIFICIAL | DAHKSEVAHRFKDLGEENFKALVLIAFAQYLQQAPFEDHVKLVNEVTEFAKTCVADESAENC |
| | variant 24 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALGVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 606 | HALB | ARTIFICIAL | DAHKSEVAHRFKDLGEENFKALVLIAFAQYLQQAPFEDHVKLVNEVTEFAKTCVADESAENC |
| | variant 25 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDKFAAFVEKC |
| | | | CKADDKETCFAEEGPKLVAASQAALGL |
| 607 | HALB | ARTIFICIAL | DAHKSEVAHRFKDLGEENFKALVLIAFAQYLQQAPFEDHVKLVNEVTEFAKTCVADESAENC |
| | variant 26 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPHLVAASQAALGL |
| 608 | HALB | ARTIFICIAL | DAHKSEVAHRFKDLGEENFKALVLIAFAQYLQQAPFEDHVKLVNEVTEFAKTCVADESAENC |
| | variant 27 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDDFAAFVEKC |
| | | | CKADDKETCFAEEGPHLVAASKAALGL |
| 609 | HALB | ARTIFICIAL | DAHKSEVAHRFKDLGEENFKALVLIAFAQYLQQAPFEDHVKLVNEVTEFAKTCVADESAENC |
| | variant 28 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALEVDET |
| | | | YVPKEFNAETFTFHADICTLSEKERQIKKQTALVELVKHKPKATKEQLKAVMDKFAAFVEKC |
| | | | CKADDKETCFAEEGPKLVAASKAALGL |
| 610 | HALB | ARTIFICIAL | DAHKSEVAHRFKDLGEENFKALVLIAFAQYLQQAPFEDHVKLVNEVTEFAKTCVADESAENC |
| | variant 29 | | DKSLHTLFGDKLCTVATLRETYGEMADCCAKQEPERNECFLQHKDDNPNLPRLVRPEVDVMC |
| | | | TAFHDNEETFLKKYLYEIARRHPYFYAPELLFFAKRYKAAFTECCQAADKAACLLPKLDELR |
| | | | DEGKASSAKQRLKCASLQKFGERAFKAWAVARLSQRFPKAEFAEVSKLVTDLTKVHTECCHG |
| | | | DLLECADDRADLAKYICENQDSISSKLKECCEKPLLEKSHCIAEVENDEMPADLPSLAADFV |
| | | | ESKDVCKNYAEAKDVFLGMFLYEYARRHPDYSVVLLLRLAKTYETTLEKCCAAADPHECYAK |
| | | | VFDEFKPLVEEPQNLIKQNCELFEQLGEYKFQNALLVRYTKKVPQVSTPTLVEVSRNLGKVG |
| | | | SKCCKHPEAKRMPCAEDYLSVVLNQLCVLHEKTPVSDRVTKCCTESLVNRRPCFSALDVDET |
| | | | YVPKEFNAETFTFHADICTLSEKERQIKKQTALVELVKHKPKATKEQLKAVMDDFAAFVEKC |
| | | | CKADDKETCFAEEGPKLVAASKAALGL |
| 611 | Cross body 1 | ARTIFICIAL | ASTKGPSVFPLAPSSKSTSGGTAALGCLVKDYFPEPVTVSWNSGALTSGVHTFPAVLQSSGL |
| | HC | | YSLSSVVTVPSSSLGTQTYICNVNHKPSNTKVDKKVEPKSCDKTHTCPPCPAPELLGGPSVF |
| | | | LFPPKPKDTLMISRTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLPPSREEMTKNQVSL |
| | | | TCLVKGFYPSDIAVEWESNGQPENNYDTTPPVLDSDGSFFLYSDLTVDKSRWQQGNVFSCSV |
| | | | MHEALHNHYTQKSLSLSPGK |
| 612 | Cross body 1 | ARTIFICIAL | GQPKAAPSVTLFPPSSEELQANKATLVCLISDFYPGAVTVAWKADSSPVKAGVETTTPSKQS |
| | LC | | NNKYAASSYLSLTPEQWKSHRSYSCQVTHEGSTVEKTVAPTECSDKTHTCPPCPAPELLGGP |
| | | | SVFLFPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSRKEMTKNQ |
| | | | VSLTCLVKGFYPSDIAVEWESNGQPENNYKTTPPVLKSDGSFFLYSKLTVDKSRWQQGNVFS |
| | | | CSVMHEALHNHYTQKSLSLSPGK |
| 613 | Cross body 2 | ARTIFICIAL | ASTKGPSVFPLAPCSRSTSESTAALGCLVKDYFPEPVTVSWNSGALTSGVHTFPAVLQSSGL |
| | HC | | YSLSSVVTVPSSNFGTQTYTCNVDHKPSNTKVDKTVEPKSSDKTHTCPPCPAPEAAGGPSVF |
| | | | LFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAKGQPREPQVYTLPPSREEMTKNQVSL |
| | | | TCLVKGFYPSDIAVEWESNGQPENNYDTTPPVLDSDGSFFLYSDLTVDKSRWQQGNVFSCSV |
| | | | MHEALHNHYTQKSLSLSPGK |
| 614 | Cross body 2 | ARTIFICIAL | GQPKAAPSVTLFPPSSEELQANKATLVCLISDFYPGAVTVAWKADSSPVKAGVETTTPSKQS |
| | LC | | NNKYAASSYLSLTPEQWKSHRSYSCQVTHEGSTVEKTVAPTECSEPKSSDKTHTCPPCPAPE |
| | | | AAGGPSVFLFPPKPKDTLMISRTPEVTCVVVDVSHEDPEVKFNWYVDGVEVHNAKTKPREEQ |
| | | | YNSTYRVVSVLTVLHQDWLNGKEYKCKVSNKALPAPIEKTISKAKGQPREPQVYTLPPSRKE |
| | | | MTKNQVSLTCLVKGFYPSDIAVEWESNGQPENNYKTTPPVLKSDGSFFLYSKLTVDKSRWQQ |
| | | | GNVFSCSVMHEALHNHYTQKSLSLSPGK |
| 615 | Hetero-Fc | ARTIFICIAL | DKTHTCPPCPAPELLGGPSVFLFPPKPKDTLMISRTPEVTCVVVDVSHEDPEVKFNWYVDGV |
| | binder Fc | | EVHNAKTKPCEEQYGSTYRCVSVLTVLHQDWLNGKEYKCKVSNKALPAPIEKTISKAKGQPR |
| | | | EPQVYTLPPSRKEMTKNQVSLTCLVKGFYPSDIAVEWESNGQPENNYKTTPPVLKSDGSFFL |
| | | | YSKLTVDKSRWQQGNVFSCSVMHEALHNHYTQKSLSLSPGK |
| 616 | Hetero-Fc | ARTIFICIAL | DKTHTCPPCPAPELLGGPSVFLFPPKPKDTLMISRTPEVTCVVVDVSHEDPEVKFNWYVDGV |
| | partner Fc | | EVHNAKTKPCEEQYGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYDTTPPVLDSDGSFFL |
| | | | YSDLTVDKSRWQQGNVFSCSVMHEALHNHYTQKSLSLSPGK |
| 617 | Maxi-body 1 | ARTIFICIAL | EPKSSDKTHTCPPCPAPELLGGPSVFLFPPKPKDTLMISRTPEVTCVVVDVSHEDPEVKENW |
| | target Fc | | YVDGVEVHNAKTKPCEEQYGSTYRCVSVLTVLHQDWLNGKEYKCKVSNKALPAPIEKTISKA |
| | | | KGQPREPQVYTLPPSRKEMTKNQVSLTCLVKGFYPSDIAVEWESNGQPENNYKTTPPVLKSD |
| | | | GSFFLYSKLTVDKSRWQQGNVFSCSVMHEALHNHYTQKSLSLSPGK |
| 618 | Maxi-body 1 | ARTIFICIAL | EPKSSDKTHTCPPCPAPELLGGPSVFLFPPKPKDTLMISRTPEVTCVVVDVSHEDPEVKENW |
| | CD3 Fc | | YVDGVEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYDTTPPVLDSD |
| | | | GSFFLYSDLTVDKSRWQQGNVFSCSVMHEALHNHYTQKSLSLSPGK |
| 619 | Maxi-body 2 | ARTIFICIAL | EPKSSDKTHTCPPCPAPEAAGGPSVFLFPPKPKDTLMISRTPEVTCVVVDVSHEDPEVKENW |
| | target Fc | | YVDGVEVHNAKTKPREEQYNSTYRVVSVLTVLHQDWLNGKEYKCKVSNKALPAPIEKTISKA |
| | | | KGQPREPQVYTLPPSRKEMTKNQVSLTCLVKGFYPSDIAVEWESNGQPENNYKTTPPVLKSD |
| | | | GSFFLYSKLTVDKSRWQQGNVFSCSVMHEALHNHYTQKSLSLSPGK |
| 620 | Maxi-body 2 | ARTIFICIAL | EPKSSDKTHTCPPCPAPEAAGGPSVFLFPPKPKDTLMISRTPEVTCVVVDVSHEDPEVKENW |
| | CD3 Fc | | YVDGVEVHNAKTKPREEQYNSTYRVVSVLTVLHQDWLNGKEYKCKVSNKALPAPIEKTISKA |
| | | | KGQPREPQVYTLPPSREEMTKNQVSLTCLVKGFYPSDIAVEWESNGQPENNYDTTPPVLDSD |
| | | | GSFFLYSDLTVDKSRWQQGNVFSCSVMHEALHNHYTQKSLSLSPGK |
| 621 | Mono Fc | ARTIFICIAL | APELLGGPSVFLFPPKPKDTLMISRTPEVTCVVVDVSHEDPEVKFNWYVDGVEVHNAKTKPR |
| | | | EEQYNSTYRVVSVLTVLHQDWLNGKEYKCKVSNKALPAPIEKTISKAKGQPREPQVTTLPPS |
| | | | REEMTKNQVSLTCLVKGFYPSDIAVEWESNGQPENNYDTTPPVLDSDGSFFLYSDLTVDKSR |
| | | | WQQGNVFSCSVMHEALHNHYTQKSLSLSPGK |
| 622 | Fc monomer-1 | ARTIFICIAL | DKTHTCPPCPAPELLGGPSVFLFPPKPKDTLMISRTPEVTCVVVDVSHEDPEVKFNWYVDGV |
| | +c/−g | | EVHNAKTKPCEEQYGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 623 | Fc monomer-2 | ARTIFICIAL | DKTHTCPPCPAPELLGGPSVFLFPPKPKDTLMISRTPEVTCVVVDVSHEDPEVKFNWYVDGV |
| | +c/−g/delGK | | EVHNAKTKPCEEQYGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSP |
| 624 | Fc monomer-3 | ARTIFICIAL | DKTHTCPPCPAPELLGGPSVFLFPPKPKDTLMISRTPEVTCVVVDVSHEDPEVKFNWYVDGV |
| | −c/+g | | EVHNAKTKPREEQYNSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 625 | Fc monomer-4 | ARTIFICIAL | DKTHTCPPCPAPELLGGPSVFLFPPKPKDTLMISRTPEVTCVVVDVSHEDPEVKFNWYVDGV |
| | −c/+g/delGK | | EVHNAKTKPREEQYNSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSP |
| 626 | Fc monomer-5 | ARTIFICIAL | DKTHTCPPCPAPELLGGPSVFLFPPKPKDTLMISRTPEVTCVVVDVSHEDPEVKFNWYVDGV |
| | −c/−g | | EVHNAKTKPREEQYGSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 627 | Fc monomer-6 | ARTIFICIAL | DKTHTCPPCPAPELLGGPSVFLFPPKPKDTLMISRTPEVTCVVVDVSHEDPEVKFNWYVDGV |
| | −c/−g/delGK | | EVHNAKTKPREEQYGSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSP |
| 628 | Fc monomer-7 | ARTIFICIAL | DKTHTCPPCPAPELLGGPSVFLFPPKPKDTLMISRTPEVTCVVVDVSHEDPEVKFNWYVDGV |
| | +c/+g | | EVHNAKTKPCEEQYNSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 629 | Fc monomer-8 | ARTIFICIAL | DKTHTCPPCPAPELLGGPSVFLFPPKPKDTLMISRTPEVTCVVVDVSHEDPEVKFNWYVDGV |
| | +c/+g/delGK | | EVHNAKTKPCEEQYNSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSP |
| 630 | scFc-1 | ARTIFICIAL | DKTHTCPPCPAPELLGGPSVFLFPPKPKDTLMISRTPEVTCVVVDVSHEDPEVKFNWYVDGV |
| | | | EVHNAKTKPCEEQYGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 631 | scFc-2 | ARTIFICIAL | DKTHTCPPCPAPELLGGPSVFLFPPKPKDTLMISRTPEVTCVVVDVSHEDPEVKFNWYVDGV |
| | | | EVHNAKTKPCEEQYGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGSGGGGSGGGGSGGGGSGGG |
| | | | GSGGGGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIEKTIS |
| | | | KAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSP |
| 632 | ScFc-3 | ARTIFICIAL | DKTHTCPPCPAPELLGGPSVFLFPPKPKDTLMISRTPEVTCVVVDVSHEDPEVKFNWYVDGV |
| | | | EVHNAKTKPREEQYNSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHEDPEV |
| | | | KFNWYVDGVEVHNAKTKPREEQYNSTYRVVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 633 | ScFc-4 | ARTIFICIAL | DKTHTCPPCPAPELLGGPSVFLFPPKPKDTLMISRTPEVTCVVVDVSHEDPEVKFNWYVDGV |
| | | | EVHNAKTKPREEQYNSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGSGGGGSGGGGSGGGGSGGG |
| | | | GSGGGGSDKTHTCPPCPAPELLGGPSVFLFPPKPKDTLMISRTPEVTCVVVDVSHEDPEVKF |
| | | | NWYVDGVEVHNAKTKPREEQYNSTYRVVSVLTVLHQDWLNGKEYKCKVSNKALPAPIEKTIS |
| | | | KAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSP |
| 634 | ScFc-5 | ARTIFICIAL | DKTHTCPPCPAPELLGGPSVFLFPPKPKDTLMISRTPEVTCVVVDVSHEDPEVKFNWYVDGV |
| | | | EVHNAKTKPREEQYGSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHEDPEV |
| | | | KFNWYVDGVEVHNAKTKPREEQYGSTYRVVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 635 | ScFc-6 | ARTIFICIAL | DKTHTCPPCPAPELLGGPSVFLFPPKPKDTLMISRTPEVTCVVVDVSHEDPEVKFNWYVDGV |
| | | | EVHNAKTKPREEQYGSTYRVVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGSGGGGSGGGGSGGGGSGGG |
| | | | GSGGGGSDKTHTCPPCPAPELLGGPSVFLFPPKPKDTLMISRTPEVTCVVVDVSHEDPEVKF |
| | | | NWYVDGVEVHNAKTKPREEQYGSTYRVVSVLTVLHQDWLNGKEYKCKVSNKALPAPIEKTIS |
| | | | KAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSP |
| 636 | ScFc-7 | ARTIFICIAL | DKTHTCPPCPAPELLGGPSVFLFPPKPKDTLMISRTPEVTCVVVDVSHEDPEVKFNWYVDGV |
| | | | EVHNAKTKPCEEQYNSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHEDPEV |
| | | | KFNWYVDGVEVHNAKTKPCEEQYNSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 637 | scFc-8 | ARTIFICIAL | DKTHTCPPCPAPELLGGPSVFLFPPKPKDTLMISRTPEVTCVVVDVSHEDPEVKFNWYVDGV |
| | | | EVHNAKTKPCEEQYNSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGSGGGGSGGGGSGGGGSGGG |
| | | | GSGGGGSDKTHTCPPCPAPELLGGPSVFLFPPKPKDTLMISRTPEVTCVVVDVSHEDPEVKF |
| | | | NWYVDGVEVHNAKTKPCEEQYNSTYRCVSVLTVLHQDWLNGKEYKCKVSNKALPAPIEKTIS |
| | | | KAKGQPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSP |
| 638 | 5x his-tag | ARTIFICIAL | HHHHH |
| 639 | 6x his-tag | ARTIFICIAL | HHHHHH |
| 640 | CL-1 x I2C | ARTIFICIAL | QVQLVQSGAEVKKPGASVKVSCKASGYTFTGYYMHWVRQAPGQCLEWMGWINPNSGGTKYAQ |
| | | | KFQGRVTMTRDTSISTAYMELSRLRSDDTAVYYCARDRITVAGTYYYYGMDVWGQGTTVTVS |
| | | | SGGGGSGGGGSGGGGSDIQMTQSPSSVSASVGDRVTITCRASQGVNNWLAWYQQKPGKAPKL |
| | | | LIYTASSLQSGVPSRFSGSGSGTDFTLTIRSLQPEDFATYYCQQANSFPITFGCGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQGT |
| | | | LVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGG |
| | | | TKLTVL |
| 641 | CL-2 x I2C | ARTIFICIAL | QVQMVQSGAEVKKHGASVKVSCKASGYTFTGYYMHWVRQAPGQCLEWMGWINPNSGGTKYAQ |
| | | | KFQGRVTMTRDTSISTAYMELSRLRSDDTAVYYCARDRITVAGTYYYYGMDVWGQGTTVTVS |
| | | | SGGGGSGGGGSGGGGSDIQMTQSPSSVSASVGDRVTITCRASQGVNNWLAWYQQKPGKAPKL |
| | | | LIYTASSLQSGVPSRFSGSGSGTDFTLTIRSLQPEDFATYYCQQANSFPITFGCGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQGT |
| | | | LVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGG |
| | | | TKLTVL |
| 642 | CL-1 x I2C- | ARTIFICIAL | QVQLVQSGAEVKKPGASVKVSCKASGYTFTGYYMHWVRQAPGQCLEWMGWINPNSGGTKYAQ |
| | 6His | | KFQGRVTMTRDTSISTAYMELSRLRSDDTAVYYCARDRITVAGTYYYYGMDVWGQGTTVTVS |
| | | | SGGGGSGGGGSGGGGSDIQMTQSPSSVSASVGDRVTITCRASQGVNNWLAWYQQKPGKAPKL |
| | | | LIYTASSLQSGVPSRFSGSGSGTDFTLTIRSLQPEDFATYYCQQANSFPITFGCGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQGT |
| | | | LVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGG |
| | | | TKLTVLHHHHHH |
| 643 | CL-2 x I2C- | ARTIFICIAL | QVQMVQSGAEVKKHGASVKVSCKASGYTFTGYYMHWVRQAPGQCLEWMGWINPNSGGTKYAQ |
| | 6His | | KFQGRVTMTRDTSISTAYMELSRLRSDDTAVYYCARDRITVAGTYYYYGMDVWGQGTTVTVS |
| | | | SGGGGSGGGGSGGGGSDIQMTQSPSSVSASVGDRVTITCRASQGVNNWLAWYQQKPGKAPKL |
| | | | LIYTASSLQSGVPSRFSGSGSGTDFTLTIRSLQPEDFATYYCQQANSFPITFGCGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQGT |
| | | | LVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGG |
| | | | TKLTVLHHHHHH |
| 644 | AU1 epitope | ARTIFICIAL | DTYRYI |
| 645 | AU5 epitope | ARTIFICIAL | TDFYLK |
| 646 | T-7 tag | ARTIFICIAL | MASMTGGQQMG |
| 647 | V-5 tag | ARTIFICIAL | GKPIPNPLLGLDST |
| 648 | B-tag | ARTIFICIAL | QYPALT |
| 649 | E2 epitope | ARTIFICIAL | SSTSSDFRDR |
| 650 | FLAG tag | ARTIFICIAL | DYKDDDK |
| 651 | Glu−glu tag | ARTIFICIAL | EYMPME |
| | 1 | | |
| 652 | Glu−glu tag | ARTIFICIAL | EFMPME |
| | 2 | | |
| 653 | Histidine | ARTIFICIAL | KDHLIHNVHKEFHAHAHNK |
| | affinity tag | | |
| 654 | HSV epitope | ARTIFICIAL | QPELAPED |
| 655 | KT3 epitope | ARTIFICIAL | KPPTPPPEPET |
| 656 | Myc epitope | ARTIFICIAL | CEQKLISEEDL |
| 657 | 7x his-tag | ARTIFICIAL | HHHHHHH |
| 658 | 8x his-tag | ARTIFICIAL | HHHHHHHH |
| 659 | S1 tag | ARTIFICIAL | NANNPDWDF |
| 660 | S-tag | ARTIFICIAL | KETAAAKFERQHMDS |
| 661 | Strep-tag 1 | ARTIFICIAL | WSHPQFEK |
| 662 | Strep-tag 2 | ARTIFICIAL | AWAHPQPGG |
| 663 | Universal | ARTIFICIAL | HTTPHH |
| | tag | | |
| 664 | VSV−g | ARTIFICIAL | YTDIEMNRLGK |
| 665 | Protein C | ARTIFICIAL | EDQVDPRLIDGK |
| 666 | Ab156 | ARTIFICIAL | RDWDFDVFGGGTPVGG |
| 667 | H−cDR3 | ARTIFICIAL | X₁LIVX₂APX₃, WHEREIN X₁ IS EITHER A OR N; X₂ IS EITHER V OR E; |
| | | | AND X₃ IS EITHER V OR A, |
| 668 | H−cDR3 | ARTIFICIAL | DX₁LIVX₂APX₃T, WHEREIN X₁ IS EITHER A OR N; X₂ IS EITHER V OR E; |
| | | | AND X₃ IS EITHER V OR A, |
| 669 | H−cDR3 | ARTIFICIAL | DX₁LIVX₂APX₃TRDYYYYGMDV, WHEREIN X₁ IS EITHER A OR N; X₂ IS |
| | | | EITHER V OR E; AND X₃ IS EITHER V OR A |
| 670 | CD3 BINDER/ | ARTIFICIAL | KYAIN |
| | I2E - HCDR1 | | |
| 671 | CD3 BINDER/ | ARTIFICIAL | RIRSKYNNYATYYADAVKD |
| | I2E - HCDR2 | | |
| 672 | CD3 BINDER/ | ARTIFICIAL | AGNFGSSYISYWAY |
| | I2E - HCDR3 | | |
| 673 | CD3 BINDER/ | ARTIFICIAL | GSSTGAVTSGNYPN |
| | I2E - LCDR1 | | |
| 674 | CD3 BINDER | ARTIFICIAL | GTKFLAP |
| | /I2E - LCDR2 | | |
| 675 | CD3 BINDER | ARTIFICIAL | VLWYSNRWV |
| | I2E - LCDR3 | | |
| 676 | CD3 BINDER/ | ARTIFICIAL | EVQLVESGGGLVQPGGSLKLSCAASGFTFNKYAINWVRQAPGKGLEWVARIRSKYNNYATYY |
| | I2E - VH | | ADAVKDRFTISRDDSKNTVYLQMNNLKTEDTAVYYCARAGNFGSSYISYWAYWGQGTLVTVS |
| | | | S |
| 677 | CD3 BINDER/ | ARTIFICIAL | QTVVTQEPSLTVSPGGTVTITCGSSTGAVTSGNYPNWVQKKPGQAPRGLIGGTKFLAPGTPA |
| | I2E - VL | | RFSGSLSGGKAALTLSGVQPEDEAEYYCVLWYSNRWVFGSGTKLTVL |
| 678 | CD3 BINDER/ | ARTIFICIAL | EVQLVESGGGLVQPGGSLKLSCAASGFTFNKYAINWVRQAPGKGLEWVARIRSKYNNYATYY |
| | I2E-SCFV | | ADAVKDRFTISRDDSKNTVYLQMNNLKTEDTAVYYCARAGNFGSSYISYWAYWGQGTLVTVS |
| | (G4S)3 | | SGGGGSGGGGSGGGGSQTVVTQEPSLTVSPGGTVTITCGSSTGAVTSGNYPNWVQKKPGQAP |
| | | | RGLIGGTKFLAPGTPARFSGSLSGGKAALTLSGVQPEDEAEYYCVLWYSNRWVFGSGTKLTV |
| | | | L |
| 679 | G4-linker | ARTIFICIAL | GGGG |
| 680 | HCDR1 | ARTIFICIAL | GYSFTGYT |
| 681 | HCDR2 | ARTIFICIAL | INPYNGGT |
| 682 | HCDR2 | ARTIFICIAL | INPYNGGS |
| 683 | HCDR2 | ARTIFICIAL | INPYNGGI |
| 684 | HCDR3 | ARTIFICIAL | ARDYGYVLDY |
| 685 | HCDR3 | ARTIFICIAL | ARDFGYVLDY |
| 686 | HCDR3 | ARTIFICIAL | ARDYGFVLDY |
| 687 | HCDR3 | ARTIFICIAL | ARDYGYVFDY |
| 688 | LCDR1 | ARTIFICIAL | SSVSY |
| 689 | LCDR1 | ARTIFICIAL | SSVNY |
| 690 | LCDR2 | ARTIFICIAL | STS |
| 691 | LCDR3 | ARTIFICIAL | QQRSIYPPWT |
| 692 | LCDR3 | ARTIFICIAL | QQRSNYPPWT |
| 693 | LCDR3 | ARTIFICIAL | QQRSTYPPWT |
| 694 | LCDR3 | ARTIFICIAL | QQRNNYPPWT |
| 695 | LCDR1 | ARTIFICIAL | RASQSVX₁SX₂YLA, X₁ is selected from S and R, X₂ is selected from |
| | | | S and T |
| 696 | LCDR3 | ARTIFICIAL | QQYX₁X₂SPX₃T, X₁ is selected from G, D, and Q, X₂ is selected |
| | | | from S, A and T, X₃ is selected from L and I |

1. A polypeptide or a polypeptide construct comprising
a domain which binds to human CLDN6 (SEQ ID NO: 1), and
a domain which binds to human CD3, and
a domain extending the half-life of the polypeptide, wherein the domain, which binds to human CLDN6, binds to amino acids 29-39 of the amino acid sequence set forth in SEQ ID NO: 1 in the extracellular loop 1 (ECL1) of CLDN6 comprising the amino acid sequence set forth in SEQ ID NO: 9, and/or to amino acids 151-160 of the amino acid sequence set forth in SEQ ID NO: 1 in the extracellular loop 2 (ECL2) of CLDN6 comprising the amino acid sequence set forth in SEQ ID NO: 10,
wherein the domain which binds to human CLDN6 comprises a variable heavy chain (VH) region comprising a CDR-H1, a CDR-H2, and a CDR-H3 and a variable light chain (VL) region comprising a CDR-L1, a CDR-L2, and a CDR-L3 selected from the group consisting of:
a) a VH region comprising a CDR-H1 comprising the amino acid sequence set forth in SEQ ID NO: 13, a CDR-H2 comprising the amino acid sequence set forth in SEQ ID NO: 14, and a CDR-H3 comprising the amino acid sequence set forth in SEQ ID NO: 15, and a VL region comprising a CDR-L1 comprising the amino acid sequence set forth in SEQ ID NO: 16, a CDR-L2 comprising the amino acid sequence set forth in SEQ ID NO: 17 and a CDR-L3 comprising the amino acid sequence set forth in SEQ ID NO: 18;
b) a VH region comprising a CDR-H1 comprising the amino acid sequence set forth in SEQ ID NO: 27, a CDR-H2 comprising the amino acid sequence set forth in SEQ ID NO: 28, and a CDR-H3 comprising the amino acid sequence set forth in SEQ ID NO: 29, and a VL region comprising a CDR-L1 comprising the amino acid sequence set forth in SEQ ID NO: 30, a CDR-L2 comprising the amino acid sequence set forth in SEQ ID NO: 31 and a CDR-L3 comprising the amino acid sequence set forth in SEQ ID NO: 32;
c) a VH region comprising a CDR-H1 comprising the amino acid sequence set forth in SEQ ID NO: 41, a CDR-H2 comprising the amino acid sequence set forth in SEQ ID NO: 42, and a CDR-H3 comprising the amino acid sequence set forth in SEQ ID NO: 43, and a VL region comprising a CDR-L1 comprising the amino acid sequence set forth in SEQ ID NO: 44, a CDR-L2 comprising the amino acid sequence set forth in SEQ ID NO: 45 and a CDR-L3 comprising the amino acid sequence set forth in SEQ ID NO: 46;
d) a VH region comprising a CDR-H1 comprising the amino acid sequence set forth in SEQ ID NO: 55, a CDR-H2 comprising the amino acid sequence set forth in SEQ ID NO: 56, and a CDR-H3 comprising the amino acid sequence set forth in SEQ ID NO: 57, and a VL region comprising a CDR-L1 comprising the amino acid sequence set forth in SEQ ID NO: 58, a CDR-L2 comprising the amino acid sequence set forth in SEQ ID NO: 59 and a CDR-L3 comprising the amino acid sequence set forth in SEQ ID NO: 60;
e) a VH region comprising a CDR-H1 comprising the amino acid sequence set forth in SEQ ID NO: 69, a CDR-H2 comprising the amino acid sequence set forth in SEQ ID NO: 70, and a CDR-H3 comprising the amino acid sequence set forth in SEQ ID NO: 71, and a VL region comprising a CDR-L1 comprising the amino acid sequence set forth in SEQ ID NO: 72, a CDR-L2 comprising the amino acid sequence set forth in SEQ ID NO: 73 and a CDR-L3 comprising the amino acid sequence set forth in SEQ ID NO: 74;
f) a VH region comprising a CDR-H1 comprising the amino acid sequence set forth in SEQ ID NO: 83, a CDR-H2 comprising the amino acid sequence set forth in SEQ ID NO: 84, and a CDR-H3 comprising the amino acid sequence set forth in SEQ ID NO: 85, and a VL region comprising a CDR-L1 comprising the amino acid sequence set forth in SEQ ID NO: 86, a CDR-L2 comprising the amino acid sequence set forth in SEQ ID NO: 87 and a CDR-L3 comprising the amino acid sequence set forth in SEQ ID NO: 88;
q) a VH region comprising a CDR-H1 comprising the amino acid sequence set forth in SEQ ID NO: 97, a CDR-H2 comprising the amino acid sequence set forth in SEQ ID NO: 98, and a CDR-H3 comprising the amino acid sequence set forth in SEQ ID NO: 99, and a VL region comprising a CDR-L1 comprising the amino acid sequence set forth in SEQ ID NO: 100, a CDR-L2 comprising the amino acid sequence set forth in SEQ ID NO: 101 and a CDR-L3 comprising the amino acid sequence set forth in SEQ ID NO: 102;
h) a VH region comprising a CDR-H1 comprising the amino acid sequence set forth in SEQ ID NO: 111, a CDR-H2 comprising the amino acid sequence set forth in SEQ ID NO: 112, and a CDR-H3 comprising the amino acid sequence set forth in SEQ ID NO: 113, and a VL region comprising a CDR-L1 comprising the amino acid sequence set forth in SEQ ID NO: 114, a CDR-L2 comprising the amino acid sequence set forth in SEQ ID NO: 115 and a CDR-L3 comprising the amino acid sequence set forth in SEQ ID NO: 116;
i) a VH region comprising a CDR-H1 comprising the amino acid sequence set forth in SEQ ID NO: 125, a CDR-H2 comprising the amino acid sequence set forth in SEQ ID NO: 126, and a CDR-H3 comprising the amino acid sequence set forth in SEQ ID NO: 127, and a VL region comprising a CDR-L1 comprising the amino acid sequence set forth in SEQ ID NO: 128, a CDR-L2 comprising the amino acid sequence set forth in SEQ ID NO: 129 and a CDR-L3 comprising the amino acid sequence set forth in SEQ ID NO: 130;
j) a VH region comprising a CDR-H1 comprising the amino acid sequence set forth in SEQ ID NO: 139, a CDR-H2 comprising the amino acid sequence set forth in SEQ ID NO: 140, and a CDR-H3 comprising the amino acid sequence set forth in SEQ ID NO: 141, and a VL region comprising a CDR-L1 comprising the amino acid sequence set forth in SEQ ID NO: 142, a CDR-L2 comprising the amino acid sequence set forth in SEQ ID NO: 143 and a CDR-L3 comprising the amino acid sequence set forth in SEQ ID NO: 144;
k) a VH region comprising a CDR-H1 comprising the amino acid sequence set forth in SEQ ID NO: 153, a CDR-H2 comprising the amino acid sequence set forth in SEQ ID NO: 154, and a CDR-H3 comprising the amino acid sequence set forth in SEQ ID NO: 155, and a VL region comprising a CDR-L1 comprising the amino acid sequence set forth in SEQ ID NO: 156, a CDR-L2 comprising the amino acid sequence set forth in SEQ ID NO: 157 and a CDR-L3 comprising the amino acid sequence set forth in SEQ ID NO: 158;
l) a VH region comprising a CDR-H1 comprising the amino acid sequence set forth in SEQ ID NO: 167, a CDR-H2 comprising the amino acid sequence set forth in SEQ ID NO: 168, and a CDR-H3 comprising the amino acid sequence set forth in SEQ ID NO: 169, and a VL region comprising a CDR-L1 comprising the amino acid sequence set forth in SEQ ID NO: 170, a CDR-L2 comprising the amino acid sequence set forth in SEQ ID NO: 171 and a CDR-L3 comprising the amino acid sequence set forth in SEQ ID NO: 172;
m) a VH region comprising a CDR-H1 comprising the amino acid sequence set forth in SEQ ID NO: 181, a CDR-H2 comprising the amino acid sequence set forth in SEQ ID NO: 182, and a CDR-H3 comprising the amino acid sequence set forth in SEQ ID NO: 183, and a VL region comprising a CDR-L1 comprising the amino acid sequence set forth in SEQ ID NO: 184, a CDR-L2 comprising the amino acid sequence set forth in SEQ ID NO: 185 and a CDR-L3 comprising the amino acid sequence set forth in SEQ ID NO: 186;
n) a VH region comprising a CDR-H1 comprising the amino acid sequence set forth in SEQ ID NO: 195, a CDR-H2 comprising the amino acid sequence set forth in SEQ ID NO: 196, and a CDR-H3 comprising the amino acid sequence set forth in SEQ ID NO: 197, and a VL region comprising a CDR-L1 comprising the amino acid sequence set forth in SEQ ID NO: 198, a CDR-L2 comprising the amino acid sequence set forth in SEQ ID NO: 199 and a CDR-L3 comprising the amino acid sequence set forth in SEQ ID NO: 200;
o) a VH region comprising a CDR-H1 comprising the amino acid sequence set forth in SEQ ID NO: 209, a CDR-H2 comprising the amino acid sequence set forth in SEQ ID NO: 210, and a CDR-H3 comprising the amino acid sequence set forth in SEQ ID NO: 211, and a VL region comprising a CDR-L1 comprising the amino acid sequence set forth in SEQ ID NO: 212, a CDR-L2 comprising the amino acid sequence set forth in SEQ ID NO: 213 and a CDR-L3 comprising the amino acid sequence set forth in SEQ ID NO: 214;
p) a VH region comprising a CDR-H1 comprising the amino acid sequence set forth in SEQ ID NO: 223, a CDR-H2 comprising the amino acid sequence set forth in SEQ ID NO: 224, and a CDR-H3 comprising the amino acid sequence set forth in SEQ ID NO: 225, and a VL region comprising a CDR-L1 comprising the amino acid sequence set forth in SEQ ID NO: 226, a CDR-L2 comprising the amino acid sequence set forth in SEQ ID NO: 227 and a CDR-L3 comprising the amino acid sequence set forth in SEQ ID NO: 228;
q) a VH region comprising a CDR-H1 comprising the amino acid sequence set forth in SEQ ID NO: 237, a CDR-H2 comprising the amino acid sequence set forth in SEQ ID NO: 238, and a CDR-H3 comprising the amino acid sequence set forth in SEQ ID NO: 239, and a VL region comprising a CDR-L1 comprising the amino acid sequence set forth in SEQ ID NO: 240, a CDR-L2 comprising the amino acid sequence set forth in SEQ ID NO: 241 and a CDR-L3 comprising the amino acid sequence set forth in SEQ ID NO: 242;
r) a VH region comprising a CDR-H1 comprising the amino acid sequence set forth in SEQ ID NO: 251, a CDR-H2 comprising the amino acid sequence set forth in SEQ ID NO: 252, and a CDR-H3 comprising the amino acid sequence set forth in SEQ ID NO: 253, and a VL region comprising a CDR-L1 comprising the amino acid sequence set forth in SEQ ID NO: 254, a CDR-L2 comprising the amino acid sequence set forth in SEQ ID NO: 255 and a CDR-L3 as comprising the amino acid sequence set forth in SEQ ID NO: 256, and
s) a VH region comprising a CDR-H1 comprising the amino acid sequence set forth in SEQ ID NO: 265, a CDR-H2 comprising the amino acid sequence set forth in SEQ ID NO: 266, and a CDR-H3 comprising the amino acid sequence set forth in SEQ ID NO: 267, and a VL region comprising a CDR-L1 comprising the amino acid sequence set forth in SEQ ID NO: 268, a CDR-L2 comprising the amino acid sequence set forth in SEQ ID NO: 269, and a CDR-L3 as comprising the amino acid sequence set forth in SEQ ID NO: 270.

2-7. (canceled)

8. The polypeptide or polypeptide construct of claim 1, wherein
a) the polypeptide or polypeptide construct is a single chain construct,
b) the domain binding to CLDN6 is in the format of an scFv,
c) the domain binding to CD3 is in the format of an scFv,
d) the domains are connected via a linker, and/or
e) the polypeptide or polypeptide construct comprises a domain providing an extended serum half-life.

9-10. (canceled)

11. The polypeptide or polypeptide construct of claim 1, wherein the domain binding to CLDN6 comprises a VH region comprising the amino acid sequence set forth in SEQ ID NO: 11, SEQ ID NO: 25, SEQ ID NO: 39, SEQ ID NO: 53, SEQ ID NO: 67, SEQ ID NO: 81, SEQ ID NO: 95, SEQ ID NO: 109, SEQ ID NO: 123, SEQ ID NO: 137, SEQ ID NO: 151, SEQ ID NO: 165, SEQ ID NO: 179, SEQ ID NO: 193, SEQ ID NO: 207, SEQ ID NO: 221, SEQ ID NO: 235, SEQ ID NO: 249, or SEQ ID NO: 263,
wherein said VH region amino acid sequence may have one or more modifications of one or several amino acid residues in the framework and/or hypervariable regions, provided said domain comprising said modified VH region selectively binds to CLDN6, and
optionally wherein said domain is part of a polypeptide or polypeptide construct that activates T cells and maintains the ability to induce T cell-dependent cytotoxicity,
further, optionally, wherein said domain is part of a polypeptide or polypeptide construct that activates T cells and maintains the ability to induce T cell-dependent cytotoxicity with more than 1000 fold efficacy than in an identical cell type which expresses CLDN9, but does not express CLDN6, and
still further optionally, wherein said domain is part of a polypeptide or polypeptide construct that is not capable of activating T cells and inducing T cell-dependent cytotoxicity in CLDN6-negative cells of the same cell type, preferably when tested in an in vitro cytotoxicity assay.

12. The polypeptide or polypeptide construct of claim 1, wherein the domain binding to CLDN6 comprises a VL region comprising the amino acid sequence set forth in SEQ ID NO: 12, SEQ ID NO: 26, SEQ ID NO: 40, SEQ ID NO: 54, SEQ ID NO: 68, SEQ ID NO: 82, SEQ ID NO: 96, SEQ ID NO: 110, SEQ ID NO: 124, SEQ ID NO: 138, SEQ ID NO: 152, SEQ ID NO: 166, SEQ ID NO: 180, SEQ ID NO: 194, SEQ ID NO: 208, SEQ ID NO: 222, SEQ ID NO: 236, SEQ ID NO: 250, or SEQ ID NO: 264,
wherein said VL region amino acid sequence may have one or more modifications of one or several amino acid residues in the framework and/or hypervariable regions, provided said domain comprising said modified VL region selectively binds to CLDN6, and
optionally wherein said domain is part of a polypeptide or polypeptide construct that activates T cells and maintains the ability to induce T cell-dependent cytotoxicity in target cells,
further, optionally, wherein said domain is part of a polypeptide or polypeptide construct that activates T cells and maintains the ability to induce T cell-dependent cytotoxicity with more than 500 fold efficacy than in control cells which do not express CLDN6, wherein these cells optionally express CLDN9, but
still further optionally, wherein said domain is part of a polypeptide or polypeptide construct that is not capable of activating T cells and inducing T cell-dependent cytotoxicity in CLDN6-negative cells of the same cell type, preferably when tested in a in vitro cytotoxicity assay.

13. The polypeptide or polypeptide construct of claim 1, wherein the domain binding to CLDN6 comprises a pair of a VH region and a VL region comprising the amino acid sequences set forth in SEQ ID NOs: 11 and 12, SEQ ID NO: 25 and 26, SEQ ID NO: 39 and 40, SEQ ID NO: 53 and 54, SEQ ID NO: 67 and 68, SEQ ID NO: 81 and 82, SEQ ID NO: 95 and 96, SEQ ID NO: 109 and 110, SEQ ID NO: 123 and 124, SEQ ID NO: 137 and 138, SEQ ID NO: 151 and 152, SEQ ID NO: 165 and 166, SEQ ID NO: 179 and 180, SEQ ID NO: 193 and 194, SEQ ID NO: 207 and 208, SEQ ID NO: 221 and 222, SEQ ID NO: 235 and 236, SEQ ID NO: 249 and 250, or SEQ ID NO: 263 and 264.

14. The polypeptide or polypeptide construct of claim 1, wherein the domain binding to CLDN6 comprises the amino acid sequence set forth in SEQ ID NO: 19, SEQ ID NO: 22, SEQ ID NO: 33, SEQ ID NO: 36, SEQ ID NO: 47, SEQ ID NO: 50, SEQ ID NO: 61, SEQ ID NO: 64, SEQ ID NO: 75, SEQ ID NO: 78, SEQ ID NO: 89, SEQ ID NO: 92, SEQ ID NO: 103, SEQ ID NO: 106, SEQ ID NO: 117, SEQ ID NO: 120, SEQ ID NO: 131, SEQ ID NO: 134, SEQ ID NO: 145, SEQ ID NO: 148, SEQ ID NO: 159, SEQ ID NO: 162, SEQ ID NO: 173, SEQ ID NO: 176, SEQ ID NO: 187, SEQ ID NO: 190, SEQ ID NO: 201, SEQ ID NO: 204, SEQ ID NO: 215, SEQ ID NO: 218, SEQ ID NO: 229, SEQ ID NO: 232, SEQ ID NO: 243, SEQ ID NO: 246, SEQ ID NO: 257, or SEQ ID NO: 260, SEQ ID NO: 271 or SEQ ID NO: 274.

15. The polypeptide or polypeptide construct of claim 1, comprising:
a) an amino acid sequence comprising at least at least 90, 91, 92, 93, 94, 95, 96, 97, 98 or 99% identity to the amino acid sequence set forth in SEQ ID NO: 19, SEQ ID NO: 20, SEQ ID NO: 21, SEQ ID NO: 22, SEQ ID NO: 23, and SEQ ID NO: 24, SEQ ID NO: 33, SEQ ID NO: 34, SEQ ID NO: 35, SEQ ID NO: 36, SEQ ID NO: 37, and SEQ ID NO: 38, SEQ ID NO: 47, SEQ ID NO: 48, SEQ ID NO: 49, SEQ ID NO: 50, SEQ ID NO: 51, and SEQ ID NO: 52, SEQ ID NO: 61, SEQ ID NO: 62, SEQ ID NO: 63, SEQ ID NO: 64, SEQ ID NO: 65, and SEQ ID NO: 66, SEQ ID NO: 75, SEQ ID NO: 76, SEQ ID NO: 77, SEQ ID NO: 78, SEQ ID NO: 79, and SEQ ID NO: 80, SEQ ID NO: 89, SEQ ID NO: 90, SEQ ID NO: 91, SEQ ID NO: 92, SEQ ID NO: 93, and SEQ ID NO: 94, SEQ ID NO: 103, SEQ ID NO: 104, SEQ ID NO: 105, SEQ ID NO: 106, SEQ ID NO: 107, and SEQ ID NO: 108, SEQ ID NO: 117, SEQ ID NO: 118, SEQ ID NO: 119, SEQ ID NO: 120, SEQ ID NO: 121, and SEQ ID NO: 122, SEQ ID NO: 131, SEQ ID NO: 132, SEQ ID NO: 133, SEQ ID NO: 134, SEQ ID NO: 135, and SEQ ID NO: 136, SEQ ID NO: 145, SEQ ID NO: 146, SEQ ID NO: 147, SEQ ID NO: 148, SEQ ID NO: 149, and SEQ ID NO: 150, SEQ ID NO: 159, SEQ ID NO: 160, SEQ ID NO: 161, SEQ ID NO: 162, SEQ ID NO: 163, and SEQ ID NO: 164, SEQ ID NO: 173, SEQ ID NO: 174, SEQ ID NO: 175, SEQ ID NO: 176, SEQ ID NO: 177, and SEQ ID NO: 178, SEQ ID NO: 187, SEQ ID NO: 188, SEQ ID NO: 189, SEQ ID NO: 190, SEQ ID NO: 191, and SEQ ID NO: 192, SEQ ID NO: 201, SEQ ID NO: 202, SEQ ID NO: 203, SEQ ID NO: 204, SEQ ID NO: 205, and SEQ ID NO: 206, SEQ ID NO: 215, SEQ ID NO: 216, SEQ ID NO: 217, SEQ ID NO: 218, SEQ ID NO: 219, and SEQ ID NO: 220, SEQ ID NO: 229, SEQ ID NO: 230, SEQ ID NO: 231, SEQ ID NO: 232, SEQ ID NO: 233, and SEQ ID NO: 234, SEQ ID NO: 243, SEQ ID NO: 244, SEQ ID NO: 245, SEQ ID NO: 246, SEQ ID NO: 247, and SEQ ID NO: 248, SEQ ID NO: 257, SEQ ID NO: 258, SEQ ID NO: 259, SEQ ID NO: 260, SEQ ID NO: 261, and SEQ ID NO: 262, SEQ ID NO: 271, SEQ ID NO: 272, SEQ ID NO: 273, SEQ ID NO: 274, SEQ ID NO: 275, or SEQ ID NO: 276; and
b) the amino acid sequence set forth in SEQ ID NO: 19, SEQ ID NO: 20, SEQ ID NO: 21, SEQ ID NO: 22, SEQ ID NO: 23, and SEQ ID NO: 24, SEQ ID NO: 33, SEQ ID NO: 34, SEQ ID NO: 35, SEQ ID NO: 36, SEQ ID NO: 37, and SEQ ID NO: 38, SEQ ID NO: 47, SEQ ID NO: 48, SEQ ID NO: 49, SEQ ID NO: 50, SEQ ID NO: 51, and SEQ ID NO: 52, SEQ ID NO: 61, SEQ ID NO: 62, SEQ ID NO: 63, SEQ ID NO: 64, SEQ ID NO: 65, and SEQ ID NO: 66, SEQ ID NO: 75, SEQ ID NO: 76, SEQ ID NO: 77, SEQ ID NO: 78, SEQ ID NO: 79, and SEQ ID NO: 80, SEQ ID NO: 89, SEQ ID NO: 90, SEQ ID NO: 91, SEQ ID NO: 92, SEQ ID NO: 93, and SEQ ID NO: 94, SEQ ID NO: 103, SEQ ID NO: 104, SEQ ID NO: 105, SEQ ID NO: 106, SEQ ID NO: 107, and SEQ ID NO: 108, SEQ ID NO: 117, SEQ ID NO: 118, SEQ ID NO: 119, SEQ ID NO: 120, SEQ ID NO: 121, and SEQ ID NO: 122, SEQ ID NO: 131, SEQ ID NO: 132, SEQ ID NO: 133, SEQ ID NO: 134, SEQ ID NO: 135, and SEQ ID NO: 136, SEQ ID NO: 145, SEQ ID NO: 146, SEQ ID NO: 147, SEQ ID NO: 148, SEQ ID NO: 149, and SEQ ID NO: 150, SEQ ID NO: 159, SEQ ID NO: 160, SEQ ID NO: 161, SEQ ID NO: 162, SEQ ID NO: 163, and SEQ ID NO: 164, SEQ ID NO: 173, SEQ ID NO: 174, SEQ ID NO: 175, SEQ ID NO: 176, SEQ ID NO: 177, and SEQ ID NO: 178, SEQ ID NO: 187, SEQ ID NO: 188, SEQ ID NO: 189, SEQ ID NO: 190, SEQ ID NO: 191, and SEQ ID NO: 192, SEQ ID NO: 201, SEQ ID NO: 202, SEQ ID NO: 203, SEQ ID NO: 204, SEQ ID NO: 205, and SEQ ID NO: 206, SEQ ID NO: 215, SEQ ID NO: 216, SEQ ID NO: 217, SEQ ID NO: 218, SEQ ID NO: 219, and SEQ ID NO: 220, SEQ ID NO: 229, SEQ ID NO: 230, SEQ ID NO: 231, SEQ ID NO: 232, SEQ ID NO: 233, and SEQ ID NO: 234, SEQ ID NO: 243, SEQ ID NO: 244, SEQ ID NO: 245, SEQ ID NO: 246, SEQ ID NO: 247, and SEQ ID NO: 248, SEQ ID NO: 257, SEQ ID NO: 258, SEQ ID NO: 259, SEQ ID NO: 260, SEQ ID NO: 261, and SEQ ID NO: 262, SEQ ID NO: 271, SEQ ID NO: 272, SEQ ID NO: 273, SEQ ID NO: 274, SEQ ID NO: 275, or SEQ ID NO: 276.

16. A polypeptide or a polypeptide construct comprising a CDR-H1 comprising the amino acid sequence set forth in SEQ ID NO: 13, a CDR-H2 comprising the amino acid sequence set forth in SEQ ID NO: 14, and a CDR-H3 comprising the amino acid sequence set forth in SEQ ID NO: 15, and a VL region comprising a CDR-L1 comprising the amino acid sequence set forth in SEQ ID NO: 16, a CDR-L2 comprising the amino acid sequence set forth in SEQ ID NO: 17 and a CDR-L3 depicted comprising the amino acid sequence set forth in SEQ ID NO: 18.

17. A polypeptide or polypeptide construct comprising a VH region comprising the amino acid sequence set forth in SEQ ID NO: 11 and a VL region comprising the amino acid sequence set forth in SEQ ID NO: 12.

18. A polypeptide or polypeptide construct comprising the amino acid sequence set forth in SEQ ID NO: 19.

19. A polypeptide or polypeptide construct comprising the amino acid sequence set forth in SEQ ID NO: 19, SEQ ID NO: 20, SEQ ID NO: 21, SEQ ID NO: 22, SEQ ID NO: 23, or SEQ ID NO: 24.

20. A polypeptide or a polypeptide construct comprising a domain which binds to human CLDN6 (SEQ ID NO: 1), and a domain which binds to human CD3, and a domain extending the half-life of the polypeptide, wherein the domain that binds to CLDN6 comprises a variable heavy chain (VH) region comprising a CDR-H1 comprising the amino acid sequence set forth in SEQ ID NO: 13, a CDR-H2 comprising the amino acid sequence set forth in SEQ ID NO: 14, and a CDR-H3 comprising the amino acid sequence set forth in SEQ ID NO: 15, and a variable light chain (VL) region comprising a CDR-L1 comprising the amino acid sequence set forth in SEQ ID NO: 16, a CDR-L2 comprising the amino acid sequence set forth in SEQ ID NO: 17 and a CDR-L3 comprising the amino acid sequence set forth in SEQ ID NO: 18.

21. The polypeptide or polypeptide construct of claim 20, wherein the domain binding to CLDN6 comprises a VH region comprising the amino acid sequence set forth in SEQ ID NO: 11 and a VL region comprising the amino acid sequence set forth in SEQ ID NO: 12.

22. The polypeptide or polypeptide construct of claim 20, wherein the domain binding to CLDN6 comprises the amino acid sequence set forth in SEQ ID NO: 19.

23. The polypeptide or polypeptide construct of claim 20, comprising the amino acid sequence set forth in SEQ ID NO: 19, SEQ ID NO: 20, SEQ ID NO: 21, SEQ ID NO: 22, SEQ ID NO: 23, or SEQ ID NO: 24.

24-25. (canceled)

26. The polypeptide or polypeptide construct of claim 20, wherein the construct is a single chain construct.

27-32. (canceled)

33. The polypeptide or polypeptide construct of claim 20, wherein the polypeptide or polypeptide construct comprises any one of the amino acid sequences set forth in SEQ ID NO: 35, 38, 49, 52, 63, 66, 77, 80, 91, 94, 105, 108, 119, 122, 133, 136, 147, 150, 161, 164, 175,178, 189, 192,203,206,217,220,231,234,245, 148, 259, 262, 273, 276, 287, 290, 301, 304, 315, 318, 329, 332, 343, 346, 357, 360, 371, 374, 385, 388, 399, 402, 413, 416, 427, or 430.

34. The polypeptide or polypeptide construct of claim 20, wherein the polypeptide or polypeptide construct comprises a domain binding to CD3 comprising a VH domain comprising at least one, two, or all of the following CDR sequences as set forth in SEQ ID NO: 670, 671, and/or 672.

35. The polypeptide or polypeptide construct of claim 20, wherein the polypeptide or polypeptide construct comprises a domain binding to CD3 comprising a VL domain comprising at least one, two, or all of the following CDR sequences as set forth in SEQ ID NO: 673, 674, and/or 675.

36. The polypeptide or polypeptide construct of claim 20, wherein the polypeptide or polypeptide construct comprises a domain binding to CD3 comprising a VH domain and a VL domain comprising at least one, two, or all of the following CDR sequences as set forth in SEQ ID NO: 670, 671, 672, 673, 674, and/or 675.

37. The polypeptide or polypeptide construct of claim 20, wherein the polypeptide or polypeptide construct comprises a domain binding to CD3 comprising a VH domain as set forth in SEQ ID NO: 676.

38. The polypeptide or polypeptide construct of claim 20, wherein the polypeptide or polypeptide construct comprises a domain binding to CD3 comprising a VL domain as set forth in SEQ ID NO: 677.

39. The polypeptide or polypeptide construct of claim 20, wherein the polypeptide or polypeptide construct comprises a domain binding to CD3 comprising a VH domain as set forth in SEQ ID NO: 676 and a VL domain as depicted in SEQ ID NO: 677.

40. The polypeptide or polypeptide construct of claim 20, wherein the polypeptide or polypeptide construct comprises a domain binding to CD3 comprising a scFv domain as set forth in SEQ ID NO: 678.

41. A polynucleotide encoding the polypeptide or polypeptide construct of claim 1.

42. A vector comprising the polynucleotide of claim 41.

43. A host cell transformed or transfected with the polynucleotide of claim 41.

44. A process for producing a polypeptide or polypeptide construct comprising culturing the host cell of claim 43 under conditions allowing the expression of the polypeptide or polypeptide construct and recovering the produced polypeptide or polypeptide construct from the culture.

45. A pharmaceutical composition comprising the polypeptide or polypeptide construct of claim 1.

46-49. (canceled)

50. A method for treating or ameliorating a proliferative disease, a tumorous disease, cancer, or an immunological disorder, comprising the step of administering to a subject in need thereof the polypeptide or polypeptide construct of claim 1, wherein the disease is selected from the group consisting of germ cell cancer, ovarian cancer, uterine cancer, and lung cancer.
